(19) United States
(12) Patent Application Publication
(10) Pub. No.: US 2025/0215013 A1
(43) Pub. Date: Jul. 3, 2025

(54) SUBSTITUTED TRICYCLIC COMPOUNDS AS PARP INHIBITORS AND THE USE THEREOF

(71) Applicant: IMPACT THERAPEUTICS (SHANGHAI), INC., Shanghai (CN)

(72) Inventors: Sui Xiong Cai, Shanghai (CN); Xiaozhu Wang, Nanjing (CN); Ye Edward Tian, Tianjin (CN); Letian Zhang, Nanjing (CN)

(21) Appl. No.: 18/846,265

(22) PCT Filed: Feb. 23, 2023
(86) PCT No.: PCT/CN2023/077988

§ 371 Date: Sep. 12, 2024

(30) Foreign Application Priority Data
Mar. 11, 2022 (CN) 202210260944.7
Apr. 24, 2022 (CN) 202210448803.8
Sep. 30, 2022 (CN) 202211219599.9
Nov. 28, 2022 (CN) 202211505904.0

(51) Int. Cl. C07D 495/04; A61K 31/496; A61K 31/4985; A61K 31/519; A61K 31/5383; A61K 45/06; A61P 35/00; C07D 401/14; C07D 471/04; C07D 487/04; C07D 491/048; C07D 498/04; C07D 513/04; C07D 519/00

(52) U.S. Cl. CPC C07D 495/04; A61K 31/496; A61K 31/4985; A61K 31/519; A61K 31/5383; A61K 45/06; A61P 35/00; C07D 401/14; C07D 471/04; C07D 487/04; C07D 491/048; C07D 498/04; C07D 513/04; C07D 519/00

(57) ABSTRACT

Provided are substituted tricyclic compounds as PARP inhibitors and the use thereof. The compounds represented by Formula (I) as below, wherein, the ring Z, Z1, Z2, Z3, Z4, Z5, A1, A2, A3, L and Cy are defined herein. The compounds of Formula I are PARP inhibitors and thus are useful in the treatment of diseases, disorders and conditions, such as cancer, responsive to the inhibition of PARP activity.

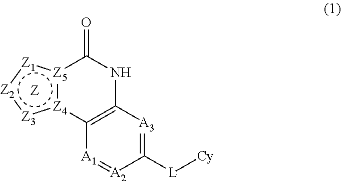

### FIELD OF THE DISCLOSURE

The disclosure relates to substituted tricyclic compounds as PARP inhibitors and the use thereof.

### BACKGROUND OF THE INVENTION

Poly(ADP-ribose) polymerase (PARP) is a family of proteins, which transfer negatively charged ADP-ribose groups from donor NAD⁺ onto target proteins. That is one of many post-transcriptional modifications. Therefore, PARP also is termed as ADP-ribose transferase.

Humans are thought to express 17 PARPs identified based on amino acid sequence homology to the catalytic domain (Vyas et al., 2013 Nature Communication, 4: 3240/1-3240/13). PARPs either catalyze the addition of a single ADP-ribose unit on target proteins or catalyze the polymerization of ADP-ribose units to form poly ADP-ribose, also known as poly(ADP-ribose) modification. As a result, the PARP family is further grouped into two subfamilies accordingly. Post-translational modification of poly(ADP-ribose) regulate many aspects of protein function and the physiological function of many PARPs have not been established.

The most characteristic member of the PARP family is PARP1, which was found to have the highest intracellular levels. PARP1 consists of 1014 amino acids (NCBI Accession P09874) with a total molecular weight of approximately 116 kDa. Structurally, this enzyme is composed of two main domains including an N-terminal DNA-binding domain and a catalytic domain. PARP1 is known to play an important role in many cellular functions, including gene expression, transcription, cell division, cell differentiation, cell apoptosis, DNA damage response and repair. PARP1 is activated when DNA damage occurs and is involved in base excision repair (BER) which is a major mechanism of DNA single-strand damage repair. PARP1 binds to the site of Single Strand Break (SSB), and subsequently repair DNA via BER. In response to DNA damage, cells also have evolved two main repair pathways: Homologous Recombination (HR) and Non-Homologous End Joining (NHEJ), in addition to BER repair mechanisms. HR deficient tumors have been found to be sensitive to PARP inhibitors, indicating homologous recombination defects and PARP1 inhibition formed a pair of synthetic lethality, which has been validated by clinical studies. Several PARP inhibitors are currently approved for the treatment of ovarian, breast, pancreatic and prostate cancers with DNA damage repair deficient such as BRCA1/2 mutation.

PARP2 is a protein of 559 amino acids with molecular mass of approximately 62 kDa and composed of DNA binding domain and catalytic domain (Ame et al., 1999 J Biol Chem 274: 17860-17868). The catalytic domain of PARP2 is very similar to that of PARP1. PARP2 is also found to have similar functions to PARP1 and is involved in the repair of DNA damage through BER mechanism (Schreiber et al., 2002 J Biol Chem 277: 23028-23036). Marketed PARP inhibitors, such as Olaparib, Niraparib, Talazoparib and Rucaparib, not only have inhibitory activities against PARP1, but also have similar inhibitory activities against PARP2. Based on the results of clinical trials, the therapeutic effects of these marketed PARP inhibitors are comparable whereas their toxicity profiles are quite different. For example, Talazoparib has toxicity similar to chemotherapy drugs such as hair loss. Talazoparib also shows more potent inhibitory activity against TNKS1/2 (Tankyrase 1 or Tankyrase 2) than other PARP inhibitors (PARPi) in biochemical assay (Ryan et al., 2021 J Biol Chem 296: 100251/1-100251/13). TNKS1 and TNKS2 share 83% sequence identity overall, and their catalytic domain sequences are 89% identical. They play roles in DNA repair, telomere maintenance, and Wnt/p-catenin signaling. Targeting PARPs other than PARP1 may be the reason why PARP inhibitors cause off-targeted toxicity, such as hair loss and diarrhea. In addition, inhibition of PARP2 activity has been found to lead to hematotoxicity (Farrés et al., 2013 Blood 122: 44-54; Farrés et al., 2015 Cell Death and Differentiation 22: 1144-1157). The toxicity of these PARP inhibitors limits their clinical application as well as in combination with other targeted drugs.

Therefore, to improve, enhance and expand the clinical application of PARP inhibitors, it is important to explore highly selective PAPR1 inhibitor to reduce mechanism-related or mechanism-independent toxicity.

Various PARP1 inhibitors have been disclosed in, for example, WO2011006803, WO2013014038, WO2021013735, WO2021260092, CN115232129A, WO2022225934, WO2022222921, WO2022222964, WO2022222965, WO2022222966, WO202228387, WO2022247816, WO2022223025 and WO2022222995.

### SUMMARY OF THE DISCLOSURE

The disclosure provides compounds and analogues thereof as represented by Formula I (including Formulae II, III, IV, V, VI, VII, VIII and IX). The compounds can be used as PARP inhibitors. In particular, the compounds of the disclosure are selective PARP1 inhibitors relative to PARP2.

The disclosure also provides pharmaceutical compositions comprising an effective amount of the compound of Formula I (including Formulae II, III, IV, V, VI, VII, VIII and IX). The pharmaceutical compositions can be used for the treatment of cancer.

In a specific embodiment, the pharmaceutical composition may further contain one or more pharmaceutically acceptable carriers, excipients or diluents. The pharmaceutical compositions can be used for the treatment of cancer.

In a specific embodiment, the pharmaceutical composition may further contain at least one known anticancer drug or pharmaceutically acceptable salts thereof.

The disclosure is also directed to methods for the preparation of novel compounds of Formula I (including Formulae II, III, IV, V, VI, VII, VIII and IX).

### DETAILED DESCRIPTION OF THE DISCLOSURE

It should be understood that the characteristics of the embodiments described herein can be arbitrarily combined to form the technical solution of this disclosure. The definition of each group herein can apply to any of the embodiments described herein. For example, the definitions of the substituents of alkyl herein apply to any of the embodiments described herein unless the substituents of alkyl are clearly defined in the embodiment.

The term “hydrogen (H)” as employed herein includes its isotopes D and T.

The term “heteroatoms” as employed herein includes O, S and N.

The term “alkyl” as used herein refers to alkyl itself or a straight or branched chain radical of up to ten carbons. Useful alkyl groups include straight-chain or branched C₁₋₁₀ alkyl groups, preferably C₁₋₆ alkyl groups. In some embodiments, alkyl is C₁₋₄ alkyl. In some embodiments, alkyl is C₁₋₃ alkyl. In some embodiments, alkyl is deuterated C₁₋₃ alkyl. Typical C₁₋₁₀ alkyl groups include methyl, methyl-d3, ethyl, propyl, isopropyl, butyl, sec-butyl, tert-butyl, pentyl (such as 3-pentyl), hexyl and octyl groups, which may be optionally substituted.

The term “alkenyl” as used herein refers to a straight or branched chain radical of 2-10 carbon atoms, unless the chain length is limited thereto, there is at least one double bond between two of the carbon atoms in the chain; preferably, the alkenyl is a C₂₋₆ alkenyl, more preferably a C₂₋₄ alkenyl. Typical alkenyl groups include ethenyl, 1-propenyl, 2-propenyl, 2-methyl-1-propenyl, 1-butenyl and 2-butenyl.

The term “alkynyl” as used herein refers to a straight or branched chain radical of 2-10 carbon atoms, unless the chain length is limited thereto, there is at least one triple bond between two of the carbon atoms in the chain; preferably, the alkynyl is a C₂₋₆ alkynyl, more preferably a C₂₋₄ alkynyl. Typical alkynyl groups include ethynyl, 1-propynyl, 1-methyl-2-propynyl, 2-propynyl, 1-butynyl and 2-butynyl.

Useful alkoxy groups include oxygen substituted by the above mentioned C₁₋₁₀ alkyl groups, preferred C₁₋₆ alkyl groups or C₁₋₄ alkyl groups or C₁₋₃ alkyl groups, e.g., methoxy, ethoxy, etc. The alkyl in the alkoxy groups may be optionally substituted. Substituents of alkoxy groups include, without limitation, halogen, morpholino, amino (including alkylamino and dialkylamino), and carboxy (including esters thereof).

Useful amino and optionally substituted amino groups are —NR′R″, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl, an optionally substituted 3-6 membered heterocyclic group or an optionally substituted 5 membered heteroaryl, or R′ and R″ together with the N to which they are attached form an optionally substituted 4-7 membered cyclic amino group, which optionally comprises one or more (such as 2, 3) additional heteroatoms selected from a group consisting of O, N and S. Preferably, R′ and R″ are each independently selected from a group consisting of hydrogen, an optionally substituted C₁₋₄ alkyl, an optionally substituted C₃₋₆ cycloalkyl or an optionally substituted 3-6 membered heterocyclic group.

The term “oxo” as used herein refers to ═O.

The term “aryl” as used herein by itself or as part of another group refers to monocyclic, bicyclic or tricyclic aromatic groups containing 6 to 14 carbon atoms. Aryl may be substituted by one or more substituents as described herein.

Useful aryl groups include C₆₋₁₄ aryl groups, preferably C₆₋₁₀ aryl groups. Typical C₆₋₁₄ aryl groups include phenyl, naphthyl, phenanthryl, anthracyl, indenyl, azulyl, biphenyl, biphenylene and fluorenyl.

The term “carbocyclic group” as used herein include cycloalkyl and partially saturated carbocyclic groups. Useful cycloalkyl groups are C₃₋₈ cycloalkyl. In some preferred embodiments, cycloalkyl groups are C₃₋₆ cycloalkyl. Typical cycloalkyl groups include cyclopropyl, cyclobutyl, cyclopentyl, cyclohexyl and cycloheptyl. Useful partially saturated carbocyclic groups are cycloalkenyl, such as C₃₋₈ cycloalkenyl, which include cyclopentenyl, cycloheptenyl and cyclooctenyl. Carbocyclic group may be substituted by one or more substituents as described herein.

Useful halo or halogen groups include fluoro, chloro, bromo and iodo.

Useful acylamino (amido) groups are any C₁₋₆ acyl (alkanoyl) attached to an amino nitrogen, e.g., acetamino, propionamido, butanoylamido, pentanoylamido and hexanoylamido, as well as aryl-substituted C₁₋₆ acylamino groups, e.g., benzoylamido.

Useful acyl groups include C₁₋₆ acyl groups, such as acetyl. Acyl may be optionally substituted by group selected from halo, amino and aryl, wherein the amino and aryl may be optionally substituted. When acyl is substituted by halo, the number of halogen substituents may be in the range of 1-5. Examples of substituted acyls include chloroacetyl and pentafluorobenzoyl. When acyl is substituted by amino, amino group may be substituted by one or two substituents as described herein. In some embodiments, aminoacyl is —C(O)—NR′R″, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl or an optionally substituted 3-6 membered heterocyclic group.

The term “heterocyclic group” as used herein refers to a saturated or partially saturated 3-7 membered monocyclic, or 7-10 membered bicyclic ring, spirocyclic ring or bridged ring system, which consists of carbon atoms and one to four heteroatoms independently selected from a group consisting of O, N, and S, wherein the nitrogen and/or sulfur heteroatoms can be optionally oxidized and the nitrogen can be optionally quaternized, and the term also includes any bicyclic ring system in which any of the above-defined heterocyclic rings is fused to a benzene ring. The heterocyclic group can be substituted on carbon atom or nitrogen atom if the resulting compound is stable. The heterocyclic group may be substituted by one or more substituents as described herein.

Useful saturated or partially saturated heterocyclic groups include tetrahydrofuranyl, tetrahydropyranyl, piperidinyl, piperazinyl, 1,4-diazepanyl, azetidinyl, oxetanyl, pyrrolidinyl, imidazolidinyl, imidazolinyl, indoline, isoindoline, quinuclidinyl, morpholinyl, isochromanyl, chromanyl, pyrazolidine, pyrazolinyl, tetrahydroisoquinolyl, tetronoyl and tetramoyl, which may be optionally substituted by one or more substituents as described herein.

The term “heteroaryl” as used herein refers to a group having 5 to 14 ring atoms, preferably 5 to 10 ring atoms, with 6, 10 or 14 electrons shared in a cyclic array. Ring atoms are carbon atoms and 1-3 heteroatoms selected from a group consisting of oxygen, nitrogen and sulfur. Heteroaryl may be optionally substituted by one or more substituents as described herein.

Useful heteroaryl groups include thienyl (thiophenyl), benzo[d]isothiazol-3-yl, benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, imidazolyl, pyrazolyl, pyridyl (pyridinyl, including without limitation 2-pyridyl, 3-pyridyl, and 4-pyridyl), pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, P-carbolinyl, phenanthridinyl, acridinyl, perimidinyl, phenanthrolinyl, phenazinyl, isothiazolyl, phenothiazinyl, isoxazolyl, furazanyl, phenoxazinyl, tetrahydrocyclopenta[c]pyrazol-3-yl, benzoisoxazolyl such as 1,2-benzoisoxazol-3-yl, benzimidazolyl, 2-oxindolyl, thiadiazolyl, 2-oxobenzimidazolyl, imidazopyridazinyl, imidazopyridyl, triazolopyridazinyl, pyrazolopyrimidinyl, pyrrolopyrimidinyl, pyrrolopyridyl, pyrrolopyrazinyl or triazolopyrazinyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

In this disclosure, unless otherwise described, when substituted, the alkyl, cycloalkyl, heterocycloalkyl, alkoxy, heterocycloalkoxy, alkenyl, heterocycloalkenyl, alkynyl, amino, amido, acyloxy, carboxyl, hydroxyl, mercapto, alkylthio sulfonyl, sulfonyl, sulfinyl, aminoacyl, silyl, phosphinecarboxy, phosphono, carbocyclic group, heterocyclic group, aryl or heteroaryl as described in any embodiment herein may be substituted by one or more (such as 1, 2, 3, 4, 5 or 6) substituents selected from the group consisting of halogen, hydroxyl, carboxyl, amino, nitro, cyano, C₁₋₆ amido, C₁₋₆ acyloxy, C₁₋₆ alkoxy, aryloxy, alkylthio, C₁₋₆ alkyl, C₁₋₆ acyl, C₆₋₁₀ aryl, C₃₋₈ cycloalkyl, C₂₋₆ alkenyl, C₂₋₆ alkynyl, heterocyclic or heteroaryl, methylenedioxy, urea group, mercapto group, azide group, carbonyl, alkanesulfonyl, sulfamoyl, dialkylsulfamoyl and alkylsulfinyl, etc. The substituent itself may also be optionally substituted. Preferred substituents include without limitation halogen, hydroxyl, carboxyl, amino, C₁₋₆ amido, C₁₋₆ acyloxy, C₁₋₆ alkoxy, C₁₋₆ alkyl, C₁₋₆ acyl and alkanesulfonyl.

It should be understood that in each embodiment, when the substituent is a heterocyclic group, aryl or heteroaryl, the number thereof is usually 1.

Specifically, the disclosure provides compounds represented by Formula I:

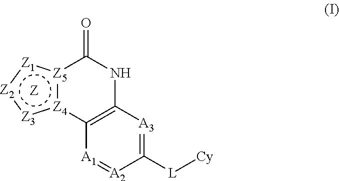

or stereoisomers, tautomers, N-oxides, hydrates, solvates, isotope-substituted derivatives, or pharmaceutically acceptable salts thereof, or mixtures thereof, or prodrugs thereof, wherein:
A₁, A₂ and A₃ are each independently selected from N and CR₁;
the Z ring shown as

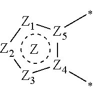

is an optionally substituted 5 membered heteroaryl group, an optionally substituted 5 membered carbocyclic group or an optionally substituted 5 membered heterocyclic group, * indicates the position at which the Z ring is attached to the rest of the compound, the dashed lines indicate optional presence of unsaturated bond(s), wherein, when the Z ring is an optionally substituted 5 membered heteroaryl group, Z₁, Z₂ and Z₃ are each independently CR₂, NR₃, O, N or S, Z₄ and Z₅ are each independently C or N, wherein Z₄ and Z₅ are not N at the same time; when the Z ring is an optionally substituted 5 membered carbocyclic group or an optionally substituted 5 membered heterocyclic group, Z₁, Z₂ and Z₃ are each independently CR₂R₂′, CR₂, NR₃, O or S, Z₄ and Z₅ are each independently C, CH or N; and
when Z₅ is N, at least one of Z₁, Z₂ and Z₃ is N, or when Z₅ is N and all of Z₁, Z₂ and Z₃ are CR₂ or CR₂R₂′, A₁ is CR₁;
L is selected from a bond and an alkylene optionally substituted by R₄ and/or R₅;
Cy is selected from a group consisting of an optionally substituted heterocyclic group, an optionally substituted aryl, and an optionally substituted heteroaryl;
R₁ is selected from a group consisting of hydrogen, halogen, an optionally substituted alkyl, an optionally substituted alkoxy and an optionally substituted carbocyclic group;
R₂ and R₂′ are each independently selected from a group consisting of hydrogen, hydroxyl, halogen, cyano, an optionally substituted alkyl, an optionally substituted alkoxy, an optionally substituted cycloalkyl, an optionally substituted alkenyl, and an optionally substituted alkynyl;
R₃ is selected from a group consisting of hydrogen, an optionally substituted alkyl and an optionally substituted cycloalkyl;
R₄ and R₅ are each independently selected from a group consisting of halogen, cyano, an optionally substituted alkyl, an optionally substituted alkoxy, an optionally substituted cycloalkyl, an optionally substituted alkenyl, and an optionally substituted alkynyl; or R₄ and R₅ together with the attached C form a ring.

In Formula I and each formula of the disclosure, unless otherwise described, each alkyl is independently a C₁₋₆ alkyl, preferably a C₁₋₄ alkyl; each alkylene is a C₁₋₆ alkylene, preferably a C₁₋₃ alkylene; each alkenyl is independently a C₂₋₆ alkenyl, preferably C₂₋₄ alkenyl; each alkynyl is independently C₂₋₆ alkynyl, preferably C₂₋₄ alkynyl; each alkoxy is independently C₁₋₆ alkoxy, preferably C₁₋₄ alkoxy. Preferably, when the alkyl, alkenyl, alkynyl and alkoxy are substituted, the substituents can be selected from a group consisting of cyano, hydroxyl, nitro, amino (—NR′R″), aryl, heterocyclic group, heteroaryl, halogen and carboxyl, etc. The number of substituents may be 1-5, R′ and R″ are preferably each independently H, an optionally substituted C₁₋₄ alkyl, an optionally substituted C₃₋₆ cycloalkyl or an optionally substituted 3-6 membered heterocyclic group. For example, the substituted alkyl per se or as a substituent of other groups may be hydroxyalkyl, dihydroxyalkyl, alkylaminoalkyl, dialkylaminoalkyl, heterocyclic alkyl, arylalkyl, heteroarylalkyl and haloalkyl, etc. It should be understood, when the substituent is aryl, heteroaryl, heterocyclic group, cyano, nitro and carboxyl, the number thereof is usually 1. When the substituent is halogen, the number of substituents can be up to 5 depending on the carbon chain length of the alkyl, alkenyl, alkynyl and alkoxy groups; exemplary substituents are trifluoromethyl and pentafluoroethyl, etc.

In Formula I and each formula of the disclosure, unless otherwise described, the number of ring carbon atoms of each carbocyclic group is preferably 3-8. Preferred carbocyclic groups are C₃₋₈ cycloalkyl groups or C₃₋₈ cycloalkenyl. The substituents on the carbocyclic group are preferably C₁₋₄ alkyl, halogenated C₁₋₄ alkyl, C₁₋₄ alkoxy, halogen, hydroxyl, carboxyl, amino (—NR′R″), aryl, heterocyclic group, heteroaryl and carboxyl, etc. The number of substituents may be 1-5, R′ and R″ are preferably each independently H, an optionally substituted C₁₋₄ alkyl, an optionally substituted C₃₋₆ cycloalkyl or an optionally substituted 3-6 membered heterocyclic group. It should be understood, when the substituent is aryl, heteroaryl, heterocyclic group, cyano, nitro and carboxyl, the number thereof is usually 1. When the substituent is halogen, the number of substituents can be up to 5.

In Formula I and each formula of the disclosure, unless otherwise described, the aryl refers to C₆₋₁₄ aryl, the heteroaryl refers to 5-10 membered heteroaryl, and the heterocyclic group refers to 4-10 membered heterocyclic group. The substituents on each of the aryl, heteroaryl and heterocyclic group can each be independently selected from 1-5 groups consisting of C₁₋₄ alkyl, halogenated C₁₋₄ alkyl, C₁₋₄ alkoxy, halogen, hydroxyl, carboxyl, amino (—NR′R″), an optionally substituted aryl, an optionally substituted heteroaryl, an optionally substituted heterocyclic group, halogen, amido, aminoacyl (—C(O)—NR′R″) and carboxyl, etc.; wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl or an optionally substituted 3-6 membered heterocyclic group. The said optionally substituted aryl, optionally substituted heteroaryl and optionally substituted heterocyclic group may be optionally substituted by 1-5 groups selected from C₁₋₄ alkyl, halogenated C₁₋₄ alkyl, C₁₋₄ alkoxy, halogen, hydroxyl, carboxyl, amino (—NR′R″), aminoacyl (—C(O)—NR′R″) and carboxyl, wherein, the said R′ and R″ are preferably each independently H, optionally substituted C₁₋₄ alkyl, optionally substituted C₃₋₆ cycloalkyl or an optionally substituted 3-6 membered heterocyclic group. It should be understood, when the substituent is aryl, heteroaryl, heterocyclic group, cyano, nitro and carboxyl, the number thereof is usually 1. When the substituent is halogen, the number of substituents can be up to 5.

In some embodiments, the compounds of the present disclosure do not include compounds represented by the following structural formula, or stereoisomers, solvates or pharmaceutically acceptable salts thereof:

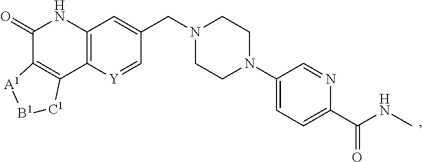

wherein Y is N, CH or CF; A¹ is CH₂, CF₂, CHF, CHCH₃ or C(CH₃)₂; B¹ is CH₂, CF₂, CHF, CHCH₃ or C(CH₃)₂; C is O or S; and

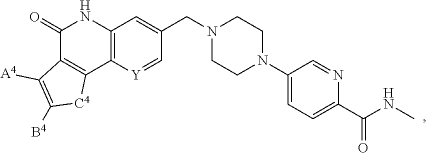

wherein Y is N, CH or CF; A⁴ is H, C₁₋₆ alkyl or halogenated C₁₋₆ alkyl; B⁴ is H, C₁₋₆ alkyl or halogenated C₁₋₆ alkyl; C⁴ is O or S; and

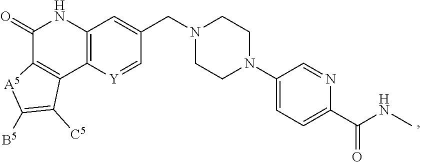

wherein Y is N, CH or CF; A⁵ is O or S; B⁵ is H, C₁₋₆ alkyl or halogenated C₁₋₆ alkyl; C⁵ is H, C₁₋₆ alkyl or halogenated C₁₋₆ alkyl; and

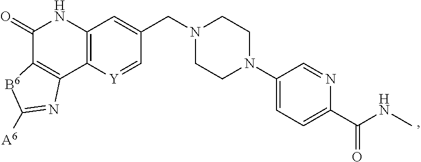

wherein Y is N, CH or CF; A⁶ is H, C₁₋₆ alkyl or halogenated C₁₋₆ alkyl; B⁶ is O or S; and

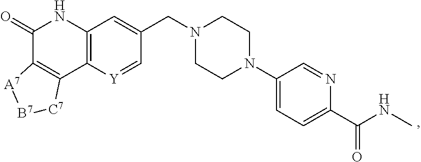

wherein Y is N, CH or CF; A⁷ is O or S; B⁷ is CH₂, CF₂, CHF, CHCH₃ or C(CH₃)₂; C⁷ is CH₂, CF₂, CHF, CHCH₃ or C(CH₃)₂; and

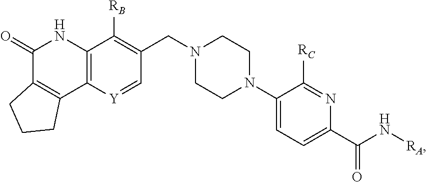

wherein, R_{A} is C₁₋₃ alkyl, deuterated C₁₋₃ alkyl, halogenated C₁₋₃ alkyl or C₁₋₃ alkoxy; R_{B} and R_{C} each are independently H, halogen, C₁₋₃ alkyl, deuterated C₁₋₃ alkyl, halogenated C₁₋₃ alkyl or C₁₋₃ alkoxy; Y is CH or N.

In some embodiments, the compounds of the present disclosure do not include the following compounds:

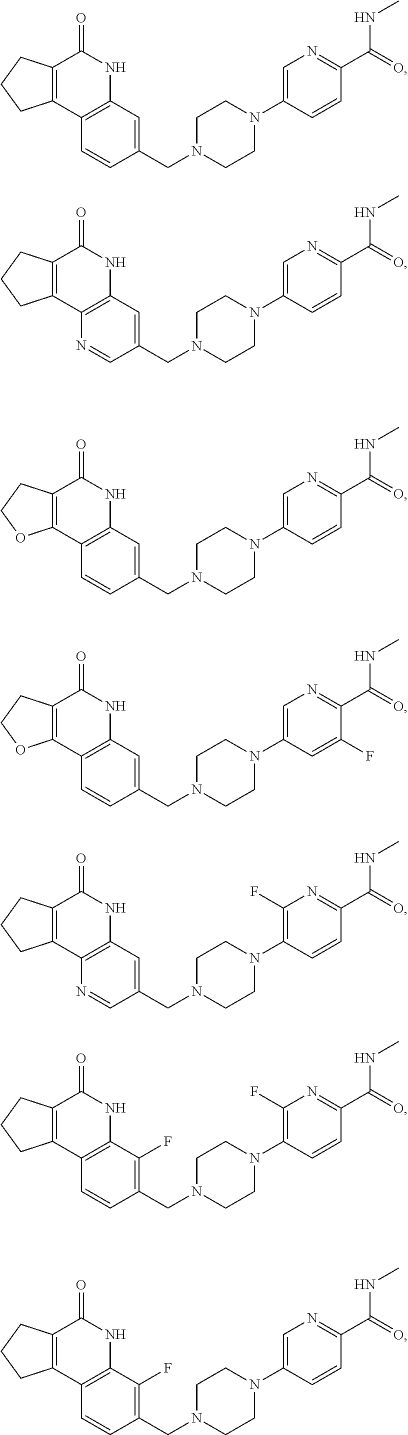

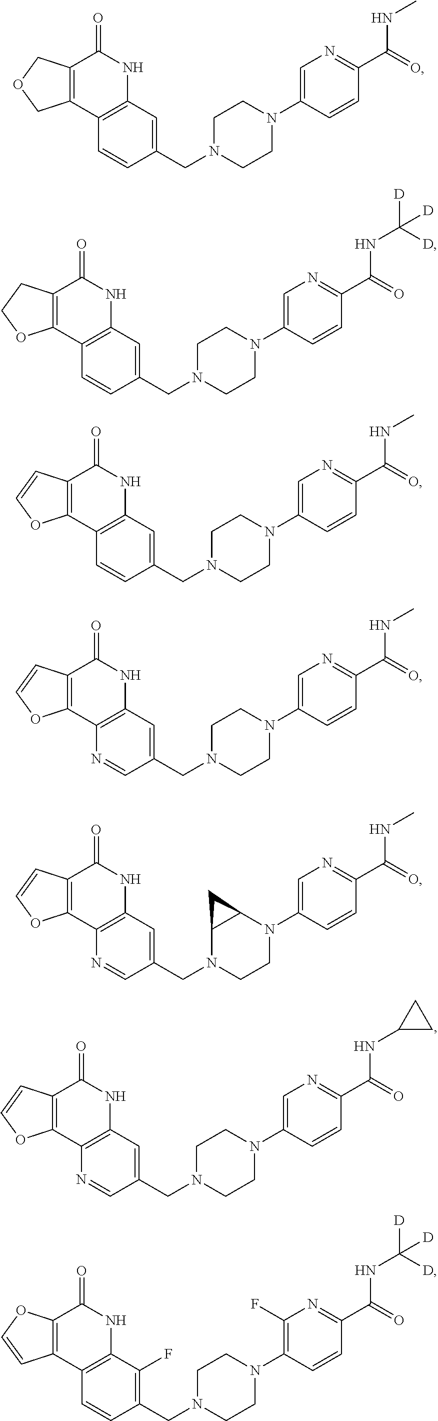

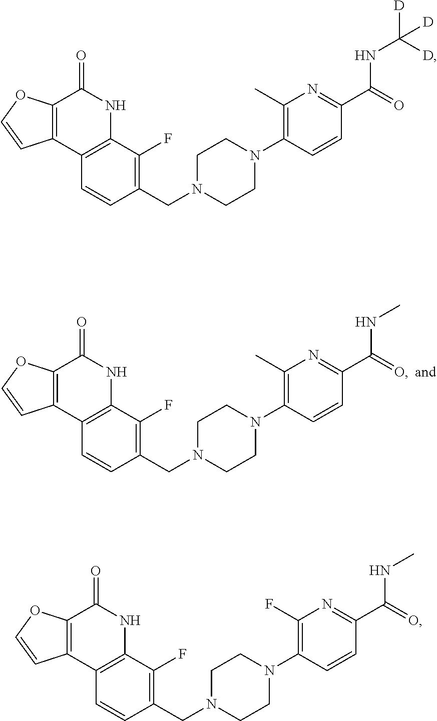

In some embodiments, the compounds of the present disclosure do not include the following compounds:

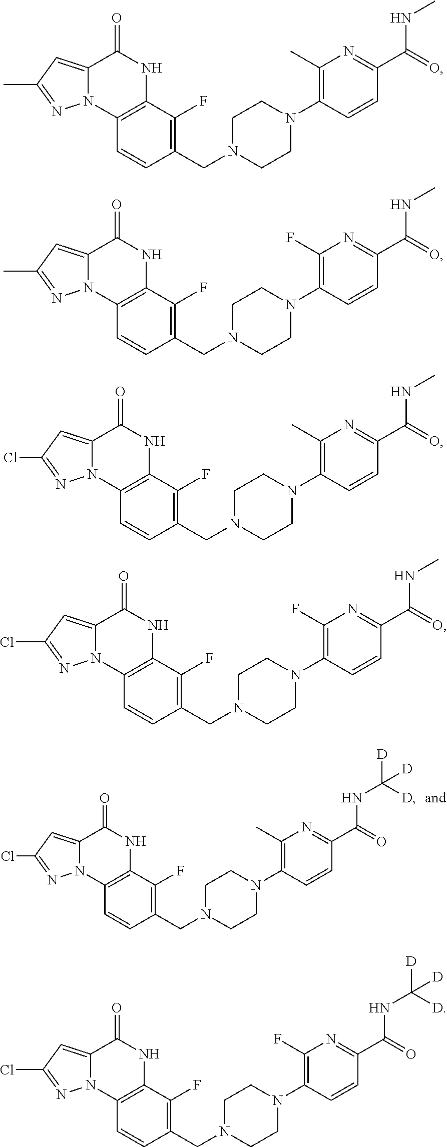

In one or more embodiments of the compound of Formula I, preferred compounds are presented by Formula Ia, Ib, Ic or Id, or stereoisomers, tautomers, N-oxides, hydrates, solvates, isotope-substituted derivatives, or pharmaceutically acceptable salts thereof, or mixtures thereof, or prodrugs thereof,

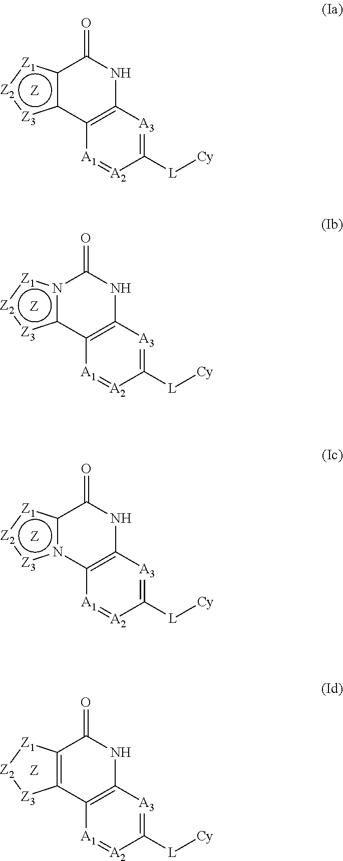

wherein, A₁, A₂, A₃, Z ring (including Z₁, Z₂ and Z₃), L and Cy are as defined in Formula I.

In one or more embodiments of the compound of Formula Ia:
A₁, A₂ and A₃ are each independently selected from N and CR₁;
the Z ring shown as

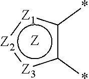

is an optionally substituted 5 membered heteroaryl group, * indicates the position at which the Z ring is attached to the rest of the compound, wherein Z₁, Z₂ and Z₃ are each independently CR₂, NR₃, O, N or S;
L is selected from a bond and an alkylene optionally substituted by R₄ and/or R₅;
Cy is selected from a group consisting of an optionally substituted heterocyclic group, an optionally substituted aryl, and an optionally substituted heteroaryl;
R₁ is selected from a group consisting of hydrogen, halogen, an optionally substituted alkyl, an optionally substituted alkoxy and an optionally substituted carbocyclic group;
R₂ is selected from a group consisting of hydrogen, hydroxyl, halogen, cyano, an optionally substituted alkyl, an optionally substituted alkoxy, an optionally substituted cycloalkyl, an optionally substituted alkenyl, and an optionally substituted alkynyl;
R₃ is selected from a group consisting of hydrogen, an optionally substituted alkyl and an optionally substituted cycloalkyl;
R₄ and R₅ are each independently selected from a group consisting of halogen, cyano, an optionally substituted alkyl, an optionally substituted alkoxy, an optionally substituted cycloalkyl, an optionally substituted alkenyl, and an optionally substituted alkynyl; or R₄ and R₅ together with the attached C form a ring.

In one or more embodiments of the compound of Formula I (including Formulae Ia, Ib, Ic and Id), the Z ring is selected from the following groups:

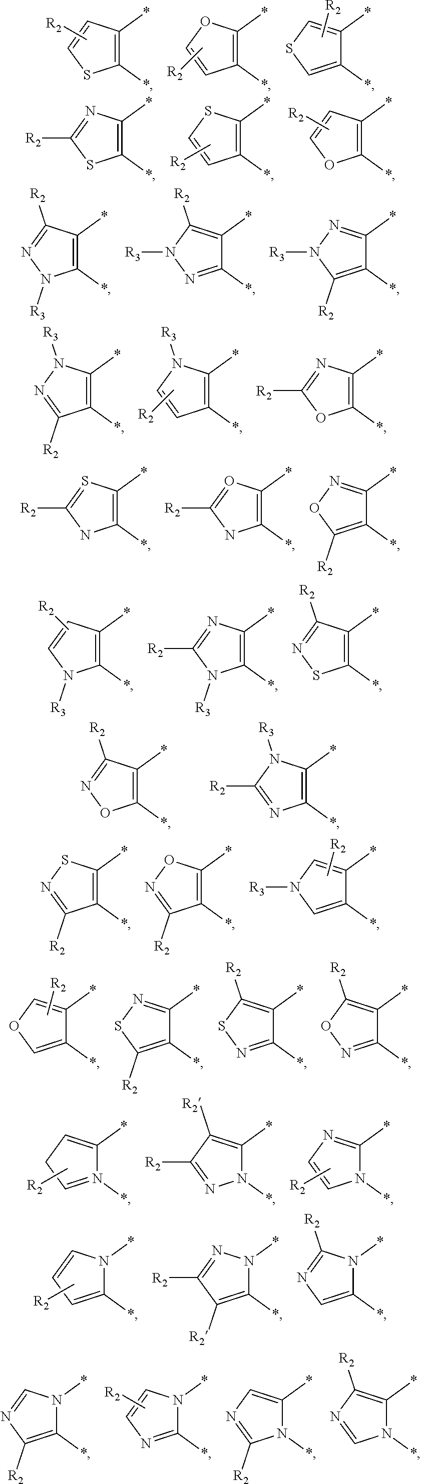

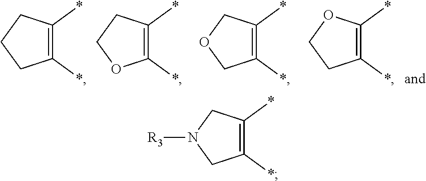

wherein, * indicates the position at which the Z ring is attached to the rest of the compound; R₂ is selected from hydrogen, halogen, cyano, an optionally substituted C₁₋₆ alkyl, an optionally substituted C₁₋₃ alkoxy and an optionally substituted C₃₋₆ cycloalkyl, preferably hydrogen, halogen and an optionally substituted C₁₋₃ alkyl; R₂′ is selected from hydrogen, halogen, cyano, an optionally substituted C₁₋₆ alkyl, an optionally substituted C₁₋₃ alkoxy and an optionally substituted C₃₋₆ cycloalkyl, preferably hydrogen, halogen and an optionally substituted C₁₋₃ alkyl; R₃ is selected from a group consisting of hydrogen, an optionally substituted alkyl and an optionally substituted cycloalkyl, preferably hydrogen and an optionally substituted C₁₋₃ alkyl.

In one or more embodiments of the compound of Formula Ia, the Z ring is selected from the following groups:

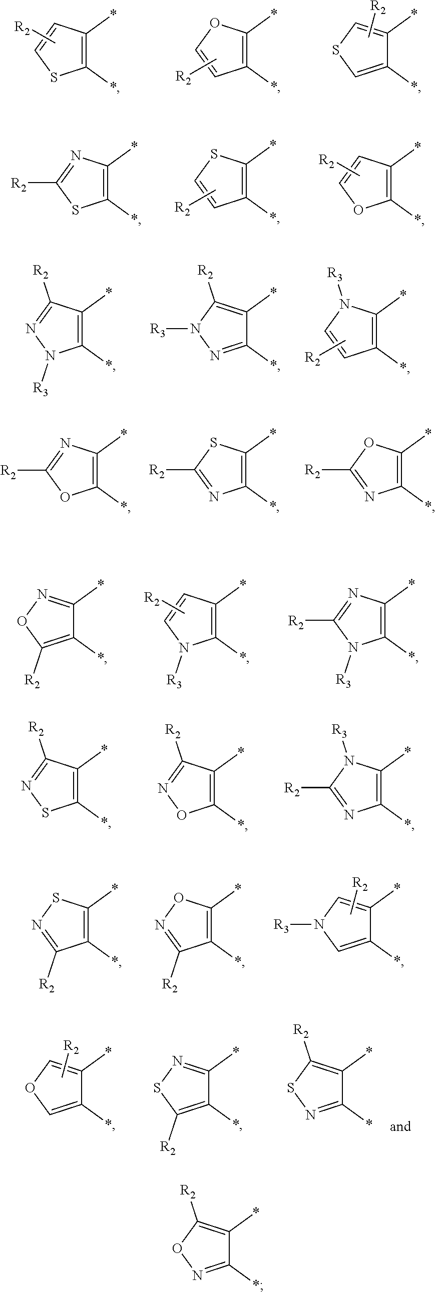

wherein, * indicates the position at which the Z ring is attached to the rest of the compound; R₂ is selected from hydrogen, halogen, an optionally substituted C₁₋₆ alkyl and an optionally substituted C₁₋₃ alkoxy, preferably hydrogen, halogen and an optionally substituted C₁₋₃ alkyl; R₃ is selected from a group consisting of hydrogen, an optionally substituted alkyl and an optionally substituted cycloalkyl, preferably hydrogen and an optionally substituted C₁₋₃ alkyl.

In one or more embodiments of the compound of Formulae Ib and Ic, the Z ring is selected from the following groups:

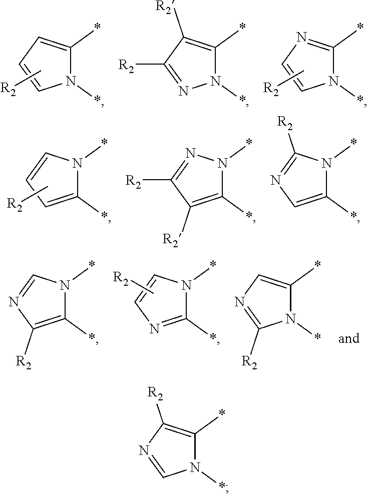

wherein R₂ and R₂′ are as defined in any of the foregoing embodiments; preferably, in these embodiments, at least one of A₂ and A₃ is CR₁, and R₁ is halogen; in some embodiments, both A₂ and A₃ are CR₁, and at least one of the R₁ groups is halogen. In some embodiments, A₃ is CR₁, and R₁ is halogen. In some embodiments, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen.

In this disclosure, the substituents on the Z ring can be 1-2 groups selected from a group consisting of hydroxy, halogen, C₁₋₄ alkyl, C₁₋₄ alkoxy, C₃₋₆ cycloalkyl, halogenated C₁₋₄ alkyl, halogenated C₁₋₄ alkoxy, C₁₋₄ alkyl substituted with hydroxy, C₁₋₄ alkoxy substituted with hydroxy and amino (—NR′R″), wherein R′ and R″ each are preferably independently H or C₁₋₄ alkyl. In some embodiments, the substituents on the Z ring can be 1-2 groups selected from halogen, C₁₋₄ alkyl or C₃₋₆ cycloalkyl.

In one or more embodiments of the compound of Formula I (including Formulae Ia, Ib, Ic and Id), A₁, A₂ and A₃ are each independently selected from N and CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. In some embodiments, at least one of A₁ and A₃ is CR₁, and R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In some embodiments, A₃ is CR₁, and R₁ is halogen. In one or more embodiments, all of A₁, A₂ and A₃ are CR₁, each R₁ is independently hydrogen, halogen or C₁₋₃ alkyl. In some embodiments, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula I (including Formulae Ia, Ib, Ic and Id), when the Z ring is not

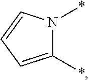

A₁, A₂ and A₃ are each independently selected from N and CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. Preferably, at least one of A₁ and A₃ is CR₁, and R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In some embodiments, A₃ is CR₁, and R₁ is halogen. In one or more embodiments, all of A₁, A₂ and A₃ are CR₁, each R₁ is independently hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula Ib, when the Z ring is

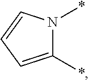

A₁ is CR₁, A₂ and A₃ each are independently N or CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. Preferably, A₃ is CR₁, wherein R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In one or more embodiments, all of A₁, A₂ and A₃ are CR₁, wherein R₁ is hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula I (including Formulae Ia, Ib, Ic and Id), R₁ is hydrogen, halogen, an optionally substituted C₁₋₃ alkyl or an optionally substituted C₁₋₃ alkoxy. Preferably, when R₁ is substituted, the substituents can be 1-5 groups selected from halogen, hydroxy, amino (—NR′R″), etc., wherein R′ and R″ each are preferably independently H, an optionally substituted C₁₋₄ alkyl or an optionally substituted C₃₋₆ cycloalkyl. Preferably, R₁ is hydrogen, halogen or C₁₋₃ alkyl.

In one or more embodiments of the compound of Formula I (including Formulae Ia, Ib and Ic), R₂ is hydrogen, halogen, cyano, C₁₋₃ alkyl or C₃₋₆ cycloalkyl.

In one or more embodiments of the compound of Formula I (including Formulae Ia, Ib, Ic and Id), R₃ is hydrogen or C₁₋₃ alkyl.

In one or more embodiments of the compound of Formula I (including Formulae Ia, Ib, Ic and Id), R₂′ is hydrogen, halogen, cyano, C₁₋₃ alkyl or C₃₋₆ cycloalkyl.

In one or more embodiments of the compound of Formula Id, both of R₂ and R₂′ are hydrogen.

In one or more embodiments of the compound of Formula I (including Formulae Ia, Ib, Ic and Id), R₄ and R₅ are each independently selected from halogen and C₁₋₃ alkyl. In some embodiments, R₄ and R₅ together with the attached C form a 3-6 membered ring.

In one or more embodiments of the compound of Formula I (including Formulae Ia, Ib, Ic and Id), L is an alkylene optionally substituted by 1-2 C₁₋₃ alkyl, more preferably C₁₋₃ alkylene optionally substituted by 1-2 C₁₋₃ alkyl, preferably methylene optionally substituted by 1-2 C₁₋₃ alkyl.

In one or more embodiments of the compound of Formula I (including Formulae Ia, Ib, Ic and Id), L is an unsubstituted alkylene, preferably an unsubstituted C₁₋₃ alkylene, more preferably methylene.

In one or more embodiments of the compound of Formula I (including Formulae Ia, Ib, Ic and Id), the said aryl is preferably a phenyl. The said heteroaryl is a 5-10 membered heteroaryl containing 1 or 2 nitrogen atoms, including but is not limited to pyridyl, pyrazinyl, pyrrolyl, imidazolyl, pyrazolyl, pyrimidinyl, pyridazinyl and indolyl, etc. The said carbocyclic group is preferably a C₃₋₈ cycloalkyl or a C₃₋₈ cycloalkenyl. The said heterocyclic group is preferably a 4-10 membered heterocyclic group containing O, S and/or N, including but not limited to azetidinyl, oxetanyl, pyrrolidinyl, piperazinyl, piperidinyl, dihydropyridinyl, dihydrofuranyl, tetrahydrofuranyl, tetrahydroisoquinolyl and morpholinyl, etc.

In one or more embodiments of the compound of Formula I (including Formulae Ia, Ib, Ic and Id), Cy is an optionally substituted 5-7 membered nitrogen-containing heterocyclic group. Preferably, the 5-7 membered nitrogen-containing heterocyclic group is covalently attached to L through its ring nitrogen atom. Further preferably, Cy is an optionally substituted piperazinyl, piperidinyl, dihyropyridinyl, or pyrrolidinyl. Preferably, the substituents on Cy of the compound of Formula I (including Formulae Ia, Ib, Ic and Id) are selected from a group consisting of halogen, optionally substituted C₁₋₄ alkyl, optionally substituted C₁₋₄ alkoxy, halogenated C₁₋₄ alkyl, halogenated C₁₋₄ alkoxy, cyano, hydroxyl, amino (—NR′R″), an optionally substituted 6-14 membered aryl, an optionally substituted 5-10 membered heteroaryl, an optionally substituted 4-10 membered heterocyclic group and an optionally substituted C₃₋₈ cycloalkyl; wherein, the optionally substituted 6-14 membered aryl, the optionally substituted 5-10 membered heteroaryl, the optionally substituted 4-10 membered heterocyclic group and the optionally substituted C₃₋₈ cycloalkyl each can be independently substituted by 1-5 substituents selected from a group consisting of halogen, cyano, C₁₋₄ alkyl, C₁₋₄ alkoxy, halogenated C₁₋₄ alkyl, halogenated C₁₋₄ alkoxy, C₃₋₆ cycloalkyl, amino (—NR′R″), aminoacyl (—C(O)—NR′R″) and carboxyl; wherein the said R′ and R″ each are preferably independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably H, an optionally substituted C₁₋₄ alkyl, an optionally substituted C₃₋₈ cycloalkyl or an optionally substituted 3-6 membered heterocyclic group. In some preferred embodiments, the said substituents on 6-14 membered aryl, 5-10 membered heteroaryl, 4-10 membered heterocyclic group and C₃₋₈ cycloalkyl include at least an aminoacyl (—C(O)—NR′R″), and optionally include one or two of substituents selected from a group consisting of halogen, cyano, C₁₋₄ alkyl, halogenated C₁₋₄ alkyl and C₃₋₆ cycloalkyl. In some preferred embodiments, Cy is substituted by an optionally substituted 5-10 membered heteroaryl, preferably an optionally substituted 5-10 membered nitrogen-containing heteroaryl (such as pyridyl, pyrazinyl, pyrrolyl, imidazolyl, pyrazolyl, pyrimidinyl, etc.). Preferably, the 5-10 membered heteroaryl or 5-10 membered nitrogen-containing heteroaryl is at least substituted by an aminoacyl (—C(O)—NR′R″), and optionally further substituted by one or two substituents selected from a group consisting of halogen, cyano, C₁₋₄ alkyl, halogenated C₁₋₄ alkyl and C₃₋₆ cycloalkyl. Further preferably, the said aminoacyl (—C(O)—NR′R″) is at the para position. In some particularly preferred embodiments, Cy is piperazinyl substituted with an optionally substituted pyridyl, piperidinyl substituted with an optionally substituted pyridyl, or dihydropyridinyl substituted with an optionally substituted pyridyl, and the said pyridyl is at least substituted with an aminoacyl (—C(O)—NR′R″). Preferably, in the embodiments as described herein, when the said R′ and R″ are substituted, the substituents can be 1-5 groups selected from a group consisting of halogen, hydroxyl, C₁₋₄ alkyl, C₁₋₄ alkoxy, halogenated C₁₋₄ alkyl, halogenated C₁₋₄ alkoxy and amino, etc. In some embodiments, Cy is substituted by the said amino (—NR′R″), one of substituents on the amino is an optionally substituted 5-10 membered nitrogen-containing heteroaryl. Preferably, Cy is pyrrolidinyl substituted with an optionally substituted pyridylamino.

In one or more embodiments of the compound of Formula I or Ia, the Z ring is:

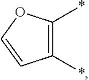

wherein, * indicates the position at which the Z ring is attached to the rest of the compound; A₁ is CH, A₂ is CR₁ and A₃ is CH, or both of A₁ and A₂ are CH, A₃ is CR₁, wherein R₁ is halogen, an optionally substituted C₁₋₃ alkyl or an optionally substituted C₁₋₃ alkoxy, preferably halogen, more preferably F; L and Cy are as defined in any of the foregoing embodiments.

In one or more embodiments of the compound of Formula I or Ic, the Z ring is:

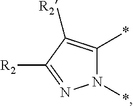

wherein, * indicates the position at which the Z ring is attached to the rest of the compound; R₂ is selected from hydrogen, halogen, cyano, optionally substituted alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; R₂′ is selected from hydrogen, halogen, cyano, optionally substituted C₁₋₆ alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; moreover, at least one of R₂ and R₂′ are non-hydrogen substituent, preferably, the non-hydrogen substituent is halogen, cyano, C₁₋₃ alkyl or C₃₋₆ cycloalkyl; A₁ is CH, A₂ is CR₁ and A₃ is CH, or both of A₁ and A₂ are CH, A₃ is CR₁, wherein R₁ is halogen, an optionally substituted C₁₋₃ alkyl or an optionally substituted C₁₋₃ alkoxy, preferably halogen, more preferably F; L and Cy are as defined in any of the foregoing embodiments.

In one or more embodiments of the compound of Formula I or Ic, the Z ring is:

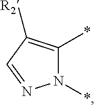

wherein, * indicates the position at which the Z ring is attached to the rest of the compound; R₂′ is selected from halogen, cyano, optionally substituted C₁₋₆ alkyl, optionally substituted C₁₋₃ alkoxy and optionally substituted C₃₋₆ cycloalkyl, preferably halogen, optionally substituted C₁₋₃ alkyl and optionally substituted C₃₋₆ cycloalkyl; A₁ is CH, A₂ is CR₁ and A₃ is CH, or both of A₁ and A₂ are CH, A₃ is CR₁, wherein R₁ is halogen, an optionally substituted C₁₋₃ alkyl or an optionally substituted C₁₋₃ alkoxy, preferably halogen, more preferably F; L and Cy are as defined in any of the foregoing embodiments.

In one or more embodiments of the compound of Formula I, preferred compounds are presented by Formula II (including Formulae IIa, IIb, IIc and IId), or stereoisomers, tautomers, N-oxides, hydrates, solvates, isotope-substituted derivatives, or pharmaceutically acceptable salts thereof, or mixtures thereof, or prodrugs thereof,

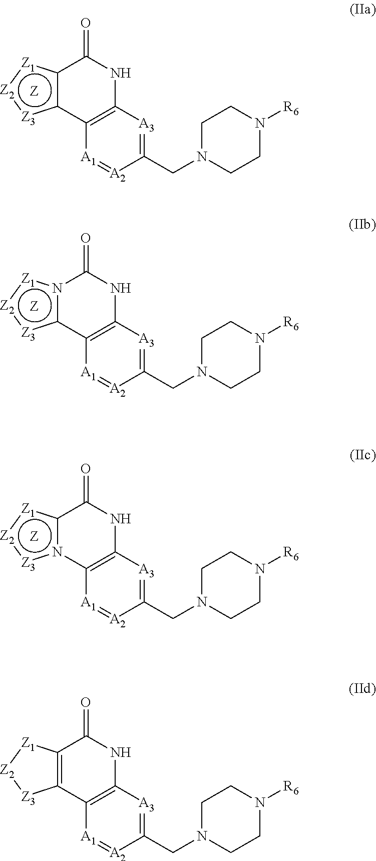

wherein, A₁, A₂, A₃, Z₁, Z₂ and Z₃ are as defined in any embodiments of Formula I, Ia, Ib, Ic and Id; R₆ is selected from an optionally substituted aryl and an optionally substituted heteroaryl.

In one or more embodiments of the compound of Formula II (including Formulae IIa, IIb, IIc and IId), the Z ring is selected from the following groups:

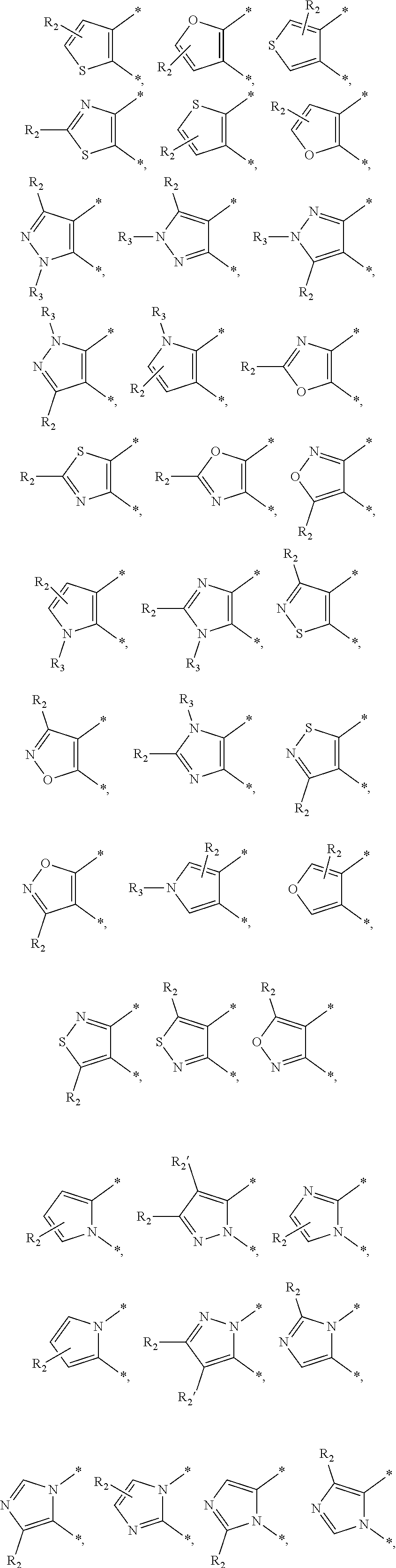

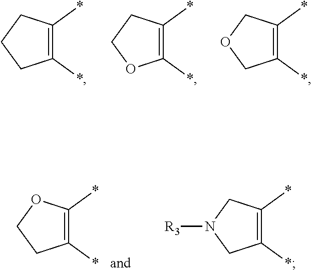

wherein, * indicates the position at which the Z ring is attached to the rest of the compound; R₂ is selected from hydrogen, halogen, cyano, an optionally substituted C₁₋₆ alkyl, an optionally substituted C₁₋₃ alkoxy and an optionally substituted C₃₋₆ cycloalkyl, preferably hydrogen, halogen and an optionally substituted C₁₋₃ alkyl; R₂′ is selected from hydrogen, halogen, cyano, an optionally substituted C₁₋₆ alkyl, an optionally substituted C₁₋₃ alkoxy and an optionally substituted C₃₋₆ cycloalkyl, preferably hydrogen, halogen and an optionally substituted C₁₋₃ alkyl; R₃ is selected from a group consisting of hydrogen, an optionally substituted alkyl and an optionally substituted cycloalkyl, preferably hydrogen and an optionally substituted C₁₋₃ alkyl.

In one or more embodiments of the compound of Formula IIa, the Z ring is selected from the following groups:

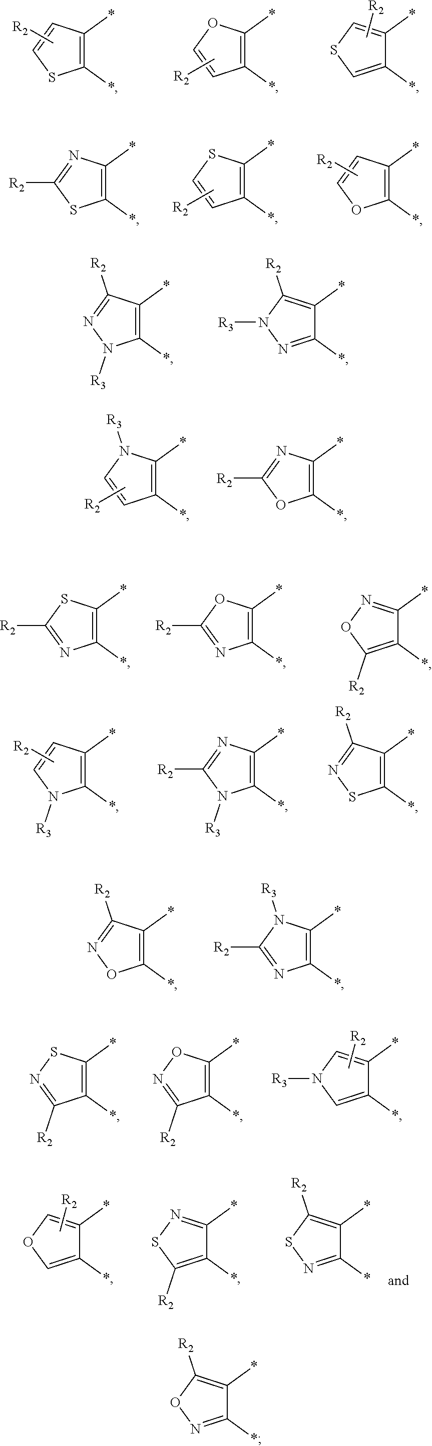

wherein, * indicates the position at which the Z ring is attached to the rest of the compound; R₂ is selected from hydrogen, halogen, an optionally substituted C₁₋₆ alkyl and an optionally substituted C₁₋₃ alkoxy, preferably hydrogen, halogen and an optionally substituted C₁₋₃ alkyl; R₃ is selected from a group consisting of hydrogen, an optionally substituted alkyl and an optionally substituted cycloalkyl, preferably hydrogen and an optionally substituted C₁₋₃ alkyl.

In one or more embodiments of the compound of Formulae IIb and IIc, the Z ring is selected from the following groups:

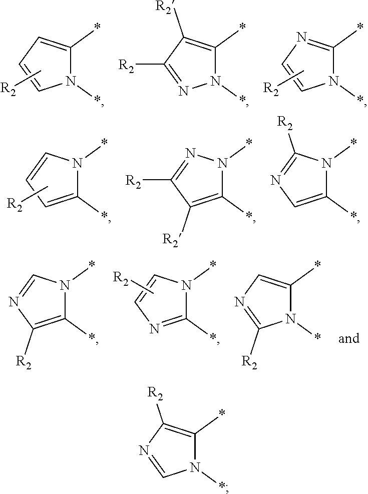

wherein R₂ and R₂′ are as defined in any of the foregoing embodiments; preferably, in these embodiments, at least one of A₂ and A₃ is CR₁, and R₁ is halogen; in some embodiments, both A₂ and A₃ are CR₁, and at least one of the R₁ groups is halogen. In some embodiments, A₃ is CR₁, and R₁ is halogen. In some embodiments, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen.

In one or more embodiments of the compound of Formula II (including Formulae IIa, IIb, IIc and IId), A₁, A₂ and A₃ are each independently selected from N and CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. In some embodiments, at least one of A₁ and A₃ is CR₁, and R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In some embodiments, A₃ is CR₁, and R₁ is halogen. In one or more embodiments, all of A₁, A₂ and A₃ are CR₁, each R₁ is independently hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula II (including Formulae IIa, IIb, IIc and IId), when the Z ring is not

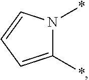

A₁, A₂ and A₃ are each independently selected from N and CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. In some embodiments, at least one of A₁ and A₃ is CR₁, and R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In some embodiments, A₃ is CR₁, and R₁ is halogen. In one or more embodiments, all of A₁, A₂ and A₃ are CR₁, each R₁ is independently hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula IIb, when the Z ring is

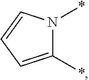

A₁ is CR₁, A₂ and A₃ each are independently N or CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. Preferably, A₃ is CR₁, wherein R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In one or more embodiments, all of A₁, A₂ and A₃ are CR₁, wherein R₁ is hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula II (including Formulae IIa, IIb, Ic and IId), R₆ is an optionally substituted 6-14 membered aryl or an optionally substituted 5-10 membered heteroaryl, wherein the said optionally substituted 6-14 membered aryl and optionally substituted 5-10 membered heteroaryl each can be independently substituted by 1-5 substituents selected from a group consisting of halogen, cyano, C₁₋₄ alkyl, C₁₋₄ alkoxy, halogenated C₁₋₄ alkyl, halogenated C₁₋₄ alkoxy, C₃₋₆ cycloalkyl, amino (—NR′R″), aminoacyl (—C(O)—NR′R″), carboxyl, and heteroaryl (such as 5-10 membered nitrogen-containing heteroaryl, especially 5-membered or 6-membered nitrogen-containing heteroaryl) optionally substituted by 1-3 substituents selected from a group consisting of C₁₋₄ alkyl; wherein the said R′ and R″ each are preferably independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably H, an optionally substituted C₁₋₄ alkyl, an optionally substituted C₃₋₆ cycloalkyl or an optionally substituted 3-6 membered heterocyclic group. In some preferred embodiments, the substituents of R₆ include at least an aminoacyl (—C(O)—NR′R″), and optionally include one or two of substituents selected from a group consisting of halogen, cyano, C₁₋₄ alkyl, halogenated C₁₋₄ alkyl and C₃₋₆ cycloalkyl. In some preferred embodiments, R₆ is an optionally substituted 5-10 membered heteroaryl, preferably an optionally substituted 5-10 membered nitrogen-containing heteroaryl. Preferably, the 5-10 membered heteroaryl or 5-10 membered nitrogen-containing heteroaryl is at least substituted by an aminoacyl (—C(O)—NR′R″), and optionally further substituted by one or two substituents selected from a group consisting of halogen, cyano, C₁₋₄ alkyl, halogenated C₁₋₄ alkyl and C₃₋₆ cycloalkyl. Preferably, when the said R′ and R″ are substituted, the substituents can be 1-5 groups selected from a group consisting of halogen, hydroxyl and amino.

Preferably, R₆ is an optionally substituted phenyl, an optionally substituted pyridyl, an optionally substituted pyrimidinyl, an optionally substituted pyrazinyl or an optionally substituted pyridazinyl. Preferably, when R₆ is substituted, the substituents can be 1-5 groups selected from a group consisting of halogen, cyano, an optionally substituted alkyl, an optionally substituted alkoxy, C₃₋₆ cycloalkyl, aminoacyl and carboxyl. More preferably, R₆ is at least substituted with an aminoacyl (—C(O)—NR′R″), preferably, the said aminoacyl is at the para position of R₆. Preferably, the said aminoacyl is —C(O)—NR′R″, wherein, the R′ and R″ each are preferably independently H, an optionally substituted C₁₋₁₀ alkyl, an optionally substituted C₃₋₈ cycloalkyl, an optionally substituted heterocyclic group, an optionally substituted aryl or an optionally substituted heteroaryl, preferably H, an optionally substituted C₁₋₄ alkyl, an optionally substituted C₃₋₆ cycloalkyl or an optionally substituted 3-6 membered heterocyclic group. Preferably, when the said R′ and R″ are substituted, the substituents can be 1-5 groups selected from a group consisting of halogen, hydroxyl, oxygen and amino. Preferably, the said optionally substituted alkyl and optionally substituted alkoxy can be substituted by 1-5 groups selected from a group consisting of halogen, hydroxyl, oxygen and amino. In preferred embodiments, the substituents on R₆ include at least an aminoacyl (—C(O)—NR′R″), and optionally include one or two of substituents selected from a group consisting of halogen, cyano, C₁₋₄ alkyl, halogenated C₁₋₄ alkyl and C₃₋₆ cycloalkyl. Further preferably, the said aminoacyl (—C(O)—NR′R″) is at the para position.

In one or more embodiments of the compound of Formula II (including Formulae IIa, IIb, IIc and IId), R₆ is preferably the following group:

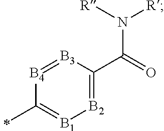

wherein, B₁, B₂, B₃ and B₄ are independently selected from a group consisting of N and CR₇; R₇ is selected from a group consisting of hydrogen, halogen, cyano, an optionally substituted alkyl, an optionally substituted alkoxy, an optionally substituted carbocyclic group, an optionally substituted alkenyl and an optionally substituted alkynyl; wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl, preferably hydrogen, an optionally substituted C₁₋₄ alkyl, an optionally substituted C₃₋₆ cycloalkyl and an optionally substituted 3-6 membered heterocyclic group. * indicates the position at which the said group is attached to the rest of the compound. Preferably, the group containing B₁, B₂, B₃ and B₄ is phenyl, pyridyl, pyrimidinyl, pyrazinyl or pyridazinyl. Preferably, R₇ is H, halogen, cyano, C₁₋₃ alkyl, C₁₋₃ alkoxy, halogenated C₁₋₃ alkyl or C₃₋₆ cycloalkyl. Preferably, B₃ is N, B₄ is CR₇, both of B₁ and B₂ are CH, wherein R₇ is H, halogen, cyano, C₁₋₃ alkyl, C₁₋₃ alkoxy, halogenated C₁₋₃ alkyl or C₃₋₆ cycloalkyl. Preferably, when the said R′ and R″ are substituted, the substituents can be 1-5 groups selected from a group consisting of halogen, hydroxy and amino. Preferably, R′ is hydrogen, R″ is hydrogen, C₁₋₃ alkyl, deuterated C₁₋₃ alkyl, C₃₋₆ cycloalkyl, halogenated C₁₋₃ alkyl, C₁₋₃ alkyl substituted with hydroxy or C₃₋₆ heterocyclic group.

Preferably, in the embodiments as described herein, the said R₇ is an optionally substituted alkyl, an optionally substituted alkoxy, an optionally substituted carbocyclic group, an optionally substituted alkenyl or an optionally substituted alkynyl, which can be independently substituted by 1-5 substituents selected from a group consisting of halogen, hydroxy and amino.

In one or more embodiments of the compound of Formula IIa, the Z ring is:

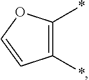

wherein, * indicates the position at which the Z ring is attached to the rest of the compound; A₁ is CH, A₂ is CR₁ and A₃ is CH, or both of A₁ and A₂ are CH, A₃ is CR₁, wherein, R₁ is halogen, an optionally substituted C₁₋₃ alkyl or an optionally substituted C₁₋₃ alkoxy, preferably halogen, more preferably F; R₆ is as defined in any of the foregoing embodiments.

In one or more embodiments of the compound of Formula IIc, the Z ring is:

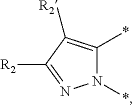

wherein, * indicates the position at which the Z ring is attached to the rest of the compound; R₂ is selected from hydrogen, halogen, cyano, optionally substituted alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; R₂′ is selected from hydrogen, halogen, cyano, optionally substituted C₁₋₆ alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; moreover, at least one of R₂ and R₂′ are non-hydrogen substituent, preferably, the non-hydrogen substituent is halogen, cyano, C₁₋₃ alkyl or C₃₋₆ cycloalkyl; A₁ is CH, A₂ is CR₁ and A₃ is CH, or both of A₁ and A₂ are CH, A₃ is CR₁, wherein, R₁ is halogen, an optionally substituted C₁₋₃ alkyl or an optionally substituted C₁₋₃ alkoxy, preferably halogen, more preferably F; R₆ is as defined in any of the foregoing embodiments.

In one or more embodiments of the compound of Formula IIc, the Z ring is:

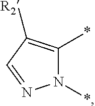

wherein, * indicates the position at which the Z ring is attached to the rest of the compound; R₂′ is selected from halogen, cyano, optionally substituted C₁₋₆ alkyl, optionally substituted C₁₋₃ alkoxy and optionally substituted C₃₋₆ cycloalkyl, preferably halogen, optionally substituted C₁₋₃ alkyl and optionally substituted C₃₋₆ cycloalkyl; A₁ is CH, A₂ is CR₁ and A₃ is CH, or both of A₁ and A₂ are CH, A₃ is CR₁, wherein, R₁ is halogen, an optionally substituted C₁₋₃ alkyl or an optionally substituted C₁₋₃ alkoxy, preferably halogen, more preferably F; R₆ is as defined in any of the foregoing embodiments.

In one or more embodiments of the compound of Formula I, preferred compounds are presented by Formula III (including Formulae IIIa, IIIb, IIIc and IIId), or stereoisomers, tautomers, N-oxides, hydrates, solvates, isotope-substituted derivatives, or pharmaceutically acceptable salts thereof, or mixtures thereof, or prodrugs thereof,

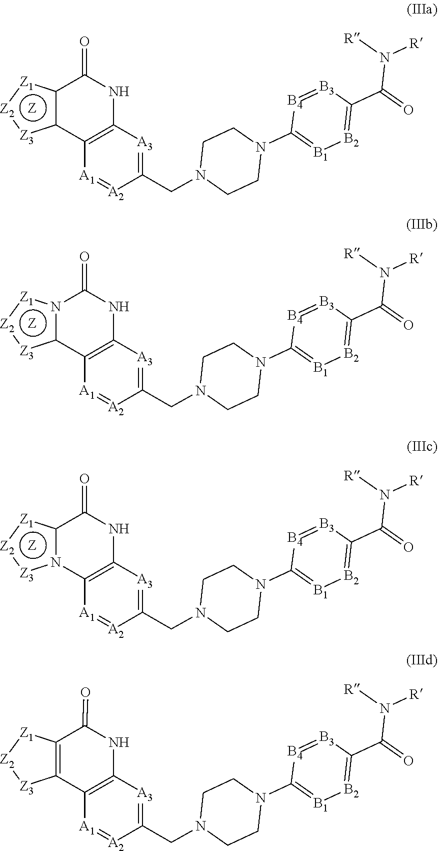

wherein, Z₁, Z₂, Z₃, A₁, A₂ and A₃ are as defined in any of the foregoing embodiments;
B₁, B₂, B₃ and B₄ are independently selected from N and CR₇;
R₇ is selected from hydrogen, halogen, cyano, optionally substituted alkyl, optionally substituted alkoxy, optionally substituted carbocyclic group, optionally substituted alkenyl and optionally substituted alkynyl;
R′ and R″ are independently selected from hydrogen, an optionally substituted C₁₋₁₀ alkyl, an optionally substituted cycloalkyl, an optionally substituted heterocyclic group, an optionally substituted aryl and an optionally substituted heteroaryl.

In one or more embodiments of the compound of Formula III (including Formulae IIIa, IIlb, IIIc and IIId), the Z ring is selected from the following groups:

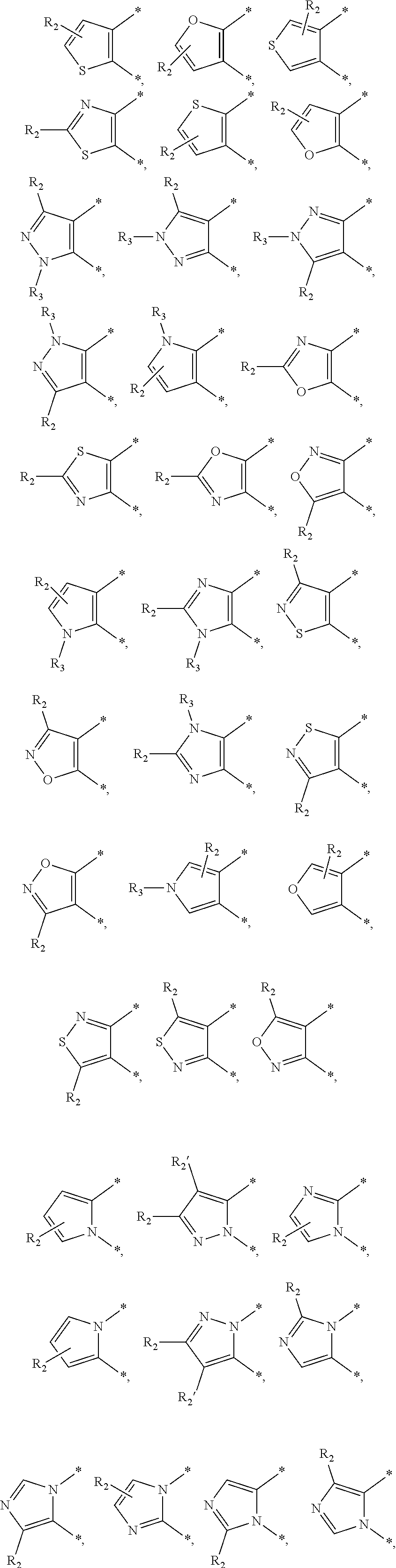

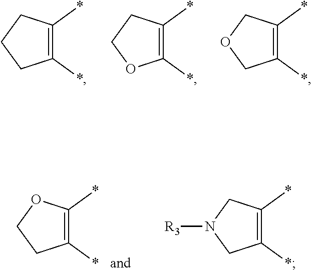

wherein, * indicates the position at which the Z ring is attached to the rest of the compound; R₂ is selected from hydrogen, halogen, cyano, an optionally substituted C₁₋₆ alkyl, an optionally substituted C₁₋₃ alkoxy and optionally substituted C₃₋₆ cycloalkyl, preferably hydrogen, halogen and an optionally substituted C₁₋₃ alkyl and optionally substituted C₃₋₆ cycloalkyl; R₂′ is selected from hydrogen, halogen, cyano, an optionally substituted C₁₋₆ alkyl, an optionally substituted C₁₋₃ alkoxy and optionally substituted C₃₋₆ cycloalkyl, preferably hydrogen, halogen and an optionally substituted C₁₋₃ alkyl and optionally substituted C₃₋₆ cycloalkyl; R₃ is selected from a group consisting of hydrogen, an optionally substituted alkyl and an optionally substituted cycloalkyl, preferably hydrogen and an optionally substituted C₁₋₃ alkyl.

In one or more embodiments of the compound of Formula IIIa, the Z ring is selected from the following groups:

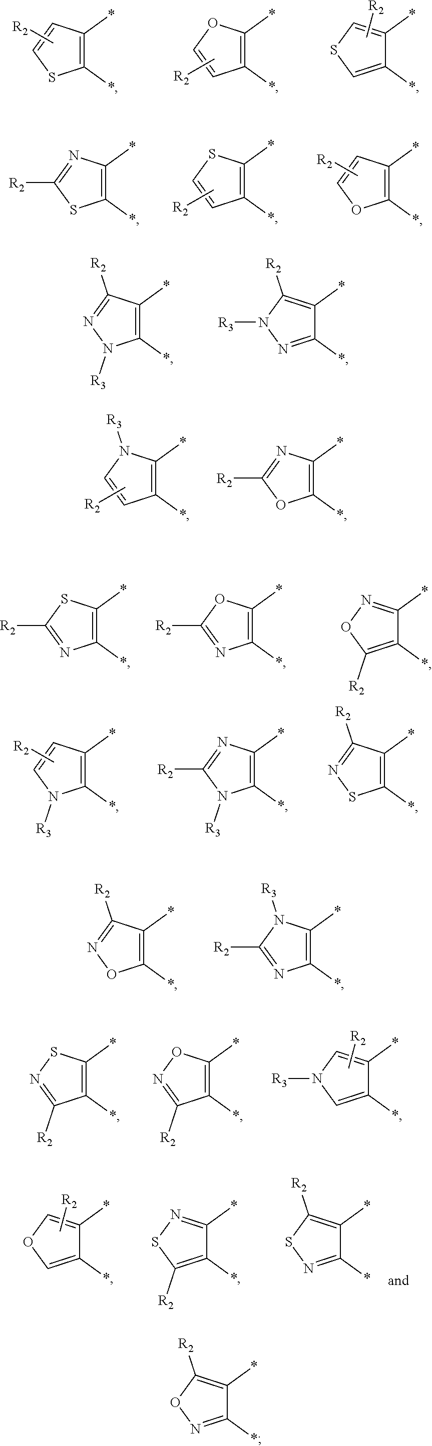

wherein, * indicates the position at which the Z ring is attached to the rest of the compound; R₂ is selected from hydrogen, halogen, cyano, an optionally substituted alkyl, an optionally substituted C₁₋₃ alkoxy and an optionally substituted C₃₋₆ cycloalkyl, preferably hydrogen, halogen cyano, an optionally substituted C₁₋₃ alkyl and an optionally substituted C₃₋₆ cycloalkyl; R₃ is selected from a group consisting of hydrogen, an optionally substituted alkyl and an optionally substituted cycloalkyl, preferably hydrogen and an optionally substituted C₁₋₃ alkyl.

In one or more embodiments of the compound of Formulae IIIb and IIIc, the Z ring is selected from the following groups:

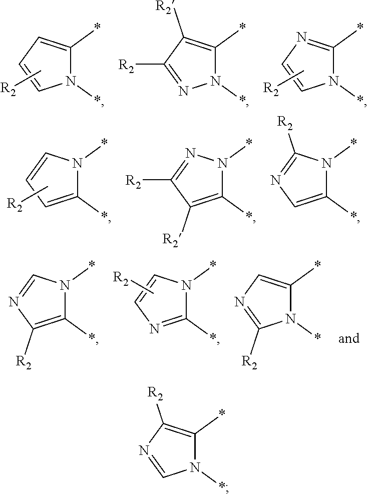

wherein R₂ and R₂′ are as defined in any of the foregoing embodiments; preferably, in these embodiments, at least one of A₂ and A₃ is CR₁, and R₁ is halogen; in some embodiments, both A₂ and A₃ are CR₁, and at least one of the R₁ groups is halogen. In some embodiments, A₃ is CR₁, and R₁ is halogen. In some embodiments, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen.

In one or more embodiments of the compound of Formula III (including Formulae IIIa, IIIb, IIIc and IIId), A₁, A₂ and A₃ are each independently selected from N and CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. In some embodiments, at least one of A₁ and A₃ is CR₁, and R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In some embodiments, A₃ is CR₁, and R₁ is halogen. In one or more embodiments, all of A₁, A₂ and A₃ are CR₁, each R₁ is independently hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula III (including Formulae IIIa, IIIb, IIIc and IIId), when the Z ring is not

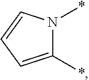

A₁, A₂ and A₃ are each independently selected from N and CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. In some embodiments, at least one of A₁ and A₃ is CR₁, and R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In some embodiments, A₃ is CR₁, and R₁ is halogen. In one or more embodiments, all of A₁, A₂ and A₃ are CR₁, each R₁ is independently hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula IIIb, when the Z ring is

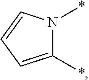

A₁ is CR₁, A₂ and A₃ each are independently N or CR₁, wherein R₁ is preferably hydrogen, halogen, an optionally substituted C₁₋₃ alkyl or an optionally substituted C₁₋₃ alkoxy, more preferably, R₁ is hydrogen, halogen or C₁₋₃ alkyl. Preferably, A₃ is CR₁, wherein R₁ is halogen, such as fluoro. In some embodiments, at least one of A₂ and A₃ is CR₁, and R₁ is halogen. In some embodiments, both A₂ and A₃ are CR₁, and at least one of the R₁ groups is halogen. In some embodiments, A₃ is CR₁, and R₁ is halogen. In one or more embodiments, all of A₁, A₂ and A₃ are CR₁, wherein R₁ is hydrogen, halogen or C₁₋₃ alkyl. Preferably, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl.

In one or more embodiments of the compound of Formula III (including Formulae IIIa, IIIb, IIIc and IIId), B₁, B₂, B₃ and B₄ are independently selected from a group consisting of N and CR₇; wherein R₇ is preferably hydrogen, halogen, cyano, optionally substituted C₁₋₃ alkyl, optionally substituted C₁₋₃ alkoxy or optionally substituted C₃₋₆ cycloalkyl, more preferably, R₇ is hydrogen, C₁₋₃ alkyl, halogenated C₁₋₃ alkyl, halogen, cyano or C₃₋₆ cycloalkyl. In preferred embodiments, both of B₁ and B₂ are CH, B₃ is N, B₄ is CR₇, wherein R₇ is preferably hydrogen, halogen, optionally substituted C₁₋₃ alkyl, optionally substituted C₁₋₃ alkoxy or optionally substituted C₃₋₆ cycloalkyl, more preferably, R₇ is hydrogen, C₁₋₃ alkyl, halogenated C₁₋₃ alkyl, halogen, cyano or C₃₋₆ cycloalkyl.

In one or more embodiments of the compound of Formula III (including Formulae IIIa, IIIb, IIIc and IIId), R′ and R″ each are independently hydrogen, an optionally substituted C₁₋₃ alkyl, an optionally substituted C₃₋₆ cycloalkyl or an optionally substituted 5-6 membered heterocyclic group. Preferably, R′ is hydrogen, R″ is hydrogen, C₁₋₃ alkyl, deuterated C₁₋₃ alkyl, C₃₋₆ cycloalkyl, halogenated C₁₋₃ alkyl, hydroxy C₁₋₃ alkyl, oxo C₁₋₃ alkyl or 3-6 membered heterocyclic group.

In preferred embodiments, the —C(O)—NR′R″ group described herein, R′ and R″ each are independently hydrogen, an optionally substituted C₁₋₄ alkyl and an optionally substituted C₃₋₆ cycloalkyl. In further preferred embodiments, R′ is hydrogen, R″ is hydrogen, C₁₋₃ alkyl, halogenated C₁₋₃ alkyl, or deuterated C₁₋₃ alkyl.

In one or more embodiments of the compound of Formula IIIa, the Z ring is:

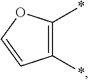

wherein, * indicates the position at which the Z ring is attached to the rest of the compound; A₁ is CH, A₂ is CR₁ and A₃ is CH, or both of A₁ and A₂ are CH, A₃ is CR₁, wherein, R₁ is halogen, an optionally substituted C₁₋₃ alkyl or an optionally substituted C₁₋₃ alkoxy, preferably halogen, more preferably F; B₁, B₂, B₃, B₄, R′ and R″ are as defined in any of the foregoing embodiments.

In one or more embodiments of the compound of Formula IIIc, the Z ring is:

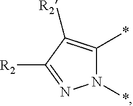

wherein, * indicates the position at which the Z ring is attached to the rest of the compound; R₂ is selected from hydrogen, halogen, cyano, optionally substituted alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; R₂′ is selected from hydrogen, halogen, cyano, optionally substituted C₁₋₆ alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; moreover, at least one of R₂ and R₂′ are non-hydrogen substituent, preferably, the non-hydrogen substituent is halogen, cyano, C₁₋₃ alkyl or C₃₋₆ cycloalkyl; A₁ is CH, A₂ is CR₁ and A₃ is CH, or both of A₁ and A₂ are CH, A₃ is CR₁, wherein, R₁ is halogen, an optionally substituted C₁₋₃ alkyl or an optionally substituted C₁₋₃ alkoxy, preferably halogen, more preferably F; B₁, B₂, B₃, B₄, R′ and R″ are as defined in any of the foregoing embodiments.

In one or more embodiments of the compound of Formula IIIc, the Z ring is:

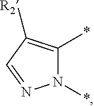

wherein, * indicates the position at which the Z ring is attached to the rest of the compound; R₂′ is selected from halogen, cyano, optionally substituted C₁₋₆ alkyl, optionally substituted C₁₋₃ alkoxy and optionally substituted C₃₋₆ cycloalkyl, preferably halogen, optionally substituted C₁₋₃ alkyl and optionally substituted C₃₋₆ cycloalkyl; A₁ is CH, A₂ is CR₁ and A₃ is CH, or both of A₁ and A₂ are CH, A₃ is CR₁, wherein, R₁ is halogen, an optionally substituted C₁₋₃ alkyl or an optionally substituted C₁₋₃ alkoxy, preferably halogen, more preferably F; B₁, B₂, B₃, B₄, R′ and R″ are as defined in any of the foregoing embodiments.

In one or more embodiments of the compound of Formula I, preferred compounds are presented by Formula IV (including Formulae IVa, IVb, IVc and IVd), or stereoisomers, tautomers, N-oxides, hydrates, solvates, isotope-substituted derivatives, or pharmaceutically acceptable salts thereof, or mixtures thereof, or prodrugs thereof,

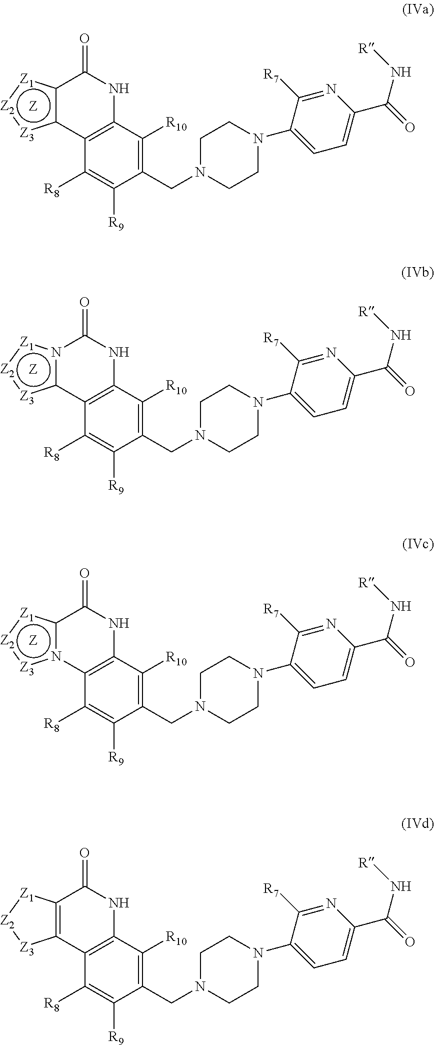

wherein, Z₁, Z₂, Z₃ and R″ are as defined in any of the foregoing embodiments;
R₇ is selected from hydrogen, halogen, cyano, optionally substituted alkyl, optionally substituted alkoxy, optionally substituted carbocyclic group, optionally substituted alkenyl and optionally substituted alkynyl;
R₈, R₉ and R₁₀ are independently selected from hydrogen, halogen, optionally substituted alkyl, optionally substituted alkoxy and optionally substituted carbocyclic group.

In one or more embodiments of the compound of Formula IV (including Formulae IVa, IVb, IVc and IVd), the Z ring is selected from the following groups:

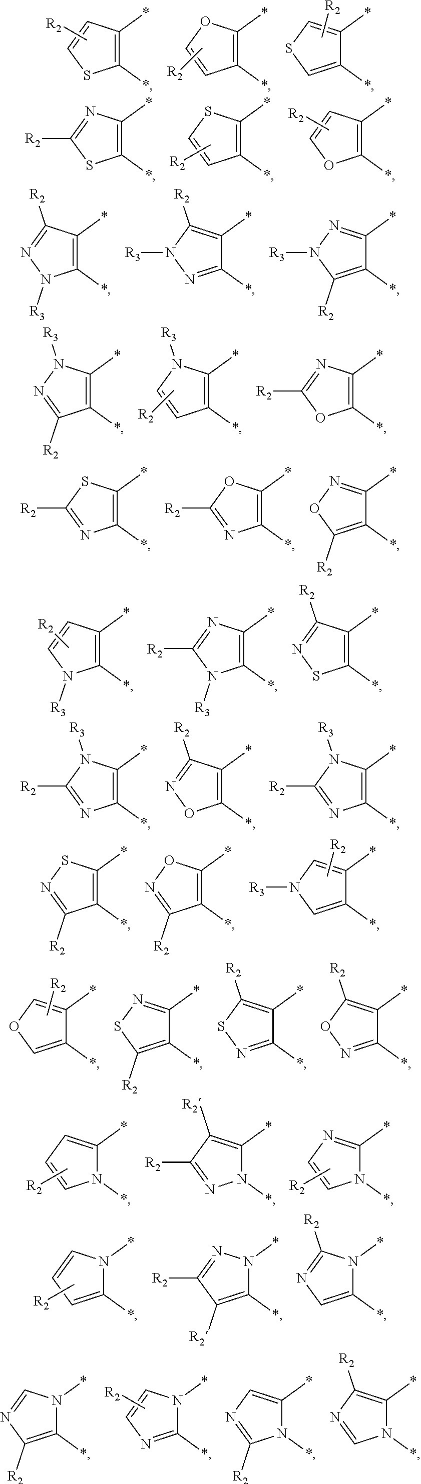

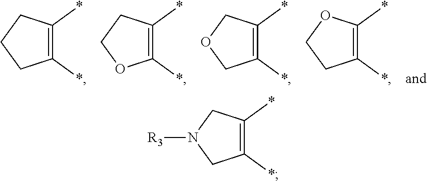

wherein, * indicates the position at which the Z ring is attached to the rest of the compound; R₂ is selected from hydrogen, halogen, cyano, an optionally substituted C₁₋₆ alkyl, an optionally substituted C₁₋₃ alkoxy and optionally substituted C₃₋₆ cycloalkyl, preferably hydrogen, halogen, an optionally substituted C₁₋₃ alkyl and optionally substituted C₃₋₆ cycloalkyl; R₂′ is selected from hydrogen, halogen, cyano, an optionally substituted C₁₋₆ alkyl, an optionally substituted C₁₋₃ alkoxy and optionally substituted C₃₋₆ cycloalkyl, preferably hydrogen, halogen, an optionally substituted C₁₋₃ alkyl and optionally substituted C₃₋₆ cycloalkyl; R₃ is selected from a group consisting of hydrogen, an optionally substituted alkyl and an optionally substituted cycloalkyl, preferably hydrogen and an optionally substituted C₁₋₃ alkyl. Preferably, R₂, R₂′ and R₃ are as defined in any of the foregoing embodiments.

In one or more embodiments of the compound of Formulae IVb and IVc, the Z ring is selected from the following groups:

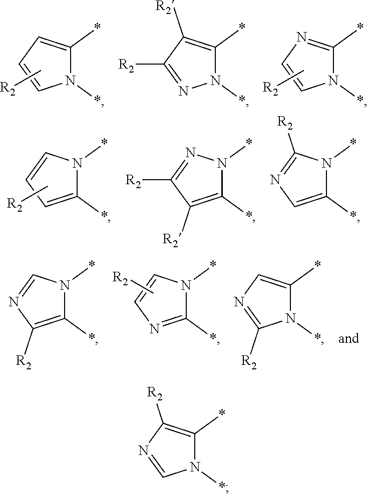

wherein R₂ and R₂′ are as defined in any of the foregoing embodiments; preferably, in these embodiments, at least one of A₂ and A₃ is CR₁, and R₁ is halogen; in some embodiments, both A₂ and A₃ are CR₁, and at least one of the R₁ groups is halogen. In some embodiments, A₃ is CR₁, and R₁ is halogen. In some embodiments, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen.

In one or more embodiments of the compound of Formula IV (including Formulae IVa, IVb, IVc and IVd), R₇ is hydrogen, halogen, cyano, optionally substituted C₁₋₃ alkyl or optionally substituted C₁₋₃ alkoxy, preferably hydrogen, C₁₋₃ alkyl, halogenated C₁₋₃ alkyl or halogen.

In one or more embodiments of the compound of Formula IV (including Formulae IVa, IVb, IVc and IVd), R₈, R₉ and R₁₀ are each independently hydrogen, halogen, optionally substituted C₁₋₃ alkyl or optionally substituted C₁₋₃ alkoxy, preferably, R₈, R₉ and R₁₀ are each independently hydrogen, halogen or C₁₋₃ alkyl; preferably, at least one of R₈, R₉ and R₁₀ are non-hydrogen substituent, preferably, the non-hydrogen substituent is halogen. In one or more embodiments, R₈ is halogen or C₁₋₃ alkyl, both of R₉ and R₁₀ are CH; or R₈ is H, R₉ is halogen or C₁₋₃ alkyl, R₁₀ is CH; or both of R₈ and R₉ are CH, R₁₀ is halogen or C₁₋₃ alkyl. In some embodiments, at least one of R₉ and R₁₀ is halogen, preferably at least R₁₀ is halogen, such as fluoro.

In one or more embodiments of the compound of Formula IV (including Formulae IVa, IVb, IVc and IVd), R″ is hydrogen, an optionally substituted C₁₋₄ alkyl, an optionally substituted C₃₋₆ cycloalkyl and an optionally substituted 3-6 membered heterocyclic group, preferably hydrogen, C₁₋₃ alkyl, deuterated C₁₋₃ alkyl, C₃₋₆ cycloalkyl, halogenated C₁₋₃ alkyl or 3-6 membered heterocyclic group.

In one or more embodiments of the compound of Formula IVa, the Z ring is:

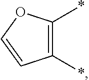

wherein, * indicates the position at which the Z ring is attached to the rest of the compound; R₈ is H, one of R₉ and R₁₀ is halogen or C₁₋₃ alkyl, the other one is hydrogen; R₇ and R″ are as defined in any of the foregoing embodiments.

In some embodiments of the compound of Formula IVc, the Z ring is:

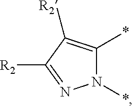

wherein, * indicates the position at which the Z ring is attached to the rest of the compound; R₂ is selected from hydrogen, halogen, cyano, optionally substituted alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; R₂′ is selected from hydrogen, halogen, cyano, optionally substituted C₁₋₆ alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; moreover, at least one of R₂ and R₂′ is non-hydrogen substituent, preferably, the non-hydrogen substituent is halogen, cyano, C₁₋₃ alkyl or C₃₋₆ cycloalkyl; any one of R₈, R₉ and R₁₀ is halogen or C₁₋₃ alkyl, others are hydrogen, preferably, R₈ is halogen or C₁₋₃ alkyl, both of R₉ and R₁₀ are CH or R₈ is H, R₉ is halogen or C₁₋₃ alkyl, R₁₀ is CH or both of R₈ and R₉ are CH, R₁₀ is halogen or C₁₋₃ alkyl; R₇ and R″ are as defined in any of the foregoing embodiments.

In one or more embodiments of the compound of Formula IVc, the Z ring is:

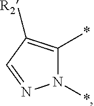

wherein, * indicates the position at which the Z ring is attached to the rest of the compound; R₂′ is selected from halogen, cyano, optionally substituted C₁₋₆ alkyl, optionally substituted C₁₋₃ alkoxy and optionally substituted C₃₋₆ cycloalkyl, preferably halogen, optionally substituted C₁₋₃ alkyl and optionally substituted C₃₋₆ cycloalkyl; R₈ is H, one of R₉ and R₁₀ is halogen or C₁₋₃ alkyl, the other one is hydrogen; R₇ and R″ are as defined in any of the foregoing embodiments.

In one or more embodiments of the compound of Formula I, preferred compounds are presented by Formula V (including Formulae Va, Vb, Vc and Vd), or stereoisomers, tautomers, N-oxides, hydrates, solvates, isotope-substituted derivatives, or pharmaceutically acceptable salts thereof, or mixtures thereof, or prodrugs thereof,

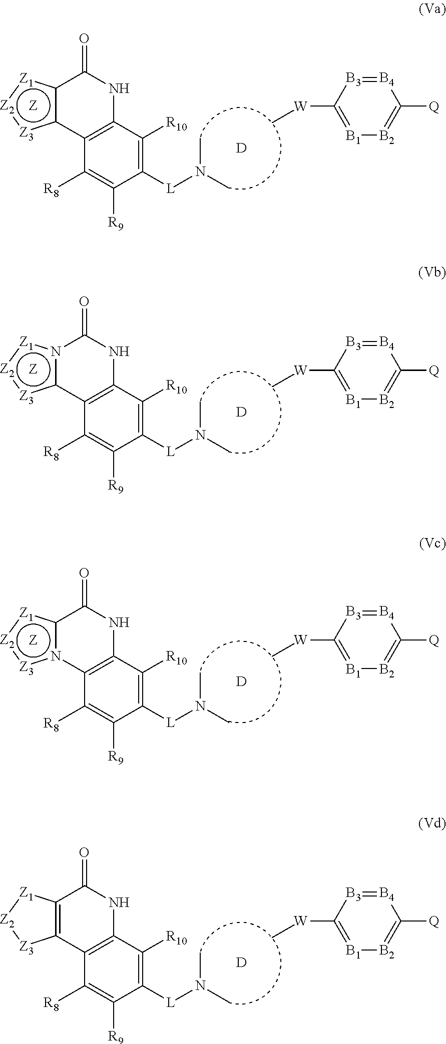

wherein, Z₁, Z₂, Z₃, R₈, R₉, R₁₀, B₁, B₂, B₃ and B₄ are as defined in any of the foregoing embodiments;
The D ring is an optionally substituted 4 to 12 membered N-containing heterocyclic group; the said N-containing heterocyclic group containing at least 1 N atom is selected from 4-12 membered monocyclic group, or 5-12 membered spirocyclic group, the said N-containing heterocyclic group can also include 1-4 heteroatoms selected from N, O or S, and can be optionally further substituted by 1 or more R″′;
W is a bond, O or —NR′—;
Q is an optionally substituted aminoacyl (—C(O)—NR′R″) or an optionally substituted heteroaryl;
R′ and R″ are each independently selected from hydrogen, optionally substituted C₁₋₁₀ alkyl, optionally substituted cycloalkyl, optionally substituted heterocyclic group, optionally substituted aryl and optionally substituted heteroaryl;
R″′ is selected from halogen, hydroxyl, cyano or C₁₋₆ alkyl, and the said C₁₋₆ alkyl is optionally further substituted by one or more substituents selected from hydroxyl, halogen or cyano; or any two R″′ can form a 3-8 membered ring.

In one or more embodiments of the compound of Formula V (including Formulae Va, Vb, Vc and Vd), the Z ring is selected from the following groups:

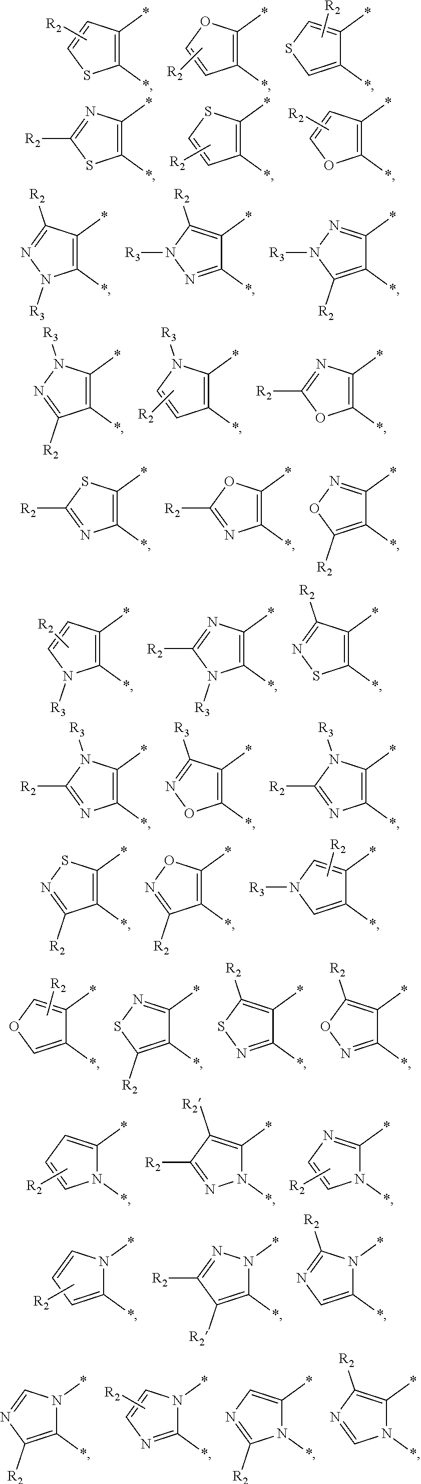

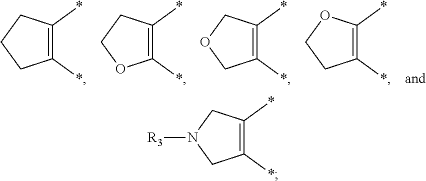

wherein, * indicates the position at which the Z ring is attached to the rest of the compound; R₂ is selected from hydrogen, halogen, cyano, an optionally substituted alkyl, an optionally substituted C₁₋₃ alkoxy and optionally substituted C₃₋₆ cycloalkyl, preferably hydrogen, halogen, an optionally substituted C₁₋₃ alkyl and optionally substituted C₃₋₆ cycloalkyl; R₂′ is selected from hydrogen, halogen, cyano, an optionally substituted C₁₋₆ alkyl, an optionally substituted C₁₋₃ alkoxy and optionally substituted C₃₋₆ cycloalkyl, preferably hydrogen, halogen, an optionally substituted C₁₋₃ alkyl and optionally substituted C₃₋₆ cycloalkyl; R₃ is selected from a group consisting of hydrogen, an optionally substituted alkyl and an optionally substituted cycloalkyl, preferably hydrogen and an optionally substituted C₁₋₃ alkyl. Preferably, R₂, R₂′ and R₃ are as defined in any of the foregoing embodiments.

In one or more embodiments of the compound of Formulae Vb and Vc, the Z ring is selected from the following groups:

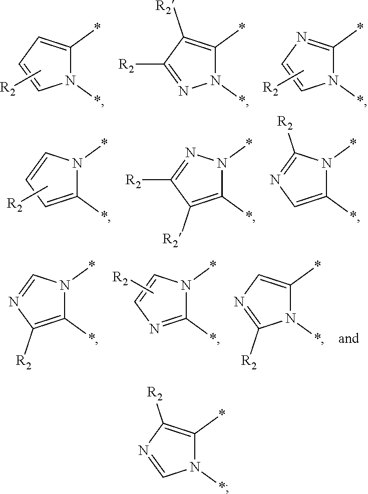

wherein R₂ and R₂′ are as defined in any of the foregoing embodiments; preferably, in these embodiments, at least one of A₂ and A₃ is CR₁, and R₁ is halogen; in some embodiments, both A₂ and A₃ are CR₁, and at least one of the R₁ groups is halogen. In some embodiments, A₃ is CR₁, and R₁ is halogen. In some embodiments, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen.

In one or more embodiments of the compound of Formula V (including Formulae Va, Vb, Vc and Vd), R₈, R₉ and R₁₀ are each independently hydrogen, halogen, optionally substituted C₁₋₃ alkyl or optionally substituted C₁₋₃ alkoxy, preferably, R₈, R₉ and R₁₀ are each independently hydrogen, halogen or C₁₋₃ alkyl; preferably, at least one of R₈, R₉ and R₁₀ are non-hydrogen substituent, preferably, the non-hydrogen substituent is halogen. In one or more embodiments, R₈ is halogen or C₁₋₃ alkyl, both of R₉ and R₁₀ are CH; or R₈ is H, R₉ is halogen or C₁₋₃ alkyl, R₁₀ is CH; or both of R₈ and R₉ are CH, R₁₀ is halogen or C₁₋₃ alkyl. In some embodiments, at least one of R₉ and R₁₀ are halogen, preferably at least R₁₀ is halogen, such as fluoro.

In one or more embodiments of the compound of Formula V (including Formulae Va, Vb, Vc and Vd), L is an alkylene optionally substituted by 1-2 C₁₋₃ alkyl groups, more preferably a C₁₋₃ alkylene optionally substituted by 1-2 C₁₋₃ alkyl groups, preferably a methylene optionally substituted by 1-2 C₁₋₃ alkyl.

In one or more embodiments of the compound of Formula V (including Formulae Va, Vb, Vc and Vd), the D ring is an optionally substituted 4-7 membered monocyclic ring containing at least 1 N atom; preferably ring D is an optionally substituted piperazinyl, an optionally substituted piperidinyl, an optionally substituted dihyropyridinyl, or an optionally substituted pyrrolidinyl. When the ring D is substituted, the substituents can be 1-3 groups selected from a group consisting of halogen, hydroxyl, cyano, optionally substituted C₁₋₃ alkyl, optionally substituted C₁₋₃ alkoxy and optionally substituted C₃₋₆ cycloalkyl.

In one or more embodiments of the compound of Formula V (including Formulae Va, Vb, Vc and Vd), the D ring is selected from the following groups:

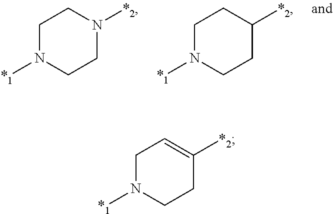

preferably, the D ring is

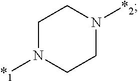

wherein *1 indicates the position at which the D ring is attached to L; *2 indicates the position at which the D ring is attached to W.

In one or more embodiments of the compound of Formula V (including Formulae Va, Vb, Vc and Vd), W is a bond or —NH—.

In one or more embodiments of the compound of Formula V (including Formulae Va, Vb, Vc and Vd), the ring D is optionally substituted piperazinyl, optionally substituted piperidinyl or optionally substituted dihyropyridinyl, and W is a bond. In one or more embodiments, the ring D is optional substituted pyrrolidinyl, W is —NH—.

In one or more embodiments of the compound of Formula V (including Formulae Va, Vb, Vc and Vd), B₁, B₂, B₃ and B₄ are independently selected from a group consisting of N and CR₇; wherein R₇ is preferably hydrogen, halogen, cyano, optionally substituted C₁₋₃ alkyl, optionally substituted C₁₋₃ alkoxy or optionally substituted C₃₋₆ cycloalkyl, more preferably, R₇ is hydrogen, C₁₋₃ alkyl, halogenated C₁₋₃ alkyl, halogen, cyano or C₃₋₆ cycloalkyl. In preferred embodiments, both of B₁ and B₂ are CH, B₃ is N, B₄ is CR₇, wherein R₇ is preferably hydrogen, halogen, optionally substituted C₁₋₃ alkyl, optionally substituted C₁₋₃ alkoxy or optionally substituted C₃₋₆ cycloalkyl, more preferably, R₇ is hydrogen, C₁₋₃ alkyl, halogenated C₁₋₃ alkyl, halogen, cyano or C₃₋₆ cycloalkyl.

In one or more embodiments of the compound of Formula V (including Formulae Va, Vb, Vc and Vd), Q is —C(O)—NR′R″, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or optionally substituted heteroaryl, preferably hydrogen, an optionally substituted C₁₋₄ alkyl, an optionally substituted C₃₋₆ cycloalkyl or an optionally substituted 3-6 membered heterocyclic group. In one or more embodiments, Q is a 5 membered heteroaryl optionally substituted by 1-3 substituents selected from halogen, cyano, C₁₋₃ alkyl, halogenated C₁₋₃ alkyl, C₁₋₃ alkoxy and halogenated C₁₋₃ alkoxy, the 5 membered heteroaryl is preferably pyrrolyl, pyrazolyl, imidazolyl or triazolyl.

In one or more embodiments of the compound of Formula Va, the Z ring is:

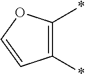

wherein, * indicates the position at which the Z ring is attached to the rest of the compound; R₈ is H, one of R₉ and R₁₀ is halogen or C₁₋₃ alkyl, the other one is hydrogen; L, ring D, W, B₁, B₂, B₃, B₄ and Q are as defined in any of the foregoing embodiments.

In some embodiments of the compound of Formula Vc, the Z ring is:

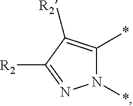

wherein, * indicates the position at which the Z ring is attached to the rest of the compound; R₂ is selected from hydrogen, halogen, cyano, optionally substituted alkyl, optionally substituted C₁₋₃ alkoxy and optionally substituted C₃₋₆ cycloalkyl, preferably hydrogen, halogen, optionally substituted C₁₋₃ alkyl and optionally substituted C₃₋₆ cycloalkyl; R₂′ is selected from hydrogen, halogen, cyano, optionally substituted C₁₋₆ alkyl, optionally substituted C₁₋₃ alkoxy and optionally substituted C₃₋₆ cycloalkyl, preferably hydrogen, halogen, cyano, optionally substituted C₁₋₃ alkyl and optionally substituted C₃₋₆ cycloalkyl; moreover, at least one of R₂ and R₂′ are non-hydrogen substituent, preferably, the non-hydrogen substituent is halogen, cyano, C₁₋₃ alkyl or C₃₋₆ cycloalkyl; any one of R₈, R₉ and R₁₀ is halogen or C₁₋₃ alkyl, others are hydrogen, preferably, R₈ is halogen or C₁₋₃ alkyl, both of R₉ and R₁₀ are CH or R₈ is H, R₉ is halogen or C₁₋₃ alkyl, R₁₀ is CH or both of R₈ and R₉ are CH, R₁₀ is halogen or C₁₋₃ alkyl; L, ring D, W, B₁, B₂, B₃, B₄ and Q are as defined in any of the foregoing embodiments.

In one or more embodiments of the compound of Formula Vc, the Z ring is:

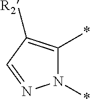

wherein, * indicates the position at which the Z ring is attached to the rest of the compound; R₂′ is selected from halogen, cyano, optionally substituted C₁₋₆ alkyl, optionally substituted C₁₋₃ alkoxy and optionally substituted C₃₋₄₁ cycloalkyl, preferably halogen, cyano, optionally substituted C₁₋₃ alkyl and optionally substituted C₃₋₆ cycloalkyl; R₈ is H, one of R₉ and R₁₀ is halogen or C₁₋₃ alkyl, the other one is hydrogen; L, ring D, W, B₁, B₂, B₃, B₄ and Q are as defined in any of the foregoing embodiments.

In one or more embodiments of the compound of Formula I, preferred compounds are presented by Formula VI (including Formulae VIa, VIb and VIc), or stereoisomers, tautomers, N-oxides, hydrates, solvates, isotope-substituted derivatives, or pharmaceutically acceptable salts thereof, or mixtures thereof, or prodrugs thereof,

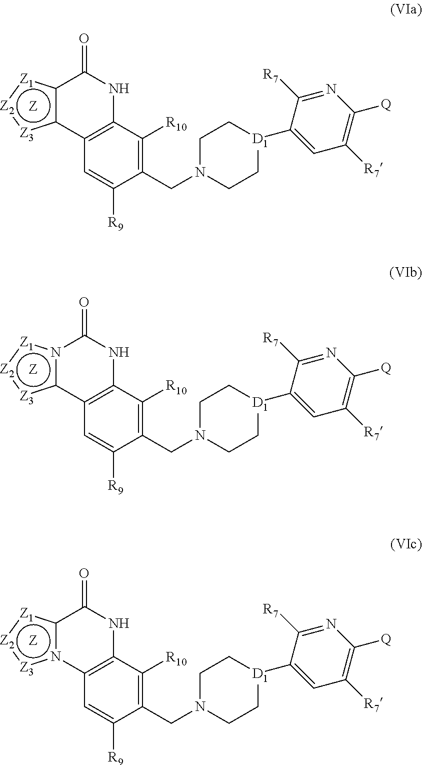

wherein, Z₁, Z₂, Z₃, R₇, D₁ and Q are as defined in any of the foregoing embodiments;
R₇′ is hydrogen, halogen, cyano, optionally substituted C₁₋₃ alkyl, optionally substituted C₁₋₃ alkoxy or optionally substituted C₃₋₆ cycloalkyl;
R₉ and R₁₀ are each independently hydrogen, halogen, optionally substituted C₁₋₃ alkyl, or optionally substituted C₁₋₃ alkoxy, and at least one of R₉ and R₁₀ is not hydrogen;
D₁ is N or CR₁₁;
R₁₁ is H, halogen, cyano, hydroxyl, optionally substituted C₁₋₃ alkyl or optionally substituted C₁₋₃ alkoxy.

In one or more embodiments of the compound of Formula VI (including Formulae VIa, VIb and VIc), the Z ring is selected from the following groups:

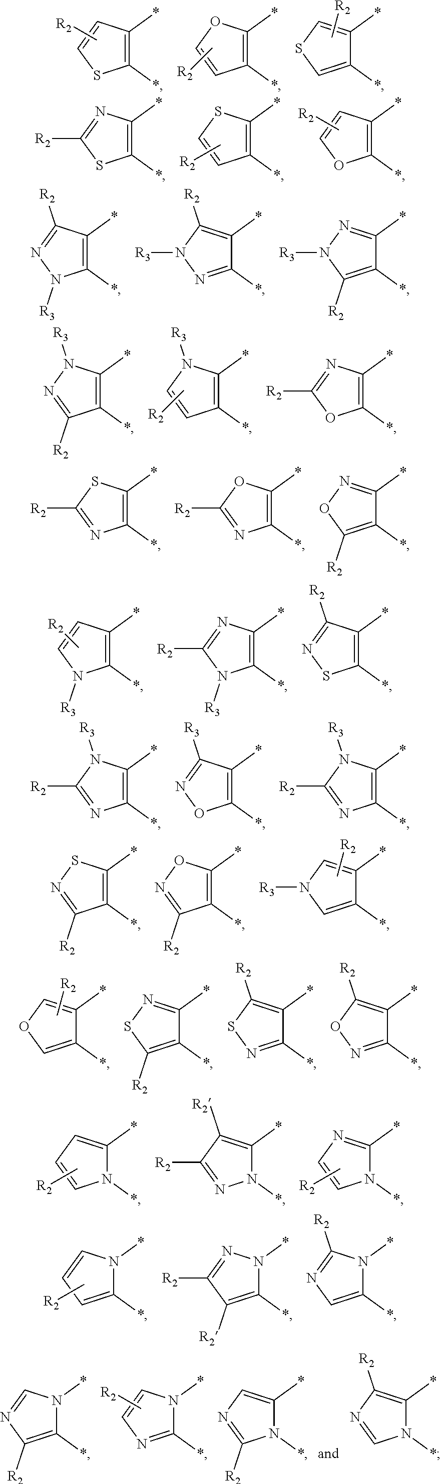

wherein, * indicates the position at which the Z ring is attached to the rest of the compound; R₂ is selected from hydrogen, halogen, cyano, an optionally substituted C₁₋₆ alkyl, an optionally substituted C₁₋₃ alkoxy and optionally substituted C₃₋₆ cycloalkyl, preferably hydrogen, halogen, cyano, an optionally substituted C₁₋₃ alkyl and optionally substituted C₃₋₆ cycloalkyl; R₂′ is selected from hydrogen, halogen, cyano, an optionally substituted C₁₋₆ alkyl, an optionally substituted C₁₋₃ alkoxy and optionally substituted C₃₋₆ cycloalkyl, preferably hydrogen, halogen, cyano, an optionally substituted C₁₋₃ alkyl and optionally substituted C₃₋₆ cycloalkyl; R₃ is selected from a group consisting of hydrogen, an optionally substituted alkyl and an optionally substituted cycloalkyl, preferably hydrogen and an optionally substituted C₁₋₃ alkyl. Preferably, R₂, R₂′ and R₃ are as defined in any of the foregoing embodiments.

In one or more embodiments of the compound of Formulae IVb and IVc, the Z ring is selected from the following groups:

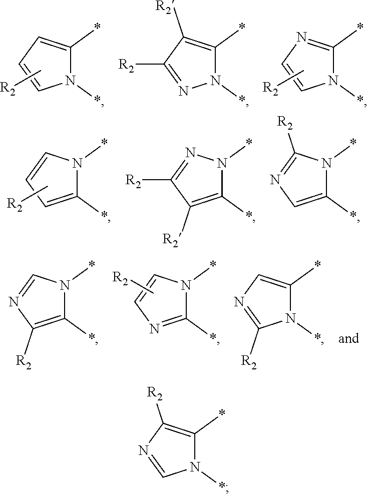

wherein R₂ and R₂′ are as defined in any of the foregoing embodiments; preferably, in these embodiments, at least one of A₂ and A₃ is CR₁, and R₁ is halogen; in some embodiments, both A₂ and A₃ are CR₁, and at least one of the R₁ groups is halogen. In some embodiments, A₃ is CR₁, and R₁ is halogen. In some embodiments, A₁ is CR₁, both of A₂ and A₃ are CH; or A₁ is CH, A₂ is CR₁, A₃ is CH; or both of A₁ and A₂ are CH, A₃ is CR₁, wherein R₁ is halogen.

In one or more embodiments of the compound of Formula VI (including Formulae Via, VIb and Vic), R₉ is hydrogen, R₁₀ is halogen or C₁₋₃ alkyl. In one or more embodiments, R₉ is halogen or C₁₋₃ alkyl, R₁₀ is hydrogen. In some embodiments, R₉ is hydrogen, R₁₀ is halogen; or R₉ is halogen, R₁₀ is hydrogen. Halogen is preferably F.

In one or more embodiments of the compound of Formula VI (including Formulae VIa, VIb and VIc), D₁ is N or CH.

In one or more embodiments of the compound of Formula VI (including Formulae VIa, VIb and VIc), R₇ and R₇′ are each independently hydrogen, halogen, cyano, optionally substituted C₁₋₃ alkyl, optionally substituted C₁₋₃ alkoxy or optionally substituted C₃₋₆ cycloalkyl, preferably, R₇ and R₇′ are each independently hydrogen, C₁₋₃ alkyl, halogenated C₁₋₃ alkyl, halogen, cyano or C₃₋₆ cycloalkyl, more preferably, R₇ and R₇′ are independently hydrogen, C₁₋₃ alkyl, halogenated C₁₋₃ alkyl, cyano or halogen; preferably, at least one of R₇ and R₇′ is not hydrogen. Preferably, R₇ is C₁₋₃ alkyl, halogenated C₁₋₃ alkyl, halogen, cyano or C₃₋₆ cycloalkyl, R₇′ is hydrogen; or R₇ is hydrogen, R₇′ is C₁₋₃ alkyl, halogenated C₁₋₃ alkyl, halogen, cyano or C₃₋₆ cycloalkyl. More preferably, R₇ is C₁₋₃ alkyl, halogenated C₁₋₃ alkyl, halogen, cyano or C₃₋₆ cycloalkyl, R₇′ is hydrogen.

In one or more embodiments of the compound of Formula VI (including Formulae VIa, VIb and VIc), Q is —C(O)—NR′R″ as described in any of the foregoing embodiments, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or optionally substituted heteroaryl, preferably hydrogen, an optionally substituted C₁₋₄ alkyl, an optionally substituted C₃₋₆ cycloalkyl or an optionally substituted 3-6 membered heterocyclic group. In one or more embodiments, Q is a 5 membered heteroaryl optionally substituted by 1-3 substituents selected from halogen, cyano, C₁₋₃ alkyl, halogenated C₁₋₃ alkyl, C₁₋₃ alkoxy and halogenated C₁₋₃ alkoxy, the 5 membered heteroaryl is preferably pyrrolyl, pyrazolyl, imidazolyl or triazolyl.

In one or more embodiments of the compound of Formula VIa, one of Z₁, Z₂ and Z₃ is O, the other two are CR₁, wherein, R₁ is selected from hydrogen, halogen and C₁₋₃ alkyl. Preferably, Z₁ is O, both of Z₂ and Z₃ are CH. Preferably, in some embodiments, R₉ is non-hydrogen substituent, such as halogen, optionally substituted C₁₋₃ alkyl or optionally substituted C₁₋₃ alkoxy, preferably halogen, more preferably F. Further preferably, in some embodiments, R₁₀ is hydrogen, halogen or C₁₋₃ alkyl, preferably hydrogen; D₁ is N or CH; R₇ is hydrogen, C₁₋₃ alkyl, halogenated C₁₋₃ alkyl, halogen, cyano or C₃₋₆ cycloalkyl, preferably hydrogen, halogen, C₁₋₃ alkyl or halogenated C₁₋₃ alkyl; R₇′ is hydrogen, C₁₋₃ alkyl, halogenated C₁₋₃ alkyl, halogen, cyano or C₃₋₆ cycloalkyl, preferably hydrogen or halogen. Q is —C(O)—NR′R″ or pyrrolyl, pyrazolyl, imidazolyl or triazolyl optionally substituted by 1-2 groups selected from halogen and C₁₋₃ alkyl, wherein, R′ and R″ are each independently selected from hydrogen, C₁₋₄ alkyl or C₃₋₆ cycloalkyl.

In one or more embodiments of the compound of Formula VIa, one of Z₁, Z₂ and Z₃ is O, the other two are CR₁, preferably, Z₁ is O, both of Z₂ and Z₃ are CH; R₁ is selected from hydrogen, halogen and C₁₋₃ alkyl; R₁₀ is non-hydrogen substituent, such as halogen, optionally substituted C₁₋₃ alkyl or optionally substituted C₁₋₃ alkoxy, preferably halogen, more preferably F; R₉ is hydrogen, halogen or C₁₋₃ alkyl, preferably hydrogen; D₁ is N or CH; R₇ is hydrogen, C₁₋₃ alkyl, halogenated C₁₋₃ alkyl, halogen, cyano or C₃₋₆ cycloalkyl, preferably hydrogen, halogen, C₁₋₃ alkyl or halogenated C₁₋₃ alkyl; R₇′ is hydrogen, C₁₋₃ alkyl, halogenated C₁₋₃ alkyl, halogen, cyano or C₃₋₆ cycloalkyl, preferably hydrogen or halogen. Q is —C(O)—NR′R″ or pyrrolyl, pyrazolyl, imidazolyl or triazolyl optionally substituted by 1-2 groups selected from halogen and C₁₋₃ alkyl, wherein, R′ and R″ are each independently selected from hydrogen, C₁₋₄ alkyl or C₃₋₆ cycloalkyl; preferably, in these embodiments, excluded compounds where R₉ is H, R₁₀ is F, R₇ is methyl, D₁ is N, R₇′ is H, Q is —C(O)—NH(CH₃) or —C(O)—NH(CD₃), and where R₉ is H, R₁₀ is F, R₇ is F, D₁ is N, R₇′ is H, Q is —C(O)—NH(CH₃) or —C(O)—NH(CD₃).

In some embodiments of the compound of Formula VIc, the Z ring is:

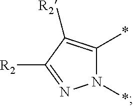

wherein, * indicates the position at which the Z ring is attached to the rest of the compound; R₂ is selected from hydrogen, halogen, cyano, optionally substituted alkyl, optionally substituted C₁₋₃ alkoxy and optionally substituted C₃₋₆ cycloalkyl, preferably hydrogen, halogen, cyano, optionally substituted C₁₋₃ alkyl and optionally substituted C₃₋₆ cycloalkyl; R₂′ is selected from hydrogen, halogen, cyano, optionally substituted C₁₋₆ alkyl, optionally substituted C₁₋₃ alkoxy and optionally substituted C₃₋₆ cycloalkyl, preferably hydrogen, halogen, cyano, optionally substituted C₁₋₃ alkyl and optionally substituted C₃₋₆ cycloalkyl; moreover, at least one of R₂ and R₂′ are non-hydrogen substituent, preferably, the non-hydrogen substituent is halogen, cyano, C₁₋₃ alkyl or C₃₋₆ cycloalkyl; one of R₉ and R₁₀ is halogen or C₁₋₃ alkyl, the other is hydrogen; D₁ is N or CH; R₇ is hydrogen, C₁₋₃ alkyl, halogenated C₁₋₃ alkyl, halogen, cyano or C₃₋₆ cycloalkyl, preferably hydrogen, halogen, C₁₋₃ alkyl or halogenated C₁₋₃ alkyl; R₇′ is hydrogen, C₁₋₃ alkyl, halogenated C₁₋₃ alkyl, halogen, cyano or C₃₋₆ cycloalkyl, preferably hydrogen or halogen; Q is —C(O)—NR′R″ or pyrrolyl, pyrazolyl, imidazolyl or triazolyl optionally substituted by 1-2 groups selected from halogen and C₁₋₃ alkyl, wherein, R′ and R″ are each independently selected from hydrogen, C₁₋₄ alkyl or C₃₋₆ cycloalkyl.

In one or more embodiments of the compound of Formula VIc, the Z ring is:

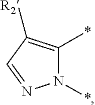

wherein, R₂′ is selected from halogen, cyano, optionally substituted C₁₋₆ alkyl, optionally substituted C₁₋₃ alkoxy and optionally substituted C₃₋₆ cycloalkyl, preferably halogen, cyano, optionally substituted C₁₋₃ alkyl and optionally substituted C₃₋₆ cycloalkyl; R₈ is H, one of R₉ and R₁₀ is halogen or C₁₋₃ alkyl, the other one is hydrogen; D₁ is N or CH; R₇ is hydrogen, C₁₋₃ alkyl, halogenated C₁₋₃ alkyl, halogen, cyano or C₃₋₆ cycloalkyl, preferably hydrogen, halogen, C₁₋₃ alkyl or halogenated C₁₋₃ alkyl; R₇′ is hydrogen, C₁₋₃ alkyl, halogenated C₁₋₃ alkyl, halogen, cyano or C₃₋₆ cycloalkyl, preferably hydrogen or halogen; Q is —C(O)—NR′R″ or pyrrolyl, pyrazolyl, imidazolyl or triazolyl optionally substituted by 1-2 groups selected from halogen and C₁₋₃ alkyl, wherein, R′ and R″ are each independently selected from hydrogen, C₁₋₄ alkyl or C₃₋₆ cycloalkyl.

In one or more embodiments of the compound of Formula I, preferred compounds are presented by Formula VII, or stereoisomers, tautomers, N-oxides, hydrates, solvates, isotope-substituted derivatives, or pharmaceutically acceptable salts thereof, or mixtures thereof, or prodrugs thereof,

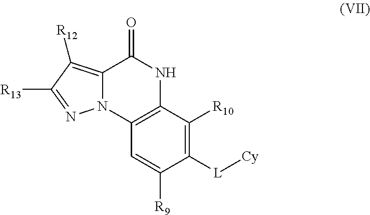

wherein, R₉ and R₁₀ are as defined in Formula VI; L and Cy are as defined in any of the foregoing embodiments.

R₁₂ and R₁₃ are each independently selected from hydrogen, halogen, cyano, an optionally substituted C₁₋₆ alkyl, an optionally substituted C₁₋₃ alkoxy and optionally substituted C₃₋₆ cycloalkyl, and at least one of R₁₂ and R₁₃ is not hydrogen.

In one or more embodiments of the compound of Formula VII, R₁₂ and R₁₃ are each independently hydrogen, halogen, cyano, an optionally substituted C₁₋₃ alkyl or optionally substituted C₃₋₆ cycloalkyl. In one or more embodiments, R₁₂ is hydrogen, and R₁₃ is halogen, cyano, an optionally substituted C₁₋₃ alkyl or optionally substituted C₃₋₆ cycloalkyl. In one or more embodiments, R₁₂ is halogen, cyano, an optionally substituted C₁₋₃ alkyl or optionally substituted C₃₋₆ cycloalkyl, and R₁₃ is hydrogen. In preferred embodiments, R₁₂ is halogen, cyano, an optionally substituted C₁₋₃ alkyl or optionally substituted C₃₋₆ cycloalkyl, and R₁₃ is hydrogen. More preferably, R₁₂ is halogen, C₁₋₃ alkyl, halogenated C₁₋₃ alkyl; and R₁₃ is hydrogen.

In one or more embodiments of the compound of Formula VII, R₉ is hydrogen, R₁₀ is halogen or C₁₋₃ alkyl. In one or more embodiments, R₉ is halogen or C₁₋₃ alkyl, R₁₀ is hydrogen. In some embodiments, R₉ is hydrogen, R₁₀ is halogen; or R₉ is halogen, R₁₀ is hydrogen. Preferably, the halogen is F.

In one or more embodiments of the compound of Formula VII, L is an alkylene optionally substituted by 1-2 C₁₋₃ alkyl, more preferably C₁₋₃ alkylene optionally substituted by 1-2 C₁₋₃ alkyl, preferably methylene optionally substituted by 1-2 C₁₋₃ alkyl.

In one or more embodiments of the compound of Formula VII, L is an unsubstituted alkylene, preferably an unsubstituted C₁₋₃ alkylene, more preferably methylene.

In one or more embodiments of the compound of Formula VII, Cy is an optionally substituted 5-7 membered nitrogen-containing heterocyclic group. Preferably, the 5-7 membered nitrogen-containing heterocyclic group is covalently attached to L through its ring nitrogen atom. Further preferably, Cy is an optionally substituted piperazinyl, piperidinyl, or dihyropyridinyl. Preferably, the substituents on Cy of the compound of Formula VII are selected from a group consisting of halogen, optionally substituted C₁₋₄ alkyl, optionally substituted C₁₋₄ alkoxy, halogenated C₁₋₄ alkyl, halogenated C₁₋₄ alkoxy, cyano, hydroxyl, amino (—NR′R″), an optionally substituted 6-14 membered aryl, an optionally substituted 5-10 membered heteroaryl, an optionally substituted 4-10 membered heterocyclic group and an optionally substituted C₃₋₈ cycloalkyl; wherein, the optionally substituted 6-14 membered aryl, the optionally substituted 5-10 membered heteroaryl, the optionally substituted 4-10 membered heterocyclic group and the optionally substituted C₃₋₈ cycloalkyl each can be independently substituted by 1-5 substituents selected from a group consisting of halogen, cyano, C₁₋₄ alkyl, C₁₋₄ alkoxy, halogenated C₁₋₄ alkyl, halogenated C₁₋₄ alkoxy, C₃₋₆ cycloalkyl, amino (—NR′R″), aminoacyl (—C(O)—NR′R″) and carboxyl; wherein the said R′ and R″ each are preferably independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably H, an optionally substituted C₁₋₄ alkyl, an optionally substituted C₃₋₈ cycloalkyl or an optionally substituted 3-6 membered heterocyclic group. In some preferred embodiments, the said substituents on 6-14 membered aryl, 5-10 membered heteroaryl, 4-10 membered heterocyclic group and C₃₋₈ cycloalkyl include at least an aminoacyl (—C(O)—NR′R″), and optionally include one or two of substituents selected from a group consisting of halogen, cyano, C₁₋₄ alkyl, halogenated C₁₋₄ alkyl and C₃₋₆ cycloalkyl. In some preferred embodiments, Cy is substituted by an optionally substituted 5-10 membered heteroaryl, preferably an optionally substituted 5-10 membered nitrogen-containing heteroaryl (such as pyridyl, pyrazinyl, pyrrolyl, imidazolyl, pyrazolyl, pyrimidinyl, etc.). Preferably, the 5-10 membered heteroaryl or 5-10 membered nitrogen-containing heteroaryl is at least substituted by an aminoacyl (—C(O)—NR′R″), and optionally further substituted by one or two substituents selected from a group consisting of halogen, cyano, C₁₋₄ alkyl, halogenated C₁₋₄ alkyl and C₃₋₆ cycloalkyl. Further preferably, the said aminoacyl (—C(O)—NR′R″) is at the para position. In some particularly preferred embodiments, Cy is a piperazinyl substituted with an optionally substituted pyridyl, a piperidinyl substituted with an optionally substituted pyridyl, or a dihydropyridinyl substituted with an optionally substituted pyridyl, and the said pyridyl is at least substituted with an aminoacyl (—C(O)—NR′R″). Preferably, in the embodiments as described herein, when the said R′ and R″ are substituted, the substituents can be 1-5 groups selected from a group consisting of halogen, hydroxyl, C₁₋₄ alkyl, C₁₋₄ alkoxy, halogenated C₁₋₄ alkyl, halogenated C₁₋₄ alkoxy and amino, etc.

In one or more embodiments of the compound of Formula I, preferred compounds are presented by Formula VIII, or stereoisomers, tautomers, N-oxides, hydrates, solvates, isotope-substituted derivatives, or pharmaceutically acceptable salts thereof, or mixtures thereof, or prodrugs thereof,

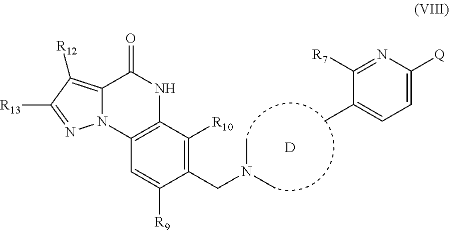

wherein, R₉ and R₁₀ are as defined in Formulae VI and VII; R₁₂ and R₁₃ are as defined in Formula VII; D ring and Q are as defined in Formula V; R₇ is as defined in Formulae IV and VI.

In one or more embodiments of the compound of Formula VIII, R₁₂ and R₁₃ are each independently hydrogen, halogen, cyano, an optionally substituted C₁₋₃ alkyl or optionally substituted C₃₋₆ cycloalkyl. In one or more embodiments, R₁₂ is hydrogen, and R₁₃ is halogen, cyano, an optionally substituted C₁₋₃ alkyl or optionally substituted C₃₋₆ cycloalkyl. In one or more embodiments, R₁₂ is halogen, cyano, an optionally substituted C₁₋₃ alkyl or optionally substituted C₃₋₆ cycloalkyl, and R₁₃ is hydrogen. In preferred embodiments, R₁₂ is halogen, cyano, an optionally substituted C₁₋₃ alkyl or optionally substituted C₃₋₆ cycloalkyl, and R₁₃ is hydrogen. More preferably, R₁₂ is halogen, C₁₋₃ alkyl, or halogenated C₁₋₃ alkyl; and R₁₃ is hydrogen.

In one or more embodiments of the compound of Formula VIII, R₉ is hydrogen, R₁₀ is halogen or C₁₋₃ alkyl. In one or more embodiments, R₉ is halogen or C₁₋₃ alkyl, R₁₀ is hydrogen. In some embodiments, R₉ is hydrogen, R₁₀ is halogen; or R₉ is halogen, R₁₀ is hydrogen. Preferably, the halogen is F.

In one or more embodiments of the compound of Formula VIII, the D ring is an optionally substituted 4-7 membered monocyclic ring containing at least 1 N atom; preferably the D ring is an optionally substituted piperazinyl, an optionally substituted piperidinyl, or an optionally substituted dihyropyridinyl. When the ring D is substituted, the substituents can be 1-3 groups selected from a group consisting of halogen, hydroxyl, cyano, optionally substituted C₁₋₃ alkyl, optionally substituted C₁₋₃ alkoxy and optionally substituted C₃₋₆ cycloalkyl.

In one or more embodiments of the compound of Formula VIII, the D ring is selected from the following groups:

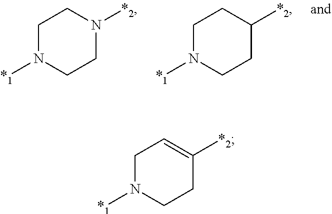

preferably, the D ring is

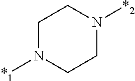

wherein *1 indicates the position at which the D ring is attached to methylene; *2 indicates the position at which the D ring is attached to the pyridyl.

In one or more embodiments of the compound of Formula VIII, R₇ is hydrogen, halogen, cyano, optionally substituted C₁₋₃ alkyl, optionally substituted C₁₋₃ alkoxy or optionally substituted C₃₋₆ cycloalkyl, preferably, R₇ is hydrogen, halogen, cyano, C₁₋₃ alkyl, halogenated C₁₋₃ alkyl or C₃₋₆ cycloalkyl.

In one or more embodiments of the compound of Formula VIII, Q is —C(O)—NR′R″ as described in any of the foregoing embodiments, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or optionally substituted heteroaryl, preferably hydrogen, an optionally substituted C₁₋₄ alkyl, an optionally substituted C₃₋₆ cycloalkyl or an optionally substituted 3-6 membered heterocyclic group. In one or more embodiments, Q is a 5 membered heteroaryl optionally substituted by 1-3 substituents selected from halogen, cyano, C₁₋₃ alkyl, halogenated C₁₋₃ alkyl, C₁₋₃ alkoxy and halogenated C₁₋₃ alkoxy, the 5 membered heteroaryl is preferably pyrrolyl, pyrazolyl, imidazolyl or triazolyl.

In one or more embodiments of the compound of Formula I, preferred compounds are presented by Formula IX, or stereoisomers, tautomers, N-oxides, hydrates, solvates, isotope-substituted derivatives, or pharmaceutically acceptable salts thereof, or mixtures thereof, or prodrugs thereof,

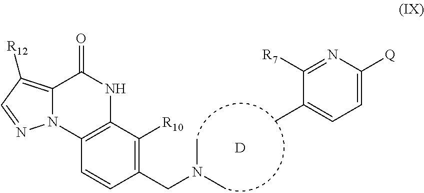

wherein, D ring and Q are as defined in Formula V; R₇ is as defined in Formulae IV and VI;
R₁₀ is halogen, optionally substituted C₁₋₃ alkyl, or optionally substituted C₁₋₃ alkoxy;
R₁₂ is halogen, cyano, an optionally substituted C₁₋₆ alkyl, an optionally substituted C₁₋₃ alkoxy and optionally substituted C₃₋₆ cycloalkyl.

In one or more embodiments of the compound of Formula IX, R₁₀ is halogen or C₁₋₃ alkyl. In one or more embodiments, R₁₀ is halogen, preferably F.

In one or more embodiments of the compound of Formula IX, R₁₂ is halogen, cyano, an optionally substituted C₁₋₃ alkyl or optionally substituted C₃₋₆ cycloalkyl. In preferred embodiments, R₁₂ is halogen, C₁₋₃ alkyl, or halogenated C₁₋₃ alkyl.

In one or more embodiments of the compound of Formula IX, the D ring is an optionally substituted 4-7 membered monocyclic ring containing at least 1 N atom; preferably the D ring is an optionally substituted piperazinyl, an optionally substituted piperidinyl, or an optionally substituted dihyropyridinyl. When the ring D is substituted, the substituents can be 1-3 groups selected from a group consisting of halogen, hydroxyl, cyano, optionally substituted C₁₋₃ alkyl, optionally substituted C₁₋₃ alkoxy and optionally substituted C₃₋₆ cycloalkyl.

In one or more embodiments of the compound of Formula IX, the D ring is selected from the following groups:

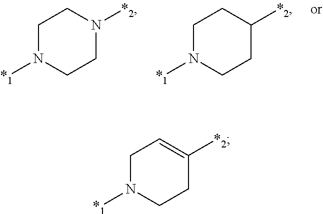

preferably, the D ring is

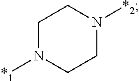

wherein *1 indicates the position at which the D ring is attached to methylene; *2 indicates the position at which the D ring is attached to the pyridyl.

In one or more embodiments of the compound of Formula IX, R₇ is hydrogen, halogen, cyano, optionally substituted C₁₋₃ alkyl, optionally substituted C₁₋₃ alkoxy or optionally substituted C₃₋₆ cycloalkyl, preferably, R₇ is hydrogen, halogen, cyano, C₁₋₃ alkyl, halogenated C₁₋₃ alkyl or C₃₋₆ cycloalkyl. More preferably, R₇ is halogen or C₁₋₃ alkyl.

In one or more embodiments of the compound of Formula IX, Q is —C(O)—NR′R″ as described in any of the foregoing embodiments, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or optionally substituted heteroaryl, preferably hydrogen, an optionally substituted C₁₋₄ alkyl, an optionally substituted C₃₋₆ cycloalkyl or an optionally substituted 3-6 membered heterocyclic group. More preferably, R′ is H, R″ is a C₁₋₄ alkyl optionally substituted by 1-5 halogen or a C₃₋₆ cycloalkyl.

It should be understood that although Z₁, Z₂, Z₃, Z₄, Z₅, A₁, A₂, A₃, L, Cy, R₆, R₇, R₈, R₉, R₁₀, R₁₂, R₁₃, D₁, B₁, B₂, B₃, B₄, W, Q, R′ and R″ are described separately above, the described features, especially the preferred features, can be arbitrarily combined to form the scope of different compounds of Formula I (including Formulae II, III, IV, V, VI, VII, VIII and IX) in this disclosure. For example, in some embodiments of compounds of Formula I (including Formulae II, III, IV, V, VI, VII, VIII and IX) of this disclosure.

The preferred compounds of Formula I include, without limitation:
7-((4-(2-methyl-6-(methylcarbamoyl)pyridin-3-yl)piperazin-1-yl)methyl)thieno[3,2-c]quinolin-4(5H)-one (Example 1);
7-((4-(2-methyl-6-(methylcarbamoyl)pyridin-3-yl)piperazin-1-yl)methyl)furo[2,3-c]quinolin-4(5H)-one (Example 2);
7-((4-(2-methyl-6-(methylcarbamoyl)pyridin-3-yl)piperazin-1-yl)methyl)thieno[3,4-c]quinolin-4(5H)-one (Example 3);
7-((4-(2-methyl-6-(methylcarbamoyl)pyridin-3-yl)piperazin-1-yl)methyl)thiazolo[4,5-c]quinolin-4(5H)-one (Example 4);
7-((4-(2-methyl-6-(methylcarbamoyl)pyridin-3-yl)piperazin-1-yl)methyl)thieno[2,3-c]quinolin-4(5H)-one (Example 5);
7-((4-(2-methyl-6-(methylcarbamoyl)pyridin-3-yl)piperazin-1-yl)methyl)furo[3,2-c]quinolin-4(5H)-one (Example 6);
7-((4-(2-methyl-6-(methylcarbamoyl)pyridin-3-yl)piperazin-1-yl)methyl)-1,5-dihydro-4H-pyrazolo[4,3-c]quinolin-4-one (Example 7);
7-((4-(2-methyl-6-(methylcarbamoyl)pyridin-3-yl)piperazin-1-yl)methyl)-1-methyl-1,5-dihydro-4H-pyrazolo[4,3-c]quinolin-4-one (Example 8);
7-((4-(2-methyl-6-(methylcarbamoyl)pyridin-3-yl)piperazin-1-yl)methyl)-2-methyl-2,5-dihydro-4H-pyrazolo[4,3-c]quinolin-4-one (Example 9);
7-((4-(2-methyl-6-(methylcarbamoyl)pyridin-3-yl)piperazin-1-yl)methyl)-3,5-dihydro-4H-pyrrolo[2,3-c]quinolin-4-one (Example 10);
7-((4-(2-methyl-6-(methylcarbamoyl)pyridin-3-yl)piperazin-1-yl)methyl)-3-methyl-3,5-dihydro-4H-pyrrolo[2,3-c]quinolin-4-one (Example 11);
7-((4-(2-methyl-6-(methylcarbamoyl)pyridin-3-yl)piperazin-1-yl)methyl)oxazolo[4,5-c]quinolin-4(5H)-one (Example 12);
7-((4-(2-methyl-6-(methylcarbamoyl)pyridin-3-yl)piperazin-1-yl)methyl)thiazolo[5,4-c]quinolin-4(5H)-one (Example 13);
7-((4-(2-methyl-6-(methylcarbamoyl)pyridin-3-yl)piperazin-1-yl)methyl)oxazolo[5,4-c]quinolin-4(5H)-one (Example 14);
7-((4-(2-methyl-6-(methylcarbamoyl)pyridin-3-yl)piperazin-1-yl)methyl)isoxazolo[3,4-c]quinolin-4(5H)-one (Example 15);
7-((4-(2-fluoro-6-(methylcarbamoyl)pyridin-3-yl)piperazin-1-yl)methyl)thieno[3,2-c]quinolin-4(5H)-one (Example 16);
7-((4-(2-fluoro-6-(methylcarbamoyl)pyridin-3-yl)piperazin-1-yl)methyl)thieno[3,4-c]quinolin-4(5H)-one (Example 17);
7-((4-(2-chloro-6-(methylcarbamoyl)pyridin-3-yl)piperazin-1-yl)methyl)thieno[3,2-c]quinolin-4(5H)-one (Example 18);
7-((4-(2-chloro-6-(methylcarbamoyl)pyridin-3-yl)piperazin-1-yl)methyl)thieno[3,4-c]quinolin-4(5H)-one (Example 19);
7-((4-(2-methyl-6-(methylcarbamoyl)pyridin-3-yl)piperazin-1-yl)methyl)-9-fluorothieno[3,2-c]quinolin-4(5H)-one (Example 20);
7-((4-(2-methyl-6-(methylcarbamoyl)pyridin-3-yl)piperazin-1-yl)methyl)-9-fluorothieno[3,4-c]quinolin-4(5H)-one (Example 21);
7-((4-(2-methyl-6-(methylcarbamoyl)pyridin-3-yl)piperazin-1-yl)methyl)-9-fluorofuro[3,2-c]quinolin-4(5H)-one (Example 22);
7-((4-(2-methyl-6-(methylcarbamoyl)pyridin-3-yl)piperazin-1-yl)methyl)-1,5-dihydro-4H-pyrrolo[3,2-c]quinolin-4-one (Example 23);
7-((4-(2-fluoro-6-(methylcarbamoyl)pyridin-3-yl)piperazin-1-yl)methyl)-1-methyl-1,5-dihydro-4H-pyrrolo[3,2-c]quinolin-4-one (Example 24);
7-((4-(2-methyl-6-(methylcarbamoyl)pyridin-3-yl)piperazin-1-yl)methyl)-1,5-dihydro-4H-imidazo[4,5-c]quinolin-4-one (Example 25);
7-((4-(2-methyl-6-(methylcarbamoyl)pyridin-3-yl)piperazin-1-yl)methyl)-1-methyl-1,5-dihydro-4H-imidazo[4,5-c]quinolin-4-one (Example 26);
7-((4-(2-methyl-6-(methylcarbamoyl)pyridin-3-yl)piperazin-1-yl)methyl)isothiazolo[4,5-c]quinolin-4(5H)-one (Example 27);
7-((4-(2-methyl-6-(methylcarbamoyl)pyridin-3-yl)piperazin-1-yl)methyl)-3-methyl-3,5-dihydro-4H-imidazo[4,5-c]quinolin-4-one (Example 28);
7-((4-(2-methyl-6-(methylcarbamoyl)pyridin-3-yl)piperazin-1-yl)methyl)isothiazolo[5,4-c]quinolin-4(5H)-one (Example 29);
7-((4-(2-methyl-6-(methylcarbamoyl)pyridin-3-yl)piperazin-1-yl)methyl)isoxazolo[5,4-c]quinolin-4(5H)-one (Example 30);
7-((4-(2-methyl-6-(methylcarbamoyl)pyridin-3-yl)piperazin-1-yl)methyl)isothiazolo[3,4-c]quinolin-4(5H)-one (Example 31);
7-((4-(2-methyl-6-(methylcarbamoyl)pyridin-3-yl)piperazin-1-yl)methyl)isothiazolo[4,3-c]quinolin-4(5H)-one (Example 32);
7-((4-(2-methyl-6-(methylcarbamoyl)pyridin-3-yl)piperazin-1-yl)methyl)isoxazolo[4,5-c]quinolin-4(5H)-one (Example 33);
7-((4-(2-methyl-6-(methylcarbamoyl)pyridin-3-yl)piperazin-1-yl)methyl)-2,5-dihydro-4H-pyrrolo[3,4-c]quinolin-4-one (Example 34);
7-((4-(2-methyl-6-(methylcarbamoyl)pyridin-3-yl)piperazin-1-yl)methyl)-2-methyl-2,5-dihydro-4H-pyrrolo[3,4-c]quinolin-4-one (Example 35);
7-((4-(2-methyl-6-(methylcarbamoyl)pyridin-3-yl)piperazin-1-yl)methyl)furo[3,4-c]quinolin-4(5H)-one (Example 36);
7-((4-(2-methyl-6-(methylcarbamoyl)pyridin-3-yl)piperazin-1-yl)methyl)isoxazolo[4,3-c]quinolin-4(5H)-one (Example 37);
9-fluoro-7-((4-(2-methyl-6-(methylcarbamoyl)pyridin-3-yl)piperazin-1-yl)methyl)thieno[2,3-c]quinolin-4(5H)-one (Example 38);
9-fluoro-7-((4-(2-methyl-6-(methylcarbamoyl)pyridin-3-yl)piperazin-1-yl)methyl)thieno[3,2-c]quinolin-4(5H)-one (Example 39);
9-fluoro-7-((4-(2-methyl-6-(methylcarbamoyl)pyridin-3-yl)piperazin-1-yl)methyl)furo[2,3-c]quinolin-4(5H)-one (Example 40);
9-fluoro-7-((4-(2-methyl-6-(methylcarbamoyl)pyridin-3-yl)piperazin-1-yl)methyl)thiazolo[4,5-c]quinolin-4(5H)-one (Example 41);
9-fluoro-7-((4-(2-methyl-6-(methylcarbamoyl)pyridin-3-yl)piperazin-1-yl)methyl)-1-methyl-1,5-dihydro-4H-pyrazolo[4,3-c]quinolin-4-one (Example 42);
7-((4-(2-methyl-6-(methylcarbamoyl)pyridin-3-yl)piperazin-1-yl)methyl)-6-fluoro-furo[3,2-c]quinolin-4(5H)-one (Example 43);
7-((4-(2-fluoro-6-(methylcarbamoyl)pyridin-3-yl)piperazin-1-yl)methyl)-6-fluoro-furo[3,2-c]quinolin-4(5H)-one (Example 44);
7-((4-(2-methyl-6-(methylcarbamoyl)pyridin-3-yl)piperazin-1-yl)methyl)-6-fluoro-1,5-dihydro-4H-pyrazolo[4,3-c]quinolin-4-one (Example 45);
7-((4-(2-fluoro-6-(methylcarbamoyl)pyridin-3-yl)piperazin-1-yl)methyl)-6-fluoro-1,5-dihydro-4H-pyrazolo[4,3-c]quinolin-4-one (Example 46);
7-((4-(2-methyl-6-(methylcarbamoyl)pyridin-3-yl)piperazin-1-yl)methyl)-3-methylisoxazolo[4,5-c]quinolin-4(5H)-one (Example 47);
8-((4-(2-methyl-6-(methylcarbamoyl)pyridin-3-yl)piperazin-1-yl)methyl)-7-fluoropyrrolo[1,2-c]quinazolin-5(6H)-one (Example 48);
7-((4-(2-fluoro-6-(methylcarbamoyl)pyridin-3-yl)piperazin-1-yl)methyl)-3-fluoropyrazolo[1,5-a]quinoxalin-4(5H)-one (Example 49);
8-((4-(2-methyl-6-(methylcarbamoyl)pyridin-3-yl)piperazin-1-yl)methyl)-7-fluoroimidazo[1,2-c]quinazolin-5(6H)-one (Example 50);
7-((4-(2-methyl-6-(methylcarbamoyl)pyridin-3-yl)piperazin-1-yl)methyl)-3-fluoro-2-methylpyrazolo[1,5-a]quinoxalin-4(5H)-one (Example 51);
7-((4-(2-fluoro-6-(methylcarbamoyl)pyridin-3-yl)piperazin-1-yl)methyl)-3-fluoro-2-methylpyrazolo[1,5-a]quinoxalin-4(5H)-one (Example 52);
7-((4-(2-fluoro-6-(methylcarbamoyl)pyridin-3-yl)piperazin-1-yl)methyl)-9-fluoroimidazo[1,5-a]quinoxalin-4(5H)-one (Example 53);
7-((4-(2-chloro-6-(methylcarbamoyl)pyridin-3-yl)piperazin-1-yl)methyl)-9-fluoroimidazo[1,5-a]quinoxalin-4(5H)-one (Example 54);
7-((4-(2-fluoro-6-(methylcarbamoyl)pyridin-3-yl)piperazin-1-yl)methyl)-6-fluoroimidazo[1,5-a]quinoxalin-4(5H)-one (Example 55);
7-((4-(2-chloro-6-(methylcarbamoyl)pyridin-3-yl)piperazin-1-yl)methyl)-6-fluoroimidazo[1,5-a]quinoxalin-4(5H)-one (Example 56);
7-((4-(2-methyl-6-(methylcarbamoyl)pyridin-3-yl)piperazin-1-yl)methyl)-6-fluoro-3-methylpyrazolo[1,5-a]quinoxalin-4(5H)-one (Example 57);
7-((4-(2-fluoro-6-(methylcarbamoyl)pyridin-3-yl)piperazin-1-yl)methyl)-6-fluoro-3-methylpyrazolo[1,5-a]quinoxalin-4(5H)-one (Example 58);
7-((4-(2-methyl-6-(methylcarbamoyl)pyridin-3-yl)piperazin-1-yl)methyl)-6-fluoro-3-chloro-pyrazolo[1,5-a]quinoxalin-4(5H)-one (Example 59);
7-((4-(2-fluoro-6-(methylcarbamoyl)pyridin-3-yl)piperazin-1-yl)methyl)-6-fluoro-3-chloro-pyrazolo[1,5-a]quinoxalin-4(5H)-one (Example 60);
8-((4-(2-fluoro-6-(methylcarbamoyl)pyridin-3-yl)piperazin-1-yl)methyl)-7-fluoroimidazo[1,2-c]quinazolin-5(6H)-one (Example 61);
8-((4-(2-fluoro-6-(ethylcarbamoyl)pyridin-3-yl)piperazin-1-yl)methyl)-7-fluoroimidazo[1,2-c]quinazolin-5(6H)-one (Example 62);
8-((4-(2-methyl-6-(ethylcarbamoyl)pyridin-3-yl)piperazin-1-yl)methyl)-7-fluoroimidazo[1,2-c]quinazolin-5(6H)-one (Example 63);
8-((4-(2-fluoro-6-(cyclopropylcarbamoyl)pyridin-3-yl)piperazin-1-yl)methyl)-7-fluoroimidazo[1,2-c]quinazolin-5(6H)-one (Example 64);
8-((4-(2-methyl-6-(cyclopropylcarbamoyl)pyridin-3-yl)piperazin-1-yl)methyl)-7-fluoroimidazo[1,2-c]quinazolin-5(6H)-one (Example 65);
7-((4-(2-fluoro-6-(ethylcarbamoyl)pyridin-3-yl)piperazin-1-yl)methyl)-6-fluorofuro[2,3-c]quinolin-4(5H)-one (Example 66);
7-((4-(2-fluoro-6-(cyclopropylcarbamoyl)pyridin-3-yl)piperazin-1-yl)methyl)-6-fluorofuro[2,3-c]quinolin-4(5H)-one (Example 67);
7-((4-(2-chloro-6-(methylcarbamoyl)pyridin-3-yl)piperazin-1-yl)methyl)-6-fluorofuro[2,3-c]quinolin-4(5H)-one (Example 68);
7-((4-(2-methyl-6-(methylcarbamoyl)pyridin-3-yl)piperazin-1-yl)methyl)-2-methyl-2,5-dihydro-4H-pyrazolo[3,4-c]quinolin-4-one (Example 69);
7-((4-(2-methyl-6-(methylcarbamoyl)pyridin-3-yl)piperazin-1-yl)methyl)-2,5-dihydro-4H-pyrazolo[3,4-c]quinolin-4-one (Example 70);
7-((4-(2-methyl-6-(methylcarbamoyl)pyridin-3-yl)piperazin-1-yl)methyl)-3-methyl-3,5-dihydro-4H-pyrazolo[3,4-c]quinolin-4-one (Example 71);
8-((4-(2-methyl-6-(methylcarbamoyl)pyridin-3-yl)piperazin-1-yl)methyl)-7-fluoropyrazolo[1,5-c]quinazolin-5(6H)-one (Example 72);
8-((4-(2-fluoro-6-(methylcarbamoyl)pyridin-3-yl)piperazin-1-yl)methyl)-7-fluoropyrazolo[1,5-c]quinazolin-5(6H)-one (Example 73);
7-((4-(6-methylcarbamoyl-2-methylpyridin-3-yl)piperazin-1-yl)methyl)-1,2,3,5-tetrahydro-4H-pyrrolo[3,4-c]quinolin-4-one (Example 74);
7-((4-(6-methylcarbamoyl-2-methylpyridin-3-yl)piperazin-1-yl)methyl)-2-methyl-1,2,3,5-tetrahydro-4H-pyrrolo[3,4-c]quinolin-4-one (Example 75);
7-((4-(6-methylcarbamoyl-2-methylpyridin-3-yl)piperazin-1-yl)methyl)-6-fluoro-1,2,3,5-tetrahydro-4H-pyrrolo[3,4-c]quinolin-4-one (Example 76);
7-((4-(6-methylcarbamoyl-2-fluoropyridin-3-yl)piperazin-1-yl)methyl)-6-fluoro-1,2,3,5-tetrahydro-4H-pyrrolo[3,4-c]quinolin-4-one (Example 77);
7-((4-(6-methylcarbamoyl-2-methylpyridin-3-yl)piperazin-1-yl)methyl)-6-fluoro-2-methyl-1,2,3,5-tetrahydro-4H-pyrrolo[3,4-c]quinolin-4-one (Example 78);
7-((4-(6-methylcarbamoyl-2-fluoropyridin-3-yl)piperazin-1-yl)methyl)-6-fluoro-2-methyl-1,2,3,5-tetrahydro-4H-pyrrolo[3,4-c]quinolin-4-one (Example 79);
7-((4-(6-methylcarbamoyl-2-methylpyridin-3-yl)piperazin-1-yl)methyl)-6-fluoro-1,2,3,5-tetrahydro-4H-cyclopenta[c]quinolin-4-one (Example 80);
7-((4-(6-methylcarbamoyl-2-fluoropyridin-3-yl)piperazin-1-yl)methyl)-6-fluoro-1,2,3,5-tetrahydro-4H-cyclopenta[c]quinolin-4-one (Example 81);
7-((4-(6-methylcarbamoyl-2-methylpyridin-3-yl)piperazin-1-yl)methyl)-6-fluoro-3,5-dihydrofuro[3,2-c]quinolin-4(2H)-one (Example 82);
7-((4-(6-methylcarbamoyl-2-fluoropyridin-3-yl)piperazin-1-yl)methyl)-6-fluoro-3,5-dihydrofuro[3,2-c]quinolin-4(2H)-one (Example 83);
7-((4-(6-methylcarbamoyl-2-methylpyridin-3-yl)piperazin-1-yl)methyl)-6-fluoro-3,5-dihydrofuro[3,4-c]quinolin-4(1H)-one (Example 84);
7-((4-(6-methylcarbamoyl-2-fluoropyridin-3-yl)piperazin-1-yl)methyl)-6-fluoro-3,5-dihydrofuro[3,4-c]quinolin-4(1H)-one (Example 85);
7-((4-(6-methylcarbamoyl-2-methylpyridin-3-yl)piperazin-1-yl)methyl)-6-fluoro-1,2-dihydrofuro[2,3-c]quinolin-4(5H)-one (Example 86);
7-((4-(6-methylcarbamoyl-2-fluoropyridin-3-yl)piperazin-1-yl)methyl)-6-fluoro-1,2-dihydrofuro[2,3-c]quinolin-4(5H)-one (Example 87);
7-((4-(2-methyl-6-(methylcarbamoyl)pyridin-3-yl)piperazin-1-yl)methyl)-6-fluoro-2-methyl-2,5-dihydro-4H-pyrazolo[4,3-c]quinolin-4-one (Example 88);
7-((4-(2-methyl-6-(methylcarbamoyl)pyridin-3-yl)piperazin-1-yl)methyl)-8-fluoro-2-methyl-2,5-dihydro-4H-pyrazolo[4,3-c]quinolin-4-one (Example 89);
7-((4-(2-fluoro-6-(methylcarbamoyl)pyridin-3-yl)piperazin-1-yl)methyl)-6-fluoro-2-methyl-2,5-dihydro-4H-pyrazolo[4,3-c]quinolin-4-one (Example 90);
7-((4-(2-fluoro-6-(methylcarbamoyl)pyridin-3-yl)piperazin-1-yl)methyl)-8-fluoro-2-methyl-2,5-dihydro-4H-pyrazolo[4,3-c]quinolin-4-one (Example 91);
6-fluoro-7-((4-(2-fluoro-6-(methylcarbamoyl)pyridin-3-yl)piperazin-1-yl)methyl)-1-methyl-1,5-dihydro-4H-pyrazolo[4,3-c]quinolin-4-one (Example 92);
6-fluoro-7-((4-(2-fluoro-6-(methylcarbamoyl)pyridin-3-yl)piperazin-1-yl)methyl)-2-methyl-2,5-dihydro-4H-pyrazolo[3,4-c]quinolin-4-one (Example 93);
7-((4-(2-fluoro-6-(methylcarbamoyl)pyridin-3-yl)piperazin-1-yl)methyl)-8-fluorooxazolo[5,4-c]quinolin-4(5H)-one (Example 94);
7-((4-(2-fluoro-6-(methylcarbamoyl)pyridin-3-yl)piperazin-1-yl)methyl)-6-fluorooxazolo[5,4-c]quinolin-4(5H)-one (Example 95);
7-((4-(2-fluoro-6-(methylcarbamoyl)pyridin-3-yl)piperazin-1-yl)methyl)-3-methyl-6-fluoroisoxazolo[4,5-c]quinolin-4(5H)-one (Example 96);
7-((4-(2-fluoro-6-(methylcarbamoyl)pyridin-3-yl)piperazin-1-yl)methyl)-3-methyl-8-fluoroisoxazolo[4,5-c]quinolin-4(5H)-one (Example 97);
7-((4-(2-fluoro-6-(methylcarbamoyl)pyridin-3-yl)piperazin-1-yl)methyl)isoxazolo[4,5-c]quinolin-4(5H)-one (Example 98);
7-((4-(2-fluoro-6-(methylcarbamoyl)pyridin-3-yl)piperazin-1-yl)methyl)-3,5-dihydro-4H-pyrrolo[2,3-c]quinolin-4-one (Example 99);
7-((4-(2-fluoro-6-(methylcarbamoyl)pyridin-3-yl)piperazin-1-yl)methyl)-6-fluoro-1-methyl-1,5-dihydro-4H-imidazo[4,5-c]quinolin-4-one (Example 100);
7-((4-(2-(difluoromethyl)-6-(methylcarbamoyl)pyridin-3-yl)piperazin-1-yl)methyl)-6-fluorofuro[2,3-c]quinolin-4(5H)-one (Example 101);
7-((4-(2-fluoro-6-(methylcarbamoyl)pyridin-3-yl)piperazin-1-yl)methyl)-8-fluorofuro[2,3-c]quinolin-4(5H)-one (Example 102);
7-((4-(2-fluoro-6-(methylcarbamoyl)pyridin-3-yl)piperazin-1-yl)methyl)-6-methylfuro[2,3-c]quinolin-4(5H)-one (Example 103);
7-((4-(2-fluoro-6-(methylcarbamoyl)pyridin-3-yl)piperazin-1-yl)methyl)-8-methylfuro[2,3-c]quinolin-4(5H)-one (Example 104);
7-((4-(2-fluoro-6-(methylcarbamoyl)pyridin-3-yl)piperazin-1-yl)methyl)-6-chlorofuro[2,3-c]quinolin-4(5H)-one (Example 105);
7-((4-(2-fluoro-6-carbamoylpyridin-3-yl)piperazin-1-yl)methyl)-6-fluorofuro[2,3-c]quinolin-4(5H)-one (Example 106);
6-fluoro-7-((4-(5-fluoro-6-(methylcarbamoyl)pyridin-3-yl)piperazin-1-yl)methyl)furo[2,3-c]quinolin-4(5H)-one (Example 107);
6-fluoro-7-((4-(6-(methylcarbamoyl)-2-(trifluoromethyl)pyridin-3-yl)piperazin-1-yl)methyl)furo[2,3-c]quinolin-4(5H)-one (Example 108);
7-((4-(2-fluoro-6-(methylcarbamoyl)pyridin-3-yl)piperidin-1-yl)methyl)-6-fluorofuro[2,3-c]quinolin-4(5H)-one (Example 109);
7-((4-(2-fluoro-6-(4H-1,2,4-triazol-3-yl)pyridin-3-yl)piperazin-1-yl)methyl)-6-fluorofuro[2,3-c]quinolin-4(5H)-one (Example 110);
7-((4-(2-fluoro-6-(5-methyl-1H-imidazol-2-yl)pyridin-3-yl)piperazin-1-yl)methyl)-6-fluorofuro[2,3-c]quinolin-4(5H)-one (Example 111);
7-((4-(2-fluoro-6-(methylcarbamoyl)pyridin-3-yl)piperazin-1-yl)methyl)furo[2,3-c]quinolin-4(5H)-one (Example 112);
7-((4-(6-(methylcarbamoyl)pyridin-3-yl)piperazin-1-yl)methyl)furo[2,3-c]quinolin-4(5H)-one (Example 113);
7-((4-(2-fluoro-6-(methylcarbamoyl)pyridin-3-yl)piperazin-1-yl)methyl)furo[3,2-c]quinolin-4(5H)-one (Example 114);
7-((4-(2-fluoro-6-(methylcarbamoyl)pyridin-3-yl)piperazin-1-yl)methyl)-6-fluorofuro[3,4-c]quinolin-4(5H)-one (Example 115);
7-((4-(2-fluoro-6-(methylcarbamoyl)pyridin-3-yl)piperazin-1-yl)methyl)-6-fluoro-2-methylfuro[2,3-c]quinolin-4(5H)-one (Example 116);
7-((4-(2-fluoro-6-(methylcarbamoyl)pyridin-3-yl)piperazin-1-yl)methyl)-2,6-difluorofuro[2,3-c]quinolin-4(5H)-one (Example 117);
7-((4-(6-(methylcarbamoyl)pyridin-3-yl)piperazin-1-yl)methyl)furo[3,2-c]quinolin-4(5H)-one (Example 118);
8-((4-(2-methyl-6-(methylcarbamoyl)pyridin-3-yl)piperazin-1-yl)methyl)-7-fluoropyrazolo[1,5-c]quinazolin-5(6H)-one (Example 119);
8-((4-(2-fluoro-6-(methylcarbamoyl)pyridin-3-yl)piperazin-1-yl)methyl)-9-fluoropyrazolo[1,5-c]quinazolin-5(6H)-one (Example 120);
7-((4-(2-methyl-6-(methylcarbamoyl)pyridin-3-yl)piperazin-1-yl)methyl)-3,6-difluoropyrazolo[1,5-a]quinoxalin-4(5H)-one (Example 121);
7-((4-(2-fluoro-6-(methylcarbamoyl)pyridin-3-yl)piperazin-1-yl)methyl)-3,6-difluoropyrazolo[1,5-a]quinoxalin-4(5H)-one (Example 122);
7-((4-(2-fluoro-6-(methylcarbamoyl)pyridin-3-yl)piperazin-1-yl)methyl)-2,6-difluoropyrazolo[1,5-a]quinoxalin-4(5H)-one (Example 123);
6-fluoro-7-((4-(2-fluoro-6-(methylcarbamoyl)pyridin-3-yl)piperidin-1-yl)methyl)-3-methylpyrazolo[1,5-a]quinoxalin-4(5H)-one (Example 124);
7-((4-(2-fluoro-6-(ethylcarbamoyl)pyridin-3-yl)piperazin-1-yl)methyl)-6-fluoro-3-methylpyrazolo[1,5-a]quinoxalin-4(5H)-one (Example 125);
7-((4-(2-fluoro-6-(cyclopropylcarbamoyl)pyridin-3-yl)piperazin-1-yl)methyl)-6-fluoro-3-methylpyrazolo[1,5-a]quinoxalin-4(5H)-one (Example 126);
7-((4-(5-fluoro-6-(methylcarbamoyl)pyridin-3-yl)piperazin-1-yl)methyl)-6-fluoro-3-methylpyrazolo[1,5-a]quinoxalin-4(5H)-one (Example 127);
7-((4-(2-cyano-6-(methylcarbamoyl)pyridin-3-yl)piperazin-1-yl)methyl)-6-fluoro-3-methylpyrazolo[1,5-a]quinoxalin-4(5H)-one (Example 128);
7-((4-(2-fluoro-6-(2,2-difluoroethylcarbamoyl)pyridin-3-yl)piperazin-1-yl)methyl)-6-fluoro-3-methylpyrazolo[1,5-a]quinoxalin-4(5H)-one (Example 129);
7-((4-(2-fluoro-6-(1H-imidazol-5-yl)pyridin-3-yl)piperazin-1-yl)methyl)-6-fluoro-3-methylpyrazolo[1,5-a]quinoxalin-4(5H)-one (Example 130);
7-((4-(2-chloro-6-(methylcarbamoyl)pyridin-3-yl)piperazin-1-yl)methyl)-6-fluoro-3-methylpyrazolo[1,5-a]quinoxalin-4(5H)-one (Example 131);
7-((4-(2-difluoromethyl-6-(methylcarbamoyl)pyridin-3-yl)piperazin-1-yl)methyl)-6-fluoro-3-methylpyrazolo[1,5-a]quinoxalin-4(5H)-one (Example 132);
7-((4-(2-trifluoromethyl-6-(methylcarbamoyl)pyridin-3-yl)piperazin-1-yl)methyl)-6-fluoro-3-methylpyrazolo[1,5-a]quinoxalin-4(5H)-one (Example 133);
(R)-6-fluoro-3-methyl-7-((3-((6-(methylcarbamoyl)pyridin-3-yl)amino)pyrrolidin-1-yl)methyl)pyrazolo[1,5-a]quinoxalin-4(5H)-one (Example 134);
(R)-6-fluoro-7-((3-((2-fluoro-6-(methylcarbamoyl)pyridin-3-yl)amino)pyrrolidin-1-yl)methyl)-3-methylpyrazolo[1,5-a]quinoxalin-4(5H)-one (Example 135);
8-((4-(2-methyl-6-(methylcarbamoyl)pyridin-3-yl)piperazin-1-yl)methyl)-2-methyl-10-fluoroimidazo[1,2-c]quinazolin-5(6H)-one (Example 136);
8-((4-(2-fluoro-6-(methylcarbamoyl)pyridin-3-yl)piperazin-1-yl)methyl)-2-methyl-10-fluoroimidazo[1,2-c]quinazolin-5(6H)-one (Example 137);
8-((4-(2-fluoro-6-(methylcarbamoyl)pyridin-3-yl)piperazin-1-yl)methyl)-3,7-difluoroimidazo[1,2-c]quinazolin-5(6H)-one (Example 138);
8-((4-(2-fluoro-6-(methylcarbamoyl)pyridin-3-yl)piperazin-1-yl)methyl)-2,7-difluoroimidazo[1,2-c]quinazolin-5(6H)-one (Example 139);
8-((4-(2-fluoro-6-(methylcarbamoyl)pyridin-3-yl)piperazin-1-yl)methyl)-2-methyl-7-fluoroimidazo[1,2-c]quinazolin-5(6H)-one (Example 140);
8-((4-(2-fluoro-6-(methylcarbamoyl)pyridin-3-yl)piperazin-1-yl)methyl)-3-methyl-7-fluoroimidazo[1,2-c]quinazolin-5(6H)-one (Example 141);
7-((4-(2-fluoro-6-(methylcarbamoyl)pyridin-3-yl)piperazin-1-yl)methyl)-6-fluorooxazolo[4,5-c]quinolin-4(5H)-one (Example 142);
7-((4-(2-fluoro-6-(methylcarbamoyl)pyridin-3-yl)piperazin-1-yl)methyl)furo[3,4-c]quinolin-4(5H)-one (Example 143);
7-((4-(2-fluoro-6-(methylcarbamoyl)pyridin-3-yl)piperazin-1-yl)methyl)isoxazolo[3,4-c]quinolin-4(5H)-one (Example 144);
7-((4-(6-(methylcarbamoyl)pyridin-3-yl)piperazin-1-yl)methyl)-6-fluoroimidazo[1,5-a]quinoxalin-4(5H)-one (Example 145);
(R)-7-((3-((6-(methylcarbamoyl)pyridin-3-yl)amino)pyrrolidin-1-yl)methyl)-6-fluorofuro[2,3-c]quinolin-4(5H)-one (Example 146);
6-fluoro-7-((4-(2-fluoro-6-(2,2-difluoroethylcarbamoyl)pyridin-3-yl)piperazin-1-yl)methyl)furo[2,3-c]quinolin-4(5H)-one (Example 147);
6-fluoro-7-((4-(6-(methylcarbamoyl)pyridin-3-yl)piperazin-1-yl)methyl)furo[2,3-c]quinolin-4(5H)-one (Example 148);
6-fluoro-7-((4-(6-(methylcarbamoyl)pyridin-3-yl)piperazin-1-yl)methyl)furo[3,4-c]quinolin-4(5H)-one (Example 149);
6-fluoro-7-((4-(6-(methylcarbamoyl)pyridin-3-yl)piperazin-1-yl)methyl)furo[3,2-c]quinolin-4(5H)-one (Example 150);
7-((4-(2-chloro-6-(methylcarbamoyl)pyridin-3-yl)piperazin-1-yl)methyl)-6-fluoro-2-methylpyrazolo[1,5-a]quinoxalin-4(5H)-one (Example 151);
6-fluoro-3-methyl-7-((6-methylcarbamoyl-3′,6′-dihydro-[3,4′-bipyridin]-1′(2′H)-yl)methyl)pyrazolo[1,5-a]quinoxalin-4(5H)-one (Example 152);
6-fluoro-3-methyl-7-((4-(6-(methylcarbamoyl)pyridin-3-yl)piperazin-1-yl)methyl)pyrazolo[1,5-a]quinoxalin-4(5H)-one (Example 153);
6-fluoro-7-((4-(2-fluoro-4-(methylcarbamoyl)phenyl)piperazin-1-yl)methyl)-3-methylpyrazolo[1,5-a]quinoxalin-4(5H)-one (Example 154);
6-fluoro-7-((4-(2-chloro-4-(methylcarbamoyl)phenyl)piperazin-1-yl)methyl)-3-methylpyrazolo[1,5-a]quinoxalin-4(5H)-one (Example 155);
(R)-6-fluoro-7-((4-(2-fluoro-6-((tetrahydrofuran-3-yl)carbamoyl)pyridin-3-yl)piperazin-1-yl)methyl)-3-methylpyrazolo[1,5-a]quinoxalin-4(5H)-one (Example 156);
6-fluoro-3-methyl-7-((4-(8-(methylamino)-1,7-naphthyridin-3-yl)piperazin-1-yl)methyl)pyrazolo[1,5-a]quinoxalin-4(5H)-one (Example 157);
7-((4-(6-(1H-imidazol-2-yl)pyridin-3-yl)piperazin-1-yl)methyl)-6-fluoro-3-methylpyrazolo[1,5-a]quinoxalin-4(5H)-one (Example 158);
6-fluoro-7-((4-(2-fluoro-6-((methyl-d3)carbamoyl)pyridin-3-yl)piperazin-1-yl)methyl)-3-methylpyrazolo[1,5-a]quinoxalin-4(5H)-one (Example 169);
7-((4-(2-cyclopropyl-6-(methylcarbamoyl)pyridin-3-yl)piperazin-1-yl)methyl)-6-fluoro-3-methylpyrazolo[1,5-a]quinoxalin-4(5H)-one (Example 160);
6-fluoro-7-((4-(3-fluoro-4-(methylcarbamoyl)phenyl)piperazin-1-yl)methyl)-3-methylpyrazolo[1,5-a]quinoxalin-4(5H)-one (Example 161);
6-fluoro-7-((4-(2-methyl-4-(methylcarbamoyl)phenyl)piperazin-1-yl)methyl)-3-methylpyrazolo[1,5-a]quinoxalin-4(5H)-one (Example 162);
7-((4-(2-fluoro-6-(methylcarbamoyl)pyridin-3-yl)piperazin-1-yl)methyl)-3-ethyl-6-fluoropyrazolo[1,5-a]quinoxalin-4(5H)-one (Example 163);
6-fluoro-7-((4-(2-fluoro-6-cyanopyridin-3-yl)piperazin-1-yl)methyl)-3-methylpyrazolo[1,5-a]quinoxalin-4(5H)-one (Example 164);
6-fluoro-7-((2-fluoro-6-methylcarbamoyl-3′,6′-dihydro-[3,4′-bipyridin]-1′(2′H)-yl)methyl)-3-methylpyrazolo[1,5-a]quinoxalin-4(5H)-one (Example 165);
(R)-6-fluoro-7-((4-(2-fluoro-6-(methylcarbamoyl)pyridin-3-yl)-3-methylpiperazin-1-yl)methyl)-3-methylpyrazolo[1,5-a]quinoxalin-4(5H)-one (Example 166);
(R)-6-fluoro-3-methyl-7-((8-methylcarbamoyl-1,2,4a,5-tetrahydropyrazino[1,2-d]pyrido[2,3-b][1,4]oxazin-3(4H)-yl)methyl)pyrazolo[1,5-a]quinoxalin-4(5H)-one (Example 167);
8-fluoro-7-((4-(2-fluoro-6-(methylcarbamoyl)pyridin-3-yl)piperazin-1-yl)methyl)-3-methylpyrazolo[1,5-a]quinoxalin-4(5H)-one (Example 168);
8-fluoro-7-((4-(2-methyl-6-(methylcarbamoyl)pyridin-3-yl)piperazin-1-yl)methyl)-3-methylpyrazolo[1,5-a]quinoxalin-4(5H)-one (Example 169);
8-fluoro-7-((4-(2-chloro-6-(methylcarbamoyl)pyridin-3-yl)piperazin-1-yl)methyl)-3-methylpyrazolo[1,5-a]quinoxalin-4(5H)-one (Example 170);
7-((4-(2-fluoro-6-(methylcarbamoyl)pyridin-3-yl)piperazin-1-yl)methyl)-3,6-dimethylpyrazolo[1,5-a]quinoxalin-4(5H)-one (Example 171);
7-((4-(2-methyl-6-(methylcarbamoyl)pyridin-3-yl)piperazin-1-yl)methyl)-3,6-dimethylpyrazolo[1,5-a]quinoxalin-4(5H)-one (Example 172);
7-((4-(2-chloro-6-(methylcarbamoyl)pyridin-3-yl)piperazin-1-yl)methyl)-3,6-dimethylpyrazolo[1,5-a]quinoxalin-4(5H)-one (Example 173);
6-chloro-7-((4-(2-fluoro-6-(methylcarbamoyl)pyridin-3-yl)piperazin-1-yl)methyl)-3-methylpyrazolo[1,5-a]quinoxalin-4(5H)-one (Example 174);
6-chloro-7-((4-(2-methyl-6-(methylcarbamoyl)pyridin-3-yl)piperazin-1-yl)methyl)-3-methylpyrazolo[1,5-a]quinoxalin-4(5H)-one (Example 175);
6-chloro-7-((4-(2-chloro-6-(methylcarbamoyl)pyridin-3-yl)piperazin-1-yl)methyl)-3-methylpyrazolo[1,5-a]quinoxalin-4(5H)-one (Example 176);
3,9-difluoro-7-((4-(2-fluoro-6-(methylcarbamoyl)pyridin-3-yl)piperazin-1-yl)methyl)pyrazolo[1,5-a]quinoxalin-4(5H)-one (Example 177);
7-((4-(2-chloro-6-(methylcarbamoyl)pyridin-3-yl)piperazin-1-yl)methyl)-3,6-difluoropyrazolo[1,5-a]quinoxalin-4(5H)-one (Example 178);
7-((4-(2-fluoro-6-(methylcarbamoyl)pyridin-3-yl)piperazin-1-yl)methyl)-3-trifluoromethyl-6-fluoropyrazolo[1,5-a]quinoxalin-4(5H)-one (Example 179);
7-((4-(2-fluoro-6-(ethylcarbamoyl)pyridin-3-yl)piperazin-1-yl)methyl)-3,6-difluoropyrazolo[1,5-a]quinoxalin-4(5H)-one (Example 180);
7-((4-(2-fluoro-6-(cyclopropylcarbamoyl)pyridin-3-yl)piperazin-1-yl)methyl)-3,6-difluoropyrazolo[1,5-a]quinoxalin-4(5H)-one (Example 181);
7-((4-(2-fluoro-6-(methylcarbamoyl)pyridin-3-yl)piperazin-1-yl)methyl)-3,8-difluoropyrazolo[1,5-a]quinoxalin-4(5H)-one (Example 182);
7-((4-(2-methyl-6-(methylcarbamoyl)pyridin-3-yl)piperazin-1-yl)methyl)-3,8-difluoropyrazolo[1,5-a]quinoxalin-4(5H)-one (Example 183);
7-((4-(2-chloro-6-(methylcarbamoyl)pyridin-3-yl)piperazin-1-yl)methyl)-3,8-difluoropyrazolo[1,5-a]quinoxalin-4(5H)-one (Example 184);
7-((4-(2-fluoro-6-((methyl-d3)carbamoyl)pyridin-3-yl)piperazin-1-yl)methyl)-3,6-difluoropyrazolo[1,5-a]quinoxalin-4(5H)-one (Example 185);
7-((4-(2-fluoro-6-(2,2-difluoroethylcarbamoyl)pyridin-3-yl)piperazin-1-yl)methyl)-3,6-difluoropyrazolo[1,5-a]quinoxalin-4(5H)-one (Example 186);
7-((4-(2-cyano-6-(methylcarbamoyl)pyridin-3-yl)piperazin-1-yl)methyl)-3,6-difluoropyrazolo[1,5-a]quinoxalin-4(5H)-one (Example 187);
7-((4-(2-fluoro-6-(methylcarbamoyl)pyridin-3-yl)piperazin-1-yl)methyl)-3,6-difluoropyrazolo[1,5-a]quinoxalin-4(5H)-one (Example 188);
3-fluoro-7-((4-(2-fluoro-6-(methylcarbamoyl)pyridin-3-yl)piperazin-1-yl)methyl)-6-methylpyrazolo[1,5-a]quinoxalin-4(5H)-one (Example 189);
3-fluoro-7-((4-(2-methyl-6-(methylcarbamoyl)pyridin-3-yl)piperazin-1-yl)methyl)-6-methylpyrazolo[1,5-a]quinoxalin-4(5H)-one (Example 190);
3-fluoro-7-((4-(2-chloro-6-(methylcarbamoyl)pyridin-3-yl)piperazin-1-yl)methyl)-6-methylpyrazolo[1,5-a]quinoxalin-4(5H)-one (Example 191);
3-fluoro-7-((4-(2-fluoro-6-(methylcarbamoyl)pyridin-3-yl)piperazin-1-yl)methyl)-6-chloropyrazolo[1,5-a]quinoxalin-4(5H)-one (Example 192);
3-fluoro-7-((4-(2-methyl-6-(methylcarbamoyl)pyridin-3-yl)piperazin-1-yl)methyl)-6-chloropyrazolo[1,5-a]quinoxalin-4(5H)-one (Example 193);
3-fluoro-7-((4-(2-chloro-6-(methylcarbamoyl)pyridin-3-yl)piperazin-1-yl)methyl)-6-chloropyrazolo[1,5-a]quinoxalin-4(5H)-one (Example 194);
7-((4-(2-fluoro-6-cyanopyridin-3-yl)piperazin-1-yl)methyl)-3,6-difluoropyrazolo[1,5-a]quinoxalin-4(5H)-one (Example 195);
(R)-7-((4-(2-fluoro-6-(methylcarbamoyl)pyridin-3-yl)-3-methylpiperazin-1-yl)methyl)-3-6-difluoropyrazolo[1,5-a]quinoxalin-4(5H)-one (Example 196);
7-((2-fluoro-6-methylcarbamoyl-3′,6′-dihydro-[3,4′-bipyridin]-1′(2′H)-yl)methyl)-3,6-difluoropyrazolo[1,5-a]quinoxalin-4(5H)-one (Example 197);
(R)-7-((8-methylcarbamoyl-1,2,4a,5-tetrahydropyrazino[1,2-d]pyrido[2,3-b][1,4]oxazin-3(4H)-yl)methyl)-3,6-difluoropyrazolo[1,5-a]quinoxalin-4(5H)-one (Example 198);
6-fluoro-7-((4-(2-fluoro-6-(methylcarbamoyl)pyridin-3-yl)piperazin-1-yl)methyl)-2-methyl-3-chloropyrazolo[1,5-a]quinoxalin-4(5H)-one (Example 199);
6-fluoro-7-((4-(2-fluoro-6-(methylcarbamoyl)pyridin-3-yl)piperazin-1-yl)methyl)-2,3-dimethylpyrazolo[1,5-a]quinoxalin-4(5H)-one (Example 200);
7-((4-(2-cyano-6-(methylcarbamoyl)pyridin-3-yl)piperazin-1-yl)methyl)-6-fluoro-3-(trifluoromethyl)pyrazolo[1,5-a]quinoxalin-4(5H)-one (Example 201);
7-((4-(2-fluoro-6-(methylcarbamoyl)pyridin-3-yl)piperazin-1-yl)methyl)-3-cyano-6-fluoropyrazolo[1,5-a]quinoxalin-4(5H)-one (Example 202);
7-((4-(2-fluoro-6-(methylcarbamoyl)pyridin-3-yl)piperazin-1-yl)methyl)-3-isopropyl-6-fluoropyrazolo[1,5-a]quinoxalin-4(5H)-one (Example 203);
6-fluoro-7-(1-(4-(2-fluoro-6-(methylcarbamoyl)pyridin-3-yl)piperazin-1-yl)ethyl)-3-methylpyrazolo[1,5-a]quinoxalin-4(5H)-one (Example 204);
7-fluoro-8-((4-(2-fluoro-6-(methylcarbamoyl)pyridin-3-yl)piperazin-1-yl)methyl)imidazo[1,5-c]quinazolin-5(6H)-one (Example 205);
   or stereoisomers, tautomers, N-oxides, hydrates, isotope-substituted derivatives, solvates or pharmaceutically acceptable salts thereof, or mixtures thereof, or prodrugs thereof.

Some of the compounds of the present disclosure may exist as stereoisomers including optical isomers. The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁₋₄ alcohol according to methods known in the art); esters of hydroxy containing compounds (e.g., those obtained by condensation with a C₁₋₄ carboxylic acid, C₃₋₆ diacid or anhydride thereof, such as succinic anhydride and fumaric anhydride according to methods known in the art); imines of amino containing compounds (e.g., those obtained by condensation with a C₁₋₄ aldehyde or ketone according to methods known in the art); carbamate of amino containing compounds, such as those described by Leu, et al., (J. Med. Chem. 42: 3623-3628 (1999)) and Greenwald, et al., (J. Med. Chem. 42: 3657-3667 (1999)); and acetals and ketals of alcohol-containing compounds (e.g., those obtained by condensation with chloromethyl methyl ether or chloromethyl ethyl ether according to methods known in the art).

The compounds of this disclosure may be prepared using methods known to those skilled in the art, or the novel methods of this disclosure. Specifically, the compounds of this disclosure with Formula I (including Formulae II, III, IV, V, VI, VII, VIII and IX) can be prepared as illustrated by the exemplary reaction in Scheme 1. Amino transesterification and Suzuki coupling of methyl 2-bromothiophene-3-carboxylate and (2-amino-4-(methoxycarbonyl)phenyl)boronic acid under the catalysis of sodium acetate and Pd(dppf)Cl₂ produced methyl 4-oxo-4,5-dihydrothieno[3,2-c]quinoline-7-carboxylate. Reduction of methyl 4-oxo-4,5-dihydrothieno[3,2-c]quinoline-7-carboxylate with LiAlH₄ produced 7-(hydroxymethyl)thieno[3,2-c]quinolin-4(5H)-one. Chlorination of 7-(hydroxymethyl)thieno[3,2-c]quinolin-4(5H)-one with SOCl₂ produced 7-(chloromethyl)thieno[3,2-c]quinolin-4(5H)-one. Reaction of 7-(chloromethyl)thieno[3,2-c]quinolin-4(5H)-one and N,6-dimethyl-5-(piperazin-1-yl)picolinamide produced the target compound 7-((4-(2-methyl-6-(methylcarbamoyl)pyridin-3-yl)piperazin-1-yl)methyl)thieno[3,2-c]quinolin-4(5H)-one.

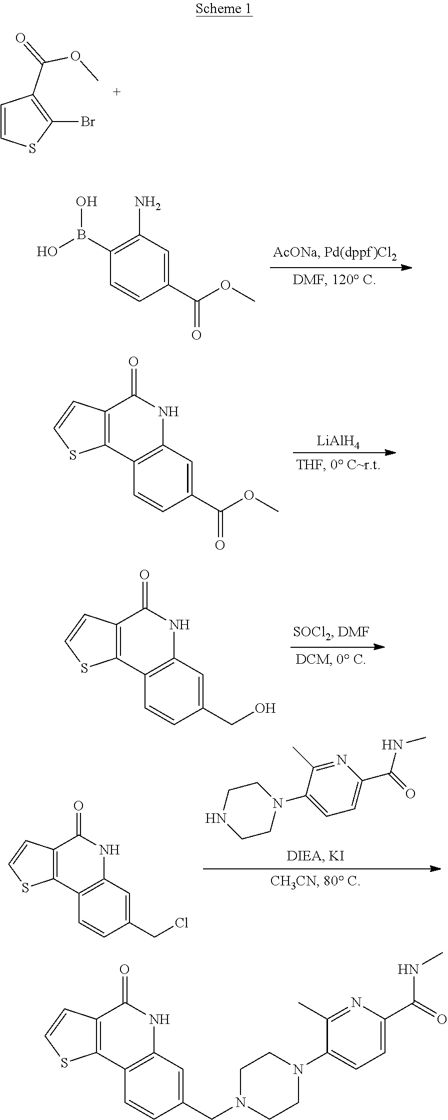

Other related compounds can be prepared using similar methods. For example, replacement of methyl 2-bromothiophene-3-carboxylate with methyl 4-bromothiophene-3-carboxylate produced the target compound 7-((4-(2-methyl-6-(methylcarbamoyl)pyridin-3-yl)piperazin-1-yl)methyl)thieno[3,4-c]quinolin-4(5H)-one. Replacement of methyl 2-bromothiophene-3-carboxylate with methyl 5-bromothiazole-4-carboxylate produced the target compound 7-((4-(2-methyl-6-(methylcarbamoyl)pyridin-3-yl)piperazin-1-yl)methyl)thiazolo[4,5-c]quinolin-4(5H)-one. Replacement of methyl 2-bromothiophene-3-carboxylate with methyl 3-bromothiophene-2-carboxylate produced the target compound 7-((4-(2-methyl-6-(methylcarbamoyl)pyridin-3-yl)piperazin-1-yl)methyl)thieno[2,3-c]quinolin-4(5H)-one. Replacement of methyl 2-bromothiophene-3-carboxylate with methyl 3-bromofuran-2-carboxylate produced the target compound 7-((4-(2-methyl-6-(methylcarbamoyl)pyridin-3-yl)piperazin-1-yl)methyl)furo[2,3-c]quinolin-4(5H)-one. Replacement of methyl 2-bromothiophene-3-carboxylate with methyl 3-bromo-1-methyl-1H-pyrrole-2-carboxylate produced the target compound 7-((4-(2-methyl-6-(methylcarbamoyl)pyridin-3-yl)piperazin-1-yl)methyl)-3-methyl-3,5-dihydro-4H-pyrrolo[2,3-c]quinolin-4-one.

The compounds of this disclosure can be prepared as illustrated by the exemplary reaction in Scheme 2. Heck coupling reaction of methyl furan-3-carboxylate and 1,4-dibromo-2-nitrobenzene under the catalysis of Pd(PPh₃)₄ produced methyl 2-(4-bromo-2-nitrophenyl)furan-3-carboxylate. Reaction of methyl 2-(4-bromo-2-nitrophenyl)furan-3-carboxylate and Fe, NH₄Cl produced 7-bromofuro[3,2-c]quinolin-4(5H)-one. Stille coupling reaction of 7-bromofuro[3,2-c]quinolin-4(5H)-one and (tributylstannyl)-methanol (Bu₃SnCH₂OH) under the catalysis of chloro(2-dicyclohexylphosphino-2′,4′,6′-tri-isopropyl-1,1′-biphenyl)(2′-amino-1,1′-biphenyl-2-yl) palladium(II) (XPhos Pd G2) produced 7-(hydroxymethyl)furo[3,2-c]quinolin-4(5H)-one. Reaction of 7-(hydroxymethyl)furo[3,2-c]quinolin-4(5H)-one and SOCl₂ produced 7-(chloromethyl)furo[3,2-c]quinolin-4(5H)-one. Reaction of 7-(chloromethyl)furo[3,2-c]quinolin-4(5H)-one and N,6-dimethyl-5-(piperazin-1-yl)picolinamide under the catalysis of DIEA and KI produced the target compound 7-((4-(2-methyl-6-(methylcarbamoyl)pyridin-3-yl)piperazin-1-yl)methyl)furo[3,2-c]quinolin-4(5H)-one.

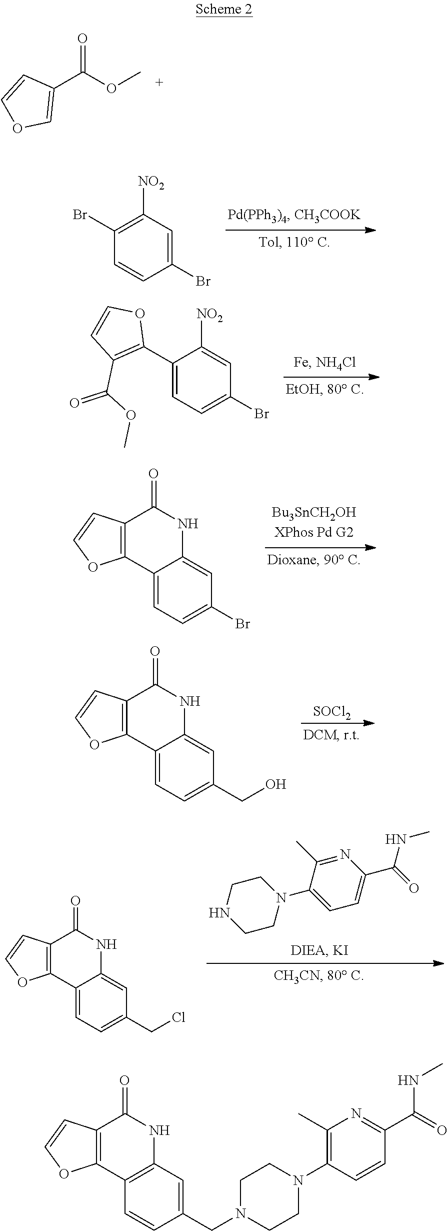

Other related compounds can be prepared using similar methods. For example, replacement of 1,4-dibromo-2-nitrobenzene with 1-bromo-2-fluoro-4-iodo-3-nitrobenzene produced the target compound 7-((4-(2-methyl-6-(methylcarbamoyl)pyridin-3-yl)piperazin-1-yl)methyl)-6-fluorofuro[3,2-c]quinolin-4(5H)-one. Replacement of N,6-dimethyl-5-(piperazin-1-yl)picolinamide with 6-fluoro-N-methyl-5-(piperazin-1-yl)picolinamide produced the target compound 7-((4-(2-fluoro-6-(methylcarbamoyl)pyridin-3-yl)piperazin-1-yl)methyl)-6-fluorofuro[3,2-c]quinolin-4(5H)-one.

The compounds of this disclosure can be prepared as illustrated by the exemplary reaction in Scheme 3. Reaction of ethyl 2-(4-bromo-2-nitrobenzoyl)-3-(dimethylamino)acrylate and hydrazinium hydroxide solution produced ethyl 5-(4-bromo-2-nitrophenyl)-1H-pyrazole-4-carboxylate. Reaction of ethyl 5-(4-bromo-2-nitrophenyl)-1H-pyrazole-4-carboxylate and Fe, AcOH produced 7-bromo-1,5-dihydro-4H-pyrazolo[4,3-c]quinolin-4-one. Reaction of 7-bromo-1,5-dihydro-4H-pyrazolo[4,3-c]quinolin-4-one and di-tert-butyl dicarbonate ((Boc)₂O) under the catalysis of 4-(dimethylamino)pyridine (DMAP) produced tert-butyl 7-bromo-4-oxo-4,5-dihydro-1H-pyrazolo[4,3-c]quinoline-1-carboxylate. Stille coupling of tert-butyl 7-bromo-4-oxo-4,5-dihydro-1H-pyrazolo[4,3-c]quinoline-1-carboxylate and Bu₃SnCH₂OH under the catalysis of XPhos Pd G2 produced 7-(hydroxymethyl)-1,5-dihydro-4H-pyrazolo[4,3-c]quinolin-4-one. Reaction of 7-(hydroxymethyl)-1,5-dihydro-4H-pyrazolo[4,3-c]quinolin-4-one and HBr produced 7-(bromomethyl)-1,5-dihydro-4H-pyrazolo[4,3-c]quinolin-4-one. Reaction of 7-(bromomethyl)-1,5-dihydro-4H-pyrazolo[4,3-c]quinolin-4-one and (Boc)₂O under the catalysis of DMAP produced tert-butyl 7-(bromomethyl)-4-oxo-4,5-dihydro-1H-pyrazolo[4,3-c]quinoline-1-carboxylate. Reaction of tert-butyl 7-(bromomethyl)-4-oxo-4,5-dihydro-1H-pyrazolo[4,3-c]quinoline-1-carboxylate and N,6-dimethyl-5-(piperazin-1-yl)picolinamide under the catalysis of DIEA and KI produced tert-butyl 7-((4-(2-methyl-6-(methylcarbamoyl)pyridin-3-yl)piperazin-1-yl)methyl)-4-oxo-4,5-dihydro-1H-pyrazolo[4,3-c]quinoline-1-carboxylate. Reaction of tert-butyl 7-((4-(2-methyl-6-(methylcarbamoyl)pyridin-3-yl)piperazin-1-yl)methyl)-4-oxo-4,5-dihydro-1H-pyrazolo[4,3-c]quinoline-1-carboxylate and HCl produced the target compound 7-((4-(2-methyl-6-(methylcarbamoyl)pyridin-3-yl)piperazin-1-yl)methyl)-1,5-dihydro-4H-pyrazolo[4,3-c]quinolin-4-one.

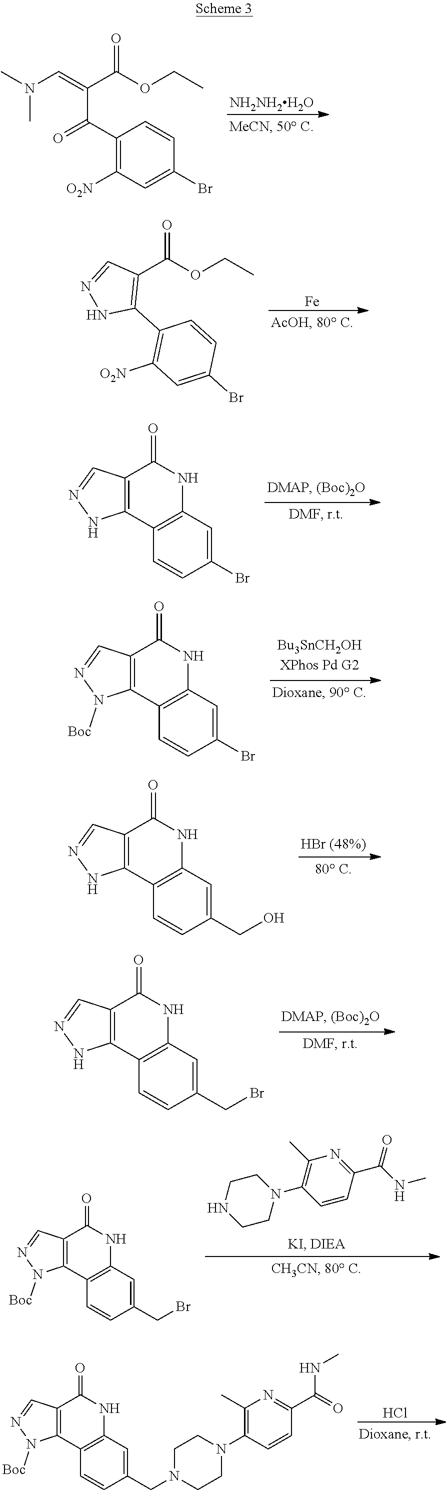

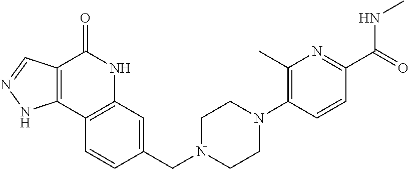 [SMILES: CNC(=O)c1ccc(N2CCN(Cc3ccc4c(c3)[nH]c(=O)c3cn[nH]c34)CC2)c(C)n1]

Other related compounds can be prepared using similar methods. For example, replacement of hydrazinium hydroxide solution with methylhydrazine produced the target compound 7-((4-(2-methyl-6-(methylcarbamoyl)pyridin-3-yl)piperazin-1-yl)methyl)-1-methyl-1,5-dihydro-4H-pyrazolo[4,3-c]quinolin-4-one. Replacement of ethyl 2-(4-bromo-2-nitrobenzoyl)-3-(dimethylamino)acrylate with ethyl 2-(4-bromo-3-fluoro-2-nitrobenzoyl)-3-(dimethylamino)acrylate produced the target compound 7-((4-(2-methyl-6-(methylcarbamoyl)pyridin-3-yl)piperazin-1-yl)methyl)-6-fluoro-1,5-dihydro-4H-pyrazolo[4,3-c]quinolin-4-one. Replacement of N,6-dimethyl-5-(piperazin-1-yl)picolinamide with 6-fluoro-N-methyl-5-(piperazin-1-yl)picolinamide produced the target compound 7-((4-(2-fluoro-6-(methylcarbamoyl)pyridin-3-yl)piperazin-1-yl)methyl)-6-fluoro-1,5-dihydro-4H-pyrazolo[4,3-c]quinolin-4-one.

The compounds of this disclosure can be prepared as illustrated by the exemplary reaction in Scheme 4. Suzuki coupling of (2-(ethoxycarbonyl)furan-3-yl)boronic acid and methyl 4-bromo-3-fluoro-5-nitrobenzoate under the catalysis of Pd(PPh₃)Cl₂ produced ethyl 3-(2-fluoro-4-(methoxycarbonyl)-6-nitrophenyl)furan-2-carboxylate. Reaction of ethyl 3-(2-fluoro-4-(methoxycarbonyl)-6-nitrophenyl)furan-2-carboxylate and Fe, AcOH produced methyl 9-fluoro-4-oxo-4,5-dihydrofuro[2,3-c]quinoline-7-carboxylate. Reduction of methyl 9-fluoro-4-oxo-4,5-dihydrofuro[2,3-c]quinoline-7-carboxylate with LiAlH₄ produced 9-fluoro-7-(hydroxymethyl)furo[2,3-c]quinolin-4(5H)-one. Reaction of 9-fluoro-7-(hydroxymethyl)furo[2,3-c]quinolin-4(5H)-one and HBr produced 7-(bromomethyl)-9-fluorofuro[2,3-c]quinolin-4(5H)-one. Reaction of 7-(bromomethyl)-9-fluorofuro[2,3-c]quinolin-4(5H)-one and N,6-dimethyl-5-(piperazin-1-yl)picolinamide under the catalysis of DIEA and KI produced the target compound 9-fluoro-7-((4-(2-methyl-6-(methylcarbamoyl)pyridin-3-yl)piperazin-1-yl)methyl)furo[2,3-c]quinolin-4(5H)-one.

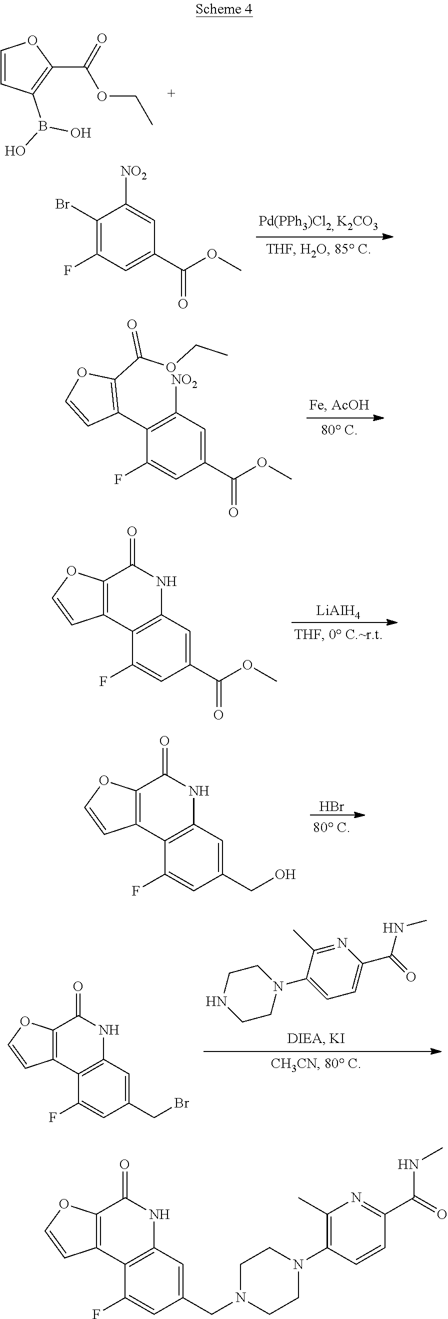

Other related compounds can be prepared using similar methods. For example, replacement of (2-(ethoxycarbonyl)furan-3-yl)boronic acid with methyl 3-(4,4,5,5-tetramethyl-1,3,2-dioxaborolan-2-yl)thiophene-2-carboxylate produced the target compound 9-fluoro-7-((4-(2-methyl-6-(methylcarbamoyl)pyridin-3-yl)piperazin-1-yl)methyl)thieno[2,3-c]quinolin-4(5H)-one.

The compounds of this disclosure can be prepared as illustrated by the exemplary reaction in Scheme 5. Reaction of 4-fluoro-1H-pyrazole-5-carboxylic acid and SOCl₂ produced 4-fluoro-1H-pyrazole-5-carbonyl chloride. Reaction of 4-fluoro-1H-pyrazole-5-carbonyl chloride and methyl 3-amino-4-fluorobenzoate under the catalysis of lithium bis(trimethylsilyl)amide (LiHMDS) produced methyl 4-fluoro-3-(4-fluoro-1H-pyrazole-5-carboxamido)benzoate. Ring closing of methyl 4-fluoro-3-(4-fluoro-1H-pyrazole-5-carboxamido)benzoate under the catalysis of K₂CO₃ produced methyl 3-fluoro-4-oxo-4,5-dihydropyrazolo[1,5-a]quinoxaline-7-carboxylate. Reaction of methyl 3-fluoro-4-oxo-4,5-dihydropyrazolo[1,5-a]quinoxaline-7-carboxylate and LiAlH₄ produced 3-fluoro-7-(hydroxymethyl)pyrazolo[1,5-a]quinoxalin-4(5H)-one. Reaction of 3-fluoro-7-(hydroxymethyl)pyrazolo[1,5-a]quinoxalin-4(5H)-one and SOCl₂ produced 7-(chloromethyl)-3-fluoropyrazolo[1,5-a]quinoxalin-4(5H)-one. Reaction of 7-(chloromethyl)-3-fluoropyrazolo[1,5-a]quinoxalin-4(5H)-one and 6-fluoro-N-methyl-5-(piperazin-1-yl)picolinamide under the catalysis of DIEA and KI produced the target compound 7-((4-(2-fluoro-6-(methylcarbamoyl)pyridin-3-yl)piperazin-1-yl)methyl)-3-fluoropyrazolo[1,5-a]quinoxalin-4(5H)-one.

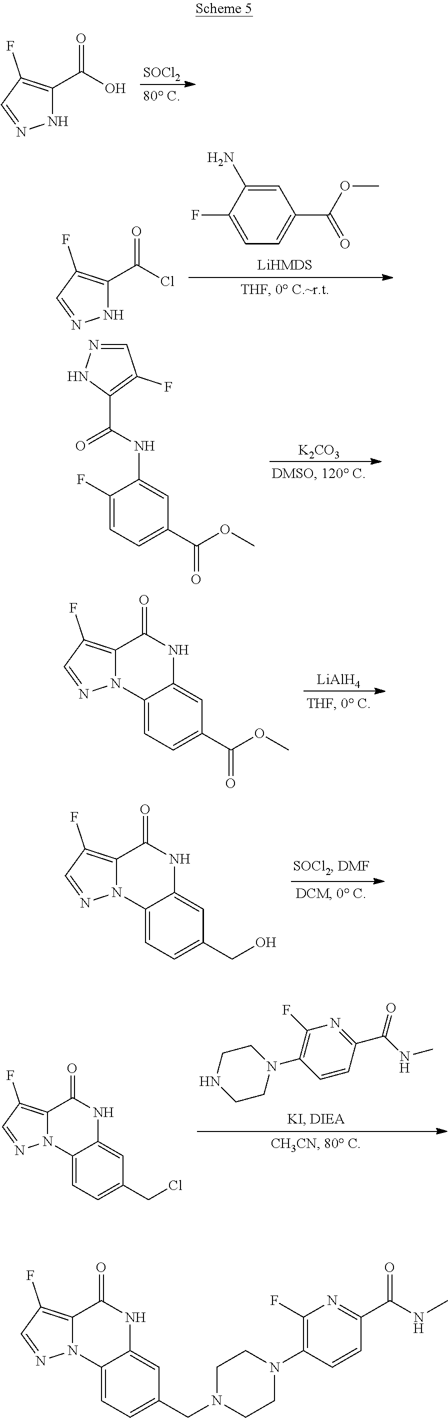

Other related compounds can be prepared using similar methods. For example, replacement of 4-fluoro-1H-pyrazole-5-carboxylic acid with 4-fluoro-3-methyl-1H-pyrazole-5-carboxylic acid produced the target compound 7-((4-(2-fluoro-6-(methylcarbamoyl)pyridin-3-yl)piperazin-1-yl)methyl)-3-fluoro-2-methylpyrazolo[1,5-a]quinoxalin-4(5H)-one. Replacement of 4-fluoro-1H-pyrazole-5-carboxylic acid with 1H-imidazole-5-carboxylic acid produced the target compound 7-((4-(2-fluoro-6-(methylcarbamoyl)pyridin-3-yl)piperazin-1-yl)methyl)-6-fluoroimidazo[1,5-a]quinoxalin-4(5H)-one.

The compounds of this disclosure can be prepared as illustrated by the exemplary reaction in Scheme 6. Reaction of 2-amino-4-bromo-3-fluorobenzonitrile and chlorosulfonyl isocyanate produced 4-amino-7-bromo-8-fluoroquinazolin-2(1H)-one. Reaction of 4-amino-7-bromo-8-fluoroquinazolin-2(1H)-one and chloroacetaldehyde under the catalysis of sodium acetate produced 8-bromo-7-fluoroimidazo[1,2-c]quinazolin-5(6H)-one. Reaction of 8-bromo-7-fluoroimidazo[1,2-c]quinazolin-5(6H)-one and Bu₃SnCH₂OH under the catalysis of XPhos Pd G2 produced 7-fluoro-8-(hydroxymethyl)imidazo[1,2-c]quinazolin-5(6H)-one. Reaction of 7-fluoro-8-(hydroxymethyl)imidazo[1,2-c]quinazolin-5(6H)-one and phosphorus tribromide produced 8-(bromomethyl)-7-fluoroimidazo[1,2-c]quinazolin-5(6H)-one. Reaction of 8-(bromomethyl)-7-fluoroimidazo[1,2-c]quinazolin-5(6H)-one and N,6-dimethyl-5-(piperazin-1-yl)picolinamide under the catalysis of DIEA and KI produced the target compound 8-((4-(2-methyl-6-(methylcarbamoyl)pyridin-3-yl)piperazin-1-yl)methyl)-7-fluoroimidazo[1,2-c]quinazolin-5(6H)-one.

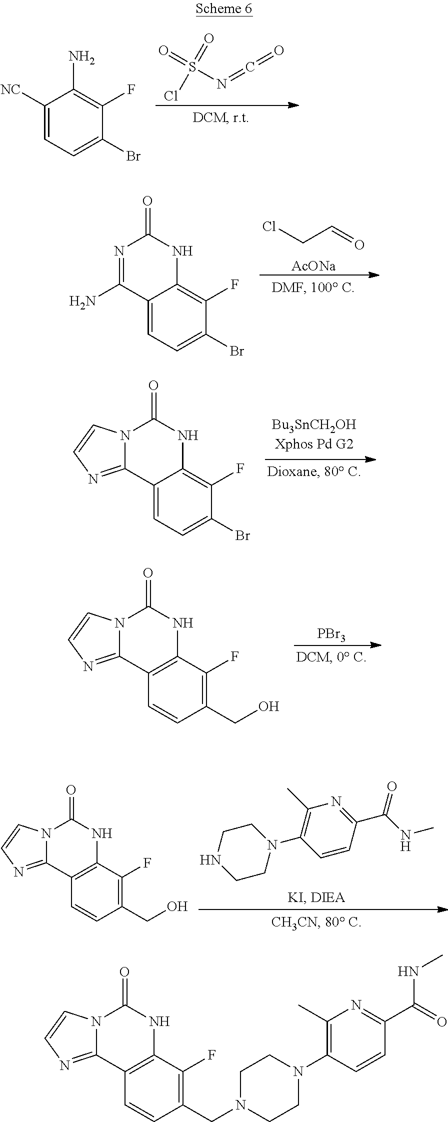

Other related compounds can be prepared using similar methods. For example, replacement of N,6-dimethyl-5-(piperazin-1-yl)picolinamide with 6-fluoro-N-methyl-5-(piperazin-1-yl)picolinamide produced the target compound 8-((4-(2-fluoro-6-(methylcarbamoyl)pyridin-3-yl)piperazin-1-yl)methyl)-7-fluoroimidazo[1,2-c]quinazolin-5(6H)-one. Replacement of N,6-dimethyl-5-(piperazin-1-yl)picolinamide with N-ethyl-6-fluoro-5-(piperazin-1-yl)picolinamide produced the target compound 8-((4-(2-fluoro-6-(ethylcarbamoyl)pyridin-3-yl)piperazin-1-yl)methyl)-7-fluoroimidazo[1,2-c]quinazolin-5(6H)-one. Replacement of N,6-dimethyl-5-(piperazin-1-yl)picolinamide with N-ethyl-6-methyl-5-(piperazin-1-yl)picolinamide produced the target compound 8-((4-(2-methyl-6-(ethylcarbamoyl)pyridin-3-yl)piperazin-1-yl)methyl)-7-fluoroimidazo[1,2-c]quinazolin-5(6H)-one. Replacement of N,6-dimethyl-5-(piperazin-1-yl)picolinamide with N-cyclopropyl-6-fluoro-5-(piperazin-1-yl)picolinamide produced the target compound 8-((4-(2-fluoro-6-(cyclopropylcarbamoyl)pyridin-3-yl)piperazin-1-yl)methyl)-7-fluoroimidazo[1,2-c]quinazolin-5(6H)-one. Replacement of N,6-dimethyl-5-(piperazin-1-yl)picolinamide with N-cyclopropyl-6-methyl-5-(piperazin-1-yl)picolinamide produced the target compound 8-((4-(2-methyl-6-(cyclopropylcarbamoyl)pyridin-3-yl)piperazin-1-yl)methyl)-7-fluoroimidazo[1,2-c]quinazolin-5(6H)-one.

The compounds of this disclosure can be prepared as illustrated by the exemplary reaction in Scheme 7. Suzuki coupling of (2-(ethoxycarbonyl)furan-3-yl)boronic acid and 1,4-dibromo-2-fluoro-3-nitrobenzene under the catalysis of Pd(PPh₃)Cl₂ produced ethyl 3-(4-bromo-3-fluoro-2-nitrophenyl)furan-2-carboxylate. Reaction of ethyl 3-(4-bromo-3-fluoro-2-nitrophenyl)furan-2-carboxylate and Fe, AcOH produced 7-bromo-6-fluorofuro[2,3-c]quinolin-4(5H)-one. Stille coupling of 7-bromo-6-fluorofuro[2,3-c]quinolin-4(5H)-one and Bu₃SnCH₂OH under the catalysis of XPhos Pd G2 produced 6-fluoro-7-(hydroxymethyl)furo[2,3-c]quinolin-4(5H)-one. Reaction of 6-fluoro-7-(hydroxymethyl)furo[2,3-c]quinolin-4(5H)-one and HBr produced 7-(bromomethyl)-6-fluorofuro[2,3-c]quinolin-4(5H)-one. Reaction of 7-(bromomethyl)-6-fluorofuro[2,3-c]quinolin-4(5H)-one and N-ethyl-6-fluoro-5-(piperazin-1-yl)picolinamide under the catalysis of DIEA and KI produced the target compound 7-((4-(2-fluoro-6-(ethylcarbamoyl)pyridin-3-yl)piperazin-1-yl)methyl)-6-fluorofuro[2,3-c]quinolin-4(5H)-one.

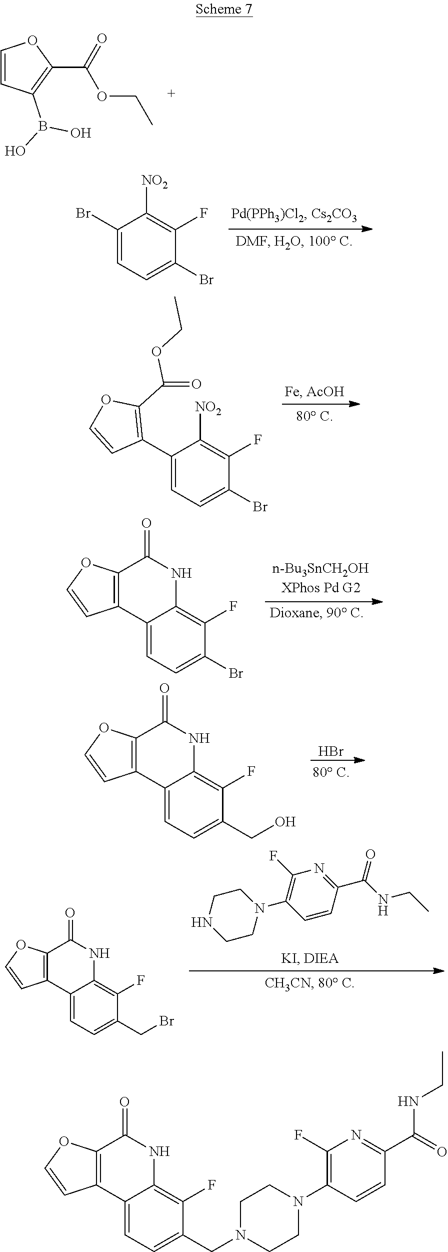

Other related compounds can be prepared using similar methods. For example, replacement of N-ethyl-6-fluoro-5-(piperazin-1-yl)picolinamide with N-cyclopropyl-6-fluoro-5-(piperazin-1-yl)picolinamide produced the target compound 7-((4-(2-fluoro-6-(cyclopropylcarbamoyl)pyridin-3-yl)piperazin-1-yl)methyl)-6-fluorofuro[2,3-c]quinolin-4(5H)-one. Replacement of N-ethyl-6-fluoro-5-(piperazin-1-yl)picolinamide with 6-chloro-N-methyl-5-(piperazin-1-yl)picolinamide produced the target compound 7-((4-(2-chloro-6-(methylcarbamoyl)pyridin-3-yl)piperazin-1-yl)methyl)-6-fluorofuro[2,3-c]quinolin-4(5H)-one.

The compounds of this disclosure can be prepared as illustrated by the exemplary reaction in Scheme 8. Reaction of 4-fluoro-1H-pyrazole-5-carboxylic acid and SOCl₂ produced 4-fluoro-1H-pyrazole-5-carbonyl chloride. Reaction of 4-fluoro-1H-pyrazole-5-carbonyl chloride and 3-bromo-2,6-difluoroaniline under the catalysis of lithium bis(trimethylsilyl)amide (LiHMDS) produced N-(3-bromo-2,6-difluorophenyl)-4-fluoro-1H-pyrazole-5-carboxamide. Reaction of N-(3-bromo-2,6-difluorophenyl)-4-fluoro-1H-pyrazole-5-carboxamide and K₂CO₃ produced 7-bromo-3,6-difluoropyrazolo[1,5-a]quinoxalin-4(5H)-one. Stille coupling of 7-bromo-3,6-difluoropyrazolo[1,5-a]quinoxalin-4(5H)-one and Bu₃SnCH₂OH under the catalysis of XPhos Pd G2 produced 3,6-difluoro-7-(hydroxymethyl)pyrazolo[1,5-a]quinoxalin-4(5H)-one. Reaction of 3,6-difluoro-7-(hydroxymethyl)pyrazolo[1,5-a]quinoxalin-4(5H)-one and HBr produced 7-(bromomethyl)-3,6-difluoropyrazolo[1,5-a]quinoxalin-4(5H)-one. Reaction of 7-(bromomethyl)-3,6-difluoropyrazolo[1,5-a]quinoxalin-4(5H)-one and N,6-dimethyl-5-(piperazin-1-yl)picolinamide under the catalysis of DIEA and KI produced the target compound 7-((4-(2-methyl-6-(methylcarbamoyl)pyridin-3-yl)piperazin-1-yl)methyl)-3,6-difluoropyrazolo[1,5-a]quinoxalin-4(5H)-one.

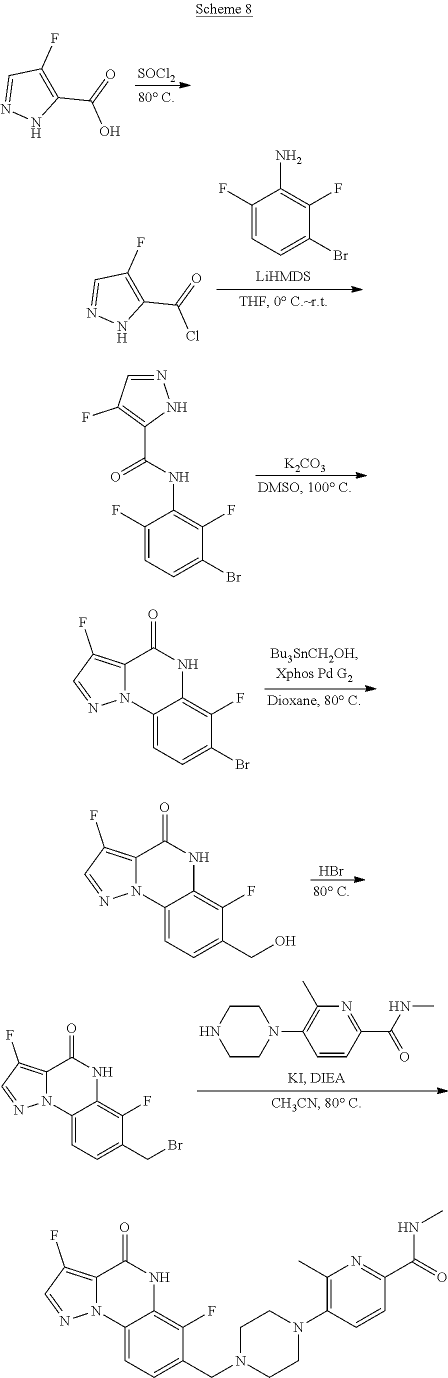

Other related compounds can be prepared using similar methods. For example, replacement of N,6-dimethyl-5-(piperazin-1-yl)picolinamide with 6-fluoro-N-methyl-5-(piperazin-1-yl)picolinamide produced the target compound 7-((4-(2-fluoro-6-(methylcarbamoyl)pyridin-3-yl)piperazin-1-yl)methyl)-3,6-difluoropyrazolo[1,5-a]quinoxalin-4(5H)-one. Replacement of N,6-dimethyl-5-(piperazin-1-yl)picolinamide with 6-chloro-N-methyl-5-(piperazin-1-yl)picolinamide produced the target compound 7-((4-(2-chloro-6-(methylcarbamoyl)pyridin-3-yl)piperazin-1-yl)methyl)-3,6-difluoropyrazolo[1,5-a]quinoxalin-4(5H)-one. Replacement of N,6-dimethyl-5-(piperazin-1-yl)picolinamide with 5-(5,6-dihydropyrrolo[3,4-c]pyrazol-2(4H)-yl)-N-methylpicolinamide produced the target compound 7-((2-(6-(methylcarbamoyl)pyridin-3-yl)-2,6-dihydropyrrolo[3,4-c]pyrazol-5(4H)-yl)methyl)-3,6-difluoropyrazolo[1,5-a]quinoxalin-4(5H)-one.

The compounds of this disclosure can be prepared as illustrated by the exemplary reaction in Scheme 9. Reaction of 4-bromo-3-fluoro-2-nitroaniline with NaNO₂, HCl and SnCl₂·2H₂O produced (4-bromo-3-fluoro-2-nitrophenyl)hydrazine. Reaction of (4-bromo-3-fluoro-2-nitrophenyl)hydrazine with methyl 4-(dimethylamino)-3-methyl-2-oxobut-3-enoate and AcOH produced methyl 1-(4-bromo-3-fluoro-2-nitrophenyl)-4-methyl-1H-pyrazole-5-carboxylate. Reaction of methyl 1-(4-bromo-3-fluoro-2-nitrophenyl)-4-methyl-1H-pyrazole-5-carboxylate with Fe and AcOH produced 7-bromo-6-fluoro-3-methylpyrazolo[1,5-a]quinoxalin-4(5H)-one. Reaction of 7-bromo-6-fluoro-3-methylpyrazolo[1,5-a]quinoxalin-4(5H)-one and Bu₃SnCH₂OH under the catalysis of XPhos Pd G2 produced 6-fluoro-7-(hydroxymethyl)-3-methylpyrazolo[1,5-a]quinoxalin-4(5H)-one. Reaction of 6-fluoro-7-(hydroxymethyl)-3-methylpyrazolo[1,5-a]quinoxalin-4(5H)-one with HBr produced 7-(bromomethyl)-6-fluoro-3-methylpyrazolo[1,5-a]quinoxalin-4(5H)-one. Reaction of 7-(bromomethyl)-6-fluoro-3-methylpyrazolo[1,5-a]quinoxalin-4(5H)-one and N-(2,2-difluoroethyl)-6-fluoro-5-(piperazin-1-yl)picolinamide under the catalysis of K₂CO₃ and KI produced the target compound 7-((4-(2-fluoro-6-(2,2-difluoroethylcarbamoyl)pyridin-3-yl)piperazin-1-yl)methyl)-6-fluoro-3-methylpyrazolo[1,5-a]quinoxalin-4(5H)-one.

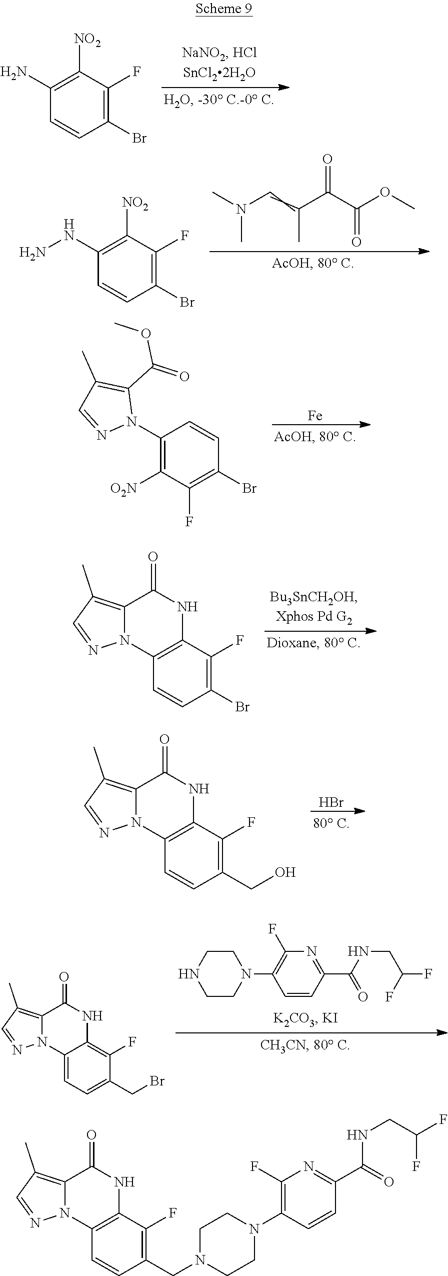

Other related compounds can be prepared using similar methods. For example, replacement of N-(2,2-difluoroethyl)-6-fluoro-5-(piperazin-1-yl)picolinamide with 6-chloro-N-methyl-5-(piperazin-1-yl)picolinamide produced the target compound 7-((4-(2-chloro-6-(methylcarbamoyl)pyridin-3-yl)piperazin-1-yl)methyl)-6-fluoro-3-methylpyrazolo[1,5-a]quinoxalin-4(5H)-one. Replacement of N-(2,2-difluoroethyl)-6-fluoro-5-(piperazin-1-yl)picolinamide with 6-(difluoromethyl)-N-methyl-5-(piperazin-1-yl)picolinamide produced the target compound 7-((4-(2-difluoromethyl-6-(methylcarbamoyl)pyridin-3-yl)piperazin-1-yl)methyl)-6-fluoro-3-methylpyrazolo[1,5-a]quinoxalin-4(5H)-one. Replacement of N-(2,2-difluoroethyl)-6-fluoro-5-(piperazin-1-yl)picolinamide with N-methyl-5-(piperazin-1-yl)-6-(trifluoromethyl)picolinamide produced the target compound 7-((4-(2-trifluoromethyl-6-(methylcarbamoyl)pyridin-3-yl)piperazin-1-yl)methyl)-6-fluoro-3-methylpyrazolo[1,5-a]quinoxalin-4(5H)-one. Replacement of N-(2,2-difluoroethyl)-6-fluoro-5-(piperazin-1-yl)picolinamide with 5-(5,6-dihydropyrrolo[3,4-c]pyrazol-2(4H)-yl)-N,6-dimethylpicolinamide produced the target compound 7-((2-(2-methyl-6-(methylcarbamoyl)pyridin-3-yl)-2,6-dihydropyrrolo[3,4-c]pyrazol-5(4H)-yl)methyl)-6-fluoro-3-methylpyrazolo[1,5-a]quinoxalin-4(5H)-one.

An important aspect of the present disclosure is the discovery that compounds of Formula I (including Formulae II, III, IV, V, VI, VII, VIII and IX) are PARP inhibitors, especially selective PARP1 inhibitors. Therefore, the compounds of Formula I (including Formulae II, III, IV, V, VI, VII, VIII and IX) or stereoisomers, tautomers, N-oxides, hydrates, isotope-substituted derivatives, solvates or pharmaceutically acceptable salts thereof, or mixtures thereof, or prodrugs thereof can be used to treat a variety of diseases or conditions responsive to the inhibition of PARP activity (especially PARP1 activity), or used to prepare a medicament for treating or preventing diseases or conditions responsive to the inhibition of PARP activity (especially PARP1 activity).

Cyclic nucleotide phosphodiesterases (PDEs) constitute a superfamily of enzymes that catalyze the degradation of the second messengers, cyclic adenosine 3′,5′-monophosphate (cAMP) and cyclic guanosine 3′,5′-monophosphate (cGMP). PDEs mediate a variety of physiological processes, including ion channel function, muscle contraction, central nervous system (CNS) function, apoptosis, glycogenolysis, and gluconeogenesis. Dysfunction of PDEs, however, has been implicated in various neurological and cardiovascular diseases, chronic obstructive pulmonary disease (COPD), and cancer development (Leroy et al., 2018 Circulation 138:2003-2006; Johnson et al., Lancet 2001, 358: 256-257; Lee et al., 2015 Nature 519: 472-476). PDE3 inhibitors are known to increase cardiac contractility and heart rate and decrease blood pressure (Young et al., 1988 Drugs 36: 158-192). Inhibition of PDE3 has also been shown to cause myocardial lesions (myocyte necrosis with inflammatory cell infiltration) and vascular lesions of the mesentery, spleen and pancreas in rats (Zhang et al., 2002 Mol Pharmacol 62: 514-520; Aguirre et al., 2010 Toxicologic Pathology, 38: 416-428). Another important aspect of the invention is that the selectivity of the compounds of Formula I (including Formulae II, III, IV, V, VI, VII, VIII and IX) to PARP1 is significantly improved and the inhibitory activity of PDE3A is significantly reduced, in particular the compounds of Formula I (including Formulae II, III, IV, V, VI, VII, VIII and IX) in which A₂ and/or A₃ is CR₁ and R₁ is halogen, especially F, and/or R₉ and/or R₁₀ is halogen, especially F. In particular, the compounds of Formulae VII, VIII and IX in which R₁₀ is halogen, especially F, have very low inhibitory activity against PDE3A. Therefore, the compounds of the invention have low off-target side effects and low toxicity, and are more suitable for clinical application and combined use with other drugs.

In the disclosure, the diseases or conditions responsive to the inhibition of PARP activity (especially PARP1 activity), include cancer. Cancer can be a solid tumor or hematological tumor, including but is not limited to liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, lung cancer (such as small cell lung cancer), Wilms tumor, cervical cancer, testicular cancer, soft tissue sarcoma, primary macroglobulinemia, bladder cancer, chronic myeloid leukemia, primary brain cancer, malignant melanoma, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer. Preferably, the cancer is responsive to the inhibition of PARP activity, especially PARP1 activity.

Therefore, the present disclosure includes methods for the treatment or prevention of diseases or conditions responsive to the inhibition of PARP activity (especially PARP1 activity), comprising administering to a subject (especially mammal, more specifically human) in need thereof an effective amount of the compound of Formula I (including Formulae II, III, IV, V, VI, VII, VIII and IX) or stereoisomers, tautomers, N-oxides, hydrates, isotope-substituted derivatives, solvates or pharmaceutically acceptable salts thereof, or mixtures thereof, or prodrugs thereof, or a pharmaceutical composition comprising an effective amount of the compound of Formula I (including Formulae II, III, IV, V, VI, VII, VIII and IX) or stereoisomers, tautomers, N-oxides, hydrates, isotope-substituted derivatives, solvates or pharmaceutically acceptable salts thereof, or mixtures thereof, or prodrugs thereof. In the disclosure, subjects include mammals, more specifically humans.

In practicing the therapeutic methods, effective amounts of pharmaceutical preparations are administered to an individual exhibiting the symptoms of one or more of these disorders. The pharmaceutic preparations comprise a therapeutically effective amount of the compound of Formula I (including Formulae II, III, IV, V, VI, VII, VIII and IX), formulated for oral, intravenous, local or topical application, for the treatment of cancer and other diseases. The amount is effective to ameliorate or eliminate one or more symptoms of the disorders. An effective amount of a compound for treating a particular disease is an amount that is sufficient to ameliorate or in some manner reduce the symptoms associated with the disease. Such amount may be administered as a single dosage or may be administered according to an effective regimen. The amount may cure the disease but, typically, is administered in order to ameliorate the symptoms of the disease. Typically, repeated administration is required to achieve the desired amelioration of symptom.

In another embodiment, there is provided a pharmaceutical composition comprising a compound of Formula I (including Formulae II, III, IV, V, VI, VII, VIII and IX) as a PARP inhibitor, or stereoisomers, tautomers, N-oxides, hydrates, solvates, isotope-substituted derivatives, or pharmaceutically acceptable salts thereof, or mixtures thereof, or prodrugs thereof.

Another embodiment of the present disclosure is directed to a pharmaceutical composition effective to treat or prevent cancer comprising a compound of Formula I (including Formulae II, III, IV, V, VI, VII, VIII and IX) as a PARP inhibitor, or stereoisomers, tautomers, N-oxides, hydrates, solvates, isotope-substituted derivatives, or pharmaceutically acceptable salts thereof and prodrugs thereof, in combination with at least one known anticancer agent or a pharmaceutically acceptable salt thereof. In particular, the compound herein can be combined with other anticancer drugs related to the mechanism of DNA damage and repair, such as ATM inhibitors, ATR inhibitors, Weel inhibitors, DNA-PK inibitors; HDAC inhibitors such as Volinota, Romididesin, Papiseta and Bailesta; other anticancer drugs related to cell division, including Chk1/2 inhibitors, CDK4/6 inhibitors such as Paposinib; other targeted anticancer agents, including USP1 inhibitors, PRMT5 inhibitors, Polθ inhibitors, RAD51 inhibitors, and so on. Other known anticancer agents which may be used for anticancer combination therapy include, but are not limited to alkylating agents, such as busulfan, melphalan, chlorambucil, cyclophosphamide, ifosfamide, temozolomide, bendamustine, cis-platin, mitomycin C, bleomycin and carboplatin; topoisomerase I inhibitors, such as camptothecin, irinotecan and topotecan; topoisomerase II inhibitors, such as doxorubicin, epirubicin, aclacinomycin, mitoxantrone, elliptinium and etoposide; RNA/DNA antimetabolites, such as 5-azacytidine, gemcitabine, 5-fluorouracil, capecitabine and methotrexate; DNA antimetabolites, such as 5-fluoro-2′-deoxy-uridine, fludarabine, nelarabine, ara-C, pralatrexate, pemetrexed, hydroxyurea and thioguanine; antimitotic agent such as colchicine, vinblastine, vincristine, vinorelbine, paclitaxel, ixabepilone, cabazitaxel and docetaxel; antibodies such as mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin and mabthera; Antibody-Drug Conjugates (ADC) such as T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin; kinase inhibitors such as imatinib, gefitinib, erlotinib, osimertinib, afatinib, ceritinib, alectinib, crizotinib, erlotinib, lapatinib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, Idelalisib, pazopanib, Torisel and everolimus. Other known anticancer agents which may be used for anticancer combination therapy include tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2) and Sipueucel-T (prostate cancer treatment vaccine).

In practicing the methods of the present disclosure, the compound of the disclosure may be administered together with at least one known anticancer agent in a unitary pharmaceutical composition. Alternatively, the compound of the disclosure may be administered separately from at least one known anticancer agent. In one embodiment, the compound of the disclosure and at least one known anticancer agent are administered substantially simultaneously, i.e. all agents are administered at the same time or one after another, provided that compounds reach therapeutic levels in the blood at the same time. In another embodiment, the compound of the disclosure and at least one known anticancer agent are administered according to individual dose schedule, provided that the compounds reach therapeutic levels in the blood.

Another embodiment of the present disclosure is directed to a bioconjugate to inhibit tumor. The bioconjugate is consisted of the compound described herein and at least one known therapeutically useful antibody, such as trastuzumab or rituximab, or growth factor, such as EGF or FGF, or cytokine, such as IL-2 or IL-4, or any molecule that can bind to cell surface. The antibodies and other molecules could deliver the compound described herein to its targets, making it an effective anticancer agent. The bioconjugates could also enhance the anticancer effect of the therapeutically useful antibodies, such as trastuzumab or rituximab.

Another embodiment of the present disclosure is directed to a pharmaceutical composition effective to inhibit tumor comprising the PARP inhibitor of Formula I (including Formulae II, III, IV, V, VI, VII, VIII and IX), or pharmaceutically acceptable salts thereof, or prodrugs thereof, in combination with radiation therapy. In this embodiment, the compound of the disclosure may be administered at the same time as the radiation therapy or at a different time.

Yet another embodiment of the present disclosure is directed to a pharmaceutical composition effective for post-surgical treatment of cancer, comprising the PARP inhibitor of Formula I (including Formulae II, III, IV, V, VI, VII, VIII and IX), or pharmaceutically acceptable salts thereof, or prodrug thereof. The disclosure also relates to a method of treating cancer by surgically removing tumor and then treating the mammal with the pharmaceutical composition described herein.

Pharmaceutical compositions of this disclosure include all pharmaceutical preparations which contain the compounds of the present disclosure in an amount that is effective to achieve its intended purpose. While individual needs vary, determination of optimal amounts of each component in the pharmaceutical preparations is within the skill of the art. Typically, the compounds or the pharmaceutically acceptable salt thereof may be administered to mammals, orally at a dose of about 0.0025 to 50 mg per kg body weight per day. Preferably, from approximately 0.01 mg/kg to approximately 10 mg/kg body weight is orally administered. If a known anticancer agent is also administered, it is administered in an amount that is effective to achieve its intended purpose. The optimal amounts of such known anticancer agents are well known to those skilled in the art.

The unit oral dose may comprise from approximately 0.01 to approximately 50 mg, preferably approximately 0.1 to approximately 10 mg of the compound of the disclosure. The unit dose may be administered one or more times, with one or more tablets daily, each containing from approximately 0.1 to approximately 50 mg, conveniently approximately 0.25 to 10 mg of the compound of the disclosure or its solvates.

In a topical formulation, the compound of the disclosure may be present at a concentration of approximately 0.01 to 100 mg per gram of carrier.

The compound of the disclosure may be administered as a raw chemical. The compounds of the disclosure may also be administered as part of a suitable pharmaceutical preparation containing pharmaceutically acceptable carriers (comprising excipients and auxiliaries), which facilitate the processing of the compounds into pharmaceutically acceptable preparations. Preferably, the pharmaceutical preparations, particularly oral preparations and those used for the preferred administration, such as tablets, draggers, and capsules, as well as solutions suitable for injection or oral administration, contain from approximately 0.01% to 99%, preferably from approximately 0.25% to 75% of active compound(s), together with excipient(s).

Also included within the scope of the present disclosure are the non-toxic pharmaceutically acceptable salts of the compounds of the present disclosure. Acid addition salts are formed by mixing a solution of the compounds of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, choline hydroxide, sodium carbonate, tris(hydroxymethyl)aminomethane, N-methyl-glucamine and the like.

The pharmaceutical preparations of the disclosure may be administered to any mammal, so long as they may experience the therapeutic effects of the compounds of the disclosure. Foremost among such mammals are humans and veterinary animals, although the disclosure is not intended to be so limited.

The pharmaceutical preparations of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively or concurrently, administration may be by oral route. The dosage administered will be dependent upon the age, health, and weight of the recipient, type of concurrent treatment, frequency of treatment, and the nature of the effect desired.

The pharmaceutical preparations of the present disclosure are manufactured in a known manner, e.g., by means of conventional mixing, granulating, dragee-making, dissolving, or lyophilizing processes. Pharmaceutical preparations for oral use may be obtained by combining the active compounds with solid excipients, optionally grinding the resulting mixture, processing the mixture of granules after adding suitable auxiliaries if desired or necessary, thereby obtaining tablets or dragee cores.

Suitable excipients are, in particular, fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, including, e.g., maize starch, wheat starch, rice starch, potato starch, gelatin, tragacanth, methylcellulose, hydroxypropyl methylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, in particular, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropyl methylcellulose phthalate, are used. Dyes or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate and stabilizers. In soft capsules, the active compounds are preferably dissolved or suspended in suitable liquids, such as fatty oils, or liquid paraffin. In addition, stabilizers may be added.

Suitable formulations for parenteral administration include aqueous solutions of the active compounds, e.g., aqueous solutions and alkaline solutions of water-soluble salts. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, suspension stabilizers may also be contained.

In accordance with one aspect of the present disclosure, compounds of the disclosure are employed in topical and parenteral formulations and are used for the treatment of skin cancer.

The topical formulations of this disclosure are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The present disclosure also involves use of the compounds of the disclosure for the manufacture of a medicament for the treatment of clinical symptoms in response to inhibition of the activity of PARP. The medicament may include the above-mentioned pharmaceutical compositions.

The following examples are illustrative, but not limiting, of the method and compositions of the present disclosure. Other suitable modifications and adaptations of the variety of conditions and parameters normally encountered in clinical therapy and which are obvious to those skilled in the art are within the spirit and scope of the disclosure.

### EXAMPLES

### General Remarks

All reagents were of commercial quality. Solvents were dried and purified by standard methods. Mass spectrum analyses were recorded on a Platform II (Agilent 6110) quadrupole mass spectrometer fitted with an electrospray interface. ¹H NMR spectra was recorded at 400 MHz, on a Brucker Ascend 400 apparatus. Chemical shifts were recorded in parts per million (ppm) downfield from TMS (0.00 ppm), and J coupling constants were reported in hertz (Hz).

### Example 1

### 7-((4-(2-methyl-6-(methylcarbamoyl)pyridin-3-yl)piperazin-1-yl)methyl)thieno[3,2-c]quinolin-4(5H)-one

a) Preparation of methyl 4-oxo-4,5-dihydrothieno[3,2-c]quinoline-7-carboxylate: To a mixture of methyl 2-bromothiophene-3-carboxylate (170.0 mg, 0.8 mmol) and (2-amino-4-(methoxycarbonyl)phenyl)boronic acid (214.0 mg, 0.9 mmol) in DMF (15.0 mL) was added sodium acetate (95.0 mg, 1.2 mmol) and Pd(dppf)Cl₂ (169.0 mg, 0.2 mmol) under nitrogen. The mixture was stirred at 120° C. overnight under nitrogen. The mixture was cooled to room temperature and filtered. The filtrate was concentrated under reduced pressure. The residue was purified by Prep-TLC (DCM/MeOH=20/1) to afford the product (76.7 mg, yellow solid, yield: 38%). MS(ESI): 260.00 [M+H]⁺.
b) Preparation of 7-(hydroxymethyl)thieno[3,2-c]quinolin-4(5H)-one: To a suspension of methyl 4-oxo-4,5-dihydrothieno[3,2-c]quinoline-7-carboxylate (75.0 mg, 0.3 mmol) in THF (8 mL) was added LiAlH₄ (1 M in THF, 1.2 mL, 1.2 mmol) under nitrogen at 0° C. The mixture was warmed to room temperature and stirred for 1 hour. Then the reaction mixture was quenched with water (5 mL) and extracted with EA (15 mL×3). The combined organic phase was washed with brine, dried over anhydrous sodium sulfate and concentrated under reduced pressure to give the product (60.0 mg, crude, brown solid). MS(ESI): 232.00 [M+H]⁺.
c) Preparation of 7-(chloromethyl)thieno[3,2-c]quinolin-4(5H)-one: To a suspension of 7-(hydroxymethyl)thieno[3,2-c]quinolin-4(5H)-one (60.0 mg, 0.3 mmol) in DCM (6 mL) was added DMF (2 drops) and thionyl chloride (123.5 mg, 1.0 mmol) dropwise at 0° C. The resulting mixture was stirred at room temperature for 15 min. After completion of the reaction, the mixture was concentrated to give the product (50.0 mg, crude, gray solid). MS(ESI): 250.00 [M+H]⁺.
d) Preparation of 7-((4-(2-methyl-6-(methylcarbamoyl)pyridin-3-yl)piperazin-1-yl)methyl)thieno[3,2-c]quinolin-4(5H)-one: To a solution of 7-(chloromethyl)thieno[3,2-c]quinolin-4(5H)-one (50.0 mg, 0.2 mmol), KI (3.3 mg, 0.02 mmol) and N,6-dimethyl-5-(piperazin-1-yl)picolinamide (56.0 mg, 0.24 mmol) in CH₃CN (5 mL) was added DIEA (156.0 mg, 1.2 mmol) at room temperature. The resulting solution was stirred at 80° C. for 1.5 hours under N₂. After completion of the reaction, the solvent was removed under vacuum. The residue was purified by Prep-TLC (DCM/MeOH=10/1) to give the target compound (14.5 mg, white solid, 3-step yield: 11%).

The following compounds of Examples 2-5 were prepared using a synthesis method similar to that described in Example 1.

| | | | LC-MS | |
|---|---|---|---|---|
| Example | Compound | MW | (ESI) | ¹H NMR (400 MHz) |
| 1 | 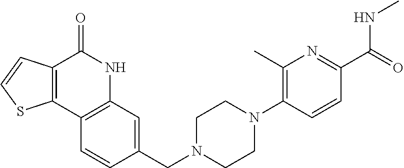 [SMILES: CNC(=O)c1ccc(N2CCN(Cc3ccc4c(c3)[nH]c(=O)c3ccsc34)CC2)c(C)n1] | 447.56 | 448.25 [M + H]⁺ | DMSO-d₆: δ 11.95 (s, 1H), 8.39 (d, J = 5.3 Hz, 1H), 7.91 (d, J = 8.0 Hz, 1H), 7.81-7.72 (m, 2H), 7.54 (d, J = 5.1 Hz, 1H), 7.51-7.43 (m, 2H), 7.33 (d, J = 8.0 Hz, 1H), 4.43 (s, 2H), 3.47-3.39 (m, 4H), 3.03-2.87 (m, 4H), 2.72 (d, J = 4.7 Hz, 3H), 2.44 (s, 3H). |
| 2 | 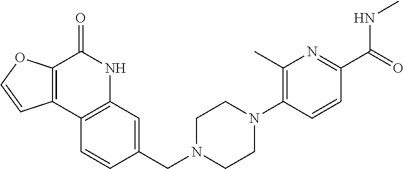 [SMILES: CNC(=O)c1ccc(N2CCN(Cc3ccc4c(c3)[nH]c(=O)c3occc34)CC2)c(C)n1] | 431.50 | 432.20 [M + H]⁺ | DMSO-d₆: δ 11.81 (s, 1H), 8.38 (q, J = 5.8, 5.4 Hz, 1H), 8.20 (d, J = 1.9 Hz, 1H), 7.94 (d, J = 8.1 Hz, 1H), 7.76 (d, J = 8.2 Hz, 1H), 7.48-7.38 (m, 3H), 7.23 (dd, J = 8.0, 1.5 Hz, 1H), 3.61 (s, 2H), 2.98-2.87 (m, 4H), 2.76 (d, J = 4.9 Hz, 3H), 2.62-2.51 (m, 4H), 2.48 (s, 3H). |
| 3 | 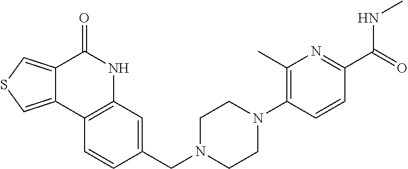 [SMILES: CNC(=O)c1ccc(N2CCN(Cc3ccc4c(c3)[nH]c(=O)c3cscc34)CC2)c(C)n1] | 447.56 | 448.15 [M + H]⁺ | CDCl₃: δ 9.61 (s, 1H), 8.47 (d, J = 4.0 Hz, 1H), 7.83 (d, J = 5.2 Hz, 1H), 7.74 (d, J = 5.2 Hz, 1H), 7.44 (s, 1H), 7.39-7.30 (m, 2H), 3.74 (s, 2H), 3.11-2.90 (m, 7H), 2.79-2.64 (m, 4H), 2.50 (s, 3H). |
| 4 | 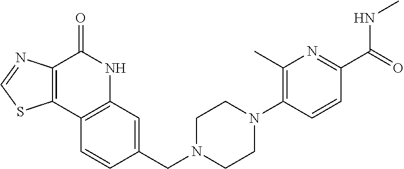 [SMILES: CNC(=O)c1ccc(N2CCN(Cc3ccc4c(c3)[nH]c(=O)c3ncsc34)CC2)c(C)n1] | 448.55 | 449.15 [M + H]⁺ | DMSO-d₆: δ 11.93 (s, 1H), 9.27 (s, 1H), 8.40- 8.37 (m, 1H), 7.78 (dd, J = 18.7, 8.2 Hz, 2H), 7.47-7.42 (m, 2H), 7.24 (d, J = 8.1 Hz, 1H), 3.62 (s, 2H), 2.95-2.89 (m, 4H), 2.76 (d, J = 4.8 Hz, 3H), 2.59-2.53 (m, 4H), 2.50 (s, 3H). |
| 5 | 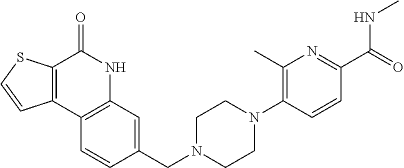 [SMILES: CNC(=O)c1ccc(N2CCN(Cc3ccc4c(c3)[nH]c(=O)c3sccc34)CC2)c(C)n1] | 447.56 | 448.15 [M + H]⁺ | CDCl₃: δ 10.31 (s, 1H), 8.00-7.88 (m, 3H), 7.83 (d, J = 5.2 Hz, 1H), 7.74 (d, J = 5.2 Hz, 1H), 7.44 (s, 1H), 7.39-7.30 (m, 2H), 3.74 (s, 2H), 3.11-2.90 (m, 7H), 2.79-2.64 (m, 4H), 2.50 (s, 3H). |

### Example 6

### 7-((4-(2-methyl-6-(methylcarbamoyl)pyridin-3-yl)piperazin-1-yl)methyl)furo[3,2-c]quinolin-4(5H)-one

a) Preparation of methyl 2-(4-bromo-2-nitrophenyl)furan-3-carboxylate: To a solution of methyl furan-3-carboxylate (1.0 g, 8.0 mmol) in toluene (10 mL) was added 1,4-dibromo-2-nitrobenzene (2.7 g, 9.6 mmol) and potassium acetate (2.4 g, 24.0 mmol) and Pd(PPh₃)₄ (924.0 mg, 0.8 mmol). The mixture was stirred at 110° C. overnight under nitrogen. After completion of the reaction, the mixture was poured into water (100 mL) and extracted with EA (20 mL×3), and the organic phase was washed with brine, dried over anhydrous Na₂SO₄ and concentrated under reduced pressure. The residue was purified by silica gel column (PE:EA=30:1) to give the product (610 mg, yellow solid, yield: 22.6%).
b) Preparation of 7-bromofuro[3,2-c]quinolin-4(5H)-one: To a solution of methyl 2-(4-bromo-2-nitrophenyl)furan-3-carboxylate (610 mg, 1.8 mmol) in EtOH (10 mL) was added Fe (503 mg, 9.0 mmol) and the solution of NH₄Cl (481 mg, 9.0 mmol) and H₂O (5 mL). The mixture was stirred at 80° C. overnight. After completion of the reaction, the mixture was filtered with diatomite. The filtrate was concentrated, and the residue was diluted with H₂O and extracted with EA, the organic phase was washed with brine, dried over anhydrous Na₂SO₄ and concentrated under reduced pressure. Then the residue was purified with silica gel column (PE:EA=5:1) to give the product (270 mg, white oil, yield: 57.1%). MS(ESI): 263.96 [M+H]⁺.
c) Preparation of 7-(hydroxymethyl)furo[3,2-c]quinolin-4(5H)-one: To a solution of 7-bromofuro[3,2-c]quinolin-4(5H)-one (270 mg, 1.03 mmol) in dioxane (5 mL) was added Bu₃SnCH₂OH (660 mg, 2.06 mmol) and Xphos Pd G2 (78.7 mg, 0.1 mmol). The mixture was stirred at 90° C. under nitrogen atmosphere overnight. After completion of the reaction, a solution of KF (1 M, 10 mL) was added at room temperature, and the mixture was stirred at room temperature for 10 minutes and filtered. The filtrate was extracted with EA (20 mL×3). The combined organic phase was washed with brine, dried over anhydrous Na₂SO₄ and concentrated under reduced pressure. The residue was triturated with the mixed solvent of PE and EA (PE:EA=1:1) and the solid was collected by filtration to afford the product as a white solid (120 mg, grey solid, yield: 54.3%). MS(ESI): 216.25 [M+H]⁺.
d) Preparation of 7-(chloromethyl)furo[3,2-c]quinolin-4(5H)-one: To a solution of 7-(hydroxymethyl)furo[3,2-c]quinolin-4(5H)-one (100 mg, 0.46 mmol) in DCM (2 mL) was added SOCl₂ (276 mg, 2.33 mmol). The mixture was stirred at room temperature for 2 hours. After completion of the reaction, the mixture was concentrated under reduced pressure to afford the product (110 mg, crude, white solid). MS(ESI): 234.00 [M+H]⁺.
e) Preparation of 7-((4-(2-methyl-6-(methylcarbamoyl)pyridin-3-yl)piperazin-1-yl)methyl)furo[3,2-c]quinolin-4(5H)-one: To a solution of 7-(chloromethyl)furo[3,2-c]quinolin-4(5H)-one (110 mg, 0.47 mmol) in acetonitrile (5 mL) was added N,6-dimethyl-5-(piperazin-1-yl)picolinamide (134 mg, 0.56 mmol), KI (7.8 mg, 0.05 mmol) and DIEA (181.9 mg, 1.41 mmol). The mixture was stirred at 80° C. under nitrogen atmosphere for 1 hour. After completion of the reaction, the solvent was removed under reduced pressure. The residue was purified by Prep-TLC (DCM:MeOH=10:1) to give the target compound (7.0 mg, white solid, yield: 3.5%).

### Example 7

### 7-((4-(2-methyl-6-(methylcarbamoyl)pyridin-3-yl)piperazin-1-yl)methyl)-1,5-dihydro-4H-pyrazolo[4,3-c]quinolin-4-one

a) Preparation of ethyl 5-(4-bromo-2-nitrophenyl)-1H-pyrazole-4-carboxylate: To a solution of ethyl 2-(4-bromo-2-nitrobenzoyl)-3-(dimethylamino)acrylate (1.0 g, 2.69 mmol) in acetonitrile (8 mL) was added hydrazine hydrate (50% in H₂O, 323 mg, 2.69 mmol). The resulting mixture was stirred at 50° C. for 1 hour. After completion of the reaction, the reaction mixture was concentrated under reduced pressure. The residue was diluted with water (20 mL) and extracted with EA (20 mL×3). The combined organic phase was washed with brine, dried over anhydrous sodium sulfate and concentrated under reduced pressure. The residue was purified chromatography over silica gel (PE:EA=4:1) to give the product (800 mg, yellow solid, yield: 88%). MS(ESI): 339.90 [M+H]⁺.
b) Preparation of 7-bromo-1,5-dihydro-4H-pyrazolo[4,3-c]quinolin-4-one: To a solution of ethyl 5-(4-bromo-2-nitrophenyl)-1H-pyrazole-4-carboxylate (800 mg, 2.36 mmol) in acetic acid (6 mL) was added iron dust (662 mg, 11.83 mmol) under nitrogen. The resulting mixture was stirred at 80° C. for 2 hours. After completion of the reaction, the mixture was cooled to room temperature and filtered. The solid was dried under reduced pressure to give the product (400 mg, white solid, yield: 67.6%). MS(ESI): 261.95 [M+H]⁺.
c) Preparation of tert-butyl 7-bromo-4-oxo-4,5-dihydro-1H-pyrazolo[4,3-c]quinoline-1-carboxylate: To a solution of 7-bromo-1,5-dihydro-4H-pyrazolo[4,3-c]quinolin-4-one (400 mg, 1.4 mmol) in DMF (4 mL) was added di-tert-butyl dicarbonate (325 mg, 1.4 mmol) and DMAP (20 mg, 0.14 mmol) at room temperature. The mixture was stirred at room temperature for 2 hours. To the mixture was added water (20 mL) and extracted with EA (40 mL×2). The combined organic phase was washed with brine, dried over anhydrous Na₂SO₄ and concentrated under reduced pressure. The residue was purified by silica gel (DCM:MeOH=10:1) to afford the product (500 mg, yellow solid, yield: 73%). MS(ESI): 364.00 [M+H]⁺.
d) Preparation of tert-butyl 7-(hydroxymethyl)-4-oxo-4,5-dihydro-1H-pyrazolo[4,3-c]quinoline-1-carboxylate: To a solution of tert-butyl 7-bromo-4-oxo-4,5-dihydro-1H-pyrazolo[4,3-c]quinoline-1-carboxylate (500 mg, 1.37 mmol) in dioxane (20 mL) was added Bu₃SnCH₂OH (881 mg, 2.7 mmol) and Xphos Pd G2 (80 mg, 0.13 mmol, 0.1 eq) at room temperature. The mixture was stirred at 90° C. overnight under nitrogen atmosphere. After completion of the reaction, a solution of KF aqueous solution (1 M, 10 mL) was added at room temperature. The mixture was stirred at room temperature for 10 mins and filtered, and the filtrate was extracted with EA (50 mL×3). The combined organic phase was washed with brine, dried over anhydrous Na₂SO₄ and concentrated under reduced pressure. The residue was triturated with a mixed solvent of PE and EA (PE:EA=1:1, 10 mL) and the solid was collected by filtration to afford the product (450 mg, crude, white solid, yield: 90%). MS(ESI): 316.05 [M+H]⁺.
e) Preparation of 7-(bromomethyl)-1,5-dihydro-4H-pyrazolo[4,3-c]quinolin-4-one: A solution of tert-butyl 7-(hydroxymethyl)-4-oxo-4,5-dihydro-1H-pyrazolo[4,3-c]quinoline-1-carboxylate (450 mg, 1.3 mmol) in HBr (48% aq., 10 mL) was stirred at 85° C. for 3 hours. The reaction mixture was concentrated under reduced pressure. Acetonitrile (3 mL) was added to the residues and the mixture was concentrated under reduced pressure to give the product (300 mg, crude, off-white solid). MS(ESI): 275.85 [M+H]⁺.
f) Preparation of tert-butyl 7-(bromomethyl)-4-oxo-4,5-dihydro-1H-pyrazolo[4,3-c]quinoline-1-carboxylate: To a solution of 7-(bromomethyl)-1,5-dihydro-4H-pyrazolo[4,3-c]quinolin-4-one (353 mg, 1.2 mmol) in DMF (4 mL) was added di-tert-butyl dicarbonate (287 mg, 1.3 mmol) and DMAP (20 mg, 0.14 mmol) at room temperature. The mixture was stirred at room temperature for 2 hours. To the mixture was added water (20 mL), and the resulting mixture was extracted with EA (40 mL×2). The combined organic phase was washed with brine, dried over anhydrous Na₂SO₄ and concentrated under reduced pressure. The residue was purified by silica gel (DCM:MeOH=10:1) to afford the product (131 mg, yellow solid, 33%). MS(ESI): 378.00 [M+H]⁺.
g) Preparation of tert-butyl 7-((4-(2-methyl-6-(methylcarbamoyl)pyridin-3-yl)piperazin-1-yl)methyl)-4-oxo-4,5-dihydro-1H-pyrazolo[4,3-c]quinoline-1-carboxylate: To a solution of 7-(bromomethyl)-1-methyl-1,5-dihydro-4H-pyrazolo[4,3-c]quinolin-4-one (13 mg, 0.34 mmol), KI (10.0 mg, 0.1 mmol) and 7-(chloromethyl)-3-ethylquinazoline-2,4(1H,3H)-dione (81 mg, 0.34 mmol) in acetonitrile (3 mL) was added DIEA (140.0 mg, 1.0 mmol) at room temperature under N₂. The resulting suspension was stirred at 80° C. for 30 min. After completion of the reaction, the reaction mixture was concentrated under reduced pressure. The residue was washed with acetonitrile to give the product (95.0 mg, white solid, yield: 52%). MS(ESI): 532.25 [M+H]⁺.
h) Preparation of 7-((4-(2-methyl-6-(methylcarbamoyl)pyridin-3-yl)piperazin-1-yl)methyl)-1,5-dihydro-4H-pyrazolo[4,3-c]quinolin-4-one: To solution of tert-butyl 7-((4-(2-methyl-6-(methylcarbamoyl)pyridin-3-yl)piperazin-1-yl)methyl)-4-oxo-4,5-dihydro-1H-pyrazolo[4,3-c]quinoline-1-carboxylate (95 mg, 0.17 mmol) in dioxane (5 mL) was added HCl/dioxane solution (5 mL) under N₂. The resulting suspension was stirred at room temperature for 2 hours. After completion of the reaction, the solvent was removed under reduced pressure. The residue was purified by Prep-TLC (DCM:MeOH=10:1) to afford the target compound (5.0 mg, white solid, yield: 6%).

The following compound of Examples 8 was prepared using a synthesis method similar to that described in Example 7 (Scheme 3); the compound of Example 9 was prepared using a synthesis method similar to that described in Example 1 (Scheme 1).

| | | | LC-MS | |
|---|---|---|---|---|
| Example | Compound | MW | (ESI) | ¹H NMR (400 MHz) |
| 6 | 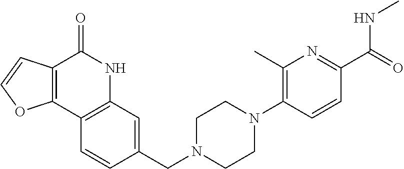 [SMILES: CNC(=O)c1ccc(N2CCN(Cc3ccc4c(c3)[nH]c(=O)c3ccoc34)CC2)c(C)n1] | 431.50 | 432.10 [M + H]⁺ | DMSO-d₆: δ11.67 (s, 1H), 8.39 (d, J = 4.9 Hz, 1H), 8.03 (d, J = 2.0 Hz, 1H), 7.85 (d, J = 7.9 Hz, 1H), 7.76 (d, J = 8.2 Hz, 1H), 7.49- 7.37 (m, 1H), 7.24 (d, J = 8.6 Hz, 1H), 7.02 (d, J = 2.0 Hz, 1H), 3.61 (s, 2H), 2.96-2.90 (m, 4H), 2.76 (d, J = 4.9 Hz, 3H), 2.59-2.52 (m, 4H), 2.46 (s, 3H). |
| 7 | 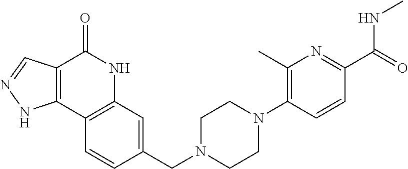 [SMILES: CNC(=O)c1ccc(N2CCN(Cc3ccc4c(c3)[nH]c(=O)c3cn[nH]c34)CC2)c(C)n1] | 431.50 | 432.15 [M + H]⁺ | DMSO-d₆: δ 11.29 (s, 1H), 8.44 (s, 1H), 8.03 (s, 1H), 7.78 (s, 1H), 7.44 (d, J = 30.3 Hz, 2H), 7.24 (s, 1H), 3.62 (s, 2H), 2.98-2.95 (m, 4H), 2.80 (s, 3H), 2.50-2.42 (m, 4H), 2.50 (s, 3H). |
| 8 | 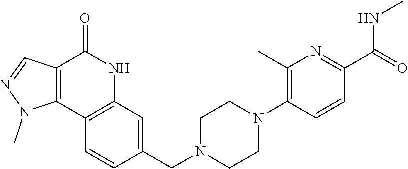 [SMILES: CNC(=O)c1ccc(N2CCN(Cc3ccc4c(c3)[nH]c(=O)c3cnn(C)c34)CC2)c(C)n1] | 445.53 | 446.15 [M + H]⁺ | DMSO-d₆: δ 11.39 (s, 1H), 8 8.43 (q, J = 4.8 Hz, 1H), 8.17 (d, J = 8.3 Hz, 1H), 8.07 (s, 1H), 7.79 (d, J = 8.3 Hz, 1H), 7.56-7.44 (m, 2H), 7.28 (d, J = 8.2 Hz, 1H), 4.35 (s, 3H), 3.64 (s, 2H), 3.40-3.31(m, 4H), 2.99-2.92 (m, 4H), 2.79 (d, J = 4.8 Hz, 3H), 2.59 (s, 3H). |
| 9 | 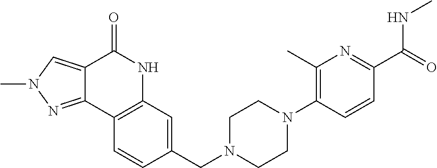 | 445.53 | 446.15 [M + H]⁺ | DMSO-d₆: δ 11.08 (s, 1H), 8.52 (s, 1H), 8.41 (d, J = 5.5 Hz, 1H), 7.95 (d, J = 8.0 Hz, 1H), 7.79 (d, J = 8.3 Hz, 1H), 7.48 (d, J = 8.4 Hz, 1H), 7.35 (s, 1H), 7.18 (d, J = 8.0 Hz, 1H), 4.08 (s, 3H), 3.60 (s, 2H), 2.99-2.92 (m, 4H), 2.79 (d, J = 4.8 Hz, 3H), 2.62-2.53 (m, 4H), 2.49 (s, 3H). |

### Example 10

### 7-((4-(2-methyl-6-(methylcarbamoyl)pyridin-3-yl)piperazin-1-yl)methyl)-3,5-dihydro-4H-pyrrolo[2,3-c]quinolin-4-one

a) Preparation of methyl 3-(2-amino-4-(hydroxymethyl)phenyl)-1H-pyrrole-2-carboxylate: To a solution of (3-amino-4-(4,4,5,5-tetramethyl-1,3,2-dioxaborolan-2-yl)phenyl) methanol (100 mg, 0.40 mmol) in the mixed solvent of dioxane and H₂O (v/v=10/1, 5.5 mL) was added methyl 3-bromo-1H-pyrrole-2-carboxylate (82 mg, 0.40 mmol), K₂CO₃ (111 mg, 0.80 mmol) and Pd(dppf)Cl₂ (29 mg, 0.04 mmol) at room temperature. The mixture was stirred at 100° C. for 16 hours under N₂ atmosphere. After completion of the reaction, the mixture was cooled to room temperature and filtered through celite. The filtrate was concentrated under reduced pressure to give the product (270 mg, crude, brown solid), which was used for the next step directly without further purification. MS(ESI): 247.10 [M+H]⁺.
b) Preparation of 7-(hydroxymethyl)-3H-pyrrolo[2,3-c]quinolin-4(5H)-one: A solution of methyl 3-(2-amino-4-(hydroxyl methyl)phenyl)-1H-pyrrole-2-carboxylate (270 mg, 1.1 mmol) in AcOH (3.0 mL) was stirred at room temperature under N₂ atmosphere for 3 hours. After completion of the reaction, the mixture was concentrated under reduced pressure. The residue was triturated with a mixed solvent of PE and EA (PE/EA=1/1, 10 mL×3) to give the product (72 mg, black solid, yield: 30%). MS(ESI): 215.10 [M+H]⁺.
c) Preparation of 7-(bromomethyl)-3H-pyrrolo[2,3-c]quinolin-4(5H)-one: A solution of 7-(hydroxymethyl)-3H-pyrrolo[2,3-c]quinolin-4(5H)-one (64 mg, 0.3 mmol) in HBr (48% in H₂O, 5.0 mL) was stirred at 85° C. for 3 hours. After completion of the reaction, the reaction mixture was concentrated under reduced pressure. The residue was dissolved with MeCN (3.0 mL), and concentrated under reduced pressure to give the product (90 mg, crude, gray solid). MS(ESI): 555.50 [2M+H]⁺.
d) Preparation of 7-((4-(2-methyl-6-(methylcarbamoyl)pyridin-3-yl)piperazin-1-yl)methyl)-3,5-dihydro-4H-pyrrolo[2,3-c]quinolin-4-one: To a solution of 7-(bromomethyl)-3H-pyrrolo[2,3-c]quinolin-4(5H)-one (90 mg, 0.3 mmol) and N,6-dimethyl-5-(piperazin-1-yl) picolinamide (76 mg, 0.32 mmol) in MeCN (2.0 mL) was added KI (5.4 mg, 0.03 mmol) and DIEA (126 mg, 0.97 mmol). The mixture was stirred at 80° C. under N₂ for 0.5 hour. After completion of the reaction, the mixture was concentrated under vacuum. The residue was purified by Prep-HPLC (C18, CH₃CN/H₂O, 30˜55%) to give the target compound (16.2 mg, off-white solid, yield: 12%).

The following compound of Example 11 was prepared using a synthesis method similar to that described in Example 10; the compounds of Examples 12-13 were prepared using a synthesis method similar to that described in Example 1 (Scheme 1).

| | | | LC-MS | |
|---|---|---|---|---|
| Example | Compound | MW | (ESI) | ¹H NMR (400 MHz) |
| 10 | 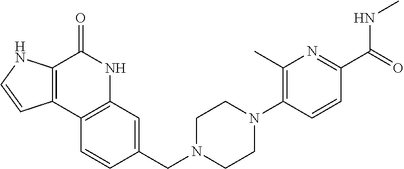 [SMILES: CNC(=O)c1ccc(N2CCN(Cc3ccc4c(c3)[nH]c(=O)c3[nH]ccc34)CC2)c(C)n1] | 430.51 | 431.10 [M + H]⁺ | DMSO-d₆: δ 12.18 (s, 1H), 11.29 (s, 1H), 8.43 (d, J = 5.7 Hz, 1H), 7.88 (d, J = 8.0 Hz, 1H), 7.79 (d, J = 8.2 Hz, 1H), 7.48 (d, J = 8.3 Hz, 1H), 7.36 (d, J = 2.7 Hz, 2H), 7.16 (d, J = 8.0 Hz, 1H), 6.85 (d, J = 2.8 Hz, 1H), 3.60 (s, 2H), 3.01-2.91 (d, J = 5.9 Hz, 4H), 2.79 (d, J = 4.8 Hz, 3H), 2.63-2.51 (s, 4H), 2.49 (s, 3H). |
| 11 | 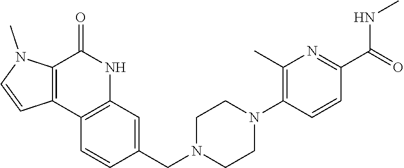 [SMILES: CNC(=O)c1ccc(N2CCN(Cc3ccc4c(c3)[nH]c(=O)c3c4ccn3C)CC2)c(C)n1] | 444.54 | 445.25 [M + H]⁺ | CDCl₃: δ 9.19 (s, 1H), 7.97-7.91 (m, 2H), 7.79 (d, J = 7.9 Hz, 1H), 7.32 (d, J = 8.3 Hz, 1H), 7.23-7.20 (m, 2H), 7.06 (d, J = 2.8 Hz, 1H), 6.69 (d, J = 2.8 Hz, 1H), 4.20 (s, 3H), 3.67 (s, 2H), 3.01-2.99 (m, 7H), 2.68 (s, 4H), 2.49 (s, 3H). |
| 12 | 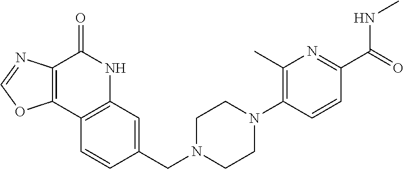 [SMILES: CNC(=O)c1ccc(N2CCN(Cc3ccc4c(c3)[nH]c(=O)c3ncoc34)CC2)c(C)n1] | 432.48 | 433.15 [M + H]⁺ | DMSO-d₆: δ 11.95 (br, 1H), 8.77 (s, 1H), 8.41-8.39 (m, 1H), 7.87 (d, J = 8.4 Hz, 1H), 7.75 (d, J = 8.0 Hz, 1H), 7.48 (s, 1H), 7.44 (d, J = 8.4 Hz, 1H), 7.29 (d, J = 8.0 Hz, 1H), 3.63 (s, 2H), 2.94-2.92 (m, 4H), 2.75 (d, J = 4.8 Hz, 3H), 2.58-2.56 (m, 4H), 2.45 (s, 3H) |
| 13 | 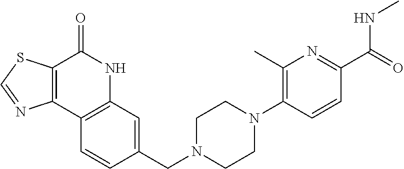 [SMILES: CNC(=O)c1ccc(N2CCN(Cc3ccc4c(c3)[nH]c(=O)c3scnc34)CC2)c(C)n1] | 448.55 | / | / |

### Example 14

### 7-((4-(2-methyl-6-(methylcarbamoyl)pyridin-3-yl)piperazin-1-yl)methyl)oxazolo[5,4-c]quinolin-4(5H)-one

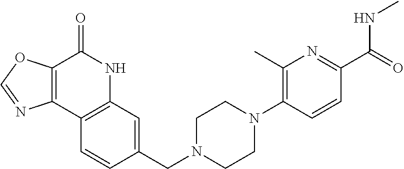 [SMILES: CNC(=O)c1ccc(N2CCN(Cc3ccc4c(c3)[nH]c(=O)c3ocnc34)CC2)c(C)n1]

a) Preparation of 4-amino-3-hydroxy-7-(hydroxymethyl)quinolin-2(1H)-one: To a solution of (3-amino-4-(4,4,5,5-tetramethyl-1,3,2-dioxaborolan-2-yl)phenyl)methanol (1.6 g, 7.2 mmol) in dioxane (20.0 mL) and H₂O (4.0 mL) was added ethyl 4-bromooxazole-5-carboxylate (1.8 g, 7.2 mmol), KCO₃ (1.987 g, 14.4 mmol), Pd(dppf)Cl₂ (522 mg, 0.72 mmol). The resulting mixture was stirred at 100° C. under nitrogen atmosphere overnight. The reaction mixture was slowly cooled to room temperature and concentrated under reduced pressure to give the product (1.6 g, crude, black oil). MS (ESI): 207.00[M+H].
b) Preparation of 4-amino-7-(bromomethyl)-3-hydroxyquinolin-2(1H)-one: A solution of 4-amino-3-hydroxy-7-(hydroxymethyl)quinolin-2(1H)-one (1.6 mg, 7.2 mmol) in HBr (48% in H₂O, 20 mL) was stirred at 85° C. for 1 hour. The reaction mixture was concentrated under reduced pressure. Acetonitrile (10 ml) was added to the residue and the mixture was concentrated under reduced pressure to give the product (1.6 g, crude, black oil). MS (ESI): 268.95[M+H]⁺.
c) Preparation of 7-(bromomethyl)oxazolo[5,4-c]quinolin-4(5H)-one: A solution of 4-amino-7-(bromomethyl)-3-hydroxyquinolin-2(1H)-one (1.6 g, 7.2 mmol) in trimethoxymethane (20 mL) was stirred at 85° C. for 18 hours. After completion of the reaction, the solvent was removed under vacuum. The residue was purified by Prep-TLC (PA:EA=1:1) to give the product (180 mg, crude, white solid, yield: 10%). MS (ESI): 280.95[M+H]⁺.
d) Preparation of 7-((4-(2-methyl-6-(methylcarbamoyl)pyridin-3-yl)piperazin-1-yl)methyl)oxazolo[5,4-c]quinolin-4(5H)-one: To a solution of 7-(bromomethyl)oxazolo[5,4-c]quinolin-4(5H)-one (180 mg, 0.7 mmol), KI (12 mg, 0.07 mmol) and N,6-dimethyl-5-(piperazin-1-yl)picolinamide (211 mg, 0.7 mmol) in CH₃CN (6 mL) was added DIEA (251.0 mg, 1.9 mmol) at room temperature under nitrogen. The resulting suspension was stirred at 80° C. for 1 hour. After completion of the reaction, the solvent was removed under vacuum. The residue was purified by Prep-HPLC to give the target compound (4.2 mg, white solid, yield: 1.4%). MS(ESI): 433.15[M+H]⁺. ¹H NMR (400 MHz, DMSO-d₆): δ 12.14 (s, 1H), 8.96 (s, 1H), 8.39 (s, 1H), 7.97 (d, J=8.0 Hz, 1H), 7.76 (d, J=8.1 Hz, 1H), 7.48 (s, 1H), 7.44 (d, J=8.4 Hz, 1H), 7.32 (d, J=7.9 Hz, 1H), 3.63 (s, 2H), 2.97-2.87 (m, 4H), 2.76 (d, J=4.6 Hz, 3H), 2.60-2.52 (m, 4H), 2.50 (s, 3H).

The following compounds of Examples 15-21 were prepared using a synthesis method similar to that described in Example 1 (Scheme 1); the compound of Example 22 was prepared using a synthesis method similar to that described in Example 6 (Scheme 2).

| | | | LC-MS | |
|---|---|---|---|---|
| Example | Compound | MW | (ESI) | ¹H NMR (400 MHz) |
| 15 | 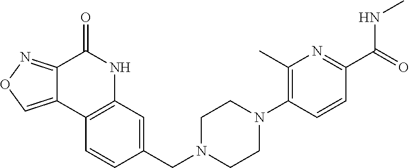 [SMILES: CNC(=O)c1ccc(N2CCN(Cc3ccc4c(c3)[nH]c(=O)c3nocc34)CC2)c(C)n1] | 432.48 | / | / |
| 16 | 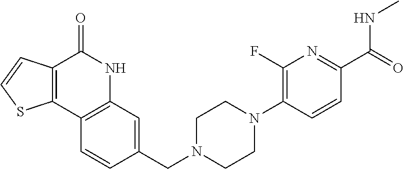 [SMILES: CNC(=O)c1ccc(N2CCN(Cc3ccc4c(c3)[nH]c(=O)c3ccsc34)CC2)c(F)n1] | 451.52 | / | / |
| 17 | 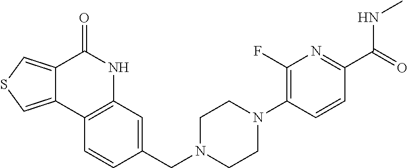 [SMILES: CNC(=O)c1ccc(N2CCN(Cc3ccc4c(c3)[nH]c(=O)c3cscc34)CC2)c(F)n1] | 451.52 | / | / |
| 18 | 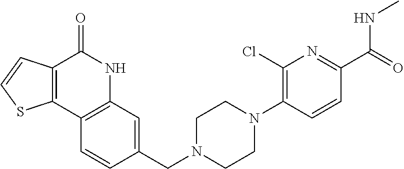 [SMILES: CNC(=O)c1ccc(N2CCN(Cc3ccc4c(c3)[nH]c(=O)c3ccsc34)CC2)c(Cl)n1] | 467.97 | / | / |
| 19 | 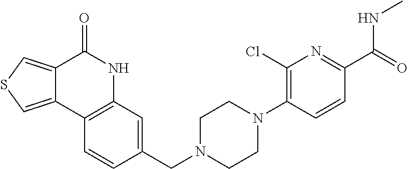 [SMILES: CNC(=O)c1ccc(N2CCN(Cc3ccc4c(c3)[nH]c(=O)c3cscc34)CC2)c(Cl)n1] | 467.97 | / | / |
| 20 | 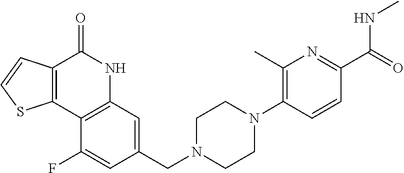 [SMILES: CNC(=O)c1ccc(N2CCN(Cc3cc(F)c4c(c3)[nH]c(=O)c3ccsc34)CC2)c(C)n1] | 465.55 | / | / |
| 21 | 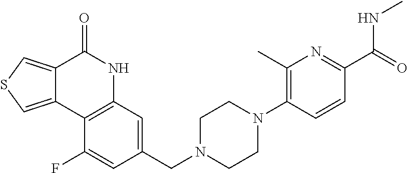 [SMILES: CNC(=O)c1ccc(N2CCN(Cc3cc(F)c4c(c3)[nH]c(=O)c3cscc34)CC2)c(C)n1] | 465.55 | 466.15 [M + H]⁺ | DMSO-d₆: δ 11.29 (s, 1H), 8.47 (s, 1H), 8.33 (s, 1H), 8.13 (s, 1H), 7.69 (d, J = 8.2 Hz, 1H), 7.38 (d, J = 8.3 Hz, 1H), 7.06 (s, 1H), 6.95 (d, J = 11.7 Hz, 1H), 3.50 (s, 2H), 2.91-2.83 (m, 4H), 2.70 (s, 3H), 2.51-2.45 (m, 4H), 2.42 (s, 3H). |
| 22 | 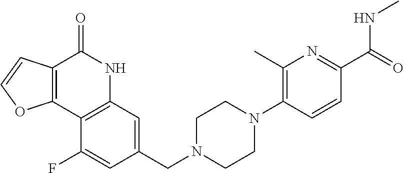 [SMILES: CNC(=O)c1ccc(N2CCN(Cc3cc(F)c4c(c3)[nH]c(=O)c3ccoc34)CC2)c(C)n1] | 449.49 | / | / |

### Example 23

### 7-((4-(2-methyl-6-(methylcarbamoyl)pyridin-3-yl)piperazin-1-yl)methyl)-1,5-dihydro-4H-pyrrolo[3,2-c]quinolin-4-one

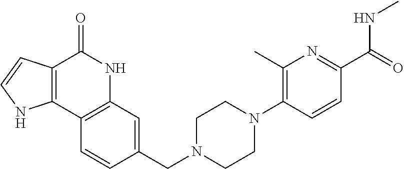 [SMILES: CNC(=O)c1ccc(N2CCN(Cc3ccc4c(c3)[nH]c(=O)c3cc[nH]c34)CC2)c(C)n1]

a) Preparation of N-(4-bromo-2-nitrobenzyl)formamide: A solution of (4-bromo-2-nitrophenyl)methanamine (11.5 g, 49.8 mmol) in ethyl format (100.0 mL) was stirred at 45° C. for 16 hours. After completion of the reaction, the mixture was cooled to room temperature and concentrated under reduced pressure. The residue was purified by column chromatography on silica gel (DCM/MeOH=1/0 to 200/1) to give the product (10.4 g, yellow solid, yield: 90%). MS (ESI): 258.95 [M+H]⁺.
b) Preparation of 1-(4-bromo-2-nitrophenyl)-N-methylidynemethanaminium: To a solution of N-4-bromo-2-nitrophenyl)methylformamide (500.0 mg, 1.9 mmol) in THF (30.0 mL), Burgess reagent (689.9 mg, 2.9 mmol) was added at room temperature. The reaction mixture was stirred at 45° C. for 1 hour. After completion of the reaction, the mixture was cooled to room temperature and concentrated under reduced pressure. The residue was purified by column chromatography on silica gel (PE/EA=1/0 to 50/1) to give the product (130.0 mg, white solid, yield: 27%). MS (ESI): 479.00 [2M+H]⁺.
c) Preparation of methyl 2-(4-bromo-2-nitrophenyl)-1H-pyrrole-3-carboxylate: To a solution of methyl prop-2-ynoate (750.2 mg, 8.9 mmol) and Ag₂CO₃ (164.0 mg, 0.6 mmol) in dioxane (42.0 mL) was added 1-(4-bromo-2-nitrophenyl)-N-methylidynemethanaminium (1.4 g, 5.9 mmol) at room temperature. The reaction mixture was stirred at 80° C. for 0.5 hour. After completion of the reaction, the mixture was cooled to room temperature, filtered and the filtrate was concentrated under reduced pressure. The residue was purified by column chromatography on silica gel (PE/EA=1/0 to 5/1) to give the product (1.5 g, yellow solid, yield: 77%). MS (ESI): 324.90 [M+H]⁺.
d) Preparation of 7-bromo-1,5-dihydro-4H-pyrrolo[3,2-c]quinolin-4-one: To a solution of methyl 2-(4-bromo-2-nitrophenyl)-1H-pyrrole-3-carboxylate (800.0 mg, 2.4 mmol) in acetic acid (20.0 mL), zinc dust (1.6 g, 24.6 mmol) was added at room temperature. The reaction mixture was stirred at 50° C. for 3 hours. After completion of the reaction, the mixture was cooled to room temperature, filtered through the celite. The filtrate was concentrated under reduced pressure. The residue was triturated with DCM (100 mL×3) to give the product (507.0 mg, white solid, yield: 78%). MS (ESI): 262.90 [M+H]⁺.
e) Preparation of 7-(hydroxymethyl)-1,5-dihydro-4H-pyrrolo[3,2-c]quinolin-4-one: To a solution of 7-bromo-1,5-dihydro-4H-pyrrolo[3,2-c]quinolin-4-one (250.0 mg, 0.9 mmol) in dioxane (20.0 mL), (tributylstannyl) methanol (1.5 g, 4.5 mmol) and Xphos Pd G2 (74.8 mg, 0.1 mmol) were added at room temperature. The reaction mixture was stirred at 110° C. under nitrogen for 16 hours. After completion of the reaction, the mixture was cooled to room temperature, quenched with saturated aqueous solution of KF (20.0 mL). The mixture was filtered. The solid was washed with MeOH (10.0 mL) to give the product (280 mg, crude, black solid). MS (ESI): 215.05 [M+H]⁺.
f) Preparation of 7-(bromomethyl)-1,5-dihydro-4H-pyrrolo[3,2-c]quinolin-4-one: To a solution of 7-(hydroxymethyl)-1H,4H,5H-pyrrolo[3,2-c]quinolin-4-one (175.0 mg, 0.8 mmol) in THF (15.0 mL), PBr₃ (442.2 mg, 1.6 mmol) was added at room temperature. The reaction mixture was stirred at room temperature for 2 hours. After completion of the reaction, the mixture was quenched with ice water (5.0 mL) and saturated aqueous solution of NaHCO₃ (10.0 mL) was added to adjust pH=7˜8, and then the mixture was extracted with DCM (100.0 mL×3). The organic phase was dried over anhydrous sodium sulfate, filtered and concentrated to give the product (230.0 mg, crude, yellow oil). MS (ESI): 276.90 [M+H]⁺.
g) Preparation of 7-((4-(2-methyl-6-(methylcarbamoyl)quinolin-3-yl)piperazin-1-yl)methyl)-1,5-dihydro-4H-pyrrolo[3,2-c]quinoline-4-one: To a solution of 7-(bromomethyl)-1,5-dihydro-4H-pyrrolo[3,2-c]quinolin-4-one (230.0 mg, 0.8 mmol) in MeCN (10.0 mL), N,6-dimethyl-5-(piperazin-1-yl)pyridine-2-carboxamide (291.6 mg, 1.2 mmol) and potassium carbonate (573.5 mg, 4.1 mmol) were added at room temperature. The reaction mixture was stirred at 80° C. for 16 hours. After completion of the reaction, the mixture was cooled to room temperature and concentrated. The residue was purified by Prep-HPLC (C18, MeCN/H₂O=0%-27%, 0.1% HCOOH) to give the title compound (42.4 mg, yellow solid, yield: 10%). MS(ESI): 431.20[M+H]⁺. ¹H NMR (400 MHz, CD₃OD): δ 8.12 (d, J=8.2 Hz, 1H), 7.89 (d, J=8.4 Hz, 1H), 7.58 (d, J=1.3 Hz, 1H), 7.55 (d, J=8.4 Hz, 1H), 7.45 (dd, J=8.2, 1.5 Hz, 1H), 7.26 (d, J=3.0 Hz, 1H), 6.84 (d, J=3.0 Hz, 1H), 4.54 (s, 2H), 3.68-3.55 (m, 2H), 3.51-3.38 (m, 4H), 3.16-3.04 (m, 2H), 2.93 (s, 3H), 2.59 (s, 3H).

### Example 24

### 7-((4-(2-fluoro-6-(methylcarbamoyl)pyridin-3-yl)piperazin-1-yl)methyl)-1-methyl-1,5-dihydro-4H-pyrrolo[3,2-c]quinolin-4-one

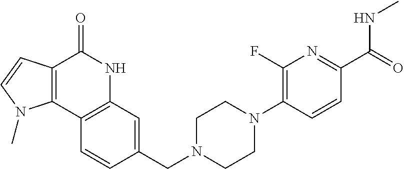 [SMILES: CNC(=O)c1ccc(N2CCN(Cc3ccc4c(c3)[nH]c(=O)c3ccn(C)c34)CC2)c(F)n1]

The compound of Example 24 was prepared using a synthesis method similar to that described in Example 23. MS(ESI): 449.20[M+H]⁺. ¹H NMR (400 MHz, DMSO-d₆): δ 11.18 (s, 1H), 8.43-8.36 (m, 1H), 8.08 (d, J=8.3 Hz, 1H), 7.81 (d, J=8.0 Hz, 1H), 7.53 (dd, J=10.6, 8.1 Hz, 1H), 7.36 (s, 1H), 7.21-7.08 (m, 2H), 6.54 (d, J=2.9 Hz, 1H), 4.11 (s, 3H), 3.56 (s, 2H), 3.22-3.12 (m, 4H), 2.72 (d, J=4.8 Hz, 3H), 2.58-2.49 (m, 4H).

### Example 25

### 7-((4-(2-methyl-6-(methylcarbamoyl)pyridin-3-yl)piperazin-1-yl)methyl)-1,5-dihydro-4H-imidazo[4,5-c]quinolin-4-one

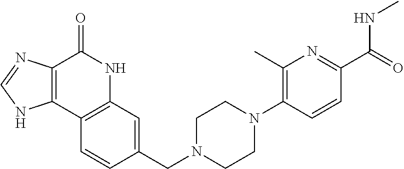 [SMILES: CNC(=O)c1ccc(N2CCN(Cc3ccc4c(c3)[nH]c(=O)c3nc[nH]c34)CC2)c(C)n1]

a) Preparation of (2-amino-4-(bromomethyl)phenyl)boronic acid: A solution of (3-amino-4-(4,4,5,5-tetramethyl-1,3,2-dioxaborolan-2-yl)phenyl)methanol (0.5 g) in HBr (48% aq, 10 mL) was stirred at 85° C. for 1 hour. The reaction mixture was concentrated under reduced pressure. Acetonitrile (5 ml) was added to the residue and the mixture was concentrated under reduced pressure three times to give the product (350 mg, crude, orange solid). MS (ESI): 232.05[M+H]⁺.
b) Preparation of (2-amino-4-((4-(2-methyl-6-(methylcarbamoyl)pyridin-3-yl)piperazin-1-yl)methyl)phenyl)boronic acid: To a solution of (2-amino-4-(bromomethyl)phenyl)boronic acid (1.0 g, crude) in acetonitrile (50 mL) was added N,6-dimethyl-5-(piperazin-1-yl)picolinamide (1.0 g, 4.4 mmol), KI (72 mg, 0.4 mmol) and DIEA (1.6 g, 13.1 mmol) at room temperature under nitrogen. The resulting mixture was stirred at 80° C. for 4 hours. The reaction mixture was concentrated under reduced pressure, and the residue was purified by Prep-HPLC to give the product (0.2 g, white solid, yield: 14%). MS (ESI): 384.00[M+H]⁺.
c) Preparation of methyl 5-(2-amino-4-((4-(2-methyl-6-(methylcarbamoyl)pyridin-3-yl)piperazin-1-yl)methyl)phenyl)-1H-imidazole-4-carboxylate: To a solution of methyl 5-bromo-1H-imidazole-4-carboxylate (100 mg, 0.5 mmol) in dioxane (10 mL) and H₂O (1 mL) was added (2-amino-4-((4-(2-methyl-6-(methylcarbamoyl)pyridin-3-yl)piperazin-1-yl)methyl)phenyl) boronic acid (187.7 mg, 0.5 mmol), K₂CO₃ (258.0 mg, 1.2 mmol), Pd(dppf)Cl₂ (72.6 mg, 0.1 mmol) at room temperature. The reaction mixture was allowed to heat to 100° C. and stirred for 18 hours under N₂. After completion of the reaction, the mixture was diluted with water (5 mL), extracted with DCM (10 mL×3). The combined organic layers were washed with brine, dried over anhydrous sodium sulfate, filtered and concentrated. The residue was purified by Prep-TLC ((DCM/MeOH=10/1) to give the product (30.0 mg, black solid). MS (ESI): 464.20[M+H]⁺.
d) Preparation of 7-((4-(2-methyl-6-(methylcarbamoyl)pyridin-3-yl)piperazin-1-yl)methyl)-1,5-dihydro-4H-imidazo[4,5-c]quinolin-4-one: A solution of methyl 5-(2-amino-4-((4-(2-methyl-6-(methylcarbamoyl)pyridin-3-yl)piperazin-1-yl)methyl)phenyl)-1H-imidazole-4-carboxylate (30.0 mg) in AcOH (10 mL) was stirred at 80° C. for 3 hours under nitrogen. The mixture was concentrated under reduced pressure. The residue was purified by Prep-HPLC (C18, CH₃CN/H₂O, 10˜40%, 0.1% HCOOH) to give the target compound (12.5 mg, white solid, 2-step yield: 6%). MS (ESI): 432.20[M+H]⁺. ¹H NMR (400 MHz, DMSO-d₆): δ 13.52 (s, 1H), 11.52 (s, 1H), 8.43-8.33 (m, 1H), 8.19 (s, 1H), 7.97 (s, 1H), 7.76 (d, J=8.1 Hz, 1H), 7.45 (d, J=8.4 Hz, 1H), 7.40 (s, 1H), 7.21 (d, J=8.3 Hz, 1H), 3.60 (s, 2H), 2.98-2.87 (m, 4H), 2.76 (d, J=4.9 Hz, 3H), 2.61-2.52 (m, 4H), 2.46 (s, 3H).

### Example 26

### 7-((4-(2-methyl-6-(methylcarbamoyl)pyridin-3-yl)piperazin-1-yl)methyl)-1-methyl-1,5-dihydro-4H-imidazo[4,5-c]quinolin-4-one

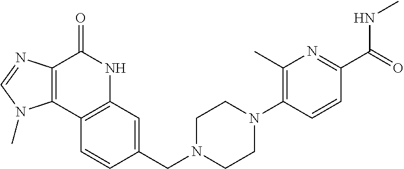 [SMILES: CNC(=O)c1ccc(N2CCN(Cc3ccc4c(c3)[nH]c(=O)c3ncn(C)c34)CC2)c(C)n1]

a) Preparation of 5-(4-(4-bromo-3-nitrobenzyl)piperazin-1-yl)-N,6-dimethylpicolinamide: To a solution of 1-bromo-4-(bromomethyl)-2-nitrobenzene (2.0 g), KI (113 mg, 0.7 mmol) and N,6-dimethyl-5-(piperazin-1-yl)picolinamide (1.6 g, 6.8 mmol) in CH₃CN (30 mL) was added DIEA (5.3 g, 40.8 mmol) at room temperature. The resulting solution was stirred at 80° C. for 2 hours. The mixture was quenched with water (50 mL) and extracted with DCM (50 mL×3). The combined organic phase was washed with brine, dried over anhydrous sodium sulfate and concentrated under reduced pressure. The residue was purified by column chromatography (SiO₂, Petroleum ether:Ethyl acetate=10/1 to 1/1) to give the product (1.1 g, yellow solid). MS (ESI): 450.30[M+H]⁺.
b) Preparation of 5-(4-(3-amino-4-bromobenzyl)piperazin-1-yl)-N,6-dimethylpicolinamide: To a solution of methyl 5-(4-(4-bromo-3-nitrobenzyl)piperazin-1-yl)-N,6-dimethylpicolinamide (1.0 g, 2.1 mmol) in EtOH (10 mL) and H₂O (10 mL) was added NH₄Cl (1.1 g, 21.0 mmol) and zinc dust (683 mg, 10.5 mmol) at room temperature. The reaction mixture was stirred at room temperature for 16 hours under N₂. The mixture was diluted with water (10 mL), and extracted with DCM (10 mL×3). The combined organic layers were washed with brine, dried over anhydrous sodium sulfate, filtered and concentrated. The residue was purified by silica gel column (DCM/MeOH=100/1˜20/1) to give the product (470 mg, yellow solid, yield: 50%). MS (ESI): 420.05 [M+H]⁺.
c) Preparation of methyl 5-(2-amino-4-((4-(2-methyl-6-(methylcarbamoyl)pyridin-3-yl)piperazin-1-yl)methyl)phenyl)-1-methyl-1H-imidazole-4-carboxylate: To a solution of methyl 1-methyl-5-(trimethylstannyl)-1H-imidazole-4-carboxylate (220 mg, 0.7 mmol) in dioxane (10 mL) was added 5-(4-(3-amino-4-bromobenzyl)piperazin-1-yl)-N,6-dimethylpicolinamide (302 mg, 0.7 mmol) and Pd(dppf)Cl₂ (73 mg, 0.1 mmol) at room temperature. The reaction mixture was allowed to heat to 100° C. and stirred for 16 hours under N₂. The mixture was diluted with water (10 mL) and extracted with DCM (10 mL×3). The combined organic layers were washed with brine, dried over anhydrous sodium sulfate and concentrated under reduced pressure. The residue was purified by Prep-TLC (DCM/MeOH=10/1) to afford the product (40 mg, grey solid). MS (ESI): 478.25[M+H]⁺.
d) Preparation of 7-((4-(2-methyl-6-(methylcarbamoyl)pyridin-3-yl)piperazin-1-yl)methyl)-1-methyl-1,5-dihydro-4H-imidazo[4,5-c]quinolin-4-one: A solution of methyl 5-(2-amino-4-((4-(2-methyl-6-(methylcarbamoyl)pyridin-3-yl)piperazin-1-yl)methyl)phenyl)-1-methyl-1H-imidazole-4-carboxylate (40 mg) in AcOH was stirred at 80° C. for 3 hours under N₂. After completion of the reaction, the mixture was concentrated. The residue was purified by Prep-HPLC (C18, CH₃CN/H₂O, 10˜40%, 0.1% HCOOH) to afford the target compound (7.5 mg, white solid, 2-step yield: 2%). MS (ESI): 446.15[M+H]⁺. ¹H NMR (400 MHz, DMSO-d₆): δ 11.46 (s, 1H), 8.40-8.36 (m, 1H), 8.24 (s, 1H), 8.06 (d, J=8.2 Hz, 1H), 7.76 (d, J=8.2 Hz, 1H), 7.44 (d, J=8 Hz, 1H), 7.42 (d, J=1.6 Hz, 1H), 7.21 (dd, J=8.2, 1.6 Hz, 1H), 4.12 (s, 3H), 3.60 (s, 2H), 2.98-2.86 (m, 4H), 2.76 (d, J=4.8 Hz, 3H), 2.59-2.50 (m, 4H), 2.46 (s, 3H).

The following compounds of Examples 27-29 were prepared using a synthesis method similar to that described in Example 1 (Scheme 1).

| | | | LC-MS | |
|---|---|---|---|---|
| Example | Compound | MW | (ESI) | ¹H NMR (400 MHz) |
| 27 | 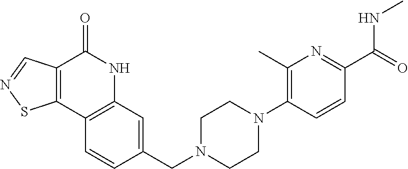 [SMILES: CNC(=O)c1ccc(N2CCN(Cc3ccc4c(c3)[nH]c(=O)c3cnsc34)CC2)c(C)n1] | 448.55 | / | / |
| 28 | 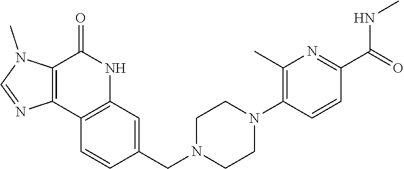 [SMILES: CNC(=O)c1ccc(N2CCN(Cc3ccc4c(c3)[nH]c(=O)c3c4ncn3C)CC2)c(C)n1] | 445.33 | 446.20 [M + H]⁺ | DMSO-d₆: δ 11.56 (s, 1H), 8.38 (q, J = 6.1, 5.4 Hz, 1H), 8.17 (s, 1H), 7.96 (d, J = 8.0 Hz, 1H), 7.76 (d, J = 8.3 Hz, 1H), 7.44 (d, J = 8.4 Hz, 1H), 7.37 (s, 1H), 7.25-7.15 (m, 1H), 4.02 (s, 3H), 3.59 (s, 2H), 2.99-2.82 (m, 4H), 2.76 (d, J = 4.8 Hz, 3H), 2.61-2.50 (m, 4H), 2.45 (s, 3H). |
| 29 | 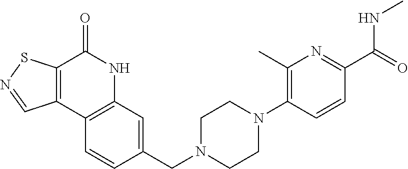 [SMILES: CNC(=O)c1ccc(N2CCN(Cc3ccc4c(c3)[nH]c(=O)c3sncc34)CC2)c(C)n1] | 448.55 | / | / |

### Example 30

### 7-((4-(2-methyl-6-(methylcarbamoyl)pyridin-3-yl)piperazin-1-yl)methyl)isoxazolo[5,4-c]quinolin-4(5H)-one

To a solution of (2-amino-4-((4-(2-methyl-6-(methylcarbamoyl)pyridin-3-yl)piperazin-1-yl)methyl)phenyl)boronic acid (64 mg, 0.2 mmol) in dioxane (4.0 mL) and H₂O (1.0 ml) was added ethyl 4-bromooxazole-5-carboxylate (45 mg, 0.2 mmol), K₂CO₃ (70 mg, 0.5 mmol) and Pd(dppf)Cl₂ (15 mg, 0.02 mmol) at room temperature under N₂ atmosphere. The resulting mixture was stirred at 100° C. overnight. The mixture was slowly cooled to room temperature and concentrated under reduced pressure. The residue was purified by Prep-HPLC to afford the target compound (6.6 mg, grey solid).

The following compounds of Examples 31-37 and 39 were prepared using a synthesis method similar to that described in Example 1 (Scheme 1); the compound of Example 38 was prepared using a synthesis method similar to that described in Example 40 (Scheme 4).

| | | | LC-MS | |
|---|---|---|---|---|
| Example | Compound | MW | (ESI) | ¹H NMR (400 MHz) |
| 30 | 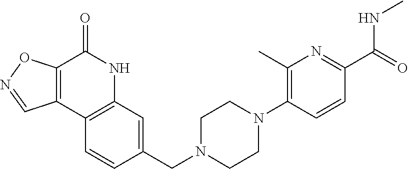 [SMILES: CNC(=O)c1ccc(N2CCN(Cc3ccc4c(c3)[nH]c(=O)c3oncc34)CC2)c(C)n1] | 432.48 | 433.10 [M + H]⁺ | DMSO-d6: δ 12.03 (br, 1H), 8.41-8.36 (m, 1H), 8.10 (s, 1H), 7.75 (d, J = 8.1 Hz, 1H), 7.43 (d, J = 8.3 Hz, 1H), 7.25-7.19 (m, 1H), 7.15 (s, 1H), 7.09 (d, J = 8.1 Hz, 1H), 3.53 (s, 2H), 2.95-2.86 (m, 4H), 2.76 (d, J = 4.8 Hz, 3H), 2.60-2.51 (m, 4H), 2.45 (s, 3H). |
| 31 | 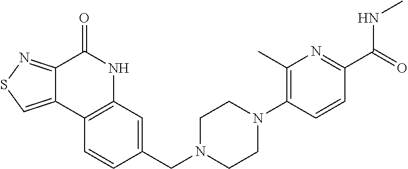 [SMILES: CNC(=O)c1ccc(N2CCN(Cc3ccc4c(c3)[nH]c(=O)c3nscc34)CC2)c(C)n1] | 448.55 | / | / |
| 32 | 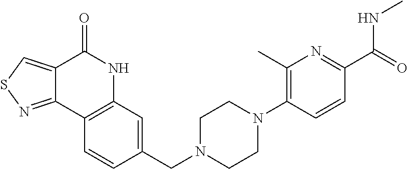 [SMILES: CNC(=O)c1ccc(N2CCN(Cc3ccc4c(c3)[nH]c(=O)c3csnc34)CC2)c(C)n1] | 448.55 | / | / |
| 33 | 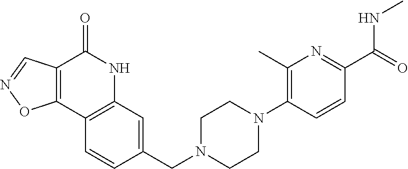 [SMILES: CNC(=O)c1ccc(N2CCN(Cc3ccc4c(c3)[nH]c(=O)c3cnoc34)CC2)c(C)n1] | 432.48 | / | / |
| 34 | 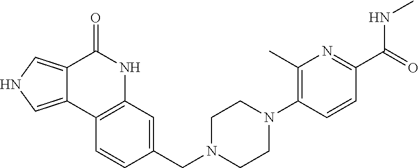 [SMILES: CNC(=O)c1ccc(N2CCN(Cc3ccc4c(c3)[nH]c(=O)c3c[nH]cc34)CC2)c(C)n1] | 430.51 | 431.15 [M + H]⁺ | DMSO-d6: δ 12.08 (s, 1H), 10.63 (s, 1H), 8.42 (d, J = 5.0 Hz, 1H), 7.80 (dd, J = 3.4 Hz, J = 8.0 Hz, 1H), 7.78 (d, J = 3.8 Hz, 1H), 7.58 (t, J = 2.3 Hz, 1H), 7.52 (t, J = 2.4 Hz, 1H), 7.47 (d, J = 8.3 Hz, 1H), 7.21 (s, 1H), 7.05 (d, J = 7.8 Hz, 1H), 3.55 (s, 2H), 2.95-2.93 (m, 4H), 2.79 (d, J = 4.8 Hz, 3H), 2.62-2.55 (m, 4H), 2.48 (s, 3H). |
| 35 | 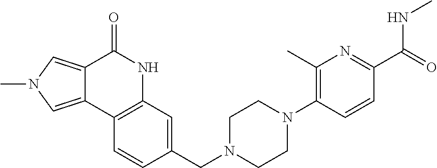 | 444.54 | 445.20 [M + H]⁺ | DMSO-d6: δ 10.65 (s, 1H), 8.42 (q, J = 4.8 Hz, 1H), 7.79 (d, J = 8.2 Hz, 1H), 7.73 (d, J = 7.9 Hz, 1H), 7.54-7.51 (m, 2H), 7.47 (d, J = 8.3 Hz, 1H), 7.21 (s, 1H), 7.05 (d, J = 7.9 Hz, 1H), 3.86 (s, 3H), 3.55 (s, 2H), 2.97-2.92 (m, 4H), 2.79 (d, J = 4.8 Hz, 3H), 2.59-2.54 (s, 4H), 2.48 (s, 3H). |
| 36 | 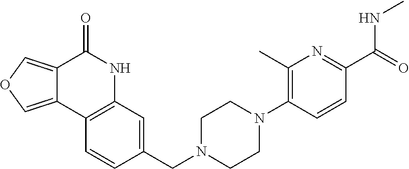 [SMILES: CNC(=O)c1ccc(N2CCN(Cc3ccc4c(c3)[nH]c(=O)c3cocc34)CC2)c(C)n1] | 431.5 | / | / |
| 37 | 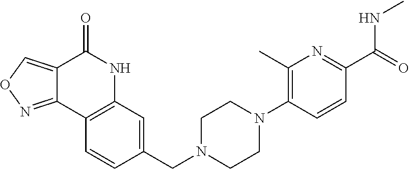 [SMILES: CNC(=O)c1ccc(N2CCN(Cc3ccc4c(c3)[nH]c(=O)c3conc34)CC2)c(C)n1] | 432.48 | / | / |
| 38 | 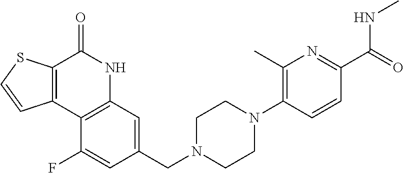 [SMILES: CNC(=O)c1ccc(N2CCN(Cc3cc(F)c4c(c3)[nH]c(=O)c3sccc34)CC2)c(C)n1] | 465.55 | 466.15 [M + H]⁺ | DMSO-d6: δ 12.09 (s, 1H), 8.44 (q, J = 4.3 Hz, 1H), 8.24 (d, J = 5.1 Hz, 1H), 7.92-7.86 (m, 1H), 7.79 (d, J = 8.2 Hz, 1H), 7.48 (d, J = 8.3 Hz, 1H), 7.31 (s, 1H), 7.13 (d, J = 12.0 Hz, 1H), 3.65 (s, 2H), 3.00-2.93 (m, 4H), 2.79 (d, J = 4.5 Hz, 3H), 2.65-2.53 (m, 4H), 2.48 (s, 3H). |
| 39 | 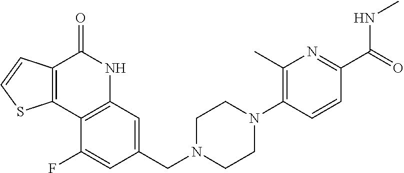 [SMILES: CNC(=O)c1ccc(N2CCN(Cc3cc(F)c4c(c3)[nH]c(=O)c3ccsc34)CC2)c(C)n1] | 465.55 | / | / |

### Example 40

### 9-fluoro-7-((4-(2-methyl-6-(methylcarbamoyl)pyridin-3-yl)piperazin-1-yl)methyl)furo[2,3-c]quinolin-4(5H)-one

a) Preparation of ethyl 3-(2-fluoro-4-(methoxycarbonyl)-6-nitrophenyl)furan-2-carboxylate: To a solution of methyl 4-bromo-3-fluoro-5-nitrobenzoate (500.0 mg, 1.8 mmol) in THF (10 mL) and H₂O (2 mL) was added (2-(ethoxycarbonyl)furan-3-yl)boronic acid (398.5 mg, 2.1 mmol), Pd(PPh₃)₂Cl₂ (126.3 mg, 0.2 mmol), and K₂CO₃ (496.8 mg, 3.6 mmol). The mixture was degassed and purged with nitrogen for 2 minutes, and stirred at 85° C. for 16 hours under nitrogen. After completion of the reaction, the mixture was quenched with water (30 mL) and extracted with DCM (30 mL×2). The combined organic phase was washed with brine, dried over anhydrous sodium sulfate and concentrated under reduced pressure. The residue was purified by Prep-TLC (PE/EA=40/1˜10/1) to give the product (450.0 mg, yellow solid, yield: 74%).
b) Preparation of methyl 9-fluoro-4-oxo-4,5-dihydrofuro[2,3-c]quinoline-7-carboxylate: To a solution of ethyl 3-(2-fluoro-4-(methoxycarbonyl)-6-nitrophenyl)furan-2-carboxylate (450.0 mg, 1.3 mmol) in AcOH (20 mL) was added iron dust (375.2 mg, 6.7 mmol). The resulting mixture was stirred at 80° C. for 2 hours. After completion of the reaction, the reaction mixture was filtered, and the filtrate was concentrated under reduced pressure. The residue was diluted with water (10 mL) and extracted with DCM (20 mL×3). The combined organic phase was washed with brine, dried over anhydrous sodium sulfate and concentrated under reduced pressure to give the product (400.0 mg, crude, brown solid). MS(ESI): 262.05 [M+H]⁺.
c) Preparation of 9-fluoro-7-(hydroxymethyl)furo[2,3-c]quinolin-4(5H)-one: To a solution of methyl 9-fluoro-4-oxo-4,5-dihydrofuro[2,3-c]quinoline-7-carboxylate (400 mg) in THF (5 mL) was added LiAlH₄ (1 M in THF, 3.1 mL, 3.1 mmol) at 0° C. under nitrogen. The resulting mixture was stirred at 0° C. for 20 minutes and then warmed to room temperature. After stirred for 2 hours, the mixture was quenched with ice-water (20 mL) and adjusted pH value to 3 with 1 M HCl aqueous solution. The resulting mixture was extracted with EA (30 mL×3). The combined organic phase was washed with brine, dried over anhydrous sodium sulfate and concentrated under reduced pressure to give the product (150.0 mg, yellow solid, 2-step yield: 48%). MS(ESI): 234.00 [M+H]⁺.
d) Preparation of 7-(bromomethyl)-9-fluorofuro[2,3-c]quinolin-4(5H)-one: A solution of 9-fluoro-7-(hydroxymethyl)furo[2,3-c]quinolin-4(5H)-one (150 mg, 0.6 mmol) in HBr (48% in water, 10 mL) was stirred at 80° C. for 2 hours. After completion of the reaction, the mixture was concentrated to give the product (150.0 mg, crude, yellow solid). MS(ESI): 297.90 [M+H]⁺.
e) Preparation of 9-fluoro-7-((4-(2-methyl-6-(methylcarbamoyl)pyridin-3-yl)piperazin-1-yl)methyl)furo[2,3-c]quinolin-4(5H)-one: To a solution of 7-(bromomethyl)-9-fluorofuro[2,3-c]quinolin-4(5H)-one (75.0 mg, crude from above), KI (16.6 mg, 0.1 mmol) and N,6-dimethyl-5-(piperazin-1-yl)picolinamide (93.2 mg, 0.4 mmol) in CH₃CN (10 mL) was added DIEA (196.7 mg, 1.5 mmol) at room temperature. The resulting suspension was stirred at 80° C. for 2 hours. After completion of the reaction, the solvent was removed under vacuum. The residue was purified by Prep-TLC (DCM/MeOH=15/1) to give the target compound (34.0 mg, white powder, 2-step yield: 16%).

The following compound of Examples 41 was prepared using a synthesis method similar to that described in Example 1 (Scheme 1); the compounds of Examples 42 and 45-46 were prepared using a synthesis method similar to that described in Example 7 (Scheme 3); the compounds of Examples 43-44 were prepared using a synthesis method similar to that described in Example 6 (Scheme 2).

| | | | LC-MS | |
|---|---|---|---|---|
| Example | Compound | MW | (ESI) | ¹H NMR (400 MHz) |
| 40 | 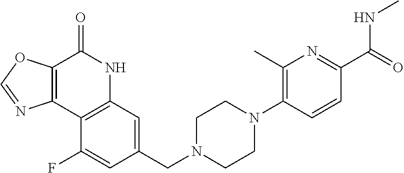 [SMILES: CNC(=O)c1ccc(N2CCN(Cc3cc(F)c4c(c3)[nH]c(=O)c3ocnc34)CC2)c(C)n1] | 449.49 | 450.20 [M + H]⁺ | DMSO-d₆: δ 12.06 (s, 1H), 8.39 (q, J = 5.3, 4.6 Hz, 1H), 8.27 (d, J = 1.9 Hz, 1H), 7.76 (d, J = 8.3 Hz, 1H), 7.45 (d, J = 8.3 Hz, 1H), 7.28- 7.18 (m, 2H), 7.10 (d, J = 11.5 Hz, 1H), 3.61 (s, 2H), 2.93 (dd, J = 6.1, 3.4 Hz, 4H), 2.76 (d, J = 4.8 Hz, 3H), 2.61-2.53 (m, 4H), 2.45 (s, 3H). |
| 41 | 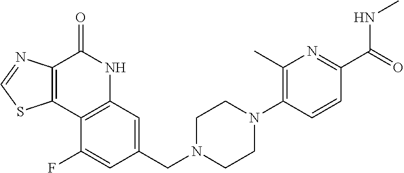 [SMILES: CNC(=O)c1ccc(N2CCN(Cc3cc(F)c4c(c3)[nH]c(=O)c3ncsc34)CC2)c(C)n1] | 466.54 | / | / |
| 42 | 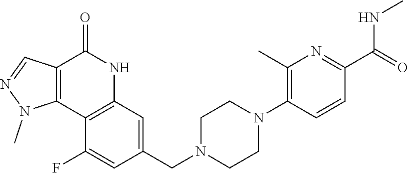 [SMILES: CNC(=O)c1ccc(N2CCN(Cc3cc(F)c4c(c3)[nH]c(=O)c3cnn(C)c34)CC2)c(C)n1] | 463.52 | / | / |
| 43 | 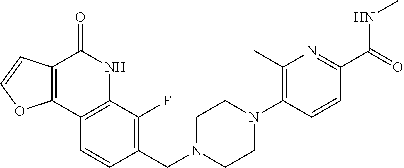 [SMILES: CNC(=O)c1ccc(N2CCN(Cc3ccc4c([nH]c(=O)c5ccoc54)c3F)CC2)c(C)n1] | 449.49 | 450.10 [M + H]⁺ | DMSO-d₆: δ 11.75 (s, 1H), 8.46-8.38 (m, 1H), 8.13 (d, J = 2.0 Hz, 1H), 7.76 (dd, J = 17.1, 8.2 Hz, 2H), 7.47 (d, J = 8.2 Hz, 1H), 7.38-7.27 (m, 1H), 7.11 (d, J = 2.1 Hz, 1H), 3.74 (s, 2H), 2.99-2.90 (m, 4H), 2.79 (d, J = 4.8 Hz, 3H), 2.66-2.59 (m, 4H), 2.48 (s, 3H). |
| 44 | 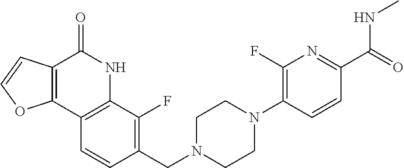 [SMILES: CNC(=O)c1ccc(N2CCN(Cc3ccc4c([nH]c(=O)c5ccoc54)c3F)CC2)c(F)n1] | 453.45 | 454.10 [M + H]⁺ | DMSO-d₆: δ 11.76 (s, 1H), 8.46-8.37 (m, 1H), 8.14 (d, J = 2.1 Hz, 1H), 7.83 (d, J = 8.0 Hz, 1H), 7.73 (d, J = 8.2 Hz, 1H), 7.55 (dd, J = 10.6, 8.1 Hz, 1H), 7.37-7.30 (m, 1H), 7.11 (d, J = 2.1 Hz, 1H), 3.72 (s, 2H), 3.20-3.13 (m, 4H), 2.75 (d, J = 4.7 Hz, 3H), 2.64-2.56 (m, 4H). |
| 45 | 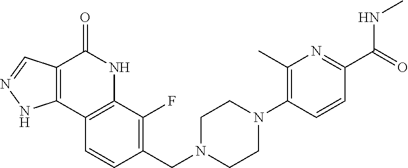 [SMILES: CNC(=O)c1ccc(N2CCN(Cc3ccc4c([nH]c(=O)c5cn[nH]c54)c3F)CC2)c(C)n1] | 449.49 | 450.20 [M + H]⁺ | DMSO-d₆: δ 11.33 (br, 1H), δ 8.42 (d, 1H), δ 7.79 (d, J = 8.1 Hz, 2H), 7.48 (d, J = 8.5 Hz, 2H), 3.68 (s, 2H), 2.98-2.92 (m, 4H), 2.79 (d, J = 4.8 Hz, 3H), 2.68-2.58 (m, 4H), 2.49 (s, 3H). |
| 46 | 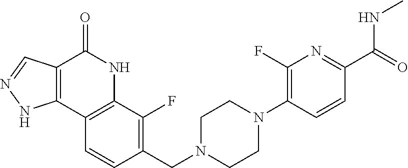 [SMILES: CNC(=O)c1ccc(N2CCN(Cc3ccc4c([nH]c(=O)c5cn[nH]c54)c3F)CC2)c(F)n1] | 453.45 | 454.15 [M + H]⁺ | DMSO-d₆: δ 11.26 (s, 1H), 8.40 (d, J = 5.1 Hz, 1H), 8.34 (s, 1H), 7.83 (t, J = 8.3 Hz, 2H), 7.57 (dd, J = 10.6, 8.0 Hz, 1H), 7.51 (d, J = 6.3 Hz, 1H), 3.66 (s, 2H), 3.21-3.16 (m, 4H), 2.76 (d, J = 4.8 Hz, 3H), 2.65-2.59 (m, 4H). |

### Example 47

### 7-((4-(2-methyl-6-(methylcarbamoyl)pyridin-3-yl)piperazin-1-yl)methyl)-3-methylisoxazolo[4,5-c]quinolin-4(5H)-one

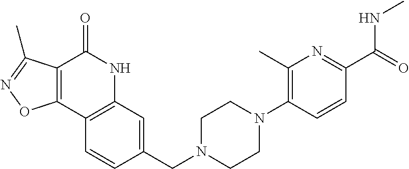 [SMILES: CNC(=O)c1ccc(N2CCN(Cc3ccc4c(c3)[nH]c(=O)c3c(C)noc34)CC2)c(C)n1]

a) Preparation of 7-(hydroxymethyl)-3-methylisoxazolo[4,5-c]quinolin-4(5H)-one: To a solution of (3-amino-4-(4,4,5,5-tetramethyl-1,3,2-dioxaborolan-2-yl)phenyl)methanol (500.0 mg, 2.0 mmol) in dioxane (5 mL) was added ethyl 5-chloro-3-methylisoxazole-4-carboxylate (378.0 mg, 2.0 mmol), K₂CO₃ (553 mg, 4.0 mmol), Pd(dppf)Cl₂ (145 mg, 0.2 mmol). The resulting mixture was stirred at 100° C. under nitrogen atmosphere overnight. The reaction mixture was slowly cooled to room temperature and concentrated under reduced pressure to give the product (350.0 mg, crude, white solid), which was used for the next step without further purification. MS(ESI): 231.05[M+H]⁺.
b) Preparation of 7-(bromomethyl)-3-methylisoxazolo[4,5-c]quinolin-4(5H)-one: A solution of 7-(bromomethyl)-3-methylisoxazolo[4,5-c]quinolin-4(5H)-one (350.0 mg, crude) in HBr (48% aqueous solution, 6 mL) was stirred at 85° C. for 1 hour. The reaction mixture was concentrated under reduced pressure. Acetonitrile (3 mL) was added to the residue and the mixture was concentrated under reduced pressure to give the product (150.0 mg, crude, orange solid). MS(ESI): 294.95[M+H]⁺.
c) Preparation of 7-((4-(2-methyl-6-(methylcarbamoyl)pyridin-3-yl)piperazin-1-yl)methyl)-3-methylisoxazolo[4,5-c]quinolin-4(5H)-one: To a solution of 7-(bromomethyl)-3-methylisoxazolo[4,5-c]quinolin-4(5H)-one (324.0 mg, crude), KI (45.0 mg) and N,6-dimethyl-5-(piperazin-1-yl)picolinamide (297.0 mg, 1.0 mmol) in acetonitrile (6 mL) was added DIEA (387.0 mg, 3.0 mmol) at room temperature under nitrogen. The resulting mixture was stirred at 80° C. for 4 hours. After completion of the reaction, the solvent was removed under vacuum. The residue was purified by Prep-HPLC to give the target compound (4.2 mg, white solid, 3-step yield: 1%). MS(ESI): 447.25[M+H]⁺. ¹H NMR (400 MHz, DMSO-d₆): δ 11.88 (s, 1H), 8.43 (d, J=5.3 Hz, 1H), 7.99 (d, J=8.1 Hz, 1H), 7.79 (d, J=8.3 Hz, 1H), 7.48 (d, J=6.8 Hz, 2H), 7.33 (d, J=8.1 Hz, 1H), 3.67 (s, 2H), 2.99-2.93 (m, 4H), 2.80 (d, J=4.8 Hz, 3H), 2.64-2.57 (m, 4H), 2.56 (s, 3H), 2.49 (s, 3H).

### Example 48

### 8-((4-(2-methyl-6-(methylcarbamoyl)pyridin-3-yl)piperazin-1-yl)methyl)-7-fluoropyrrolo[1,2-c]quinazolin-5(6H)-one

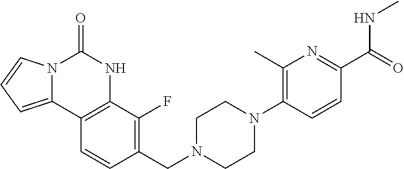 [SMILES: CNC(=O)c1ccc(N2CCN(Cc3ccc4c([nH]c(=O)n5cccc45)c3F)CC2)c(C)n1]

a) Preparation of tert-butyl 2-(4-bromo-3-fluoro-2-nitrophenyl)-1H-pyrrole-1-carboxylate: To a solution of 1,4-dibromo-2-fluoro-3-nitrobenzene (1.0 g, 3.4 mmol) in toluene (20 mL) and H₂O (4 mL) was added (1-(tert-butoxycarbonyl)-1H-pyrrol-2-yl) boronic acid (745.9 mg, 3.5 mmol), Pd(PPh₃)₄ (389.2 mg, 0.3 mmol) and Na₂CO₃ (1.3 g, 11.8 mmol). The mixture was degassed and purged with nitrogen for 2 minutes. The tube was sealed and the mixture was stirred at 85° C. for 16 hours. After completion of the reaction, the mixture was cooled to room temperature, water (50 mL) was added, and the mixture was extracted with DCM (50 mL×3). The combined organic phase was washed with brine, dried over anhydrous sodium sulfate and concentrated under reduced pressure. The residue was purified by silica gel (EtOAc/PE, 1 to 10%) to give the product (1.2 g, yellow oil, yield: 92.2%).
b) Preparation of 8-bromo-7-fluoropyrrolo[1,2-c]quinazolin-5(6H)-one: To a solution of tert-butyl 2-(4-bromo-3-fluoro-2-nitrophenyl)-1H-pyrrole-1-carboxylate (1.2 g, 3.2 mmol) in AcOH (50 mL) was added iron dust (905 mg, 16.2 mmol). The resulting mixture was stirred at 80° C. for 3 hours. After completion of the reaction, the reaction mixture was filtered and the filtrate was concentrated under reduced pressure. The residue was diluted with water (50 mL) and extracted with DCM (30 mL×3). The combined organic phase was washed with brine, dried over anhydrous sodium sulfate and concentrated under reduced pressure. The residue was purified by silica gel column (EtOAc/PE, 5 to 20%) to give the product (165 mg, yellow solid, yield: 18.9%). MS(ESI): 280.95 [M+H]⁺.
c) Preparation of 7-fluoro-8-(hydroxymethyl)pyrrolo[1,2-c]quinazolin-5(6H)-one: To a solution of 8-bromo-7-fluoropyrrolo[1,2-c]quinazolin-5(6H)-one (105 mg, 0.38 mmol) in dioxane (5 mL) was added (tributylstannyl)methanol (181 mg, 0.56 mmol) and Xphos Pd G2 (44 mg, 0.06 mmol) at room temperature. The mixture was stirred at 90° C. overnight under nitrogen atmosphere. After completion of the reaction, a solution of KF (1 M aqueous solution, 10 mL) was added at room temperature, the mixture was stirred at the same temperature for 0.5 hours and filtered, and the filtrate was extracted with EA (50 mL×3). The combined organic phase was washed with brine, dried over anhydrous Na₂SO₄ and concentrated under reduced pressure. The residue was purified by silica gel column (EtOAc/PE, 10 to 50%) to give the product (65 mg, white solid, yield: 74.9%). MS(ESI): 233.00 [M+H]⁺.
d) Preparation of 8-(chloromethyl)-7-fluoropyrrolo[1,2-c]quinazolin-5(6H)-one: To a solution of 7-fluoro-8-(hydroxymethyl)pyrrolo[1,2-c]quinazolin-5(6H)-one (57.0 mg, 0.25 mmol) in DCM (5 mL) was added SOCl₂ (58.1 mg, 0.49 mmol) and DMF (2 drops). The mixture stirred at 80° C. for 2 hours. After completion of the reaction, the mixture was concentrated to give the product (55.3 mg, crude, yellow solid). MS(ESI): 251.00 [M+H]⁺.
e) Preparation of 8-((4-(2-methyl-6-(methylcarbamoyl)pyridin-3-yl)piperazin-1-yl)methyl)-7-fluoropyrrolo[1,2-c]quinazolin-5(6H)-one: To a solution of 8-(bromomethyl)-7-fluoropyrrolo[1,2-c]quinazolin-5(6H)-one (20.0 mg), KI (1.3 mg, 0.01 mmol) and N,6-dimethyl-5-(piperazin-1-yl)picolinamide (22.5 mg, 0.10 mmol) in CH₃CN (5 mL) was added K₂CO₃ (44.2 mg, 0.32 mmol) at room temperature. The resulting suspension was stirred at 80° C. for 16 hours. After completion of the reaction, the solvent was removed under vacuum. The residue was purified by Prep-TLC (DCM/MeOH=10/1) to give the target compound (7.2 mg, white solid, yield: 19.5%). MS(ESI): 449.20[M+H]⁺. ¹H NMR (400 MHz, DMSO-d₆): δ 11.54 (s, 1H), 8.38 (d, J=4.5 Hz, 1H), 7.74 (d, J=8.2 Hz, 1H), 7.70 (d, J=8.1 Hz, 1H), 7.61 (s, 1H), 7.43 (d, J=8.3 Hz, 1H), 7.20 (t, J=7.0 Hz, 1H), 7.01 (s, 1H), 6.67 (d, J=3.4 Hz, 1H), 3.64 (s, 2H), 2.93-2.88 (m, 4H), 2.75 (d, J=4.4 Hz, 3H), 2.59-2.55 (m, 4H), 2.44 (s, 3H).

### Example 49

### 7-((4-(2-fluoro-6-(methylcarbamoyl)pyridin-3-yl)piperazin-1-yl)methyl)-3-fluoropyrazolo[1,5-a]quinoxalin-4(5H)-one

a) Preparation of 4-fluoro-1H-pyrazole-5-carbonyl chloride: A suspension of 4-fluoro-1H-pyrazole-5-carboxylic acid (175.0 mg, 1.35 mmol, 1.0 eq) in SOCl₂ (5 mL) was stirred at 80° C. under nitrogen for 2 hours. The mixture was concentrated in vacuum to give the product (160 mg, crude, white solid).
b) Preparation of methyl 4-fluoro-3-(4-fluoro-1H-pyrazole-5-carboxamido)benzoate: To a solution of 4-fluoro-1H-methyl 3-amino-4-fluorobenzoate (218 mg, 1.3 mmol) in THF (3 mL) was added LiHMDS (1.0 M in THF, 3.2 mL) at 0° C. under nitrogen. The mixture was stirred at 0° C. for 15 minutes, and then the solution of 4-fluoro-1H-pyrazole-5-carbonyl chloride (160.0 mg, 1.1 mmol, in 2 mL THF) was added dropwise and the mixture was stirred at 0° C. for 0.5 hour. After completion of the reaction, water (10 mL) was added to quench LiHMDS and the mixture was extracted with EA (5 mL×3). The combined organic phase was dried with anhydrous sodium sulfate and concentrated in vacuum. The residue was purified by Prep-TLC (DCM/MeOH=20/1) to give the product (110 mg, white solid, yield: 36%). MS(ESI): 282.00 [M+H]⁺.
c) Preparation of methyl 3-fluoro-4-oxo-4,5-dihydropyrazolo[1,5-a]quinoxaline-7-carboxylate: To a solution of methyl 4-fluoro-3-(4-fluoro-1H-pyrazole-5-carboxamido)benzoate (100 mg, 0.4 mmol) in DMSO (10 mL) was added K₂CO₃ (194 mg, 1.4 mmol) at room temperature under nitrogen. The mixture was stirred at 120° C. overnight in sealed tube. After completion of the reaction, water (20 mL) was added and the mixture was extracted with EA (10 mL×3). The combined organic phase was dried with anhydrous sodium sulfate and concentrated in vacuum to give the product (85 mg, white solid, yield: 91%). MS(ESI): 262.05 [M+H]⁺.
d) Preparation of 3-fluoro-7-(hydroxymethyl)pyrazolo[1,5-a]quinoxalin-4(5H)-one: To a solution of methyl 3-fluoro-4-oxo-4,5-dihydropyrazolo[1,5-a]quinoxaline-7-carboxylate (85 mg, 0.33 mmol) in THF (3 mL) and H₂O (1 mL) was added LiAlH₄ (1.0 M in THF, 1.3 mL) at 0° C. under nitrogen. The mixture was stirred at 0° C. for 10 minutes, the mixture was quenched with water (3 mL) and added 1 M NaOH aqueous solution (1 mL). The result mixture was stirred at room temperature for 15 minutes. The mixture was filtered and the filtrate was concentrated in vacuum. The residue was washed by (MeOH/EA=2/3) to give the product (70 mg, white solid, yield: 92%). MS(ESI): 234.00 [M+H]⁺.
e) Preparation of 7-(chloromethyl)-3-fluoropyrazolo[1,5-a]quinoxalin-4(5H)-one: To a suspension of 3-fluoro-7-(hydroxymethyl)pyrazolo[1,5-a]quinoxalin-4(5H)-one (50 mg, 0.22 mmol) in DCM (3 mL) was added DMF (1.0 mg, 0.01 mmol) and SOCl₂ (153 mg, 1.3 mmol) at 0° C. under nitrogen. The mixture was stirred at 0° C. for 10 minutes, the mixture was concentrated in vacuum to give the product (54.0 mg, crude, yellow solid). MS(ESI): 252.20 [M+H]⁺.
f) Preparation of 7-((4-(2-fluoro-6-(methylcarbamoyl)pyridin-3-yl)piperazin-1-yl)methyl)-3-fluoropyrazolo[1,5-a]quinoxalin-4(5H)-one: To a solution of 8-(chloromethyl)-10-fluoropyrrolo[1,2-c]quinazolin-5(6H)-one (54 mg, 0.2 mmol) in acetonitrile (5 mL) was added KI (3.6 mg, 0.02 mmol), 6-fluoro-N-methyl-5-(piperazin-1-yl)picolinamide (52 mg, 0.2 mmol) and K₂CO₃ (146.2 mg, 1.1 mmol) at room temperature under nitrogen. The mixture was stirred at 80° C. for 2 hours. The mixture was added water (10 mL) and extracted with EA (5 mL×3). The organic phase was concentrated and the residue was purified by Prep-TLC (DCM/MeOH=20/1) to give the target compound (20.8 mg, white solid, 2-step yield: 21%).

### Example 50

### 8-((4-(2-methyl-6-(methylcarbamoyl)pyridin-3-yl)piperazin-1-yl)methyl)-7-fluoroimidazo[1,2-c]quinazolin-5(6H)-one

a) Preparation of 4-amino-7-bromo-8-fluoroquinazolin-2(1H)-one: The mixture of 2-amino-4-bromo-3-fluorobenzonitrile (800 mg, 3.72 mmol) and chlorosulfonyl isocyanate (632 mg, 4.48 mmol) in DCM (5 mL) and was stirred for 3 hours at room temperature. The reaction was concentrated under reduced pressure. The residue was added saturated NaHCO₃ aqueous solution (10 mL) and the resulting mixture was stirred at 100° C. for 1 hour. The reaction was cooled to room temperature and filtered. The solid was washed with water (10 mL) and dried to give the product (780 mg, off-white solid, yield: 79%). MS(ESI): 257.96 [M+H]⁺.
b) Preparation of 8-bromo-7-fluoroimidazo[1,2-c]quinazolin-5(6H)-one: To the solution of 4-amino-7-bromo-8-fluoroquinazolin-2(1H)-one (190 mg, 0.74 mmol) in DMF (5 mL) was added 2-chloroacetaldehyde (287 mg, 3.68 mmol) and sodium acetate (151 mg, 1.84 mmol). The mixture was stirred at 100° C. for 3 hours. The reaction was cooled to room temperature and filtered. The solid was washed with water (5 mL) and dried to give the product (95 mg, grey solid, yield: 46%). MS(ESI): 281.96 [M+H]⁺.
c) Preparation of 7-fluoro-8-(hydroxymethyl)imidazo[1,2-c]quinazolin-5(6H)-one: To a solution of 8-bromo-7-fluoroimidazo[1,2-c]quinazolin-5(6H)-one (90 mg, 0.32 mmol) in dioxane (5 mL) was added Bu₃SnCH₂OH (123 mg, 0.38 mmol) and Xphos Pd G2 (25 mg, 0.03 mmol) at room temperature. The mixture was stirred at 90° C. overnight under N₂ atmosphere. After completion of the reaction, a solution of KF (1 M aqueous solution, 5 mL) was added at room temperature, the mixture was stirred at room temperature for 10 mins and filtered, the filtrate was extracted with EA (50 mL×3). The combined organic phase was washed with brine, dried over Na₂SO₄ and concentrated under reduced pressure. The residue was triturated with a mixed solvent of PE and EA (PE:EA=1:1, 50 mL) and the solid was collected by filtration to afford the product (61 mg, white solid, yield: 81%). MS(ESI): 234.06 [M+H]⁺.
d) Preparation of 8-(bromomethyl)-7-fluoroimidazo[1,2-c]quinazolin-5(6H)-one: To a solution of methyl 7-fluoro-8-(hydroxymethyl)imidazo[1,2-c]quinazolin-5(6H)-one (90 mg, 0.38 mmol) in DCM (5 mL) was added PBr₃ (514 mg, 1.9 mmol) under N₂ at 0° C. The mixture was warmed to room temperature and stirred for 2 hours. Then the reaction was concentrated under reduced pressure and the residue was triturated with DCM (5 mL) and the solid was collected by filtration to afford the product (65 mg, white solid, yield: 65%). MS(ESI): 295.90 [M+H]⁺.
e) Preparation of 8-((4-(2-methyl-6-(methylcarbamoyl)pyridin-3-yl)piperazin-1-yl)methyl)-7-fluoroimidazo[1,2-c]quinazolin-5(6H)-one: To a solution of 8-(bromomethyl)-7-fluoroimidazo[1,2-c]quinazolin-5(6H)-one (30 mg, 0.17 mmol), KI (3.0 mg, 0.015 mmol) and N,6-dimethyl-5-(piperazin-1-yl)picolinamide (46.1 mg, 0.18 mmol) in CH₃CN (10 mL) was added DIEA (97.1 mg, 0.75 mmol) at room temperature. The resulting solution was stirred at 80° C. for 2 hours. After completion of the reaction, the solvent was removed under vacuum. The residue was purified by Prep-TLC (DCM:MeOH=10:1) to give the target compound (36.8 mg, white solid, yield: 80%).

The following compounds of Examples 51-60 were prepared using a synthesis method similar to that described in Example 49 (Scheme 5); the compounds of Examples 61-65 were prepared using a synthesis method similar to that described in Example 50 (Scheme 6).

| | | | LC-MS | |
|---|---|---|---|---|
| Example | Compound | MW | (ESI) | ¹H NMR (400 MHz) |
| 49 | 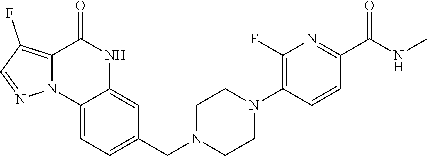 [SMILES: CNC(=O)c1ccc(N2CCN(Cc3ccc4c(c3)[nH]c(=O)c3c(F)cnn34)CC2)c(F)n1] | 453.45 | 454.15 [M + H]⁺ | DMSO-d₆: δ 11.85 (s, 1H), 8.41 (d, J = 4.5 Hz, 1H), 8.17 (d, J = 3.8 Hz, 1H), 8.07 (d, J = 8.4 Hz, 1H), 7.84 (d, J = 8.2 Hz, 1H), 7.60-7.53 (m, 1H), 7.36 (s, 1H), 7.28 (d, J = 8.7 Hz, 1H), 3.60 (s, 2H), 3.20-3.18 (m, 4H), 2.76 (d, J = 4.8 Hz, 3H), 2.59-2.56 (m, 4H). |
| 50 | 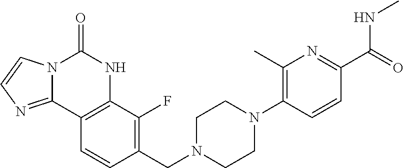 [SMILES: CNC(=O)c1ccc(N2CCN(Cc3ccc4c([nH]c(=O)n5ccnc45)c3F)CC2)c(C)n1] | 449.49 | 452.20 [M + H]⁺ | DMSO-d₆: δ 12.06 (br, 1H), 8.39-8.37 (m, 1H), 7.92-7.89 (m, 2H), 7.74 (d, J = 8.0 Hz, 1H), 7.45-7.43 (m, 2H), 7.35-7.34 (m, 1 H), 3.70 (s, 2H), 2.94-2.92 (m, 4H), 2.76 (d, J = 4.8 Hz, 3H), 2.59-2.56 (m, 4H), 2.44 (s, 3H). |
| 51 | 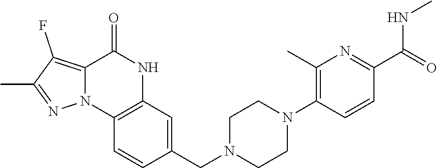 | 463.52 | 464.15 [M + H]⁺ | CD₃OD: δ 8.07 (d, J = 8.3 Hz, 1H), 7.85 (d, J = 8.3 Hz, 1H), 7.45 (d, J = 8.3 Hz, 1H), 7.38- 7.29 (m, 2H), 3.68 (s, 2H), 3.07-2.98 (m, 4H), 2.93 (s, 3H), 2.75-2.66 (m, 4H), 2.52 (s, 3H), 2.42 (s, 3H). |
| 52 | 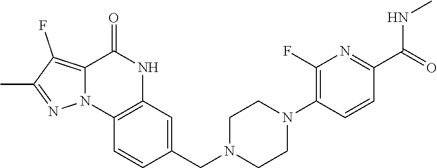 | 467.48 | / | / |
| 53 | 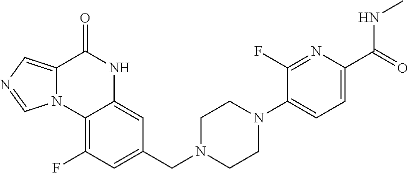 [SMILES: CNC(=O)c1ccc(N2CCN(Cc3cc(F)c4c(c3)[nH]c(=O)c3cncn34)CC2)c(F)n1] | 453.45 | 454.15 [M + H]⁺ | DMSO-d₆: δ 11.57 (s, 1H), 8.67 (d, J = 2.0 Hz, 1H),8.39-8.35 (m, 1H), 7.89 (s, 1H), 7.81 (d, J = 8.0 Hz, 1H), 7.54 (dd, J = 10.6, 8.1 Hz, 1H), 7.18-7.41 (m, 2H), 3.56 (s, 2H), 3.17-3.14 (m, 4H), 2.73 (d, J = 4.8 Hz, 3H), 2.56-2.53 (m, 4H) |
| 54 | 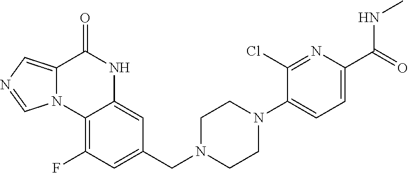 [SMILES: CNC(=O)c1ccc(N2CCN(Cc3cc(F)c4c(c3)[nH]c(=O)c3cncn34)CC2)c(Cl)n1] | 469.91 | 470.05 [M + H]⁺ | DMSO-d₆: δ 11.62 (s, 1H), 8.70 (s, 1H), 8.43 (d, J = 4.8 Hz, 1H), 7.95-7.92 (m, 2H), 7.67 (d, J = 8.4 Hz, 1H), 7.21-7.17 (m, 2H), 3.60 (s, 2H), 3.16-3.10 (m, 4H), 2.78 (d, J = 4.4 Hz, 3H), 2.64-2.58 (m, 4H). |
| 55 | 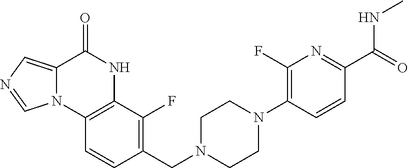 [SMILES: CNC(=O)c1ccc(N2CCN(Cc3ccc4c([nH]c(=O)c5cncn54)c3F)CC2)c(F)n1] | 453.45 | 454.15 [M + H]⁺ | DMSO-d₆: δ 11.38 (s, 1H), 8.93 (s, 1H), 8.38 (d, J = 5.2 Hz, 1H), 7.94 (s, 1H), 7.80 (d, J = 8.0 Hz, 1H), 7.58-7.50 (m, 1H), 7.32 (t, J = 9.2 Hz, 1H), 7.26-7.19 (m, 1H), 3.90 (s, 2H), 3.18-3.06 (m, 4H), 2.74 (d, J = 4.7 Hz, 3H), 2.70-2.61 (m, 4H). |
| 56 | 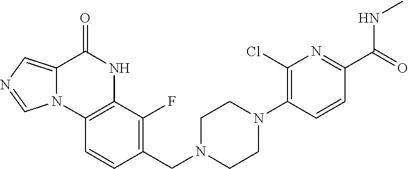 [SMILES: CNC(=O)c1ccc(N2CCN(Cc3ccc4c([nH]c(=O)c5cncn54)c3F)CC2)c(Cl)n1] | 469.91 | 470.05 [M + H]⁺ | DMSO-d₆: δ 11.45 (br, 1H), 9.03 (s, 1H), 8.41- 8.37 (m, 1H), 7.99 (d, J = 8.0 Hz, 1H), 7.90 (d, J = 8.0 Hz, 1H), 7.87 (s, 1H), 7.63 (d, J = 8.0 Hz, 1H), 7.29-7.25 (m, 1H), 3.66 (s, 2H), 3.10-3.08 (m, 4H), 2.75 (d, J = 4.0 Hz, 3H), 2.62-2.59 (m, 4H). |
| 57 | 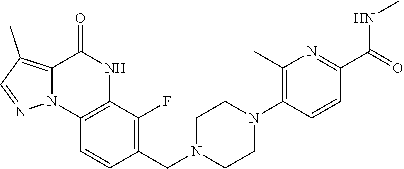 [SMILES: CNC(=O)c1ccc(N2CCN(Cc3ccc4c([nH]c(=O)c5c(C)cnn54)c3F)CC2)c(C)n1] | 463.52 | 464.20 [M + H]⁺ | DMSO-d₆: δ 8.39 (d, J = 4.7 Hz, 1H), 7.88 (s, 1H), 7.83 (d, J = 8.5 Hz, 1H), 7.74 (d, J = 8.2 Hz, 1H), 7.42 (d, J = 8.2 Hz, 1H), 7.30-7.23 (m, 1H), 3.64 (s, 2H), 2.93-2.85 (m, 4H), 2.75 (d, J = 4.8 Hz, 3H), 2.61-2.52 (m, 4H), 2.43 (s, 3H), 2.40 (s, 3H). |
| 58 | 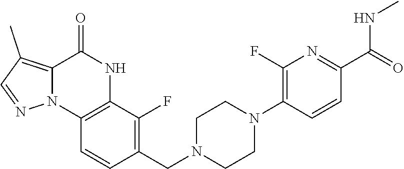 [SMILES: CNC(=O)c1ccc(N2CCN(Cc3ccc4c([nH]c(=O)c5c(C)cnn54)c3F)CC2)c(F)n1] | 467.48 | 468.20 [M + H]⁺ | DMSO-d₆: δ 8.36 (d, J = 4.9 Hz, 1H), 7.89 (s, 1H), 7.83 (d, J = 8.4 Hz, 1H), 7.79 (d, J = 8.1 Hz, 1H), 7.55-7.49 (m, 1H), 7.30-7.24 (m, 1H), 3.63 (s, 2H), 3.18-3.06 (m, 4H), 2.72 (d, J = 4.8 Hz, 3H), 2.60-2.51 (m, 4H), 2.40 (s, 3H). |
| 59 | 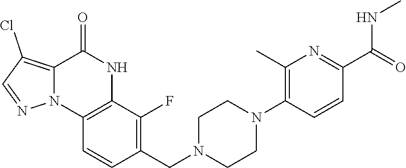 [SMILES: CNC(=O)c1ccc(N2CCN(Cc3ccc4c([nH]c(=O)c5c(Cl)cnn54)c3F)CC2)c(C)n1] | 483.93 | 484.15 [M + H]⁺ | DMSO-d₆: δ 8.42 (q, J = 5.0 Hz, 1H), 8.26 (s, 1H), 7.91 (dd, J = 8.6, 1.1 Hz, 1H), 7.78 (d, J = 8.3 Hz, 1H), 7.47 (d, J = 8.3 Hz, 1H), 7.35 (dd, J = 8.5, 6.8 Hz, 1H), 3.70 (s, 2H), 3.00 2.87 (m, 4H), 2.79 (d, J = 4.9 Hz, 3H), 2.67- 2.58 (m, 4H), 2.48 (s, 3H). |
| 60 | 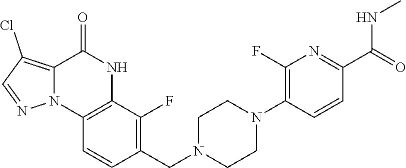 [SMILES: CNC(=O)c1ccc(N2CCN(Cc3ccc4c([nH]c(=O)c5c(Cl)cnn54)c3F)CC2)c(F)n1] | 487.90 | 488.15 [M + H]⁺ | DMSO-d₆: δ 8.41 (q, J = 5.3 Hz, 1H), 8.27 (s, 1H), 7.91 (d, J = 8.1 Hz, 1H), 7.83 (d, J = 8.1 Hz, 1H), 7.56 (dd, J = 10.6, 8.1 Hz, 1H), 7.34 (t, J = 7.7 Hz, 1H), 3.68 (s, 2H), 3.21-3.11 (m, 4H), 2.75 (d, J = 4.8 Hz, 3H), 2.64-2.56 (m, 4H). |
| 61 | 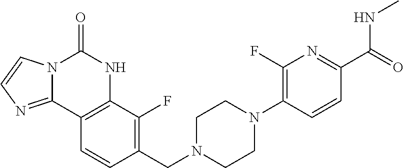 [SMILES: CNC(=O)c1ccc(N2CCN(Cc3ccc4c([nH]c(=O)n5ccnc45)c3F)CC2)c(F)n1] | 453.45 | 454.15 [M + H]⁺ | DMSO-d₆: δ 8.37-8.35 (m, 1H), 7.90-7.87 (m, 2H), 7.89 (d, J = 7.2 Hz, 1H), 7.54-7.50 (m, 1H), 7.32 (s, 1H), 7.31-7.29 (m, 1 H), 3.68 (s, 2H), 3.18-3.14 (m, 4H), 2.72 (d, J = 4.8 Hz, 3H), 2.59-2.57 (m, 4H). |
| 62 | 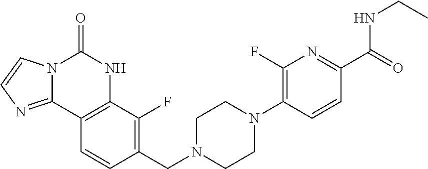 [SMILES: CCNC(=O)c1ccc(N2CCN(Cc3ccc4c([nH]c(=O)n5ccnc45)c3F)CC2)c(F)n1] | 467.48 | 468.20 [M + H]⁺ | DMSO-d₆: δ 12.06 (s, 1H), 8.41-8.38 (m, 1H), 7.91-7.89 (m, 2H), 7.80 (d, J = 8.0 Hz, 1H), 7.55-7.50 (m, 1H), 7.44 (s, 1H), 7.35-7.31 (m, 1 H), 3.68 (s, 2H), 3.24-3.14 (m, 2H), 3.18- 3.14 (m, 4H), 2.59-2.57 (m, 4H), 1.05 (t, J = 8.0 Hz, 3H). |
| 63 | 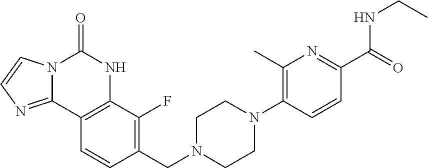 [SMILES: CCNC(=O)c1ccc(N2CCN(Cc3ccc4c([nH]c(=O)n5ccnc45)c3F)CC2)c(C)n1] | 463.52 | 464.20 [M + H]⁺ | DMSO-d₆: δ 8.43-8.38 (m, 1H), 7.91-7.88 (m, 2H), 7.75 (d, J = 8.0 Hz, 1H), 7.45-7.43 (m, 2H), 7.34-7.31 (m, 1 H), 3.69 (s, 2H), 3.27- 3.24 (m, 2H), 2.91-2.90 (m, 4H), 2.59-2.57 (m, 4H), 2.44 (s, 3H), 1.06 (t, J = 8.0 Hz, 3H). |
| 64 | 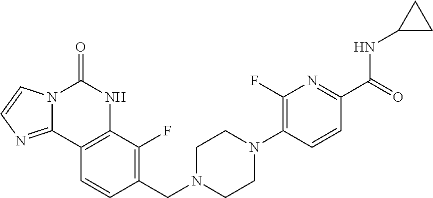 [SMILES: O=C(NC1CC1)c1ccc(N2CCN(Cc3ccc4c([nH]c(=O)n5ccnc45)c3F)CC2)c(F)n1] | 479.49 | 480.20 [M + H]⁺ | DMSO-d₆: δ 12.05 (br, 1H), 8.31 (d, J = 4.0 Hz, 1H), 7.91-7.89 (m, 2H), 7.80 (d, J = 8.0 Hz, 1H), 7.54-7.52 (m, 1H), 7.44 (s, 1H), 7.35- 7.31 (m, 1 H), 3.68 (s, 2H), 3.18-3.14 (m, 4H), 2.81-2.80 (m, 1H), 2.59-2.57 (m, 4H), 0.62- 0.60 (m, 4H). |
| 65 | 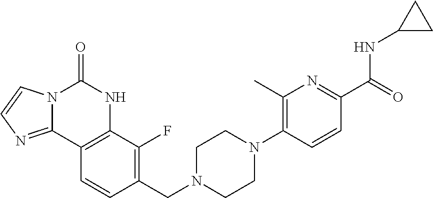 [SMILES: Cc1nc(C(=O)NC2CC2)ccc1N1CCN(Cc2ccc3c([nH]c(=O)n4ccnc34)c2F)CC1] | 475.53 | 476.20 [M + H]⁺ | DMSO-d₆: δ 12.05 (br, 1H), 8.28 (d, J = 8.0 Hz, 1H), 7.91-7.89 (m, 2H), 7.72 (d, J = 8.0 Hz, 1H), 7.44-7.42 (m, 1H), 7.44 (s, 1H), 7.35- 7.31 (m, 1 H), 3.69 (s, 2H), 2.91-2.90 (m, 4H), 2.81-2.80 (m, 1H), 2.59-2.57 (m, 4H), 0.66- 0.64 (m, 2H), 0.61-0.60 (m, 2H). |

### Example 66

### 7-((4-(2-fluoro-6-(ethylcarbamoyl)pyridin-3-yl)piperazin-1-yl)methyl)-6-fluorofuro[2,3-c]quinolin-4(5H)-one

a) Preparation of ethyl 3-(4-bromo-3-fluoro-2-nitrophenyl)furan-2-carboxylate: To a solution of 1,4-dibromo-2-fluoro-3-nitrobenzene (12.5 g, 42.09 mmol) in DMF (125 mL) was added (2-(ethoxycarbonyl)furan-3-yl)boronic acid (7.74 g, 42.09 mmol), Pd(dppf)Cl₂ dichloromethane complex (3.43 g, 4.21 mmol) and Cs₂CO₃ (41.2 g, 126.26 mmol). The mixture was stirred at 100° C. for 3 hours. To the reaction mixture was added water (150 mL) and the resulting mixture was extracted with ethyl acetate (100 mL×3). The organic phase was dried over anhydrous Na₂SO₄, filtered and concentrated. The residue was purified by flash silica chromatography (elution gradient 0 to 5% ethyl acetate in petroleum ether) to afford the product (7.16 g, yellow oil, yield: 48%). MS(ESI): 357.90 [M−H]⁺.
b) Preparation of 7-bromo-6-fluorofuro[2,3-c]quinolin-4(5H)-one: To a solution of ethyl 3-(4-bromo-3-fluoro-2-nitrophenyl)furan-2-carboxylate (5.4 g, 15.08 mmol) in AcOH (100 mL) was added Fe powder (4.2 g, 75.39 mmol). The resulting mixture was stirred at 80° C. for 3 hours. After completion of the reaction, the reaction mixture was concentrated and purified by flash silica chromatography (elution gradient 0 to 5% methanol in dichloromethane) to give the product (3.07 g, yellow solid, yield: 72%). MS(ESI): 281.95 [M−H]⁺.
c) Preparation of 6-fluoro-7-(hydroxymethyl)furo[2,3-c]quinolin-4(5H)-one: To a solution of 7-bromo-6-fluorofuro[2,3-c]quinolin-4(5H)-one (3.07 g, 10.89 mmol) in dioxane (60 mL) was added (tributylstannyl)methanol (5.24 g, 16.33 mmol) and Xphos Pd G2 (857.6 mg, 1.09 mmol) at room temperature. The mixture was stirred at 100° C. for 3 hours. After completion of the reaction, the reaction mixture was filtered. The filter cake was washed with DMF, and the DMF phase was concentrated and washed with dichloromethane to afford the product (1.2 g, white solid, yield: 47%). MS(ESI): 234.05 [M−H]⁺.
d) Preparation of 7-(bromomethyl)-6-fluorofuro[2,3-c]quinolin-4(5H)-one: To a solution of 6-fluoro-7-(hydroxymethyl)furo[2,3-c]quinolin-4(5H)-one (1.2 g, 5.15 mmol) in DCM (50 mL) was added phosphorus tribromide (2.8 g, 10.30 mmol). The mixture stirred at room temperature for 16 hours. After completion of the reaction, the mixture was concentrated to give the product (1.4 g, crude, white solid, yield: 92%), which was used in the next step without further purification. MS(ESI): 295.85 [M−H]⁺.
e) Preparation of 7-((4-(2-fluoro-6-(ethylcarbamoyl)pyridin-3-yl)piperazin-1-yl)methyl)-6-fluorofuro[2,3-c]quinolin-4(5H)-one: To a solution of 7-(bromomethyl)-6-fluorofuro[2,3-c]quinolin-4(5H)-one (52.7 mg, 0.18 mmol), KI (3.0 mg, 0.02 mmol) and N-ethyl-6-fluoro-5-(piperazin-1-yl)picolinamide (45.0 mg, 0.18 mmol) in acetonitrile (20 mL) was added K₂CO₃ (73.9 mg, 0.54 mmol) at room temperature. The resulting mixture was stirred at 80° C. for 3 hours. After completion of the reaction, the mixture was cooled to room temperature and filtered. The filter cake was purified by Prep-HPLC to give the target compound (35.2 mg, white solid, yield: 42%).

The following compounds of Examples 67 and 68 were prepared using a synthesis method similar to that described in Example 66 (Scheme 7); the compounds of Examples 69-71 were prepared using a synthesis method similar to that described in Example 1 (Scheme 1); the compounds of Examples 72-73 were prepared using a synthesis method similar to that described in Example 48; the compounds of Examples 74-87 were prepared using a synthesis method similar to that described in Example 40 (Scheme 4).

| | | | LC-MS | |
|---|---|---|---|---|
| Example | Compound | MW | (ESI) | ¹H NMR (400 MHz) |
| 66 | 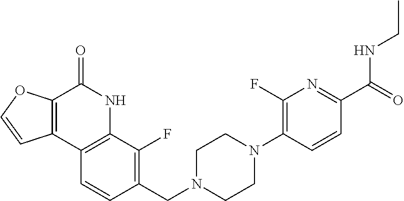 [SMILES: CCNC(=O)c1ccc(N2CCN(Cc3ccc4c([nH]c(=O)c5occc54)c3F)CC2)c(F)n1] | 467.48 | 468.15 [M + H]⁺ | DMSO-d₆: δ 8.44 (t, J = 5.8 Hz, 1H), 8.28 (d, J = 1.8 Hz, 1H), 8.21 (s, 1H), 7.83 (d, J = 8.0 Hz, 2H), 7.55 (dd, J = 10.4, 8.4 Hz, 1H), 7.50 (d, J = 1.8 Hz, 1H), 7.32 (t, J = 7.3 Hz, 1H), 3.71 (s, 2H), 3.32-3.20 (m, 2H), 3.20-3.08 (m, 4H), 2.66-2.55 (m, 4H), 1.08 (t, J = 7.1 Hz, 3H). |
| 67 | 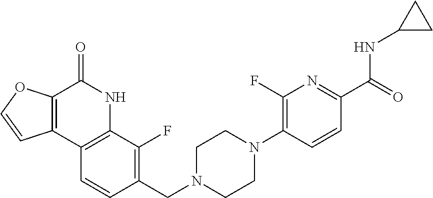 [SMILES: O=C(NC1CC1)c1ccc(N2CCN(Cc3ccc4c([nH]c(=O)c5occc54)c3F)CC2)c(F)n1] | 479.49 | 480.20 [M + H]⁺ | DMSO-d₆: δ 11.88 (s, 1H), 8.35-8.33 (m, 1H), 8.28 (d, J = 1.9 Hz, 1H), 7.83 (d, J = 8.0 Hz, 2H), 7.55 (dd, J = 10.5, 8.2 Hz, 1H), 7.50 (d, J = 2.0 Hz, 1H), 7.32 (t, J = 7.3 Hz, 1H), 3.71 (s, 2H), 3.21-3.11 (m, 4H), 2.90-2.77 (m, 1H), 2.65-2.55 (m, 4H), 0.76-0.52 (m, 4H). |
| 68 | 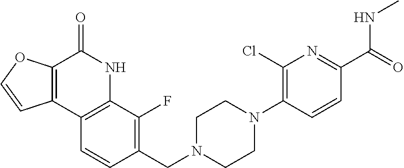 [SMILES: CNC(=O)c1ccc(N2CCN(Cc3ccc4c([nH]c(=O)c5occc54)c3F)CC2)c(Cl)n1] | 469.90 | 470.1 [M + H]⁺ | DMSO-d₆: δ 11.88 (s, 1H), 8.43 (q, J = 5.4 Hz, 1H), 8.28 (d, J = 2.0 Hz, 1H), 7.93 (d, J = 8.2 Hz, 1H), 7.83 (d, J = 8.1 Hz, 1H), 7.65 (d, J = 8.3 Hz, 1H), 7.50 (d, J = 2.0 Hz, 1H), 7.32 (dd, J = 8.1, 6.3 Hz, 1H), 3.73 (s, 2H), 3.17-3.03 (m, 4H), 2.78 (d, J = 4.8 Hz, 3H), 2.68-2.56 (m, 4H). |
| 69 | 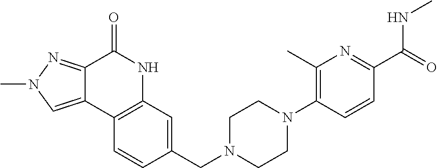 | 445.53 | 446.10 [M + H]⁺ | DMSO-d₆: δ 11.26 (s, 1H), 8.57 (s, 1H), 8.39 (d, J = 4.9 Hz, 1H), 7.76 (t, J = 8.3 Hz, 2H), 7.43 (d, J = 8.3 Hz, 1H), 7.29 (d, J = 1.5 Hz, 1H), 7.16-7.06 (m, 1H), 4.08 (s, 3H), 3.54 (s, 2H), 2.95-2.84 (s, 4H), 2.76 (d, J = 4.9 Hz, 3H), 2.59-2.48 (s, 4H), 2.45 (s, 3H). |
| 70 | 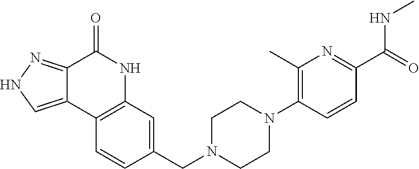 [SMILES: CNC(=O)c1ccc(N2CCN(Cc3ccc4c(c3)[nH]c(=O)c3n[nH]cc34)CC2)c(C)n1] | 431.50 | 432.10 [M + H]⁺ | DMSO-d₆: δ 11.59 (s, 1H), 8.49-8.34 (m, 2H), 7.92 (s, 1H), 7.76 (d, J = 8.3 Hz, 1H), 7.44 (d, J = 8.3 Hz, 1H), 7.36 (s, 1H), 7.18 (d, J = 7.5 Hz, 1H), 3.58 (s, 2H), 2.95-2.89 (m, 4H), 2.76 (d, J = 4.9 Hz, 3H), 2.60-2.50 (m, 4H), 2.46 (s, 3H). |
| 71 | 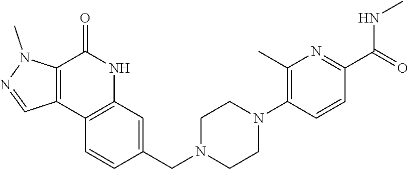 [SMILES: CNC(=O)c1ccc(N2CCN(Cc3ccc4c(c3)[nH]c(=O)c3c4cnn3C)CC2)c(C)n1] | 445.53 | 446.20 [M + H]⁺ | DMSO-d₆: δ 11.66 (s, 1H), 8.43-8.30 (m, 2H), 7.93 (d, J = 8.0 Hz, 1H), 7.76 (d, J = 8.2 Hz, 1H), 7.44 (d, J = 8.3 Hz, 1H), 7.35 (d, J = 1.5 Hz, 1H), 7.19 (dd, J = 8.0, 1.6 Hz, 1H), 4.26 (s, 3H), 3.58 (s, 2H), 2.96-2.85 (m, 4H), 2.76 (d, J = 4.9 Hz, 3H), 2.64-2.51(m, 4H), 2.45 (s, 3H).. |
| 72 | 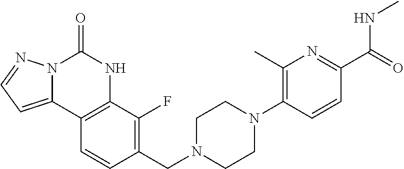 [SMILES: CNC(=O)c1ccc(N2CCN(Cc3ccc4c([nH]c(=O)n5nccc45)c3F)CC2)c(C)n1] | 449.49 | 450.10 [M + H]⁺ | DMSO-d₆: δ 11.78 (s, 1H), 8.39 (q, J = 5.1 Hz, 1H), 8.06 (d, J = 1.8 Hz, 1H), 7.96 (d, J = 10.0 Hz, 1H), 7.76 (d, J = 8.2 Hz, 1H), 7.43 (dd, J = 11.6, 7.3 Hz, 2H), 7.23 (d, J = 1.9 Hz, 1H), 3.65 (s, 2H), 3.00-2.86 (m, 4H), 2.76 (d, J = 4.8 Hz, 3H), 2.66-2.55 (m, 4H), 2.46 (s, 3H). |
| 73 | 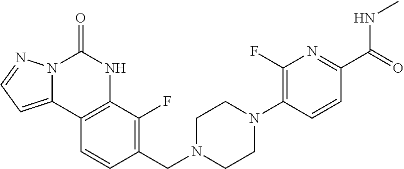 [SMILES: CNC(=O)c1ccc(N2CCN(Cc3ccc4c([nH]c(=O)n5nccc45)c3F)CC2)c(F)n1] | 453.45 | 454.1 [M + H]⁺ | DMSO-d₆: δ 8.38 (q, J = 4.4 Hz, 1H), 8.08 (d, J = 1.8 Hz, 1H), 7.83 (d, J = 8.2 Hz, 1H), 7.80 (dd, J = 8.0, 1.3 Hz, 1H), 7.52 (dd, J = 10.6, 8.1 Hz, 1H), 7.33-7.27 (m, 1H), 7.22 (d, J = 1.9 Hz, 1H), 3.67 (s, 2H), 3.16- 3.10 (m, 4H), 2.72 (d, J = 4.8 Hz, 3H), 2.59- 2.53 (m, 4H). |
| 74 | 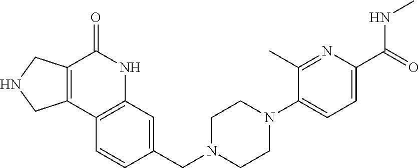 [SMILES: CNC(=O)c1ccc(N2CCN(Cc3ccc4c5c(c(=O)[nH]c4c3)CNC5)CC2)c(C)n1] | 432.53 | 433.20 [M + H]⁺ | DMSO-d₆: δ 11.70 (s, 1H), 8.38 (d, J = 4.8 Hz, 1H), 8.20 (s, 1H), 7.76 (d, J = 8.3 Hz, 1H), 7.44 (dd, J = 8.1, 5.2 Hz, 2H), 7.34 (s, 1H), 7.17 (d, J = 7.8 Hz, 1H), 4.42 (s, 2H), 4.07 (s, 2H), 3.59 (s, 2H), 2.99-2.86 (m, 4H), 2.76 (d, J = 4.8 Hz, 3H), 2.61-2.49 (m, 4H), 2.45 (s, 3H). |
| 75 | 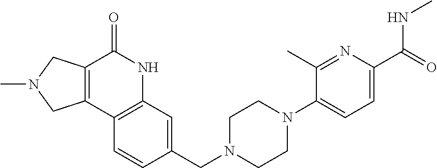 | 446.56 | 447.20 [M + H]⁺ | DMSO-d₆: δ 11.67 (s, 1H), 8.38 (q, J = 4.9 Hz, 1H), 7.75 (d, J = 8.3 Hz, 1H), 7.42 (t, J = 8.2 Hz, 2H), 7.32 (s, 1H), 7.15 (d, J = 8.1 Hz, 1H), 4.11 (t, J = 3.5 Hz, 2H), 3.78 (t, J = 3.5 Hz, 2H), 3.58 (s, 2H), 2.95-2.86 (m, 4H), 2.76 (d, J = 4.8 Hz, 3H), 2.59-2.51 (m, 4H), 2.53-2.48 (m, 3H), 2.44 (s, 3H). |
| 76 | 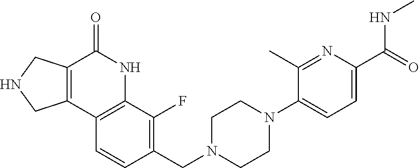 [SMILES: CNC(=O)c1ccc(N2CCN(Cc3ccc4c5c(c(=O)[nH]c4c3F)CNC5)CC2)c(C)n1] | 450.52 | / | / |
| 77 | 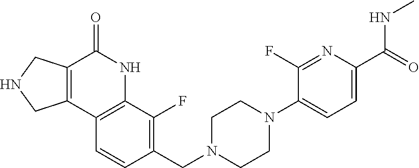 [SMILES: CNC(=O)c1ccc(N2CCN(Cc3ccc4c5c(c(=O)[nH]c4c3F)CNC5)CC2)c(F)n1] | 454.48 | / | / |
| 78 | 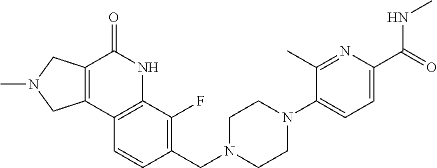 | 464.55 | / | / |
| 79 | 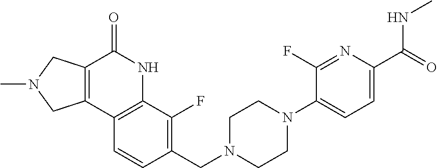 | 468.51 | / | / |
| 80 | 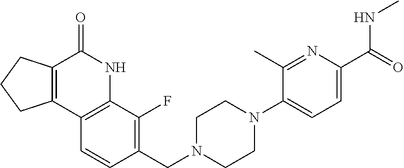 [SMILES: CNC(=O)c1ccc(N2CCN(Cc3ccc4c5c(c(=O)[nH]c4c3F)CCC5)CC2)c(C)n1] | 449.53 | / | / |
| 81 | 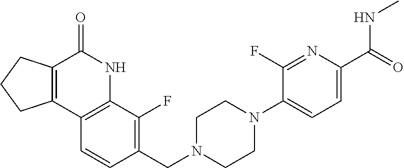 [SMILES: CNC(=O)c1ccc(N2CCN(Cc3ccc4c5c(c(=O)[nH]c4c3F)CCC5)CC2)c(F)n1] | 453.49 | / | / |
| 82 | 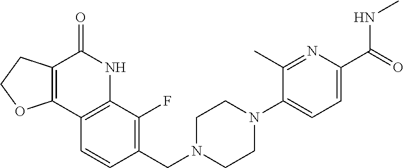 [SMILES: CNC(=O)c1ccc(N2CCN(Cc3ccc4c5c(c(=O)[nH]c4c3F)CCO5)CC2)c(C)n1] | 451.50 | / | / |
| 83 | 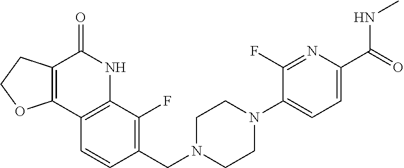 [SMILES: CNC(=O)c1ccc(N2CCN(Cc3ccc4c5c(c(=O)[nH]c4c3F)CCO5)CC2)c(F)n1] | 455.47 | / | / |
| 84 | 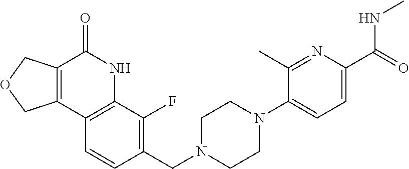 [SMILES: CNC(=O)c1ccc(N2CCN(Cc3ccc4c5c(c(=O)[nH]c4c3F)COC5)CC2)c(C)n1] | 451.50 | / | / |
| 85 | 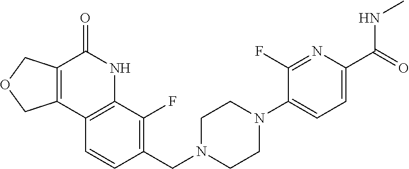 [SMILES: CNC(=O)c1ccc(N2CCN(Cc3ccc4c5c(c(=O)[nH]c4c3F)COC5)CC2)c(F)n1] | 455.47 | / | / |
| 86 | 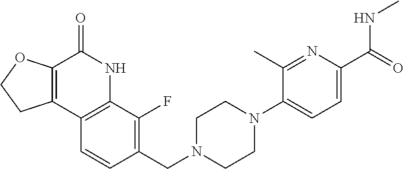 [SMILES: CNC(=O)c1ccc(N2CCN(Cc3ccc4c5c(c(=O)[nH]c4c3F)OCC5)CC2)c(C)n1] | 451.50 | / | / |
| 87 | 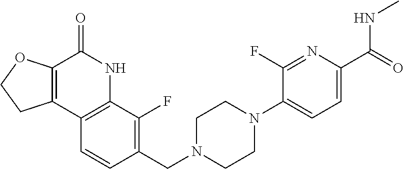 [SMILES: CNC(=O)c1ccc(N2CCN(Cc3ccc4c5c(c(=O)[nH]c4c3F)OCC5)CC2)c(F)n1] | 455.47 | / | / |

### Example 88

### 7-((4-(2-methyl-6-(methylcarbamoyl)pyridin-3-yl)piperazin-1-yl)methyl)-6-fluoro-2-methyl-2,5-dihydro-4H-pyrazolo[4,3-c]quinolin-4-one

a) Preparation of (3-amino-4-bromo-2-fluorophenyl)methanol: To a solution of (4-bromo-2-fluoro-3-nitrophenyl)methanol (1.6 g, 7.2 mmol) in AcOH (25 mL) was added iron dust (1.2 g, 21.6 mmol) at room temperature. The mixture was stirred at 85° C. for 1 hour under N₂ atmosphere. The mixture was slowly cooled to room temperature. Water (50 mL) was added and the mixture was extracted with EA (50 mL×2). The combined organic phase was washed with saturated NaHCO₃ aqueous and brine, dried over anhydrous sodium sulfate and concentrated under reduced pressure. The residue was triturated with a mixed solvent of PE and EA (PE/EA=20:1, 10 mL) to afford the product (900 mg, orange solid, yield: 56%). MS(ESI): 219.90[M+H]⁺.
b) Preparation of (3-amino-2-fluoro-4-(4,4,5,5-tetramethyl-1,3,2-dioxaborolan-2-yl)phenyl)methanol: To a solution of (3-amino-4-bromo-2-fluorophenyl)methanol (730 mg, 3.3 mmol) in dioxane (10 mL) was added bis(pinacolato)diboron (1.1 g, 4.0 mmol), KOAc (980 mg, 10.0 mmol) and Pd(dppf)Cl₂ (239 mg, 0.3 mmol) under N₂. The resulting mixture was stirred at 100° C. overnight. After the reaction was completed, the mixture was cooled to room temperature. The resulting mixture was added water (50 mL) and extracted with EA (50 mL×2). The combined organic phase was washed with brine, dried over anhydrous sodium sulfate and concentrated under reduced pressure. The residue was purified by silica gel column (PE/EA=3/1) to give the product (0.6 g, yellow oil, yield: 68%). MS(ESI): 267.85[M+H]⁺.
c) Preparation of methyl 3-(2-amino-3-fluoro-4-(hydroxymethyl)phenyl)-1-methyl-1H-pyrazole-4-carboxylate: To a solution of (3-amino-2-fluoro-4-(4,4,5,5-tetramethyl-1,3,2-dioxaborolan-2-yl)phenyl)methanol (0.4 g, 1.4 mmol) in dioxane (5.0 mL) and H₂O (1.0 ml) was added methyl 3-bromo-1-methyl-1H-pyrazole-4-carboxylate (284 mg, 1.4 mmol), K₂CO₃ (0.6 g, 4.2 mmol) and Pd(dppf)Cl₂ (80 mg, 0.1 mmol) at room temperature under N₂ atmosphere. The resulting mixture was stirred at 100° C. under N₂ atmosphere overnight. The mixture was slowly cooled to room temperature and filtered. The filtrate was concentrated under reduced pressure to give the product (0.4 g, crude, brown solid).
d) Preparation of 6-fluoro-7-(hydroxymethyl)-2-methyl-2,5-dihydro-4H-pyrazolo[4,3-c]quinolin-4-one: A solution of ethyl 3-(2-amino-3-fluoro-4-(hydroxymethyl)phenyl)-1-methyl-1H-pyrazole-4-carboxylate (0.4 g, 1.4 mmol) in AcOH (5 mL) was stirred at room temperature for 2 hours. The reaction mixture was concentrated under reduced pressure. The residue was triturated with PE: EA=1:1 (5 mL) for three times to afford the product (0.4 g, crude, brown solid). MS(ESI): 248.00 [M+H]⁺.
e) Preparation of 7-(bromomethyl)-6-fluoro-2-methyl-2,5-dihydro-4H-pyrazolo[4,3-c]quinolin-4-one: A solution of 6-fluoro-7-(hydroxymethyl)-2-methyl-2,5-dihydro-4H-pyrazolo[4,3-c]quinolin-4-one (0.4 g, 1.6 mmol) in HBr (48% in H₂O, 18 mL) was stirred at 85° C. for 1 hour. The reaction mixture was concentrated under reduced pressure to give the product (0.4 g, crude, brown solid). MS(ESI): 309.90 [M+H]⁺.
f) Preparation of 7-((4-(2-methyl-6-(methylcarbamoyl)pyridin-3-yl)piperazin-1-yl)methyl)-6-fluoro-2-methyl-2,5-dihydro-4H-pyrazolo[4,3-c]quinolin-4-one: To a solution of 7-(bromomethyl)-6-fluoro-2-methyl-2,5-dihydro-4H-pyrazolo[4,3-c]quinolin-4-one (0.1 g, 0.4 mmol), KI (7.0 mg, 0.04 mmol) and 6-fluoro-N-methyl-5-(piperazin-1-yl)picolinamide (165 mg, 1.3 mmol) was added DIEA (81 mg, 0.6 mmol) at room temperature under N₂. The resulting mixture was stirred at 80° C. for 1 hour. The solvent was removed under reduced pressure. The residue was purified by Prep-HPLC to give the target compound (10.0 mg, white solid, 4-step yield: 5.0%).

The following compounds of Examples 89-100 were prepared using a synthesis method similar to that described in Example 1 (Scheme 1); the compounds of Examples 101-118 were prepared using a synthesis method similar to that described in Example 66 (Scheme 7); the compounds of Examples 119-120 were prepared using a synthesis method similar to that described in Example 48.

| Ex- | | | | |
|---|---|---|---|---|
| am- | | | LC-MS | |
| ple | Compound | MW | (ESI) | ¹H NMR (400 MHz) |
| 88 | 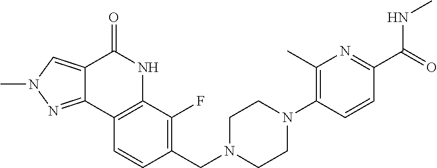 | 463.52 | 464.15 [M + H]⁺ | DMSO-d₆: δ 11.05 (br, 1H), 8.61-8.55 (m, 1H), 8.42 (s, 1H), 7.82-7.72 (m, 2H), 7.50- 7.42 (m, 1H), 7.28-7.19 (m, 1H), 4.08 (s, 3H), 3.69 (d, J = 7.7 Hz, 2H), 2.97-2.90 (m, 4H), 2.64-2.56 (m, 3H), 2.65-2.56 (m, 4H), 2.48 (s, 3H). |
| 89 | 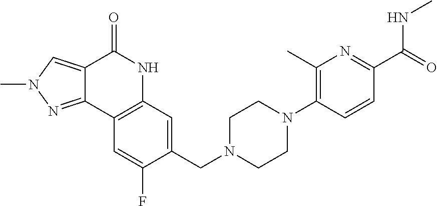 | 463.52 | 464.20 [M + H]⁺ | DMSO-d₆: δ 11.14 (s, 1H), 8.56 (s, 1H), 8.42 (d, J = 5.3 Hz, 1H), 7.79 (d, J = 8.3 Hz, 1H), 7.68 (d, J = 9.8 Hz, 1H), 7.51-7.41 (m, 2H), 4.09 (s, 3H), 3.66 (s, 2H), 3.01-2.92 (m, 4H), 2.80 (d, J = 4.8 Hz, 3H), 2.67-2.57 (m, 4H), 2.49 (s, 3H). |
| 90 | 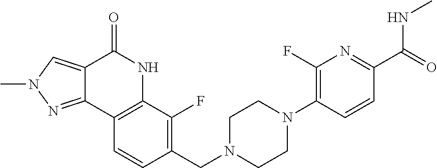 | 467.48 | 468.10 [M + H]⁺ | DMSO-d₆: δ 11.05 (s, 1H), 8.58 (d, J = 6.1 Hz, 1H), 8.43-8.35 (m, 1H), 7.85-7.74 (m, 2H), 7.63-7.47 (m, 1H), 7.27-7.20 (m, 1H), 4.08 (s,3H), 3.67 (s, 2H), 3.18-3.12 (m, 4H), 2.75 (d, J = 5.5 Hz, 3H), 2.62-2.54 (m, 4H) |
| 91 | 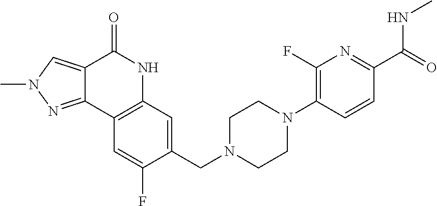 | 467.48 | 468.15 [M + H]⁺ | DMSO-d₆: δ 11.14 (s, 1H), 8.56 (s, 1H), 8.40 (s, 1H), 7.84 (d, J = 8.2 Hz, 1H), 7.68 (d, J = 10.1 Hz, 1H), 7.56 (s, 1H), 7.46 (d, J = 6.5 Hz, 1H), 4.08 (d, J = 4.3 Hz, 3H), 3.64 (d, J = 4.9 Hz, 2H), 3.19-3.14 (m, 4H), 2.76 (d, J = 4.9 Hz, 3H), 2.64-2.61 (m, 4H). |
| 92 | 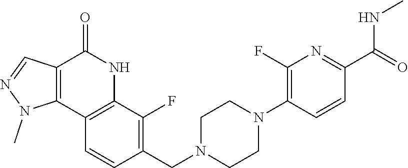 [SMILES: CNC(=O)c1ccc(N2CCN(Cc3ccc4c([nH]c(=O)c5cnn(C)c54)c3F)CC2)c(F)n1] | 467.48 | 468.15 [M + H]⁺ | DMSO-d₆: δ 11.34 (s, 1H), 8.44-8.38 (m, 1H), 8.13 (d, J = 1.7 Hz, 1H), 8.02 (d, J = 8.4 Hz, 1H), 7.83 (d, J = 8.0 Hz, 1H), 7.61-7.52 (m, 1H), 7.37-7.31 (m, 1H), 4.36 (s, 3H), 3.73 (s, 2H), 3.20-3.14 (m, 4H), 2.75 (d, J = 4.7 Hz, 3H), 2.64-2.56 (m, 4H). |
| 93 | 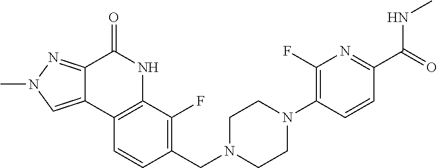 | 467.48 | 468.30 [M + H]⁺ | DMSO-d₆: δ 11.21 (s, 1H), 8.63 (s, 1H), 8.38- 8.85 (m, 1H), 7.80 (dd, J = 8.0, 1.5 Hz, 1H), 7.63 (d, J = 8.0 Hz, 1H), 7.51 (dd, J = 10.6, 8.1 Hz, 1H), 7.17 (dd, J = 8.0, 6.5 Hz, 1H), 4.09 (s, 3H), 3.63 (s, 2H), 3.18-3.08 (t, J = 4.9 Hz, 4H), 2.72 (d, J = 4.7 Hz, 3H), 2.60- 2.54 (m, 4H). |
| 94 | 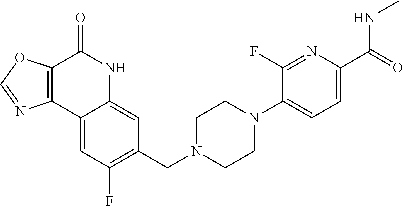 [SMILES: CNC(=O)c1ccc(N2CCN(Cc3cc4[nH]c(=O)c5ocnc5c4cc3F)CC2)c(F)n1] | 454.44 | 455.05 [M + H]⁺ | DMSO-d₆: δ 12.19 (s, 1H), 9.00 (s, 1H), 8.38 (d, J = 4.9 Hz, 1H), 7.81 (d, J = 7.9 Hz, 1H), 7.73 (d, J = 9.6 Hz, 1H), 7.61 (d, J = 6.2 Hz, 1H), 7.58-7.49 (m, 1H), 3.68 (s, 2H), 3.19- 3.13 (m, 4H), 2.73 (d, J = 4.8 Hz, 3H), 2.63- 2.57 (m, 4H). |
| 95 | 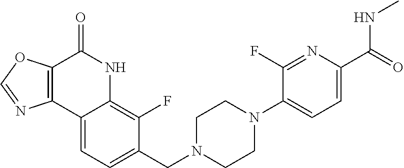 [SMILES: CNC(=O)c1ccc(N2CCN(Cc3ccc4c([nH]c(=O)c5ocnc54)c3F)CC2)c(F)n1] | 454.44 | 455.00 [M + H]⁺ | DMSO-d₆: δ 9.04 (s, 1H), 8.44-8.36 (m, 1H), 7.86-7.80 (m, 2H), 7.55f (dd, J = 10.6, 8.0 Hz, 1H), 7.40 (t, J = 7.1 Hz, 1H), 3.73 (s, 2H), 3.20-3.12 (m, 4H), 2.75 (d, J = 4.7 Hz, 3H), 2.64-2.57 (m, 4H). |
| 96 | 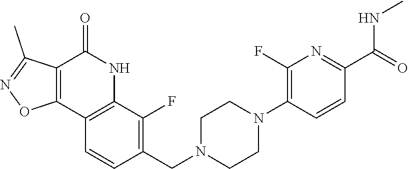 [SMILES: CNC(=O)c1ccc(N2CCN(Cc3ccc4c([nH]c(=O)c5c(C)noc54)c3F)CC2)c(F)n1] | 468.46 | 469.10 [M + H]⁺ | DMSO-d₆: δ 11.88 (br, 1H), 8.42-8.36 (m, 1H), 7.81 (d, J = 8.0 Hz, 2H), 7.56-7.50 (m, 1H), 7.40-7.34 (m, 1H), 3.71 (s, 2H), 3.17- 3.11 (m, 4H), 2.73 (d, J = 4.2 Hz, 3H), 2.61- 2.57 (m, 4H), 2.55 (s, 3H). |
| 97 | 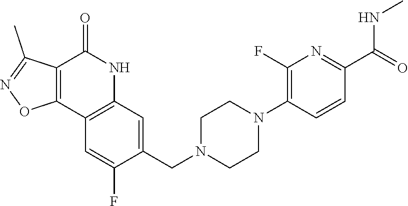 [SMILES: CNC(=O)c1ccc(N2CCN(Cc3cc4[nH]c(=O)c5c(C)noc5c4cc3F)CC2)c(F)n1] | 468.46 | 469.10 [M + H]⁺ | DMSO-d₆: δ 11.92 (s, 1H), 8.41 (d, J = 5.4 Hz, 1H), 7.86 (t, J = 8.0 Hz, 2H), 7.63 (d, J = 6.1 Hz, 1H), 7.58 (dd, J = 10.5, 8.2 Hz, 1H), 3.71 (s, 2H), 3.22-3.15 (m, 4H), 2.76 (d, J = 4.7 Hz, 3H), 2.67-2.60 (m, 4H), 2.56 (s, 3H). |
| 98 | 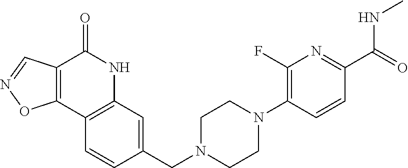 [SMILES: CNC(=O)c1ccc(N2CCN(Cc3ccc4c(c3)[nH]c(=O)c3cnoc34)CC2)c(F)n1] | 436.45 | / | / |
| 99 | 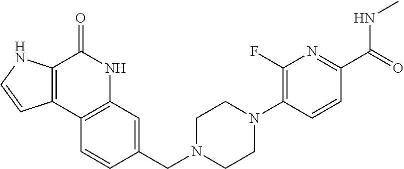 [SMILES: CNC(=O)c1ccc(N2CCN(Cc3ccc4c(c3)[nH]c(=O)c3[nH]ccc34)CC2)c(F)n1] | 434.48 | 435.10 [M + H]⁺ | DMSO-d₆: δ 12.19 (s, 1H), 11.29 (s, 1H), 8.41 (d, J = 4.9 Hz, 1H), 7.86 (dd, J = 15.7, 7.9 Hz, 2H), 7.61-7.52 (m, 1H), 7.35 (d, J = 2.2 Hz, 2H), 7.19-7.12 (m, 1H), 6.85 (d, J = 2.8 Hz, 1H), 3.58 (s, 2H), 3.23-3.15 (m, 4H),2.76 (d, J = 4.7 Hz, 3H), 2.60-2.53 (m, 4H). |
| 100 | 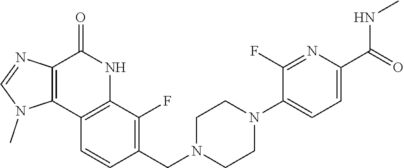 [SMILES: CNC(=O)c1ccc(N2CCN(Cc3ccc4c([nH]c(=O)c5ncn(C)c54)c3F)CC2)c(F)n1] | 467.48 | 468.15 [M + H]⁺ | DMSO-d₆: δ 11.41 (s, 1H), 8.37 (q, J = 5.4, 5.0 Hz, 1H), 8.09 (s, 1H), 7.90 (d, J = 8.4 Hz, 1H), 7.80 (d, J = 6.9 Hz, 1H), 7.52 (dd, J = 10.7, 8.0 Hz, 1H),, 7.33-7.22 (m, 1H), 4.13 (s, 3H), 3.69 (s, 2H), 3.18-3.08 (m, 4H), 2.72 (d, J = 4.8 Hz, 3H), 2.61-2.52 (m, 4H). |
| 101 | 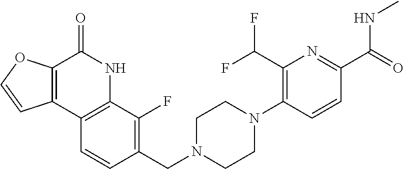 [SMILES: CNC(=O)c1ccc(N2CCN(Cc3ccc4c([nH]c(=O)c5occc54)c3F)CC2)c(C(F)F)n1] | 485.47 | 486.15 [M + H]⁺ | DMSO-d₆: δ 11.91 (s, 1H), 8.40 (q, J = 4.7 Hz, 1H), 8.28 (d, J = 1.9 Hz, 1H), 8.07 (d, J = 8.5 Hz, 1H), 7.84 (d, J = 5.5 Hz, 1H), 7.82 (d, J = 5.1 Hz, 1H), 7.50 (d, J = 2.0 Hz, 1H), 7.31 (dd, J = 8.1, 6.3 Hz, 1H), 7.13 (t, J = 53.6 Hz, 1H), 3.72 (s, 2H), 3.06-2.93 (m, 4H), 2.81 (d, J = 4.8 Hz, 3H), 2.67-2.59 (m, 4H). |
| 102 | 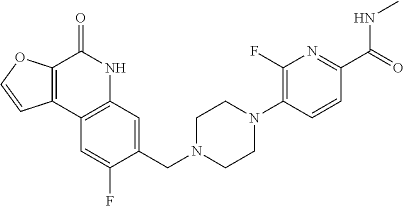 [SMILES: CNC(=O)c1ccc(N2CCN(Cc3cc4[nH]c(=O)c5occc5c4cc3F)CC2)c(F)n1] | 453.45 | 454.10 [M + H]⁺ | DMSO-d₆: δ 11.91 (s, 1H), 8.44-8.39 (m, 1H), 8.25 (d, J = 2.0 Hz, 1H), 7.93 (d, J = 10.2 Hz, 1H), 7.84 (d, J = 7.7 Hz, 1H), 7.61- 7.52 (m, 2H), 7.50 (d, J = 2.0 Hz, 1H), 3.68 (s, 2H), 3.21-3.16 (m, 4H), 2.76 (d, J = 4.8 Hz, 3H), 2.65-2.58 (m, 4H). |
| 103 | 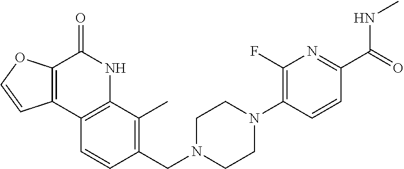 [SMILES: CNC(=O)c1ccc(N2CCN(Cc3ccc4c([nH]c(=O)c5occc54)c3C)CC2)c(F)n1] | 449.49 | 450.10 [M + H]⁺ | DMSO-d₆: δ 10.78 (s, 1H), 8.41-8.34 (m, 1H), 8.21 (d, J = 2.1 Hz, 1H), 7.87f-7.73 (m, 2H), 7.52 (dd, J = 10.6, 8.0 Hz, 1H), 7.44 (d, J = 2.1 Hz, 1H), 7.21 (d, J = 8.0 Hz, 1H), 3.59 (s, 2H), 3.1-3.08 (m, 4H), 2.72 (d, J = 4.8 Hz, 3H), 2.56-2.51 (m, 4H), 3.33 (s, 3H). |
| 104 | 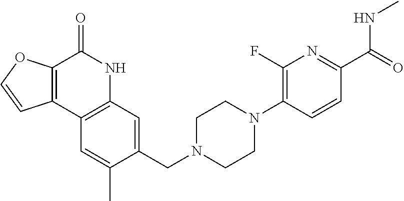 [SMILES: CNC(=O)c1ccc(N2CCN(Cc3cc4[nH]c(=O)c5occc5c4cc3C)CC2)c(F)n1] | 449.49 | 450.15 [M + H]⁺ | DMSO-d₆: δ 11.73 (s, 1H), 8.41-8.36 (m, 1H), 8.18 (d, J = 1.9 Hz, 1H), 7.83-7.79 (m, 1H), 7.78 (s, 1H), 7.57-7.51 (m, 1H), 7.40 (d, J = 2.0 Hz, 1H), 7.38 (s, 1H), 3.54 (s, 2H), 3.17-3.10 (m, 4H), 2.72 (d, J = 4.8 Hz, 3H), 2.59-2.54 (m, 4H), 2.39 (s, 3H). |
| 105 | 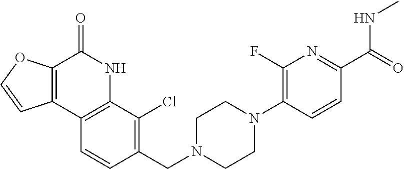 [SMILES: CNC(=O)c1ccc(N2CCN(Cc3ccc4c([nH]c(=O)c5occc54)c3Cl)CC2)c(F)n1] | 469.90 | 470.10 [M + H]⁺ | DMSO-d₆: δ 10.92 (br, 1H), 8.44-8.38 (m, 1H), 8.30 (s, 1H), 8.03 (d, J = 8.0 Hz, 1H), 7.84 (d, J = 8.1 Hz, 1H), 7.57 (t, J = 8.8 Hz, 1H), 7.53 (s, 1H), 7.47 (d, J = 8.1 Hz, 1H), 3.77 (s, 2H), 3.21-3.14 (m, 4H), 2.76 (d, J = 4.8 Hz, 3H), 2.67-2.61 (m, 4H). |
| 106 | 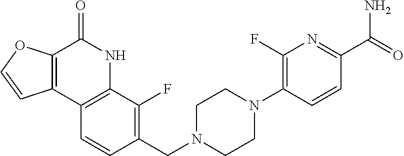 [SMILES: NC(=O)c1ccc(N2CCN(Cc3ccc4c([nH]c(=O)c5occc54)c3F)CC2)c(F)n1] | 439.42 | 440.10 [M + H]⁺ | DMSO-d₆: δ 11.84 (s, 1H), 8.24 (d, J = 2.0 Hz, 1H), 7.80 (dd, J = 8.0, 4.6 Hz, 2H), 7.74 (s, 1H), 7.51 (dd, J = 10.6, 8.1 Hz, 1H), 7.48- 7.40 (m, 2H), 7.29 (t, J = 7.3 Hz, 1H), 3.68 (s, 2H), 3.19-3.05 (m, 4H), 2.61-2.51 (m, 4H). |
| 107 | 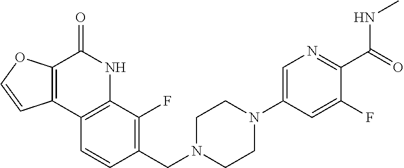 [SMILES: CNC(=O)c1ncc(N2CCN(Cc3ccc4c([nH]c(=O)c5occc54)c3F)CC2)cc1F] | 453.45 | / | / |
| 108 | 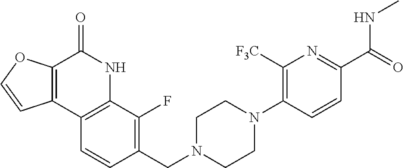 [SMILES: CNC(=O)c1ccc(N2CCN(Cc3ccc4c([nH]c(=O)c5occc54)c3F)CC2)c(C(F)(F)F)n1] | 503.46 | / | / |
| 109 | 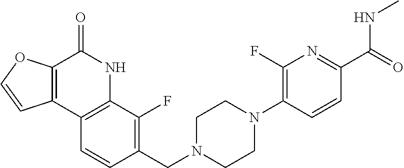 [SMILES: CNC(=O)c1ccc(N2CCN(Cc3ccc4c([nH]c(=O)c5occc54)c3F)CC2)c(F)n1] | 452.46 | 453.10 [M + H]⁺ | DMSO-d₆: δ 11.85 (s, 1H), 8.63-8.59 (m, 1H), 8.25 (d, J = 2.0 Hz, 1H), 8.06-8.01 (m, 1H), 7.85 (d, J = 8.0 Hz, 1H), 7.79 (d, J = 8.0 Hz, 1H), 7.47 (d, J = 1.6 Hz, 1H), 7.30-7.26 (m, 1H), 3.64 (s, 2H), 2.94 (d, J = 8.0 Hz, 2H), 2.74 (d, J = 8.0 Hz,4H), 2.14-2.10 (m, 2H), 1.79-1.70 (m, 4H). |
| 110 | 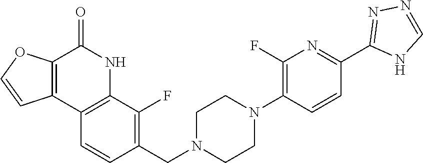 [SMILES: O=c1[nH]c2c(F)c(CN3CCN(c4ccc(-c5nnc[nH]5)nc4F)CC3)ccc2c2ccoc12] | 463.45 | 464.15 [M + H]⁺ | DMSO-d₆: δ 11.90 (s, 1H), 8.30-8.21 (m, 2H), 7.90 (d, J = 8.1 Hz, 1H), 7.84 (d, J = 8.1 Hz, 1H), 7.63-7.55 (m, 1H), 7.51 (s, 1H), 7.33 (s, 1H), 3.72 (s, 2H), 3.20-3.11 (m, 4H), 2.65-2.57 (m, 4H). |
| 111 | 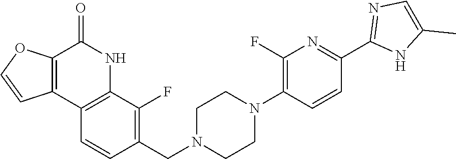 [SMILES: Cc1cnc(-c2ccc(N3CCN(Cc4ccc5c([nH]c(=O)c6occc65)c4F)CC3)c(F)n2)[nH]1] | 476.49 | / | / |
| 112 | 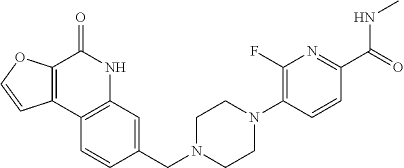 [SMILES: CNC(=O)c1ccc(N2CCN(Cc3ccc4c(c3)[nH]c(=O)c3occc34)CC2)c(F)n1] | 435.46 | 436.15 [M + H]⁺ | DMSO-d₆: δ 11.83 (s, 1H), 8.40-8.38 (m, 1H), 8.21-8.19 (m, 1H), 7.94 (d, J = 8.0 Hz, 1H), 7.81 (d, J = 8.0 Hz, 1H), 7.56-7.51 (m, 1H), 7.44 (d, J = 4.0 Hz, 1H), 7.40 (s, 1H), 7.23 (d, J = 8.0 Hz, 1H), 3.59 (s, 2H), 3.16- 3.14 (m, 4H), 2.72 (d, J = 4.0 Hz, 3H), 2.55- 2.51 (m, 4H). |
| 113 | 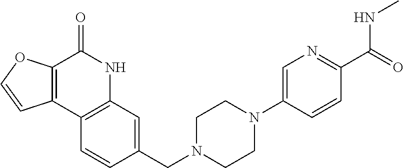 [SMILES: CNC(=O)c1ccc(N2CCN(Cc3ccc4c(c3)[nH]c(=O)c3occc34)CC2)cn1] | 417.47 | 418.15 [M + H]⁺ | DMSO-d₆: δ 11.83 (s, 1H), 8.40-8.38 (m, 1H), 8.25-8.23 (m, 1H), 8.21 (d, J = 4.0 Hz, 1H), 7.93 (d, J = 8.0 Hz, 1H), 7.79 (d, J = 8.0 Hz, 1H), 7.44 (d, J = 4.0 Hz, 1H), 7.41 (s, 1H), 7.37-7.34 (m, 1H), 7.23 (d, J = 8.0 Hz, 1H), 3.59 (s, 2H), 3.24-3.22 (m, 4H), 2.73 (d, J = 4.0 Hz, 3H), 2.55-2.51 (m, 4H). |
| 114 | 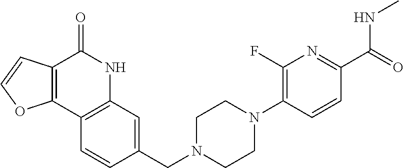 [SMILES: CNC(=O)c1ccc(N2CCN(Cc3ccc4c(c3)[nH]c(=O)c3ccoc34)CC2)c(F)n1] | 435.46 | 436.20 [M + H]⁺ | DMSO-d₆: δ 11.72 (s, 1H), 8.44-8.40 (m, 1H), 8.08 (d, J = 2.0 Hz, 1H), 7.89 (d, J = 8.0 Hz, 1H), 7.84 (d, J = 8.0 Hz, 1H), 7.59-7.55 (m, 1H), 7.45 (s, 1H), 7.27 (d, J = 8.0 Hz, 1H), 7.06 (d, J = 2.0 Hz, 1H), 3.63 (s, 2H), 3.09-3.17 (m, 4H), 2.75 (d, J = 4.0 Hz, 3H), 2.59-2.56 (m, 4H). |
| 115 | 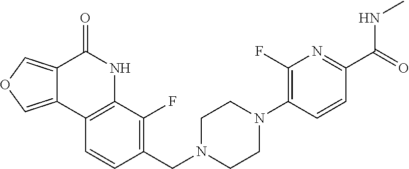 [SMILES: CNC(=O)c1ccc(N2CCN(Cc3ccc4c([nH]c(=O)c5cocc54)c3F)CC2)c(F)n1] | 453.45 | 454.20 [M + H]⁺ | DMSO-d₆: δ 11.09 (s, 1H), 8.63 (d, J = 4.0 Hz, 2H), 8.38-8.36 (m, 1H), 7.80 (d, J = 8.0 Hz, 1H), 7.67 (d, J = 8.0 Hz,, 1H), 7.53-7.51 (m, 1H), 7.17-7.16 (m, 1H), 3.62 (s, 2H), 3.15-3.12 (m, 4H), 2.72 (d, J = 4.0 Hz, 3H), 2.59-2.53 (m, 4H). |
| 116 | 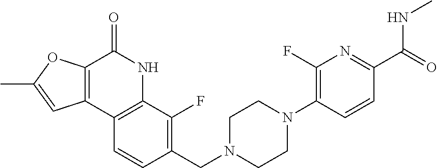 | 467.48 | 468.15 [M + H]⁺ | DMSO-d₆: δ 11.76 (s, 1H), 8.46-8.36 (m, 1H), 7.83 (d, J = 8.0 Hz, 1H), 7.74 (d, J = 8.1 Hz, 1H), 7.55 (dd, J = 10.6, 8.1 Hz, 1H), 7.29 (t, J = 7.3 Hz, 1H), 7.11 (s, 1H), 3.70 (s, 2H), 3.19-3.11 (m, 4H), 2.75 (d, J = 4.7 Hz, 3H), 2.62-2.55 (m, 4H), 2.51 (d, J = 2.7 Hz, 3H). |
| 117 | 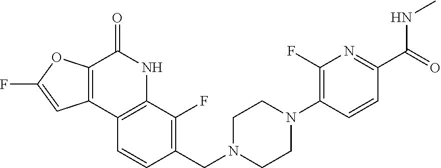 [SMILES: CNC(=O)c1ccc(N2CCN(Cc3ccc4c([nH]c(=O)c5oc(F)cc54)c3F)CC2)c(F)n1] | 471.44 | / | / |
| 118 | 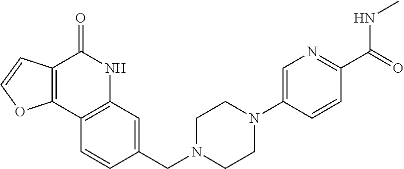 [SMILES: CNC(=O)c1ccc(N2CCN(Cc3ccc4c(c3)[nH]c(=O)c3ccoc34)CC2)cn1] | 417.47 | 418.15 [M + H]⁺ | DMSO-d₆: δ 11.69 (br, 1H), 8.40-8.38 (m, 1H), 8.23 (d, J = 4.0 Hz, 1H), 8.05 (d, J = 4.0 Hz, 1H), 7.86 (d, J = 8.0 Hz, 1H), 7.79 (d, J = 8.0 Hz, 1H), 7.42 (s, 1H), 7.37-7.34 (m, 1H), 7.24 (d, J = 8.0 Hz, 1H), 7.02 (d, J = 4.0 Hz, 1H), 3.60 (s, 2H), 3.24-3.22 (m, 4H), 2.73 (d, J = 4.0 Hz, 3H), 2.55-2.51 (m, 4H). |
| 119 | 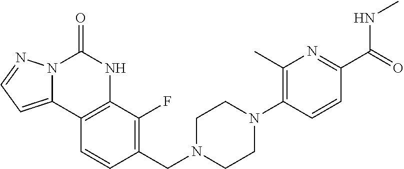 [SMILES: CNC(=O)c1ccc(N2CCN(Cc3ccc4c([nH]c(=O)n5nccc45)c3F)CC2)c(C)n1] | 449.49 | 450.15 [M + H]⁺ | DMSO-d₆: δ 11.90 (s, 1H), 8.43-8.29 (m, 1H), 8.13-8.04 (m, 1H), 7.84 (d, J = 8.1 Hz, 1H), 7.75 (d, J = 8.3 Hz, 1H), 7.44 (d, J = 8.3 Hz, 1H), 7.34-7.28 (m, 1H), 7.22 (s, 1H), 3.69 (s, 2H), 2.98-2.84 (m, 4H), 2.76 (d, J = 4.8 Hz, 3H), 2.64-2.55 (m, 4H), 2.45 (s, 3H). |
| 120 | 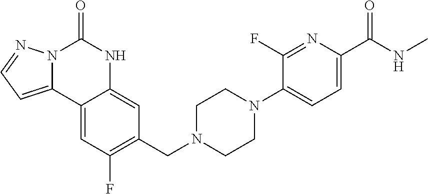 [SMILES: CNC(=O)c1ccc(N2CCN(Cc3cc4[nH]c(=O)n5nccc5c4cc3F)CC2)c(F)n1] | 453.45 | 454.15 [M + H]⁺ | DMSO-d₆: δ 11.79 (s, 1H), 8.38-8.34 (m, 1H), 8.06 (d, J = 1.8 Hz, 1H), 7.95 (d, J = 10.0 Hz, 1H), 7.81 (d, J = 7.1 Hz, 1H), 7.60- 7.48 (m, 1H), 7.41 (d, J = 6.2 Hz, 1H), 7.22 (d, J = 1.8 Hz, 1H), 3.64 (s, 2H), 3.21-3.10 (m, 4H), 2.73 (d, J = 4.8 Hz, 3H), 2.64-2.53 (m, 4H). |

### Example 121

### 7-((4-(2-methyl-6-(methylcarbamoyl)pyridin-3-yl)piperazin-1-yl)methyl)-3,6-difluoropyrazolo[1,5-a]quinoxalin-4(5H)-one

a) Preparation of 4-fluoro-1H-pyrazole-5-carbonyl chloride: A solution of 4-fluoro-1H-pyrazole-5-carboxylic acid (1.1 g, 8.5 mmol) in SOCl₂ (12 mL) was stirred at 80° C. for 2 hours. After completion of the reaction, the mixture was concentrated under reduced pressure to give the product (1.0 g, white solid, yield: 83%).
b) Preparation of N-(3-bromo-2,6-difluorophenyl)-4-fluoro-1H-pyrazole-5-carboxamide: To a solution of 3-bromo-2,6-difluoroaniline (1.5 g, 7.4 mmol) in THF (15 mL) was added LiHMDS (1 M in THE, 20.2 mL, 20.2 mmol) at 0° C. under nitrogen. The mixture was stirred at 0° C. for 30 minutes, 4-fluoro-1H-pyrazole-5-carbonyl chloride (1.0 g, 6.7 mmol, 1.0 eq) in THF (10 mL) was added dropwise. The reaction mixture was stirred at 0° C. for 30 minutes and warmed to room temperature for 1.5 hours. The mixture was quenched with water (20 ml-) and extracted with LA (20 mL×3). The combined organic phase was dried over anhydrous sodium sulfate and concentrated under reduced pressure. The residue was purified by silica gel column (PE/EA=5/1) to afford the product (1.5 g, yellow solid, yield: 72%). MS: 319.95[M+H]⁺, 321.95 [M+H+2]⁺.
c) Preparation of 7-bromo-3,6-difluoropyrazolo[1,5-a]quinoxalin-4(5H)-one: To a solution of N-(3-bromo-2,6-difluorophenyl)-4-fluoro-1H-pyrazole-5-carboxamide (1.5 g, 4.7 mmol) in DMSO (15 mL) was added K₂CO₃ (2.6 g, 18.7 mmol, 4.0 eq) under nitrogen. The mixture was stirred at 100° C. overnight. The mixture was cooled to room temperature. The mixture was quenched with water (20 mL) and extracted with EA (20 mL×3). Then the organic phase was concentrated under reduced pressure. The residue was purified by flash chromatography (C18, water/acetonitrile=55/45) to give the title compound as a white solid, which was used directly for the next step without further purification. MS(ESI): 300.00 [M+H]⁺, 302.00 [M+H+2]⁺.
d) Preparation of 3,6-difluoro-7-(hydroxymethyl)pyrazolo[1,5-a]quinoxalin-4(5H)-one: To a solution of 7-bromo-3,6-difluoropyrazolo[1,5-a]quinoxalin-4(5H)-one (1.2 g, 4.0 mmol) in dried dioxane (12 mL) was added Xphos Pd G2 (314.7 mg, 0.4 mmol) and (tributylstannyl)methanol (3.9 g, 12 mmol) under nitrogen. The mixture was stirred at 80° C. for 4 hours. The mixture was cooled to room temperature and KF aqueous solution (1 M, 24 mL) was added. The resulting mixture was filtered and the filtrate was extracted with EA (20 mL×3). The organic phase was concentrated under reduced pressure. The residue was purified by silica gel column (DCM/MeOH=1/0˜100/1˜80/1˜50/1) to give the product (260.0 mg, white solid, 2-step yield: 22%). MS(ESI): 252.10 [M+H]⁺. ¹H NMR (400 MHz, DMSO-d₆): δ 11.94 (s, 1H), 8.20 (d, J=3.8 Hz, 1H), 7.89 (d, J=8.6 Hz, 1H), 7.36 (t, J=7.7 Hz, 1H), 5.44 (t, J=5.8 Hz, 1H), 4.61 (d, J=5.5 Hz, 2H).
e) Preparation of 7-(bromomethyl)-3,6-difluoropyrazolo[1,5-a]quinoxalin-4(5H)-one: A solution of 3,6-difluoro-7-(hydroxymethyl)pyrazolo[1,5-a]quinoxalin-4(5H)-one (200.0 mg, 0.8 mmol) in HBr (48% in water, 20 mL) was stirred at 80° C. for 2 hours under nitrogen. The mixture was concentrated under reduced pressure to give the product (261.0 mg, white solid, yield: 96%). MS(ESI): 313.95 [M+H]⁺, 315.95 [M+H+2]⁺.
f) Preparation of 7-((4-(2-methyl-6-(methylcarbamoyl)quinoxal-3-yl)piperazin-1-yl)methyl)-3,6-difluoropyrazolo[1,5-a]quinoxaline-4(5H)-one: To a solution of 7-(bromomethyl)-3,6-difluoropyrazolo[1,5-a]quinoxalin-4(5H)-one (53.0 mg, 0.2 mmol) in acetonitrile (5 mL) was added KI (3.4 mg, 0.02 mmol), N,6-dimethyl-5-(piperazin-1-yl)picolinamide hydrochloride (47.0 mg, 0.2 mmol), DIEA (129.0 mg, 1.0 mmol) at room temperature under nitrogen. The mixture was stirred at 80° C. for 4 hours. The mixture was concentrated. The residue was purified by Prep-TLC (DCM/MeOH=10/1) to give the crude product. The crude product was purified by Prep-HPLC (water/acetonitrile=42/58%, 0.1% HCOOH) to give the title compound (48.8 mg, white solid, yield: 62%).

The compounds of Examples 121-128 were prepared using a synthesis method similar to that described in Example 121 (Scheme 8).

| | | | LC-MS | |
|---|---|---|---|---|
| Example | Compound | MW | (ESI) | ¹H NMR (400 MHz) |
| 121 | 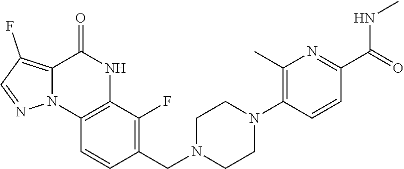 [SMILES: CNC(=O)c1ccc(N2CCN(Cc3ccc4c([nH]c(=O)c5c(F)cnn54)c3F)CC2)c(C)n1] | 467.48 | 468.10 [M + H]⁺ | DMSO-d₆: δ 11.98 (s, 1H), 8.45-8.38 (m, 1H), 8.22 (d, J = 3.8 Hz, 1H), 7.91 (d, J = 8.2 Hz, 1H), 7.78 (d, J = 8.5 Hz, 1H), 7.47 (d, J = 8.2 Hz, 1H), 7.35 (t, J = 8.1 Hz, 1H), 3.70 (s, 2H), 2.99-2.89 (m, 4H), 2.79 (d, J = 4.1 Hz, 3H), 2.66-2.56 (m, 4H), 2.46 (s, 3H). |
| 122 | 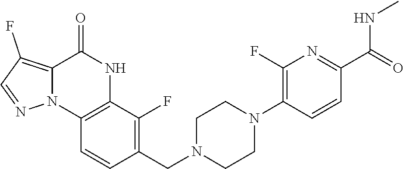 [SMILES: CNC(=O)c1ccc(N2CCN(Cc3ccc4c([nH]c(=O)c5c(F)cnn54)c3F)CC2)c(F)n1] | 471.44 | 472.15 [M + H]⁺ | DMSO-d₆: δ 8.40 (q, J = 4.8 Hz, 1H), 8.22 (d, J = 3.7 Hz, 1H), 7.91 (d, J = 8.3 Hz, 1H), 7.83 (d, J = 7.0 Hz, 1H), 7.56 (dd, J = 10.4, 8.2 Hz, 1H), 7.34 (dd, J = 8.4, 7.1 Hz, 1H), 3.68 (s, 2H), 3.21-3.11 (m, 4H), 2.76 (d, J = 4.8 Hz, 3H), 2.63-2.55 (m, 4H). |
| 123 | 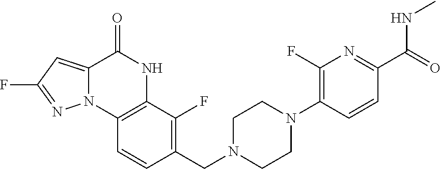 [SMILES: CNC(=O)c1ccc(N2CCN(Cc3ccc4c([nH]c(=O)c5cc(F)nn54)c3F)CC2)c(F)n1] | 471.44 | / | / |
| 124 | 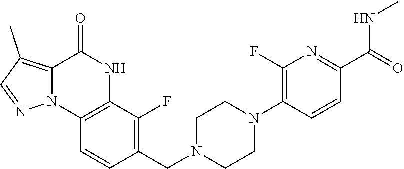 [SMILES: CNC(=O)c1ccc(N2CCN(Cc3ccc4c([nH]c(=O)c5c(C)cnn54)c3F)CC2)c(F)n1] | 466.49 | 467.30 [M + H]⁺ | DMSO-d₆: δ 11.75 (s, 1H), 8.61-8.59 (m, 1H), 8.06-8.01 (m, 1H), 7.90 (s, 1H), 7.845 (t, J = 8.0 Hz, 2H), 7.26 (t, J = 8.0 Hz, 1H), 3.59 (s, 2H), 2.95-2.92 (m, 2H), 2.74-2.73 (m ,4H), 2.40 (s, 3H), 2.14-2.09 (m, 2H), 1.72-1.66 (m, 4H). |
| 125 | 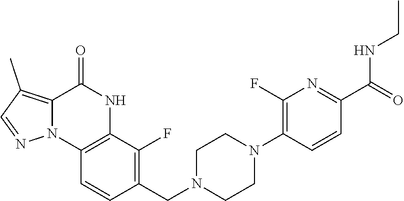 [SMILES: CCNC(=O)c1ccc(N2CCN(Cc3ccc4c([nH]c(=O)c5c(C)cnn54)c3F)CC2)c(F)n1] | 481.51 | 482.25 [M + H]⁺ | DMSO-d₆: δ 11.76 (br, 1H), δ 8.45-8.42 (m, 1H), 7.90 (s, 1H), 7.84 (d, J = 8.0 Hz, 1H), 7.80 (d, J = 8.0 Hz, 1H), 7.55-7.50 (m, 1H), 7.29-7.25 (m, 1H), 3.64 (s, 2H), 3.24- 3.22 (m, 2H), 3.14-3.11 (m, 4H), 2.58-2.56 (m, 4H), 2.40 (s, 3H), 1.07 (t, J = 8.0 Hz, 3H). |
| 126 | 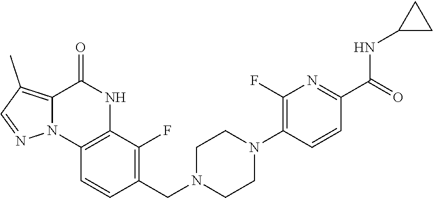 [SMILES: Cc1cnn2c1c(=O)[nH]c1c(F)c(CN3CCN(c4ccc(C(=O)NC5CC5)nc4F)CC3)ccc12] | 493.52 | 494.20 [M + H]⁺ | DMSO-d₆: δ 11.75 (br, 1H), δ 8.33 (d, J = 5.2 Hz, 1H), 7.91 (s, 1H), 7.85 (d, J = 8.0 Hz, 1H), 7.80 (d, J = 8.0 Hz, 1H), 7.53 (dd, J = 8.0 ,8.0 Hz, 1H), 7.28 (t, J = 6.8 Hz, 1H), 3.65 (s, 2H), 3.14-3.11 (m, 4H), 2.83-2.82 (m, 1H), 2.59-2.57 (m, 4H), 2.42 (s, 3H), 0.64-0.62 (m, 4H). |
| 127 | 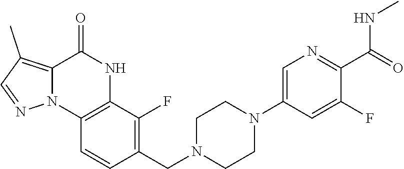 [SMILES: CNC(=O)c1ncc(N2CCN(Cc3ccc4c([nH]c(=O)c5c(C)cnn54)c3F)CC2)cc1F] | 467.48 | 468.15 [M + H]⁺ | DMSO-d₆: δ 11.76 (br, 1H), 8.26-8.24 (m, 1H), 8.09 (s, 1H), 7.90 (s, 1H), 7.84 (d, J = 8.0 Hz, 1H), 7.27 (t, J = 8.0 Hz, 1H), 7.19- 7.15 (m, 1H), 3.63 (s, 2H), 3.35-3.31 (m, 4H), 2.70 (d, J = 4.0 Hz, 3H), 2.54-2.52 (m, 4H), 2.40 (s, 3H). |
| 128 | 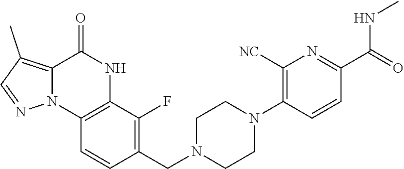 [SMILES: CNC(=O)c1ccc(N2CCN(Cc3ccc4c([nH]c(=O)c5c(C)cnn54)c3F)CC2)c(C#N)n1] | 474.5 | 475.15 [M + H]⁺ | DMSO-d₆: δ 11.76 (s, 1H), 8.59-8.52 (m, 1H), 8.07 (d, J = 8.8 Hz, 1H), 7.90 (s, 1H), 7.84 (d, J = 8.4 Hz, 1H), 7.70 (d, J = 8.9 Hz, 1H), 7.27 (t, J = 7.7 Hz, 1H), 3.66 (s, 2H), 3.29-3.19 (m, 4H), 2.74 (d, J = 4.8 Hz, 3H), 2.65-2.53 (m, 4H), 2.40 (s, 3H). |

### Example 129

### 7-((4-(2-fluoro-6-(2,2-difluoroethylcarbamoyl)pyridin-3-yl)piperazin-1-yl)methyl)-6-fluoro-3-methylpyrazolo[1,5-a]quinoxalin-4(5H)-one

a) Preparation of (4-bromo-3-fluoro-2-nitrophenyl)hydrazine: To a solution of 4-bromo-3-fluoro-2-nitroaniline (5.0 g, 21.3 mmol) in concentrated hydrochloric acid (36% aq. 50 mL), a solution of NaNO₂ (14.4 g, 64.0 mmol) in H₂O (50 ml) was added dropwise at −10° C. The mixture was stirred at −10° C. for 1 hour. The mixture was cooled to −30° C., and a solution of SnCl₂·2H₂O (1.6 g, 23.4 mmol) in concentrated hydrochloric acid (36% aq. 50 ml) was added dropwise. The mixture was stirred at −30° C. for 1 hour. After the reaction was completed, the mixture was poured into water (200 mL), and the pH of the solution was adjusted to 7˜8 with NaHCO₃ solid. The resulting mixture was extracted with EA (100 mL×3). The organic phase was combined and washed with brine, dried over anhydrous sodium sulfate and concentrated under reduced pressure to afford the product (4.3 g, crude, yellow solid, yield: 810%).
b) Preparation of methyl 1-(4-bromo-3-fluoro-2-nitrophenyl)-4-methyl-1H-pyrazole-5-carboxylate: To a solution of (4-bromo-3-fluoro-2-nitrophenyl)hydrazine (1.0 g, 4.0 mmol) in acetic acid (10 mL), a solution of methyl 4-(dimethylamino)-3-methyl-2-oxobut-3-enoate (0.82 g, 4.8 mmol) in acetic acid (10 mL) was added dropwise at room temperature. The mixture was stirred at 80° C. overnight. After the reaction was completed, the mixture was poured into ice water (100 mL) and extracted with EA (100 mL×3). The organic phase was combined and washed with brine, dried over anhydrous sodium sulfate and concentrated under reduced pressure. The residue was purified by silica gel (PE:EA=15:1) to afford the product (0.83 g, yellow solid, yield: 58%). ¹H NMR (400 MHz, DMSO-d₆): δ 8.16-8.14 (m, 1H), 7.78 (s, 1H), 7.61-7.59 (m, 1H), 3.71 (s, 3H), 2.25 (s, 3H).
c) Preparation of 7-bromo-6-fluoro-3-methylpyrazolo[1,5-a]quinoxalin-4(5H)-one: To a solution of methyl 1-(4-bromo-3-fluoro-2-nitrophenyl)-4-methyl-1H-pyrazole-5-carboxylate (0.83 g, 2.3 mmol) in acetic acid (10 mL), Fe dust (0.65 g, 11.6 mmol) was added at room temperature. The mixture was stirred at 80° C. overnight. After the reaction was completed, the mixture was filtered, the filtrate was diluted with water (50 mL) and ethyl acetate (5 mL), and then the solid was collected by filtration to afford the product (370 mg, gray white solid, yield: 55%). MS(ESI): 295.90 [M+H]⁺, 293.85 [M−H]-. ¹H NMR (400 MHz, DMSO-d₆): δ 11.90 (br, 1H), 7.91 (s, 1H), 7.77 (d, J=8.0 Hz, 1H), 7.50-7.46 (m, 1H), 2.38 (s, 3H).
d) Preparation of 6-fluoro-7-(hydroxymethyl)-3-methylpyrazolo[1,5-a]quinoxalin-4(5H)-one: To a solution of 7-bromo-6-fluoro-3-methylpyrazolo[1,5-a]quinoxalin-4(5H)-one (5.0 g, 16.9 mmol) in dioxane (100 mL) was added tributylstannylmethanol (10.9 g, 33.9 mmol) and Xphos Pd G2 (1.3 g, 1.7 mmol) at room temperature. The mixture was stirred at 90° C. under nitrogen atmosphere for 16 hours. After the reaction was completed, a solution of KF (1 M, 100 mL) was added at room temperature. The mixture was stirred at the same temperature for 10 mins and filtered. The filtrate was extracted with EA (200 mL×3). The combined organic phase was washed with brine (200 mL), dried over anhydrous sodium sulfate and concentrated under reduced pressure. The residue was triturated with a mixture of PE/EA (v/v=1:1) and the solid was collected by filtration to afford the title compound (2.8 g, grey solid, yield: 67%). MS(ESI): 248.05 [M+H]⁺, 246.00 [M−H]-.
e) Preparation of 7-(bromomethyl)-6-fluoro-3-methylpyrazolo[1,5-a]quinoxalin-4(5H)-one: A solution of 6-fluoro-7-(hydroxymethyl)-3-methylpyrazolo[1,5-a]quinoxalin-4(5H)-one (2.8 g, 11.3 mmol) in HBr (30 mL, 48% in H₂O) was stirred at 80° C. for 2 hours. After the reaction was completed, the reaction was concentrated under reduced pressure to afford the product (2.7 g, crude, yellow solid). MS(ESI): 309.90[M+H]⁺, 311.90 [M+H+2]⁺.
f) Preparation of 7-((4-(2-fluoro-6-(2,2-difluoroethylcarbamoyl)pyridin-3-yl)piperazin-1-yl)methyl)-6-fluoro-3-methylpyrazolo[1,5-a]quinoxalin-4(5H)-one: To a solution of 7-(bromomethyl)-6-fluoro-3-methylpyrazolo[1,5-a]quinoxalin-4(5H)-one (193.8 mg crude, 0.63 mmol), KI (8.7 mg, 0.05 mmol) and N-(2,2-difluoroethyl)-6-fluoro-5-(piperazin-1-yl)picolinamide (150.0 mg, 0.52 mmol) in CH₃CN (20 mL) was added K₂CO₃ (359.5 mg, 2.61 mmol, 5.0 eq) at room temperature. The resulting mixture was stirred at 80° C. for 3 hours. After completion of the reaction, the reaction mixture was filtered. The solid was purified by Prep-HPLC (C18, acetonitrile in water 5˜50%, 0.1% HCOOH) to give the target compound (21.8 mg, white solid, yield: 8.1%).

The compounds of Examples 130-135, 151-167, and 203-204 were prepared using a synthesis method similar to that described in Example 129 (Scheme 9); the compounds of Examples 136-141 were prepared using a synthesis method similar to that described in Example 50 (Scheme 6); the compounds of Examples 142-144 and 149 were prepared using a synthesis method similar to that described in Example 1 (Scheme 1); the compound of Example 145 was prepared using a synthesis method similar to that described in Example 49 (Scheme 5); the compounds of Example 146-148 were prepared using a synthesis method similar to that described in Example 66 (Scheme 7); the compound of Example 150 was prepared using a synthesis method similar to that described in Example 6 (Scheme 2); the compounds of Examples 168-202 were prepared using a synthesis method similar to that described in Example 121 (Scheme 8); the compound of Example 205 was prepared using a synthesis method similar to that described in Example 48.

| Ex- | | | | |
|---|---|---|---|---|
| am- | | | LC-MS | |
| ple | Compound | MW | (ESI) | ¹H NMR (400 MHz) |
| 129 | 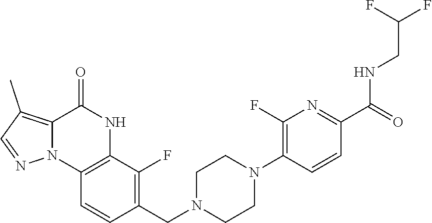 [SMILES: Cc1cnn2c1c(=O)[nH]c1c(F)c(CN3CCN(c4ccc(C(=O)NCC(F)F)nc4F)CC3)ccc12] | 517.49 | 518.25 [M + H]⁺ | DMSO-d₆: δ 8.72 (t, J = 6.1 Hz, 1H), 7.93 (s, 1H), 7.91-7.83 (m, 2H), 7.58 (dd, J = 10.6, 8.2 Hz, 2H), 7.31 (t, J = 7.7 Hz, 2H), 6.31- 5.91 (m, 1H), 3.67 (s, 2H), 3.68-3.56 (m, 2H), 3.24-3.13 (m, 4H), 2.64-2.56 (m, 4H), 2.44 (s, 3H). |
| 130 | 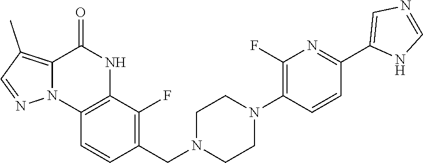 [SMILES: Cc1cnn2c1c(=O)[nH]c1c(F)c(CN3CCN(c4ccc(-c5cnc[nH]5)nc4F)CC3)ccc12] | 476.49 | / | / |
| 131 | 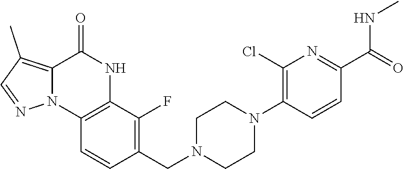 [SMILES: CNC(=O)c1ccc(N2CCN(Cc3ccc4c([nH]c(=O)c5c(C)cnn54)c3F)CC2)c(Cl)n1] | 483.93 | 484.25 [M + H]⁺ | DMSO-d₆: δ 11.73 (s, 1H), 8.39 (q, J = 5.1 Hz, 1H), 7.92-7.87 (m, 2H), 7.84 (d, J = 8.4 Hz, 1H), 7.62 (d, J = 8.2 Hz, 1H), 7.27 (dd, J = 8.5, 6.8 Hz, 1H), 3.65 (s, 2H), 3.13-3.01 (m, 4H), 2.74 (d, J = 4.8 Hz, 3H), 2.65-2.53 (m, 4H), 2.41 (s, 3H). |
| 132 | 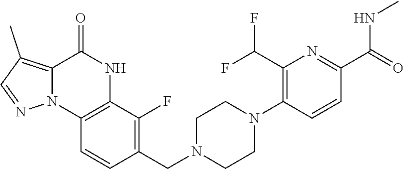 [SMILES: CNC(=O)c1ccc(N2CCN(Cc3ccc4c([nH]c(=O)c5c(C)cnn54)c3F)CC2)c(C(F)F)n1] | 499.5 | 500.20 [M + H]⁺ | DMSO-d₆: δ 11.76 (s, 1H), 8.42-8.36 (m, 1H), 8.05 (d, J = 8.5 Hz, 1H), 7.90 (s, 1H), 7.83 (t, J = 8.7 Hz, 2H), 7.28 (d, J = 7.5 Hz, 1H), 7.10 (t, J = 52 Hz, 1H), 3.65 (s, 2H), 2.99-2.94 (m, 4H), 2.78 (d, J = 4.8 Hz, 3H), 2.64-2.56 (m, 4H), 2.40 (s, 3H). |
| 133 | 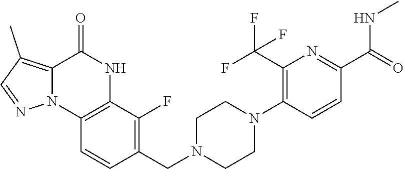 [SMILES: CNC(=O)c1ccc(N2CCN(Cc3ccc4c([nH]c(=O)c5c(C)cnn54)c3F)CC2)c(C(F)(F)F)n1] | 517.49 | 518.20 [M + H]⁺ | CDCl₃: δ 8.72 (s, 1H), 8.29 (d, J = 8.5 Hz, 1H), 7.94 (d, J = 8.6 Hz, 1H), 7.80 (d, J = 5.4 Hz, 1H), 7.76 (s, 1H), 7.83-7.77 (m, 1H), 7.31 (t, J = 7.8 Hz, 1H), 3.73 (s, 2H), 3.12- 3.06 (m, 4H), 3.03 (d, J = 5.1 Hz, 3H), 2.73- 2.67 (m, 4H), 2.56 (s, 3H). |
| 134 | 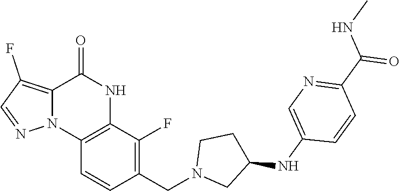 | 449.49 | 450.20 [M + H]⁺ | DMSO-d₆: δ 8.26 (q, J = 6.3 Hz, 1H), 7.90 (s, 1H), 7.88 (d, J = 2.7 Hz, 1H), 7.83 (d, J = 8.5 Hz, 1H), 7.69 (d, J = 8.6 Hz, 1H), 7.31- 7.21 (m, 1H), 6.91 (dd, J = 8.6, 2.7 Hz, 1H), 6.65 (d, J = 6.8 Hz, 1H), 4.02-3.88 (m, 1H), 3.70 (s, 2H), 2.89-2.80 (m, 1H), 2.72 (d, J = 4.8 Hz, 3H), 2.70-2.58 (m, 1H), 2.51- 2.46 (m, 1H), 2.46-2.40 (m, 1H), 2.41 (s, 3H), 2.29-2.16 (m, 1H), 1.66-1.50 (m, 1H). |
| 135 | 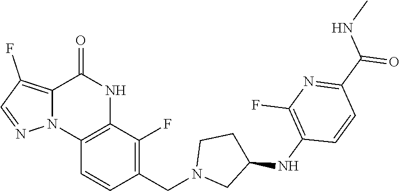 | 467.48 | 468.15 [M + H]⁺ | DMSO-d₆: δ 11.77 (s, 1H), 8.20-8.13 (m, 1H), 7.91 (s, 1H), 7.84 (d, J = 8.5 Hz, 1H), 7.70 (d, J = 8.0 Hz, 1H), 7.27 (dd, J = 8.5, 6.9 Hz, 1H), 7.14 (dd, J = 10.6, 8.1 Hz, 1H), 6.45 (d, J = 6.6 Hz, 1H), 4.04-3.95 (m, 1H), 3.71 (s, 2H), 2.93-2.87 (m, 1H), 2.72 (d, J = 4.7 Hz, 3H), 2.67-2.62 (m, 1H), 2.57- 2.46 (m, 2H), 2.42 (s, 3H), 2.25-2.15 (m, 1H), 1.80-1.70 (m, 1H). |
| 136 | 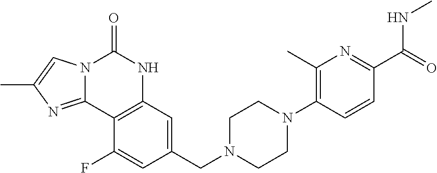 | 463.52 | 464.15 [M + H]⁺ | DMSO-d₆: δ 8.42 (d, J = 5.0 Hz, 1H), 7.79 (d, J = 8.3 Hz, 1H), 7.62 (d, J = 1.3 Hz, 1H), 7.48 (d, J = 8.3 Hz, 1H), 7.21 (s, 1H), 7.12 (d, J = 11.3 Hz, 1H), 3.63 (s, 2H), 3.13-2.97 (m, 4H), 2.79 (d, J = 4.8 Hz, 3H), 2.58-2.64 (m, 4H), 2.49 (s, 3H), 2.31 (s, 3H). |
| 137 | 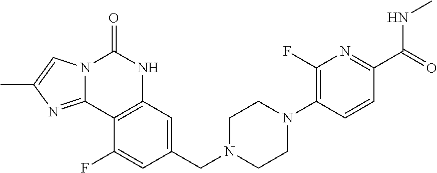 | 467.48 | 468.10 [M + H]⁺ | CD₃OD: δ 8.51 (d, J = 5.7 Hz, 1H), 7.88 (dd, J = 8.0, 1.4 Hz, 1H), 7.61 (d, J = 1.3 Hz, 1H), 7.51 (dd, J = 10.3, 8.1 Hz, 1H), 7.23-7.10 (m, 2H), 3.69 (s, 2H), 3.31-3.23 (m, 4H), 2.91 (d, J = 4.8 Hz, 3H), 2.74-2.64 (m, 4H), 2.39 (d, J = 1.2 Hz, 3H). |
| 138 | 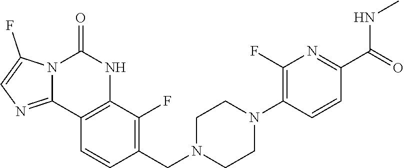 [SMILES: CNC(=O)c1ccc(N2CCN(Cc3ccc4c([nH]c(=O)n5c(F)cnc45)c3F)CC2)c(F)n1] | 471.44 | / | / |
| 139 | 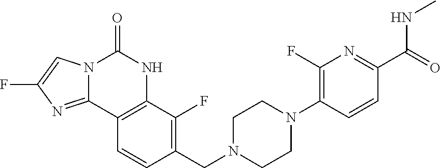 [SMILES: CNC(=O)c1ccc(N2CCN(Cc3ccc4c([nH]c(=O)n5cc(F)nc45)c3F)CC2)c(F)n1] | 471.44 | / | / |
| 140 | 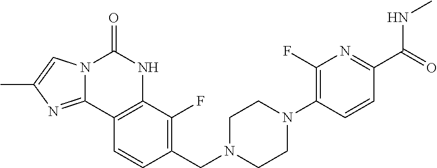 | 467.48 | 468.15 [M + H]⁺ | DMSO-d₆: δ 8.41 (q, J = 4.8 Hz, 1H), 7.88 (d, J = 8.0 Hz, 1H), 7.83 (d, J = 8.1 Hz, 1H), 7.64 (d, J = 1.6 Hz, 1H), 7.55 (dd, J = 10.6, 8.1 Hz, 1H), 7.33 (t, J = 7.2 Hz, 1H), 3.70 (s, 2H), 3.25-3.08 (m, 4H), 2.75 (d, J = 4.7 Hz, 3H), 2.65-2.55 (m, 4H), 2.31 (s, 3H). |
| 141 | 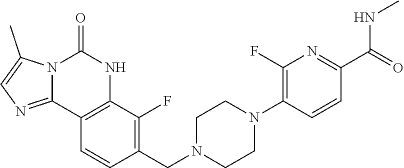 [SMILES: CNC(=O)c1ccc(N2CCN(Cc3ccc4c([nH]c(=O)n5c(C)cnc45)c3F)CC2)c(F)n1] | 467.48 | 468.20 [M + H]⁺ | DMSO-d₆: δ 8.43-8.41 (m, 1H), 7.84-7.81 (m, 2H), 7.57-7.52 (m, 1H), 7.28 (t, J = 8.0 Hz, 1H), 7.12 (s, 1H), 3.67 (s, 2H), 3.17- 3.15 (m, 4H), 2.73 (d, J = 4.0 Hz, 3H), 2.61 (s, 3H), 2.58-2.55 (m, 4H). |
| 142 | 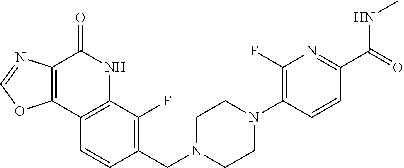 [SMILES: CNC(=O)c1ccc(N2CCN(Cc3ccc4c([nH]c(=O)c5ncoc54)c3F)CC2)c(F)n1] | 454.44 | / | / |
| 143 | 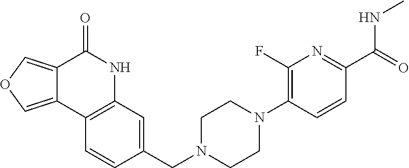 [SMILES: CNC(=O)c1ccc(N2CCN(Cc3ccc4c(c3)[nH]c(=O)c3cocc34)CC2)c(F)n1] | 435.46 | 436.10 [M + H]⁺ | DMSO-d₆: δ 10.93 (s, 1H), 8.58 (d, 1.5 Hz, 1H), 8.55 (d, 1.5 Hz, 1H), 8.38-8.36 (m, 1H), 7.82 (d, J = 8.0 Hz, 2H), 7.53 (dd, J = 10.5, 8.2 Hz,, 1H), 7.21 (s, 1H), 7.09 (d, J = 8.0 Hz, 1H), 3.52 (s, 2H), 3.19-3.12 (m, 4H), 2.72 (d, J = 4.0 Hz, 3H), 2.59-2.53 (m, 4H). |
| 144 | 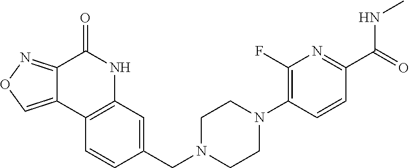 [SMILES: CNC(=O)c1ccc(N2CCN(Cc3ccc4c(c3)[nH]c(=O)c3nocc34)CC2)c(F)n1] | 436.45 | 437.10 [M + H]⁺ | DMSO-d₆: δ 11.69 (s, 1H), 10.00 (s, 1H), 8.39-8.36 (m, 1H), 7.91 (d, J = 8.0 Hz, 1H), 7.81 (d, J = 8.0 Hz, 1H), 7.54 (dd, J = 10.6, 8.1 Hz, 1H), 7.31 (s, 1H), 7.19 (d, J = 8.0 Hz, 1H), 3.56 (s, 2H), 3.16-3.15 (m, 4H), 2.72 (d, J = 4.0 Hz, 3H), 2.55-2.53 (m, 4H). |
| 145 | 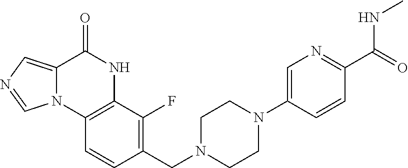 [SMILES: CNC(=O)c1ccc(N2CCN(Cc3ccc4c([nH]c(=O)c5cncn54)c3F)CC2)cn1] | 435.46 | 436.10 [M + H]⁺ | DMSO-d₆: δ 11.45 (s, 1H), 9.03 (s, 1H), 8.37-8.36 (m, 1H), 8.24-8.22 (m, 1H), 7.98 (d, J = 8.0 Hz, 1H), 7.87 (s, 1H), 7.78 (d, J = 8.0 Hz, 1H), 7.34 (dd, J = 8.8, 2.9 Hz, 1H),, 7.28-7.24 (m, 1H), 3.63 (s, 2H), 3.32-3.30 (m, 4H), 2.73 (d, J = 4.0 Hz, 3H), 2.55-2.53 (m, 4H). |
| 146 | 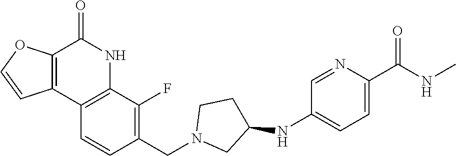 [SMILES: CNC(=O)c1ccc(N[C@@H]2CCN(Cc3ccc4c([nH]c(=O)c5occc54)c3F)C2)cn1] | 435.46 | 436.10 [M + H]⁺ | DMSO-d₆: δ 11.83 (s, 1H), 8.27-8.19 (m, 2H), 7.87 (d, J = 2.7 Hz, 1H), 7.77 (d, J = 8.1 Hz, 1H), 7.68 (d, J = 8.6 Hz, 1H), 7.45 (d, J = 2.0 Hz, 1H), 7.32-7.20 (m, 1H), 6.90 (dd, J = 8.7, 2.7 Hz, 1H), 6.62 (d, J = 6.7 Hz, 1H), 3.94 (dt, J = 9.4, 5.4 Hz, 1H), 3.73 (d, J = 3.4 Hz, 2H), 2.83 (dd, J = 9.3, 6.9 Hz, 1H), 2.71 (d, J = 4.8 Hz, 3H), 2.66 (dt, J = 8.1, 4.4 Hz, 1H), 2.50 (d, J = 8.9 Hz, 1H), 2.41 (dd, J = 9.5, 4.4 Hz, 1H), 2.22 (dq, J = 13.3, 6.9 Hz, 1H), 1.56 (dq, J = 12.9, 6.7 Hz, 1H). |
| 147 | 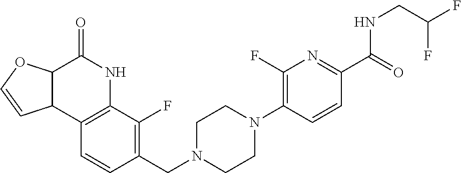 [SMILES: O=C(NCC(F)F)c1ccc(N2CCN(Cc3ccc4c(c3F)NC(=O)C3OC=CC43)CC2)c(F)n1] | 503.46 | 504.15 [M + H]⁺ | DMSO-d₆: δ 11.90 (s, 1H), 8.74 (t, J = 6.3 Hz, 1H), 8.28 (d, J = 1.8 Hz, 1H), 7.85 (dd, J = 14.1, 8.2 Hz, 2H), 7.65-7.53 (m, 1H), 7.51 (d, J = 1.8 Hz, 1H), 7.38-7.23 (m, 1H), 6.35-5.85 (m, 1H), 3.72 (s, 2H), 3.72-3.56 (m, 2H), 3.25-3.13 (m, 4H), 2.67-2.55 (m, 4H). |
| 148 | 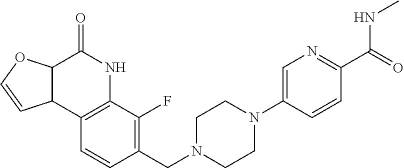 [SMILES: CNC(=O)c1ccc(N2CCN(Cc3ccc4c(c3F)NC(=O)C3OC=CC43)CC2)cn1] | 435.46 | 436.15 [M + H]⁺ | DMSO-d₆: δ 11.90 (s, 1H), 8.41 (q, J = 4.8 Hz, 1H), 8.28 (d, J = 2.0 Hz, 1H), 8.25 (d, J = 2.9 Hz, 1H), 7.88-7.78 (m, 2H), 7.51 (d, J = 2.0 Hz, 1H), 7.37 (dd, J = 8.9, 2.9 Hz, 1H), 7.33 (dd, J = 8.1, 6.4 Hz, 1H), 3.71 (s, 2H), 3.39-3.25 (m, 4H), 2.77 (d, J = 4.8 Hz, 3H), 2.63-2.53 (m, 4H). |
| 149 | 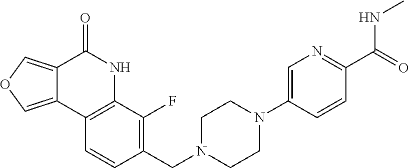 [SMILES: CNC(=O)c1ccc(N2CCN(Cc3ccc4c([nH]c(=O)c5cocc54)c3F)CC2)cn1] | 435.46 | 436.05 [M + H]⁺ | DMSO-d₆: δ 10.89 (s, 1H), 8.63 (d, J = 1.5 Hz, 1H), 8.55 (d, 1.5 Hz, 1H), 8.38-8.36 (m, 1H), 8.20 (d, J = 8.0 Hz, 1H), 7.77 (d, J = 8.0 Hz,, 1H), 7.63 (d, J = 8.0 Hz, 1H), 7.33 (d, J = 8.0 Hz, 1H), 7.15 (t, J = 8.0 Hz, 1H), 3.60 (s, 2H), 3.30-3.27 (m, 4H), 2.72 (d, J = 4.0 Hz, 3H), 2.56-2.52 (m, 4H). |
| 150 | 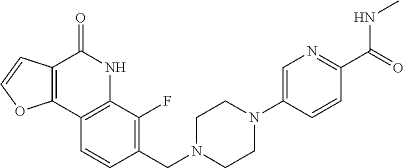 [SMILES: CNC(=O)c1ccc(N2CCN(Cc3ccc4c([nH]c(=O)c5ccoc54)c3F)CC2)cn1] | 435.46 | 436.10 [M + H]⁺ | DMSO-d₆: δ 11.76 (br, IH), 8.41-8.40 (m, 1H), 8.25 (d, J = 4.0 Hz, 1H), 8.13 (d, J = 4.0 Hz, 1H), 7.80 (d, J = 8.0 Hz, 1H), 7.43 (d, J = 8.0 Hz, 1H), 7.38-7.31 (m, 2H), 7.11 (d, J = 4.0 Hz, 1H), 3.70 (s, 2H), 3.39-3.30 (m, 4H), 2.75 (d, J = 4.0 Hz, 3H), 2.57-2.53 (m, 4H). |
| 151 | 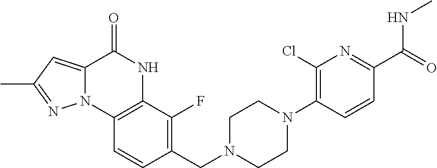 | 483.93 | 484.15 [M + H]⁺ | DMSO-d₆: δ 11.90 (s, 1H), 8.45-8.38 (m, 1H), 7.89 (d, J = 8.2 Hz, 1H), 7.83 (d, J = 8.4 Hz, 1H), 7.62 (d, J = 8.2 Hz, 1H), 7.33-7.25 (m, 1H), 6.95 (s, 1H), 3.66 (s, 2H), 3.09-3.01 (m, 4H), 2.74 (d, J = 4.8 Hz, 3H), 2.61-2.54 (m, 4H), 2.39 (s, 3H). |
| 152 | 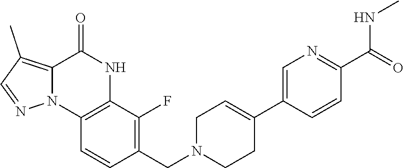 [SMILES: CNC(=O)c1ccc(C2=CCN(Cc3ccc4c([nH]c(=O)c5c(C)cnn54)c3F)CC2)cn1] | 446.49 | 447.15 [M + H]⁺ | DMSO-d₆: δ 11.80 (s, 1H), 8.75-8.69 (m, 1H), 8.68 (s, 1H), 8.28 (s, 1H), 7.98-7.94 (m, 1H), 7.93 (s, 1H), 7.87 (d, J = 8.4 Hz, 1H), 7.32 (t, J = 7.6 Hz, 1H), 6.41 (s, 1H), 3.72 (s, 2H), 3.16 (s, 2H), 2.79 (d, J = 4.8 Hz, 3H), 2.75-2.67 (m, 2H), 2.55-2.48 (m, 2H), 2.43 (s, 3H). |
| 153 | 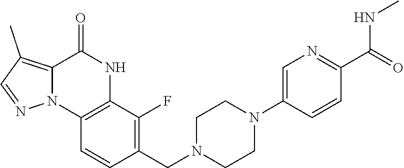 [SMILES: CNC(=O)c1ccc(N2CCN(Cc3ccc4c([nH]c(=O)c5c(C)cnn54)c3F)CC2)cn1] | 449.49 | 450.15 [M + H]⁺ | DMSO-d₆: δ 8.25-8.19 (m, 1H), 8.15 (br, 1H), 7.90 (s, 1H), 7.84 (d, J = 9.4 Hz, 1H), 7.77 (d, J = 9.0 Hz, 1H), 7.34 (dd, J = 9.2, 2.7 Hz, 1H), 7.31-7.24 (m, 1H), 3.62 (s, 2H), 3.30-3.27 (m, 4H), 2.72 (s, 3H), 2.57-2.49 (m, 4H), 2.40 (s, 3H). |
| 154 | 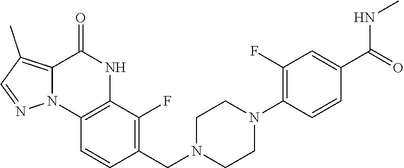 [SMILES: CNC(=O)c1ccc(N2CCN(Cc3ccc4c([nH]c(=O)c5c(C)cnn54)c3F)CC2)c(F)c1] | 466.49 | 467.10 [M + H]⁺ | DMSO-d₆: δ 11.77 (s, 1H), 8.34 (s, 1H), 7.98-7.82 (m, 2H), 7.58 (t, J = 12.3 Hz, 2H), 7.30 (s, 1H), 7.04 (s, 1H), 3.66 (s, 2H), 3.25- 3.10 (m, 4H), 2.74 (d, J = 4.6 Hz, 3H), 2.60- 2.52 (m, 4H), 2.44 (s, 3H). |
| 155 | 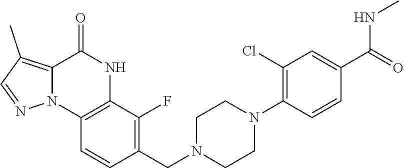 [SMILES: CNC(=O)c1ccc(N2CCN(Cc3ccc4c([nH]c(=O)c5c(C)cnn54)c3F)CC2)c(Cl)c1] | 482.94 | 483.20 [M + H]⁺ | DMSO-d₆: δ 11.76 (s, 1H), 8.39 (q, J = 5.4, 4.9 Hz, 1H), 7.90 (s, 1H), 7.87-7.79 (m, 2H), 7.75-7.65 (m, 1H), 7.27 (t, J = 7.6 Hz, 1H), 7.14 (d, J = 8.6 Hz, 1H), 3.64 (s, 2H), 3.08-2.94 (m, 4H), 2.71 (d, J = 4.2 Hz, 3H), 2.62-2.50 (m, 4H), 2.40 (s, 3H). |
| 156 | 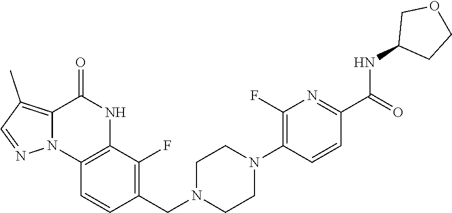 [SMILES: Cc1cnn2c1c(=O)[nH]c1c(F)c(CN3CCN(c4ccc(C(=O)N[C@@H]5CCOC5)nc4F)CC3)ccc12] | 523.54 | 524.15 [M + H]⁺ | DMSO-d₆: δ 8.34 (d, J = 8.0 Hz, 1H), 7.88 (s, 1H), 7.83-7.78 (m, 2H), 7.53-7.49 (m, 1H), 7.25 (t, J = 8.0 Hz, 1H), 4.39-4.36 (m, 1H), 3.79-3.76 (m, 2H), 3.68-3.63 (m 1H), 3.62 (s, 2H), 3.52-3.50 (m, 1H), 3.13-3.10 (m, 4H), 2.56-250 (m, 4H), 2.39 (s, 3H), 2.09- 2.04 (m, 1H), 1.93-1.90 (m, 1H). |
| 157 | 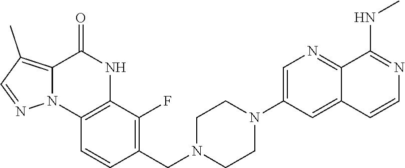 [SMILES: CNc1nccc2cc(N3CCN(Cc4ccc5c([nH]c(=O)c6c(C)cnn65)c4F)CC3)cnc12] | 472.53 | 473.15 [M + H]⁺ | DMSO-d₆: δ 11.75 (s, 1H), 8.58 (d, J = 2.8 Hz, 1H), 7.89 (s, 1H), 7.84 (d, J = 8.0 Hz, 1H), 7.72 (d, J = 8.0 Hz, 1H), 7.30-7.28 (m, 2H), 7.23 (d, J = 2.4 Hz, 1H), 6.63 (d, J = 8.0 Hz, 1H), 3.64 (s, 2H), 3.34-3.32 (m, 4H), 2.90 (d, J = 8.0 Hz, 3H), 2.58-2.56 (m, 4H), 2.40 (s, 3H). |
| 158 | 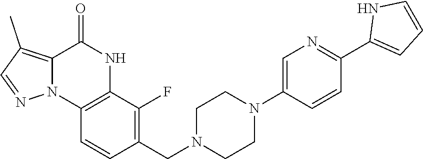 [SMILES: Cc1cnn2c1c(=O)[nH]c1c(F)c(CN3CCN(c4ccc(-c5ccc[nH]5)nc4)CC3)ccc12] | 458.50 | 459.10 [M + H]⁺ | DMSO-d₆: δ 11.75 (s, 1H), 8.22 (d, J = 2.8 Hz, 1H), 8.16 (s, 1H), 7.90 (s, 1H), 7.84 (d, J = 8.0 Hz, 1H), 7.80 (d, J = 8.0 Hz, 1H), 7.38 (d, J = 8.0 Hz, 1H), 7.28 (d, J = 8.0 Hz, 1H), 7.06-6.95 (br, 2H), 3.63 (s, 2H), 3.26- 3.22 (m, 4H), 2.58-2.56 (m, 4H), 2.40 (s, 3H) |
| 159 | 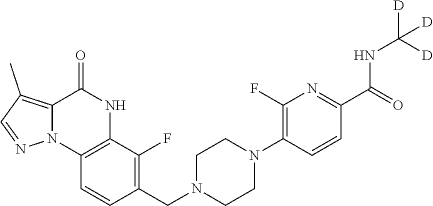 [SMILES: [2H]C([2H])([2H])NC(=O)c1ccc(N2CCN(Cc3ccc4c([nH]c(=O)c5c(C)cnn54)c3F)CC2)c(F)n1] | 470.50 | 471.15 [M + H]⁺ | DMSO-d₆: δ 11.67 (s, 1H), 8.34 (s, 1H), 7.89 (s, 1H), 7.83 (d, J = 8.0 Hz, 1H), 7.79 (d, J = 8.0 Hz, 1H), 7.54-7.49 (m, 1H0, 7.26 (t, J = 8.0 Hz, 1H), 3.63 (s, 2H), 3.14-3.12 (m, 4H), 2.57-2.55 (m, 4H), 2.40 (s, 3H). |
| 160 | 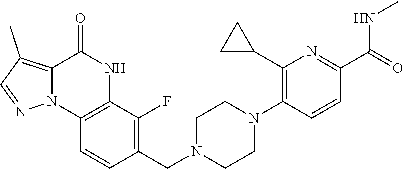 [SMILES: CNC(=O)c1ccc(N2CCN(Cc3ccc4c([nH]c(=O)c5c(C)cnn54)c3F)CC2)c(C2CC2)n1] | 489.56 | 490.20 [M + H]⁺ | DMSO-d₆: δ 11.78 (s, 1H), 8.27 (q, J = 5.0 Hz, 1H), 7.93 (s, 1H), 7.87 (d, J = 8.5 Hz, 1H), 7.68 (d, J = 8.2 Hz, 1H), 7.42 (d, J = 8.3 Hz, 1H), 7.30 (dd, J = 8.5, 6.7 Hz, 1H), 3.69 (s, 2H), 3.09-2.94 (m, 4H), 2.78 (d, J = 4.8 Hz, 3H), 2.70-2.58 (m, 4H), 2.44 (s, 3H), 2.35-2.28 (m, 1H), 1.18-1.13 (m, 2H), 1.0- 0.93 (m, 2H). |
| 161 | 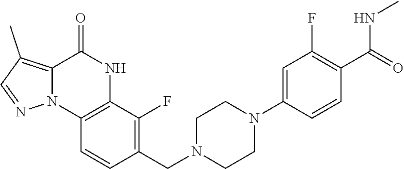 [SMILES: CNC(=O)c1ccc(N2CCN(Cc3ccc4c([nH]c(=O)c5c(C)cnn54)c3F)CC2)cc1F] | 466.49 | 467.10 [M + H]⁺ | DMSO-d₆: δ 7.93 (s, 1H), 7.87 (d, J = 8.5 Hz, 1H), 7.80-7.74 (m, 1H), 7.55 (t, J = 8.9 Hz, 1H), 7.30 (t, J = 7.7 Hz, 1H), 6.82-6.76 (m, 1H), 6.73 (d, J = 15.5 Hz, 1H), 3.64 (s, 2H), 3.31-3.24 (m, 4H), 2.73 (d, J = 4.5 Hz, 3H), 2.57-2.50 (m, 4H), 2.44 (s, 3H). |
| 162 | 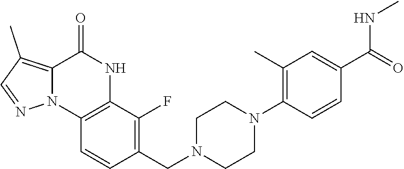 [SMILES: CNC(=O)c1ccc(N2CCN(Cc3ccc4c([nH]c(=O)c5c(C)cnn54)c3F)CC2)c(C)c1] | 462.53 | 463.15 [M + H]⁺ | DMSO-d₆: δ 11.76 (s, 1H), 8.21 (q, J = 4.8 Hz, 1H), 7.90 (s, 1H), 7.84 (d, J = 8.5 Hz, 1H), 7.62-7.53 (m, 2H), 7.27 (t, J = 7.7 Hz, 1H), 6.97 (d, J = 8.2 Hz, 1H), 3.63 (s, 2H), 2.90-2.80 (m, 4H), 2.70 (d, J = 4.4 Hz, 3H), 2.63-2.50 (m, 4H), 2.40 (s, 3H), 2.21 (s, 3H). |
| 163 | 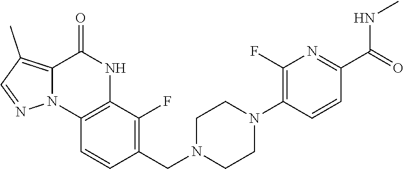 | 481.51 | 482.35 [M + H]⁺ | DMSO-d₆: δ 11.80 (s, 1H), 8.45-8.39 (m, 1H), 8.00 (s, 1H), 7.89 (d, J = 8.5 Hz, 1H), 7.83 (d, J = 8.0 Hz, 1H), 7.56 (dd, J = 10.6, 8.1 Hz, 1H), 7.31 (t, J = 7.6 Hz, 1H), 3.67 (s, 2H), 3.20-3.12 (m, 4H), 2.91 (q, J = 7.5 Hz, 2H), 2.75 (d, J = 4.7 Hz, 3H), 2.63-2.56 (m, 4H), 1.24 (t, J = 7.5 Hz, 3H). |
| 164 | 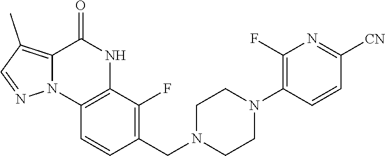 [SMILES: Cc1cnn2c1c(=O)[nH]c1c(F)c(CN3CCN(c4ccc(C#N)nc4F)CC3)ccc12] | 435.44 | 436.10 [M + H]⁺ | DMSO-d₆: δ 11.79 (s, 1H), 7.93 (s, 1H), 7.92- 7.82 (m, 2H), 7.54 (t, J = 9.3 Hz, 1H), 7.30 (t, J = 7.4 Hz, 1H), 3.66 (s, 2H), 3.29-3.22 (m, 4H), 2.64-2.54 (m, 4H), 2.44 (s, 3H). |
| 165 | 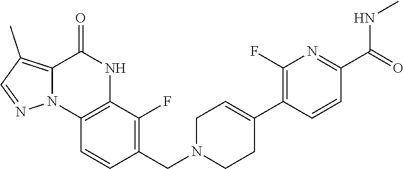 [SMILES: CNC(=O)c1ccc(C2=CCN(Cc3ccc4c([nH]c(=O)c5c(C)cnn54)c3F)CC2)c(F)n1] | 464.48 | 465.10 [M + H]⁺ | DMSO-d₆: δ 11.76 (s, 1H), 8.63-8.61 (m, 1H), 8.04-8.01 (m, 1H), 7.90-7.88 (m, 1H), 7.87-7.85 (m, 2H), 7.30-7.27 (m, 1H), 6.21 (s, 1H), 3.69 (s, 2H), 3.13 (s, 2H), 2.74 (d, J = 4.0 Hz, 3H), 2.67-2.65 (m, 2H), 2.47- 2.46 (m, 2H), 2.40 (s, 3H). |
| 166 | 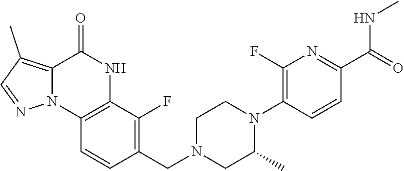 [SMILES: CNC(=O)c1ccc(N2CCN(Cc3ccc4c([nH]c(=O)c5c(C)cnn54)c3F)C[C@H]2C)c(F)n1] | 481.51 | 482.15 [M + H]⁺ | DMSO-d₆: δ 11.73 (s, 1H), 8.41-8.37 (m, 1H), 7.89 (s, 1H), 7.84 (d, J = 8.0 Hz, 1H), 7.80 (d, J = 8.0 Hz, 1H), 7.56-7.52 (m, 1H), 7.30-7.26 (m, 1H), 3.79 (br, 1H), 3.60 (s, 2H), 3.22-3.17 (m, 2H), 3.00-2.97 (m, 1H), 2.74-2.71 (m, 4H), 2.51-2.49 (m, 1H), 2.35 (s, 3H), 2.34-2.31 (m, 1H), 0.98 (d, J = 6.8 Hz, 3H). |
| 167 | 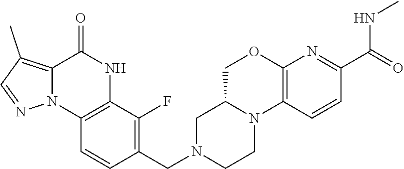 [SMILES: CNC(=O)c1ccc2c(n1)OC[C@H]1CN(Cc3ccc4c([nH]c(=O)c5c(C)cnn54)c3F)CCN21] | 477.50 | 478.25 [M + H]⁺ | DMSO-d₆: δ 11.59 (br, 1H), 8.14-8.12 (m, 1H), 7.90 (s, 1H), 7.84 (d, J = 8.0 Hz, 1H), 7.48 (d, J = 8.0 Hz, 1H), 7.28 (d, J = 8.0 Hz, 1H), 7.24 (d, J = 8.0 Hz, 1H), 4.36 (d, J = 8.0 Hz, 1H), 4.04-4.01 (m, 1H), 3.77-3.74 (m, 1H), 3.63 (d, J = 8.0 Hz, 2H), 3.18-3.16 (m, 2H), 2.95-2.87 (m, 2H), 2.69 (d, J = 4.0 Hz, 3H), 2.40 (s, 3H), 2.24-2.20 (m, 1H), 1.83- 1.78 (m, 1H). |
| 168 | 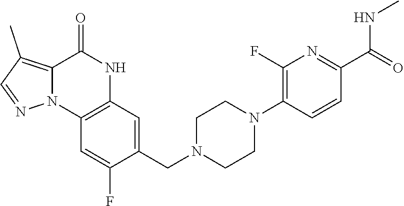 [SMILES: CNC(=O)c1ccc(N2CCN(Cc3cc4[nH]c(=O)c5c(C)cnn5c4cc3F)CC2)c(F)n1] | 467.48 | 468.15 [M + H]⁺ | DMSO-d₆: δ 11.68 (s, 1H), 8.40-8.38 (m, 1H), 7.90 (s, 1H), 7.82-7.77 (m, 2H), 7.53 (t, J = 8.0 Hz, 1H), 7.40 (d, J = 8.0 Hz, 1H), 3.61 (s, 2H), 3.16-3.14 (m, 4H), 2.72 (d, J = 8.0 Hz, 3H), 2.59-2.57 (m, 4H), 2.39 (s, 3H). |
| 169 | 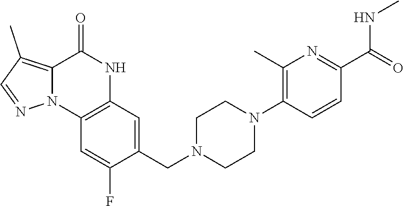 [SMILES: CNC(=O)c1ccc(N2CCN(Cc3cc4[nH]c(=O)c5c(C)cnn5c4cc3F)CC2)c(C)n1] | 463.52 | 464.15 [M + H]⁺ | DMSO-d₆: δ 11.67 (s, 1H), 8.40-8.38 (m, 1H), 7.90 (s, 1H), 7.79-7.75 (m, 2H), 7.45- 7.40 (m, 2H), 3.62 (s, 2H), 2.94-2.92 (m, 4H), 2.75 (d, J = 5.2 Hz, 3H), 2.59-2.57 (m, 4H), 2.46 (s, 3H), 2.39 (s, 3H). |
| 170 | 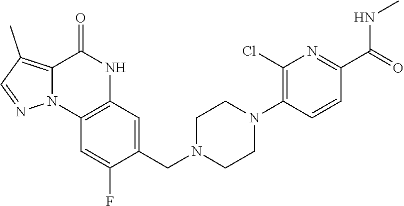 [SMILES: CNC(=O)c1ccc(N2CCN(Cc3cc4[nH]c(=O)c5c(C)cnn5c4cc3F)CC2)c(Cl)n1] | 483.93 | 484.10 [M + H]⁺ | DMSO-d₆: δ 11.67 (s, 1H), 8.40-8.38 (m, 1H), 7.91-7.89 (m, 2H), 7.78 (d, J = 8.0 Hz, 1H), 7.63 (d, J = 8.0 Hz, 1H), 7.39 (d, J = 6.8 Hz, 1H), 3.62 (s, 2H), 3.10-3.07 (m, 4H), 2.74 (d, J = 5.2 Hz, 3H), 2.59-2.57 (m, 4H), 2.39 (s, 3H). |
| 171 | 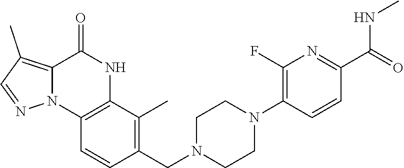 [SMILES: CNC(=O)c1ccc(N2CCN(Cc3ccc4c([nH]c(=O)c5c(C)cnn54)c3C)CC2)c(F)n1] | 463.52 | 464.20 [M + H]⁺ | DMSO-d₆: δ 10.79 (s, 1H), 8.39 (q, J = 4.7 Hz, 1H), 7.89 (d, J = 8.2 Hz, 1H), 7.86 (s, 1H), 7.80 (d, J = 7.8 Hz, 1H), 7.52 (dd, J = 10.8, 8.0 Hz, 1H), 7.19 (d, J = 8.2 Hz, 1H), 3.55 (s, 2H), 3.14-3.07 (s, 4H), 2.72 (d, J = 4.8 Hz, 3H), 2.56-2.47 (s, 4H), 2.43 (s, 3H), 2.41 (s, 3H). |
| 172 | 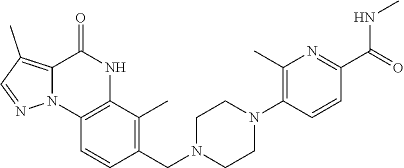 [SMILES: CNC(=O)c1ccc(N2CCN(Cc3ccc4c([nH]c(=O)c5c(C)cnn54)c3C)CC2)c(C)n1] | 459.55 | 460.15 [M + H]⁺ | DMSO-d₆: δ 10.79 (s, 1H), 8.40 (q, J = 5.1 Hz, 1H), 7.89 (d, J = 8.3 Hz, 1H), 7.85 (s, 1H), 7.74 (d, J = 8.3 Hz, 1H), 7.43 (d, J = 8.3 Hz, 1H), 7.19 (d, J = 8.4 Hz, 1H), 3.56 (s, 2H), 2.92-2.84 (m, 4H), 2.75 (d, J = 4.8 Hz, 3H), 2.58-2.49 (s, 4H), 2.47 (s, 3H), 2.44 (s, 3H), 2.41 (s, 3H). |
| 173 | 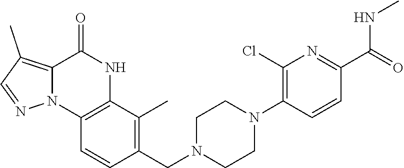 [SMILES: CNC(=O)c1ccc(N2CCN(Cc3ccc4c([nH]c(=O)c5c(C)cnn54)c3C)CC2)c(Cl)n1] | 479.97 | 481.80 [M + H]⁺ | DMSO-d₆: δ 10.79 (s, 1H), 8.42 (d, J = 4.9 Hz, 1H), 7.94-7.80 (m, 3H), 7.62 (d, J = 8.2 Hz, 1H), 7.19 (d, J = 8.4 Hz, 1H), 3.56 (s, 2H), 3.08-3.01 (m, 4H), 2.74 (d, J = 4.8 Hz, 3H), 2.57-2.52 (m, 4H), 2.44 (s, 3H), 2.41 (s, 3H). |
| 174 | 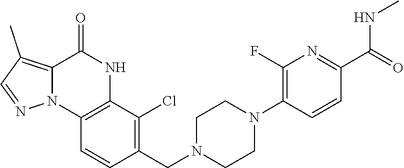 [SMILES: CNC(=O)c1ccc(N2CCN(Cc3ccc4c([nH]c(=O)c5c(C)cnn54)c3Cl)CC2)c(F)n1] | 483.93 | 484.10 [M + H]⁺ | DMSO-d₆: δ 8.46-8.39 (m, 1H), 8.08 (d, J = 11.3 Hz, 1H), 7.95 (s, 1H), 7.84 (d, J = 8.9 Hz, 1H), 7.63-7.52 (m, 1H), 7.44 (d, J = 8.0 Hz, 1H), 3.71 (s, 2H), 3.23-3.11 (m, 4H), 2.76 (s, 3H), 2.69-2.58 (m, 4H), 2.44 (s, 3H). |
| 175 | 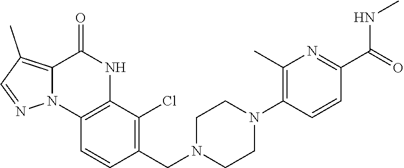 [SMILES: CNC(=O)c1ccc(N2CCN(Cc3ccc4c([nH]c(=O)c5c(C)cnn54)c3Cl)CC2)c(C)n1] | 479.97 | 480.40 [M + H]⁺ | DMSO-d₆: δ 10.96 (s, 1H), 8.40 (q, J = 4.9 Hz, 1H), 8.03 (d, J = 8.4 Hz, 1H), 7.90 (s, 1H), 7.74 (d, J = 8.3 Hz, 1H), 7.44 (d, J = 8.4 Hz, 1H), 7.40 (d, J = 8.5 Hz, 1H), 3.68 (s, 2H), 2.96-2.84 (m, 4H), 2.75 (d, J = 4.7 Hz, 3H), 2.66-2.52 (m, 4H), 2.40 (s, 3H). |
| 176 | 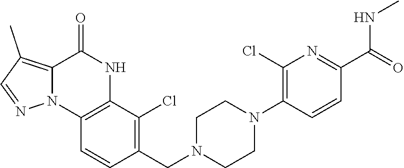 [SMILES: CNC(=O)c1ccc(N2CCN(Cc3ccc4c([nH]c(=O)c5c(C)cnn54)c3Cl)CC2)c(Cl)n1] | 500.38 | 500.10 [M + H]⁺ | DMSO-d₆: δ 10.96 (s, 1H), 8.42 (q, J = 4.7 Hz, 1H), 8.04 (d, J = 8.4 Hz, 1H), 7.94-7.85 (m, 2H), 7.63 (d, J = 8.2 Hz, 1H), 7.40 (d, J = 8.5 Hz, 1H), 3.69 (s, 2H), 3.16-3.01 (m, 4H), 2.75 (d, J = 4.8 Hz, 3H), 2.68-2.56 (m, 4H), 2.41 (s, 3H). |
| 177 | 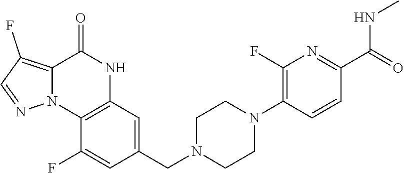 [SMILES: CNC(=O)c1ccc(N2CCN(Cc3cc(F)c4c(c3)[nH]c(=O)c3c(F)cnn34)CC2)c(F)n1] | 471.44 | 472.15 [M + H]⁺ | DMSO-d₆: δ 11.97 (s, 1H), 8.40-8.38 (m, 1H), 8.17 (d, J = 4.0 Hz, 1H), 7.80 (d, J = 8.0 Hz, 1H), 7.56-7.51 (m, 1H), 7.15-7.12 (m, 2H), 3.55 (s, 2H), 3.16-3.13 (m, 4H), 2.72 (d, J = 8.0 Hz, 3H), 2.55-2.53 (m, 4H). |
| 178 | 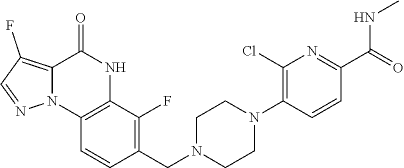 [SMILES: CNC(=O)c1ccc(N2CCN(Cc3ccc4c([nH]c(=O)c5c(F)cnn54)c3F)CC2)c(Cl)n1] | 487.90 | 488.40 [M + H]⁺ | DMSO-d₆: δ 12.00 (s, 1H), 8.45 (q, J = 4.9 Hz, 1H), 8.23 (d, J = 3.7 Hz, 1H), 8.00-7.84 (m, 2H), 7.66 (d, J = 8.3 Hz, 1H), 7.35 (t, J = 7.7 Hz, 1H), 3.71 (s, 2H), 3.18-3.04 (m, 4H), 2.78 (d, J = 4.7 Hz, 3H), 2.66-2.57 (m, 4H). |
| 179 | 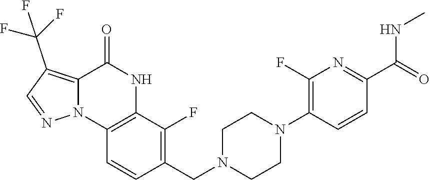 [SMILES: CNC(=O)c1ccc(N2CCN(Cc3ccc4c([nH]c(=O)c5c(C(F)(F)F)cnn54)c3F)CC2)c(F)n1] | 521.45 | 522.35 [M + H]⁺ | DMSO-d₆: δ 8.50 (s, 1H),8.40-8.33 (m, 1H), 7.97 (d, J = 8.6 Hz, 1H), 7.79 (d, J = 8.0 Hz, 1H), 7.52 (dd, J = 10.6, 8.0 Hz, 1H), 7.36 (dd, J = 8.6, 6.7 Hz, 1H), 3.67 (s, 2H), 3.15- 3.12 (m, 4H), 2.72 (d, J = 4.8 Hz, 3H), 2.59-2.54 (m, 4H). |
| 180 | 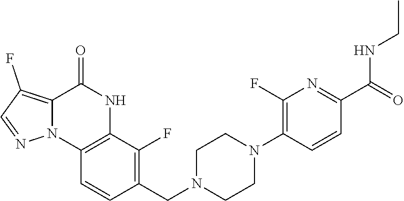 [SMILES: CCNC(=O)c1ccc(N2CCN(Cc3ccc4c([nH]c(=O)c5c(F)cnn54)c3F)CC2)c(F)n1] | 485.47 | 486.35 [M + H]⁺ | DMSO-d₆: δ 8.47 (t, J = 5.8 Hz, 1H), 8.22 (d, J = 3.7 Hz, 1H), 7.91 (d, J = 8.5 Hz, 1H), 7.83 (d, J = 8.5 Hz, 1H), 7.56 (dd, J = 10.4, 8.1 Hz, 1H), 7.34 (dd, J = 8.8, 7.2 Hz, 1H), 3.68 (s, 2H), 3.27-3.23 (m, 2H), 3.19-3.13 (s, 4H), 2.63-2.48 (s, 4H), 1.08 (t, J = 7.2 Hz, 3H). |
| 181 | 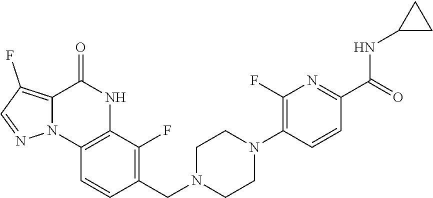 [SMILES: O=C(NC1CC1)c1ccc(N2CCN(Cc3ccc4c([nH]c(=O)c5c(F)cnn54)c3F)CC2)c(F)n1] | 497.48 | 498.60 [M + H]⁺ | DMSO-d₆: δ 8.38 (d, J = 4.8 Hz, 1H), 8.21 (d, J = 3.8 Hz, 1H), 7.90 (d, J = 8.8 Hz, 1H), 7.83 (d, J = 7.6 Hz, 1H), 7.56 (dd, J = 10.4, 8.3 Hz, 1H), 7.36-7.29 (m, 1H), 3.68 (s, 2H), 3.18-3.12 (m, 4H), 2.88-2.80 (m, 1H), 2.64-2.56 (m, 4H), 0.67-0.62 (m, 4H). |
| 182 | 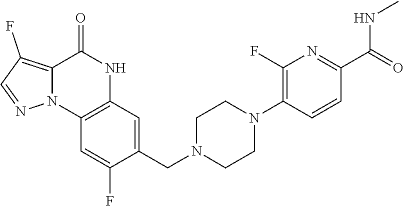 [SMILES: CNC(=O)c1ccc(N2CCN(Cc3cc4[nH]c(=O)c5c(F)cnn5c4cc3F)CC2)c(F)n1] | 471.44 | 472.10 [M + H]⁺ | DMSO-d₆: δ 11.94 (br, 1H), 8.45 (s, 1H), 8.28-8.23 (m, 1H), 7.85 (d, J = 8.0 Hz, 2H), 7.61-7.45 (m, 2H), 3.66 (s, 2H), 3.25-3.18 (m, 4H), 2.75 (d, J = 4.0 Hz, 3H), 2.61-2.49 (m, 4H). |
| 183 | 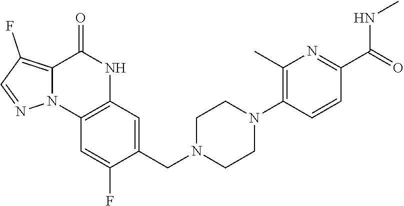 [SMILES: CNC(=O)c1ccc(N2CCN(Cc3cc4[nH]c(=O)c5c(F)cnn5c4cc3F)CC2)c(C)n1] | 467.48 | 468.15 [M + H]⁺ | DMSO-d₆: δ 11.87 (br, 1H), 8.41-8.39 (m, 1H), 8.18 (d, J = 4.0 Hz, 1H), 7.82 (d, J = 12.0 Hz, 1H), 7.76 (d, J = 8.0 Hz, 1H), 7.45- 7.42 (m, 2H), 3.63 (s, 2H), 2.93-2.91 (m, 4H), 2.75 (d, J = 4.0 Hz, 3H), 2.61-2.59 (m, 4H), 2.45 (s, 3H). |
| 184 | 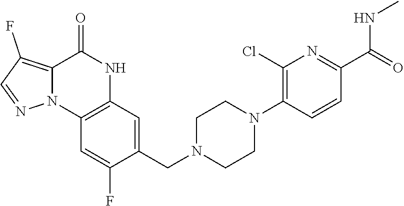 [SMILES: CNC(=O)c1ccc(N2CCN(Cc3cc4[nH]c(=O)c5c(F)cnn5c4cc3F)CC2)c(Cl)n1] | 487.90 | 488.10 [M + H]⁺ | DMSO-d₆: δ 11.89 (br, 1H), 8.43-8.40 (m, 1H), 7.90 (d, J = 8.0 Hz, 1H), 7.82 (d, J = 12.0 Hz, 1H), 7.63 (d, J = 8.0 Hz, 1H), 7.42 (d, J = 8.0 Hz, 1H), 3.63 (s, 2H), 3.10-3.07 (m, 4H), 2.74 (d, J = 4.0 Hz, 3H), 2.61-2.59 (m, 4H). |
| 185 | 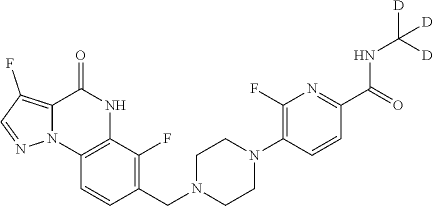 [SMILES: [2H]C([2H])([2H])NC(=O)c1ccc(N2CCN(Cc3ccc4c([nH]c(=O)c5c(F)cnn54)c3F)CC2)c(F)n1] | 474.46 | 475.25 [M + H]⁺ | DMSO-d₆: δ 8.35 (s, 1H), 8.17 (d, J = 4.0 Hz, 1H), 7.86 (d, J = 8.0 Hz, 1H), 7.79 (d, J = 8.0 Hz, 1H), 7.54-7.49 (m, 1H), 7.31-7.28 (m, 1H), 3.64 (s, 2H), 3.13-3.10 (m, 4H), 2.59- 2.54 (m, 4H). |
| 186 | 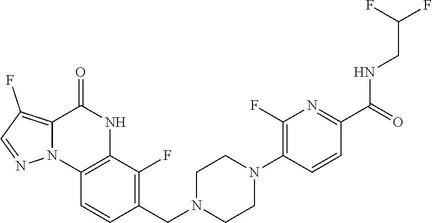 [SMILES: O=C(NCC(F)F)c1ccc(N2CCN(Cc3ccc4c([nH]c(=O)c5c(F)cnn54)c3F)CC2)c(F)n1] | 521.45 | 522.05 [M + H]⁺ | DMSO-d₆: δ 8.74 (t, J = 6.1 Hz, 1H), 8.22 (d, J = 3.8 Hz, 1H), 7.91-7.86 (m, 2H), 7.61- 7.52 (m, 1H), 7.38-7.30 (m, 1H), 6.10 (tt, J = 56.6, 4.2 Hz, 1H), 3.68 (s, 2H), 3.62 (dt, J = 15.3, 5.1 Hz, 2H), 3.22-3.14 (m, 4H), 2.62-2.57 (m, 4H). |
| 187 | 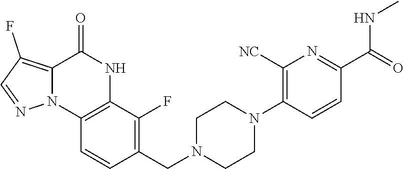 [SMILES: CNC(=O)c1ccc(N2CCN(Cc3ccc4c([nH]c(=O)c5c(F)cnn54)c3F)CC2)c(C#N)n1] | 478.46 | 479.10 [M + H]⁺ | DMSO-d₆: δ 8.56-8.54 (m, 1H), 8.18 (d, J = 3.8 Hz, 1H), 8.07 (d, J = 8.0 Hz, 1H), 7.88 (d, J = 8.0 Hz, 1H), 7.70 (d, J = 8.0 Hz, 1H), 7.32 (t, J = 8.0 Hz, 1H), 3.68 (s, 2H), 3.20-3.08 (m, 4H), 2.74 (d, J = 4.0 Hz, 3H), 2.62-2.60 (m, 4H). |
| 188 | 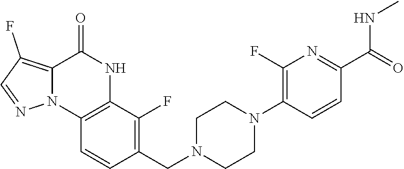 [SMILES: CNC(=O)c1ccc(N2CCN(Cc3ccc4c([nH]c(=O)c5c(F)cnn54)c3F)CC2)c(F)n1] | 470.45 | 471.05 [M + H]⁺ | DMSO-d₆: δ 8.59-8.57 (m, 1H), 8.17 (d, J = 4.0 Hz, 1H), 8.03 (t, J = 8.0 Hz, 1H), 7.85 (t, J = 8.0 Hz, 2H), 7.29 (t, J = 8.0 Hz, 1H), 3.60 (s, 2H), 2.92 (d, J = 12.0 Hz, 2H), 2.75-2.72 (m, 4H), 2.13-2.08 (m, 2H), 1.71-1.65 (m, 4H). |
| 189 | 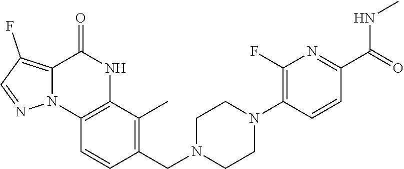 [SMILES: CNC(=O)c1ccc(N2CCN(Cc3ccc4c([nH]c(=O)c5c(F)cnn54)c3C)CC2)c(F)n1] | 467.48 | 468.05 [M + H]⁺ | DMSO-d₆: δ 11.04 (s, 1H), 8.42 (q, J = 5.3 Hz, 1H), 8.18 (d, J = 3.7 Hz, 1H), 7.95 (d, J = 8.4 Hz, 1H), 7.83 (d, J = 7.9 Hz, 1H), 7.55 (dd, J = 10.6, 8.0 Hz, 1H), 7.26 (d, J = 8.4 Hz, 1H), 3.59 (s, 2H), 3.20-3.08 (m, 4H), 2.75 (d, J = 4.7 Hz, 3H), 2.60-2.56 (m, 4H), 2.47 (s, 3H). |
| 190 | 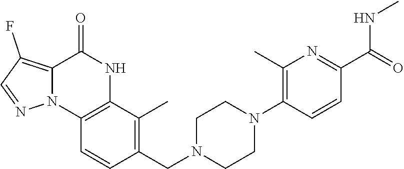 [SMILES: CNC(=O)c1ccc(N2CCN(Cc3ccc4c([nH]c(=O)c5c(F)cnn54)c3C)CC2)c(C)n1] | 463.52 | 464.10 [M + H]⁺ | DMSO-d₆: δ 11.03 (s, 1H), 8.43 (q, J = 5.2 Hz, 1H), 8.18 (d, J = 3.7 Hz, 1H), 7.96 (d, J = 8.4 Hz, 1H), 7.78 (d, J = 8.3 Hz, 1H), 7.47 (d, J = 8.3 Hz, 1H), 7.27 (d, J = 8.4 Hz, 1H), 3.61 (s, 2H), , 2.98-2.86 (m, 4H), 2.79 (d, J = 4.9 Hz, 3H), 2.62-2.54 (m, 4H), 2.49 (s, 3H), 2.48 (s, 3H). |
| 191 | 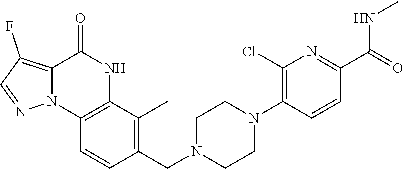 [SMILES: CNC(=O)c1ccc(N2CCN(Cc3ccc4c([nH]c(=O)c5c(F)cnn54)c3C)CC2)c(Cl)n1] | 483.93 | 484.05 [M + H]⁺ | DMSO-d₆: δ 11.03 (s, 1H), 8.44 (q, J = 5.1 Hz, 1H), 8.18 (d, J = 3.6 Hz, 1H), 7.96 (d, J = 8.3 Hz, 1H), 7.92 (d, J = 8.2 Hz, 1H), 7.65 (d, J = 8.3 Hz, 1H), 7.27 (d, J = 8.5 Hz, 1H), 3.61 (s, 2H), 3.14-3.02 (m, 4H), 2.78 (d, J = 4.7 Hz, 3H), 2.64-2.54 (m, 4H), 2.48 (s, 3H). |
| 192 | 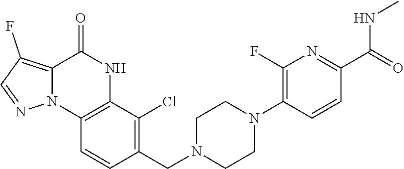 [SMILES: CNC(=O)c1ccc(N2CCN(Cc3ccc4c([nH]c(=O)c5c(F)cnn54)c3Cl)CC2)c(F)n1] | 487.90 | 488.55 [M + H]⁺ | DMSO-d₆: δ 8.39 (q, J = 5.0 Hz, 1H), 8.18 (d, J = 3.8 Hz, 1H), 8.06 (d, J = 8.5 Hz, 1H), 7.83-7.77 (m, 1H), 7.53 (dd, J = 10.7, 8.0 Hz, 1H), 7.42 (d, J = 8.5 Hz, 1H), 3.68 (s, 2H), 3.20-3.08 (m, 4H), 2.72 (d, J = 4.8 Hz, 3H), 2.64-2.52 (m, 4H). |
| 193 | 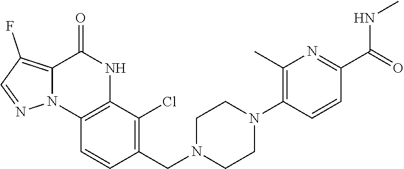 [SMILES: CNC(=O)c1ccc(N2CCN(Cc3ccc4c([nH]c(=O)c5c(F)cnn54)c3Cl)CC2)c(C)n1] | 483.93 | 484.25 [M + H]⁺ | DMSO-d₆: δ 8.44 (q, J = 5.8 Hz, 1H), 8.21 (d, J = 3.7 Hz, 1H), 8.09 (d, J = 8.5 Hz, 1H), 7.79 (d, J = 8.2 Hz, 1H), 7.47 (dd, J = 11.4, 8.4 Hz, 2H), 3.74 (s, 2H), 3.02-2.90 (m, 4H), 2.80 (d, J = 4.8 Hz, 3H), 2.72-2.58 (m, 4H), 2.51 (s, 3H). |
| 194 | 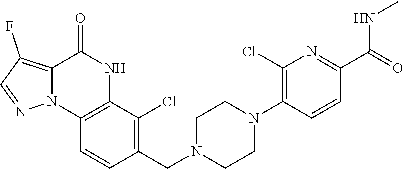 [SMILES: CNC(=O)c1ccc(N2CCN(Cc3ccc4c([nH]c(=O)c5c(F)cnn54)c3Cl)CC2)c(Cl)n1] | 504.35 | 504.10 | DMSO-d₆: δ 8.45 (q, J = 4.9 Hz, 1H), 8.22 (d, J = 3.7 Hz, 1H), 8.08 (d, J = 8.5 Hz, 1H), 7.93 (d, J = 8.1 Hz, 1H), 7.66 (d, J = 8.2 Hz, 1H), 7.45 (d, J = 8.5 Hz, 1H), 3.73 (s, 2H), 3.18-3.08 (m, 4H), 2.78 (d, J = 4.7 Hz, 3H), 2.72-2.58 (m, 4H).. |
| 195 | 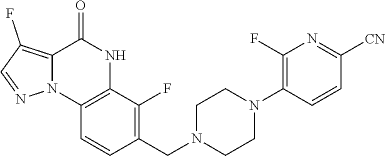 [SMILES: N#Cc1ccc(N2CCN(Cc3ccc4c([nH]c(=O)c5c(F)cnn54)c3F)CC2)c(F)n1] | 439.40 | 440.05 [M + H]⁺ | DMSO-d₆: δ 8.22 (d, J = 3.7 Hz, 1H), 7.94- 7.83 (m, 2H), 7.54 (dd, J = 10.5, 8.2 Hz, 1H), 7.33 (dd, J = 8.5, 6.8 Hz, 1H), 3.67 (s, 2H), 3.30-3.17 (m, 4H), 2.64-2.53 (m, 4H). |
| 196 | 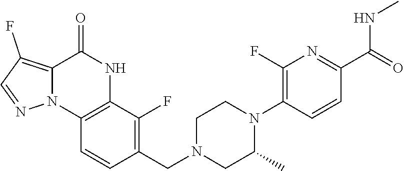 [SMILES: CNC(=O)c1ccc(N2CCN(Cc3ccc4c([nH]c(=O)c5c(F)cnn54)c3F)C[C@H]2C)c(F)n1] | 485.47 | 486.10 [M + H]⁺ | DMSO-d₆: δ 8.41-8.37 (m, 1H), 8.19-8.17 (m, 1H), 7.88 (d, J = 8.0 Hz, 1H), 7.80 (d, J = 8.0 Hz, 1H), 7.57-7.52 (m, 1H), 7.34-7.31 (m, 1H), 3.80 (br, 1H), 3.62 (s, 2H), 3.22- 3.17 (m, 2H), 3.00-2.97 (m, 1H), 2.74-2.71 (m, 4H), 2.51-2.49 (m, 1H), 2.34-2.31 (m, 1H), 0.98 (d, J = 6.8 Hz, 3H). |
| 197 | 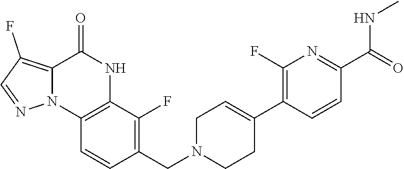 [SMILES: CNC(=O)c1ccc(C2=CCN(Cc3ccc4c([nH]c(=O)c5c(F)cnn54)c3F)CC2)c(F)n1] | 468.44 | 469.05 [M + H]⁺ | DMSO-d₆: δ 8.63- 8.61 (m, 1H), 8.17 (d, J = 2.8 Hz, 1H), 8.05-8.01 (m, 1H), 7.87-7.85 (m, 2H), 7.34-7.30 (m, 1H), 6.20 (s, 1H), 3.69 (s, 2H), 3.16-3.10 (m, 2H), 2.74 (d, J = 4.0 Hz, 3H), 2.67-2.64 (m, 2H), 2.47-2.46 (m, 2H). |
| 198 | 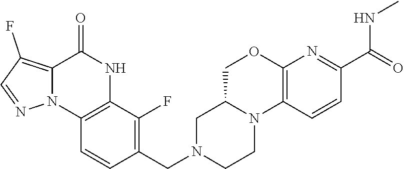 [SMILES: CNC(=O)c1ccc2c(n1)OC[C@H]1CN(Cc3ccc4c([nH]c(=O)c5c(F)cnn54)c3F)CCN21] | 481.46 | 482.05 [M + H]⁺ | DMSO-d₆: δ 8.19 (d, J = 4.0 Hz, 1Hz), 8.14- 8.12 (m, 1H), 7.87 (d, J = 8.0 Hz, 1H), 7.48 (d, J = 8.0 Hz, 1H), 7.31 (t, J = 8.0 Hz, 1H), 7.24 (d, J = 8.0 Hz, 1H), 4.36 (d, J = 8.0 Hz, 1H), 4.05-4.01 (m, 1H), 3.77-3.74 (m, 1H), 3.64 (d, J = 4.0 Hz, 2H), 3.18-3.16 (m, 2H), 2.95-2.87 (m, 2H), 2.69 (d, J = 4.0 Hz, 3H), 2.24-2.20 (m, 1H), 1.84-1.79 (m, 1H). |
| 199 | 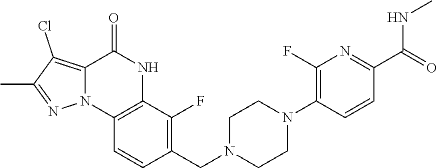 | 501.92 | 502.20 [M + H]⁺ | DMSO-d₆: δ 11.92 (s, 1H), 8.37 (d, J = 5.0 Hz, 1H), 7.79 (d, J = 8.2 Hz, 2H), 7.51 (dd, J = 10.6, 8.1 Hz, 1H), 7.28-7.25 (m, 1H), 3.63 (s, 2H), 3.17-3.08 (m, 4H), 2.72 (d, J = 4.7 Hz, 3H), 2.60-2.51 (m, 4H), 2.34 (s, 3H). |
| 200 | 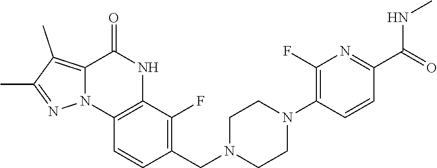 | 481.51 | 482.35 [M + H]⁺ | DMSO-d₆: δ 11.71 (s, 1H), 8.38 (q, J = 5.0 Hz, 1H), 7.79 (t, J = 8.6 Hz, 2H), 7.52 (dd, J = 10.6, 8.1 Hz, 1H), 7.24 (dd, J = 8.5, 6.9 Hz, 1H), 3.62 (s, 2H), 3.15-3.08 (m, 4H), 2.72 (d, J = 4.8 Hz, 3H), 2.50-2.61 (m, 4H), 2.34 (s, 3H), 2.30 (s, 3H). |
| 201 | 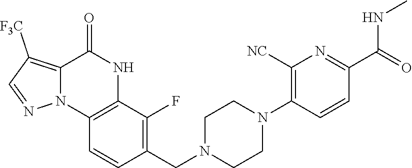 [SMILES: CNC(=O)c1ccc(N2CCN(Cc3ccc4c([nH]c(=O)c5c(C(F)(F)F)cnn54)c3F)CC2)c(C#N)n1] | 528.47 | 529.15 [M + H]⁺ | DMSO-d₆: δ 8.61-8.56 (m, 1H), 8.54 (s, 1H), 8.11 (d, J = 8.9 Hz, 1H), 8.02 (d, J = 8.5 Hz, 1H), 7.74 (d, J = 8.9 Hz, 1H), 7.40 (t, J = 7.6 Hz, 1H), 3.74 (s, 2H), 3.45-3.38 (m, 4H) 2.782(d, J = 4.7 Hz, 3H), 2.68-2.61 (m, 4H). |
| 202 | 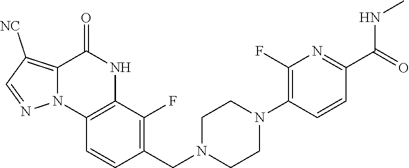 [SMILES: CNC(=O)c1ccc(N2CCN(Cc3ccc4c([nH]c(=O)c5c(C#N)cnn54)c3F)CC2)c(F)n1] | 478.46 | 479.10 [M + H]⁺ | DMSO-d₆: δ 8.71 (s, 1H), 8.41 (q, J = 4.9 Hz, 1H), 8.20 (s, 1H), 7.98 (d, J = 8.5 Hz, 1H), 7.83 (d, J = 7.4 Hz, 1H), 7.56 (dd, J = 10.6, 8.1 Hz, 1H), 7.40 (dd, J = 8.6, 6.7 Hz, 1H), 3.71 (s, 2H), 3.20-3.12 (m, 4H), 2.75 (d, J = 4.7 Hz, 3H), 2.63-2.55 (m, 4H). |
| 203 | 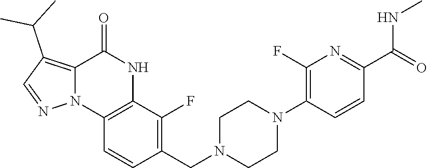 [SMILES: CNC(=O)c1ccc(N2CCN(Cc3ccc4c([nH]c(=O)c5c(C(C)C)cnn54)c3F)CC2)c(F)n1] | 495.53 | 496.30 [M + H]⁺ | DMSO-d₆: δ 11.84 (s, 1H), 8.45-8.39 (m, 1H), 8.00 (s, 1H), 7.89 (d, J = 8.5 Hz, 1H), 7.83 (d, J = 8.0 Hz, 1H), 7.56 (dd, J = 10.6, 8.1 Hz, 1H), 7.31 (t, J = 7.6 Hz, 1H), 3.68 (s, 2H), 3.65-3.57 (m, 1H), 3.20-3.14 (m, 4H)8 2.75 (d, J = 4.7 Hz, 3H), 2.63-2.56 (m, 4H), 1.24 (d, J = 6.8 Hz, 6H). |
| 204 | 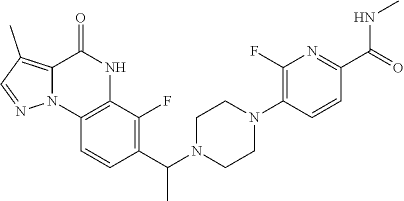 [SMILES: CNC(=O)c1ccc(N2CCN(C(C)c3ccc4c([nH]c(=O)c5c(C)cnn54)c3F)CC2)c(F)n1] | 481.51 | 482.20 [M + H]⁺ | DMSO-d₆: δ 11.78 (s, 1H), 8.42-8.38 (m, 1H), 7.93 (s, 1H), 7.89 (d, J = 8.7 Hz, 1H), 7.82 (d, J = 7.9 Hz, 1H), 7.56-7.50 (m, 1H), 7.37-7.30 (m, 1H), 4.00-3.91 (m, 1H), 3.29-3.10 (m, 4H), 2.75 (d, J = 4.7 Hz, 3H), 2.64-2.55 (m, 4H), 2.44 (s, 3H), 1.39 (d, J = 6.8 Hz, 3H). |
| 205 | 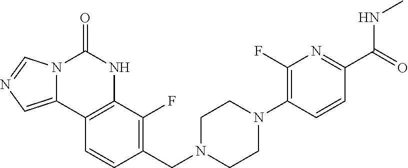 [SMILES: CNC(=O)c1ccc(N2CCN(Cc3ccc4c([nH]c(=O)n5cncc45)c3F)CC2)c(F)n1] | 453.45 | 454.05 | DMSO-d₆: δ 11.79 (s, 1H), 8.55 (s, 1H), 8.42 (q, J = 5.2 Hz, 1H), 8.56 (s, 1H), 7.84 (d, J = 8.2 Hz, 1H), 7.79 (d, J = 8.1 Hz, 1H), 7.59 7.52 (m, 1H), 7.29 (t, J = 7.2 Hz, 1H), 3.67 (s, 2H), 3.20-3.12 (m, 4H), 2.75 (d, J = 4.7 Hz, 3H), 2.62-2.54 (m, 4H). |

### Example 206

### PARP1 and PARP2 Chemiluminescent Assay

The solution of recombinant poly(ADP-ribose) polymerase 1 and 2 (PARP1 and PARP2) (40 ng enzyme/well) and the compounds to be tested were mixed, respectively. The solutions were added to a 96-well plate coated with histone mixture, incubated at room temperature for 1 h, then 50 μL 0.3 ng/mL Streptavidin-HRP was added to each well. The plates were incubated for 30 minutes at room temperature. Finally, the plates were treated with streptavidin-HRP followed by addition of the ELISA ECL substrate to produce chemiluminescence that can be measured using a chemiluminescence reader. Inhibition of the tested compound to PARP1/2 enzyme activity was calculated according to the following formula.

$$Inhibition ( \% ) = \frac{Readings of positive control - X}{Readings of positive control - Readings of negative control}$$

IC₅₀ value is obtained by fitting the s-shaped dose response curve equation by using XL Fit software. The curve equation is Y=100/(1+10\hat{ }(logC−logIC₅₀)), C is the compound concentration.

Table I summarize the inhibitory effects of compounds on PARP1 and PARP2 enzyme activity (IC₅₀).

TABLE 1

| | IC₅₀ (nM) | |
|---|---|---|
| Example | PARP1 | PARP2 |
| 1 | 1.52 | 78.02 |
| 2 | 1.42 | 598.05 |
| 3 | 1.08 | 18.32 |
| 5 | 0.77 | 10.10 |
| 6 | 0.70 | 168.25 |
| 7 | 1.16 | 910.27 |
| 8 | 1.15 | 54.87 |
| 9 | 2.45 | 3279.51 |
| 10 | 8.24 | >10000 |
| 11 | 1.28 | 597.55 |
| 12 | 1.11 | 6367.68 |
| 14 | 1.86 | >10000 |
| 21 | 0.66 | 13.26 |
| 25 | 3.39 | 2487.95 |
| 26 | 4.43 | 2422.02 |
| 28 | 2.42 | 2063.11 |
| 30 | 137.83 | >10000 |
| 34 | 2.52 | 708.72 |
| 35 | 2.92 | 584.41 |
| 38 | 0.67 | 16.90 |
| 40 | 0.60 | 459.06 |
| 43 | 0.39 | 5582.64 |
| 44 | 0.41 | 898.48 |
| 45 | 0.64 | 278.38 |
| 46 | 0.95 | 194.32 |
| 47 | 0.86 | 504.14 |
| 48 | 1.76 | 1983.97 |
| 49 | 1.15 | 69.96 |
| 50 | 2.13 | >10000 |
| 51 | 0.90 | 30.59 |
| 53 | 0.25 | 468.85 |
| 54 | 0.52 | 647.98 |
| 55 | 0.47 | 775.76 |
| 56 | 0.68 | 1567.56 |
| 57 | 1.95 | >10000 |
| 58 | 1.38 | >10000 |
| 59 | 0.43 | 697.91 |
| 60 | 0.42 | 195.17 |
| 61 | 2.23 | 7255.79 |
| 62 | 3.49 | >10000 |
| 63 | 3.86 | >10000 |
| 64 | 2.73 | 5450.65 |
| 65 | 4.53 | >10000 |
| 66 | 1.72 | 5003.88 |
| 67 | 0.72 | 1719.08 |
| 68 | 0.52 | 1874.90 |
| 69 | 0.77 | 5776.39 |
| 70 | 2.60 | >10000 |
| 71 | 3.30 | 636.35 |
| 72 | 1.08 | 1834.51 |
| 74 | 3.31 | 2978 |
| 75 | 8.38 | 2381.59 |
| 89 | 5.81 | 3411.11 |
| 91 | 4.25 | 941.60 |
| 92 | 0.44 | 199.34 |
| 93 | 0.59 | 8263.67 |
| 99 | 5.94 | >10000 |
| 102 | 1.89 | 1333.2 |
| 103 | 4.26 | 666.81 |
| 104 | 5.85 | 486.81 |
| 109 | 0.83 | 5365.50 |
| 112 | 0.26 | 157.60 |
| 113 | 0.13 | 14.96 |
| 115 | 0.47 | 340.42 |
| 118 | 0.53 | 273.36 |
| 120 | 1.87 | 683.48 |
| 121 | 1.06 | 2821.75 |
| 122 | 0.53 | 462.44 |
| 126 | 0.48 | 731.1 |
| 127 | 1.44 | 477 |
| 128 | 0.48 | 2504 |
| 129 | 0.50 | >10000 |
| 131 | 0.45 | >10000 |
| 133 | 0.57 | >10000 |
| 136 | 1.77 | 930.61 |
| 137 | 2.16 | 266.44 |
| 140 | 0.84 | 1251 |
| 141 | 1.29 | 1893 |
| 143 | 0.46 | 165.66 |
| 144 | 0.23 | 610.45 |
| 147 | 0.56 | 2397 |
| 151 | 0.50 | 1214.86 |
| 152 | 0.85 | >1111.11 |
| 165 | 0.26 | >3333 |
| 166 | 0.21 | 801 |
| 167 | 0.33 | 397 |
| 168 | 0.61 | 145.14 |
| 177 | 0.32 | 93.45 |
| 178 | 0.28 | 1389.17 |
| 179 | 0.50 | 67.13 |
| 180 | 0.24 | 431 |
| 181 | 0.23 | 314.97 |
| 182 | 0.32 | 93.45 |
| 183 | 0.28 | 1389.17 |
| 184 | 0.50 | 67.13 |
| 185 | 0.24 | 431.00 |
| 188 | 0.40 | 1543 |
| 196 | 0.15 | 371 |
| 197 | 0.15 | 774 |
| 199 | 0.16 | 17.93 |
| 204 | 0.68 | >10000 |

Relative to PARP2 enzyme, most of the compounds tested have potent and selective inhibitory effect on PARP1 enzyme.

### Example 207

### Growth Inhibition Assays Against BRCA Mutant Human Breast Cancer MDA-MB-436 Cell Line

The cells were cultured in complete medium (DMEM medium+10% FBS+Insulin+glutathione). When the confluence reached about 80%, cells were digested and gently dispensed from the bottom of the dish with a 1 mL pipette. Cell suspension was collected and centrifuged at 500 rpm for 3 min. The supernatant was discarded, and the cell pellet were re-suspended in complete medium. The cells were seeded into a culture dish at an appropriate proportion, and then cultured in a 5% CO₂ incubator at 37° C. The assay was carried out when the cells were in optimum condition and the confluence was reached 80%. Cells in the logarithmic growth phase were taken to centrifugate, and the culture supernatant was removed. The cells were resuspended in refresh complete medium and counted. The resuspended cells were seeded at 3000/well in a 96-well plate and incubated at 37° C., 5% CO₂ incubator overnight. The compound was prepared as below: 1000× dilution tested compound solution to 40× test compound solution by adding 5 μL 1000× compound solution to 120 μL Medium (25-fold dilution). The solution was mixed by oscillation. 0.1% DMSO was used as the control.

The next day, the 96-well plate inoculated with cells was taken out from the incubator, and the culture supernatant was removed. Then fresh medium of 195 uL/well and 5 μL/well of 40× test compound solution as mentioned above were added into the 96 well plate, respectively. Finally, the plate was incubated for 7 days in a 37° C. 5% CO₂ incubator. The medium containing compound was changed on the fourth day. After 7 days, 20 μL of CCK-8 was added to each well and shaken gently, then was cultured for 4 hours. The plate was shaken for 5 min after incubation, the absorbance values of 450 nm or 650 nm wavelengths were recorded respectively (OD=absorbance value of 450 nm−absorbance value of 650 nm) by using the multifunction readout instrument.

Data were analyzed by software GraphPad Prism 6.0. The inhibitory activity of compounds on cell proliferation was plotted using cell survival rate against the compound concentration as coordinates. Cell survival rate %=(OD_{compound}−OD_{background})/(OD_{DMSO}−OD_{background})×100. The IC₅₀ value was fitted by the s-shaped dose response curve equation: Y=100/(1+10\hat{ }(logC−logIC₅₀)), and C was the compound concentration.

Table 2 summarizes the inhibitory effect data (IC₅₀) of the compounds on the proliferation of human breast cancer cells MDA-MB-436.

TABLE 2

| Example | IC₅₀ (nM) |
|---|---|
| 1 | 2.46 |
| 2 | 1.15 |
| 3 | 0.74 |
| 4 | 7.69 |
| 5 | 0.74 |
| 6 | 3.90 |
| 7 | 5.22 |
| 8 | 0.77 |
| 9 | 3.20 |
| 10 | 15.52 |
| 11 | 11.27 |
| 12 | 1.15 |
| 14 | 21.33 |
| 21 | 0.87 |
| 23 | 1.74 |
| 24 | 1.11 |
| 25 | 61.80 |
| 26 | >100 |
| 28 | 19.45 |
| 30 | >100 |
| 34 | 5.06 |
| 35 | 8.24 |
| 38 | 3.73 |
| 40 | 2.48 |
| 43 | 5.86 |
| 44 | 3.85 |
| 45 | 9.02 |
| 46 | 16.48 |
| 47 | 12.16 |
| 48 | 4.58 |
| 49 | 1.04 |
| 50 | 23.55 |
| 51 | 8.45 |
| 53 | 2.01 |
| 54 | 5.85 |
| 55 | 14.03 |
| 56 | 4.08 |
| 57 | 10.59 |
| 58 | 2.99 |
| 59 | 2.13 |
| 60 | 1.41 |
| 61 | 17.38 |
| 62 | 46.67 |
| 63 | 53.7 |
| 64 | 52.6 |
| 65 | 53.83 |
| 66 | 3.40 |
| 67 | 6.56 |
| 68 | 4.72 |
| 69 | 4.66 |
| 70 | 28.91 |
| 71 | 10.99 |
| 72 | 28.97 |
| 73 | 12.30 |
| 74 | 58.21 |
| 75 | 11.09 |
| 88 | 21.73 |
| 89 | 11.89 |
| 90 | 28.71 |
| 91 | 20.75 |
| 92 | 3.48 |
| 93 | 3.76 |
| 94 | >100 |
| 95 | >100 |
| 96 | 36.73 |
| 97 | 9.79 |
| 99 | 53.83 |
| 100 | >100 |
| 101 | 22.96 |
| 102 | 5.50 |
| 103 | 12.45 |
| 104 | 31.99 |
| 105 | >100 |
| 106 | 17.50 |
| 109 | 5.09 |
| 110 | 12.94 |
| 112 | 0.58 |
| 113 | 2.19 |
| 114 | 1.32 |
| 115 | 3.41 |
| 116 | 7.50 |
| 118 | 1.56 |
| 119 | 7.87 |
| 120 | 23.55 |
| 121 | 1.72 |
| 122 | 1.32 |
| 124 | 25.47 |
| 125 | 7.24 |
| 126 | 4.50 |
| 127 | 55.34 |
| 128 | 1.58 |
| 129 | 5.02 |
| 131 | 3.12 |
| 133 | 22.96 |
| 134 | >100 |
| 135 | >100 |
| 136 | 1.04 |
| 137 | 6.85 |
| 140 | 2.24 |
| 141 | 24.55 |
| 143 | 3.50 |
| 144 | 1.90 |
| 145 | >100 |
| 146 | >100 |
| 147 | 2.00 |
| 148 | 29.24 |
| 149 | 74.47 |
| 150 | 11.48 |
| 151 | 1.19 |
| 152 | 19.32 |
| 153 | 23.61 |
| 154 | 95.94 |
| 155 | 66.53 |
| 156 | 6.08 |
| 157 | 42.27 |
| 158 | 98.17 |
| 159 | 3.91 |
| 160 | >100 |
| 161 | >100 |
| 162 | 20.09 |
| 164 | 71.29 |
| 165 | 2.75 |
| 166 | 16.22 |
| 167 | 18.71 |
| 168 | 8.02 |
| 169 | 20.28 |
| 170 | 18.49 |
| 171 | 14.42 |
| 172 | 42.45 |
| 173 | 23.23 |
| 174 | 46.99 |
| 175 | >100 |
| 176 | >100 |
| 177 | 1.58 |
| 178 | 0.79 |
| 179 | 4.49 |
| 180 | 1.24 |
| 181 | 1.9 |
| 182 | 1.4 |
| 183 | 1.76 |
| 184 | 2.41 |
| 185 | 0.78 |
| 188 | 5.27 |
| 192 | 5.58 |
| 193 | 39.72 |
| 195 | 8.59 |
| 196 | 15.45 |
| 197 | 1.19 |
| 199 | 1.11 |
| 203 | >100 |
| 204 | 12.05 |

The compounds tested have good inhibitory effect on the proliferation of BRCA mutated human breast cancer cells MDA-MB-436.

### Example 208

### The Inhibitory Effect of the Compounds on PDE3A

A PDE3A fluorescence polarization (FP) assay in a multi-step format was performed using black, round bottom 384-well plates (Corning, 4514). The testing compounds were serially diluted from stock solutions to 11 concentrations with a 3-fold dilution factor using DMSO. Fifty nl of compound dilutions was mixed with 5 μl of reaction buffer (10 mM Tris-HCl, pH 7.2, 10 mM MgCl2, 0.05% NaN3, 0.1% phosphate-free BSA) containing 0.2 μM FAM-cAMP (BPS, 60200) and 2 nM PDE3A (Sino Biological, 11908-H20B1) enzyme, and incubated for 60 min at 25° C. Afterwards, 15 μl binding agent mixture (Molecular Devices, R8124) was added to each well and the plate was incubated for 60 min at 25° C. The plate was loaded to a BMG PHERAstar FSX to read Fluorescence Polarization (FP) values at a setting of: Ex: 485 nm and Em: 520 nm. Emission light intensity with polarizers parallel (Emi) and emission light intensity with polarizers perpendicular (Emi) were recorded. The Polarization (mP) values were calculated using the following formula: mP=(Signal_{Eml-}Signal_{Em⊥})/(Signal_{Eml+}Signal_{Em⊥}).

### Data Analysis:

1) The inhibition % of a compound dose is calculated as:

$$\% Inh = ( m {P}_{Max} - m {P}_{cmpds} ) / ( {mP}_{Max} - m {P}_{Min} ) * 100 \%$$

wherein mP_{Min} and mP_{Max} refer to the polarization readings in the condition of substrate only and substrate+enzyme, respectively.
2) The IC₅₀ values were calculated from non-linear curve fitting using following regression equation using a commercial curve fitting software GraphPad Prism 9.2.0.

$$Y = Bottom + ( Top - Bottom ) / ( 1 + 10 \hat{} ( ( Log {IC}_{50} - X ) * Hillslope ) )$$

wherein, X refers to Log of compound concentration and Y refers to % Inhibition.

Table 3 summarize the inhibitory effects of compounds on PDE3A enzyme activity (IC₅₀).

TABLE 3

| Example | IC₅₀ (μM) |
|---|---|
| 2 | 1.46 |
| 6 | 0.58 |
| 40 | 0.69 |
| 43 | 8.57 |
| 44 | 16.5 |
| 46 | >30 |
| 48 | >30 |
| 49 | 0.88 |
| 57 | >30 |
| 58 | >30 |
| 59 | 9.4 |
| 60 | >30 |
| 66 | 16.65 |
| 67 | 13.87 |
| 68 | 22.0 |
| 92 | >30 |
| 93 | >30 |
| 102 | >30 |
| 112 | 1.3 |
| 115 | 13.8 |
| 121 | 10.5 |
| 122 | >30 |
| 126 | >25 |
| 128 | >30 |
| 129 | >30 |
| 131 | >30 |
| 133 | >30 |
| 140 | 28.9 |
| 141 | >25 |
| 143 | 1.2 |
| 147 | 10.9 |
| 152 | >25 |
| 159 | >30 |
| 168 | >30 |
| 177 | 0.63 |
| 179 | >30 |
| 180 | >30 |
| 181 | >30 |
| 182 | >30 |
| 183 | 1.59 |
| 192 | >30 |

Compounds of this disclosure have low inhibitory effect on PDE3A, with some compounds IC₅₀>30 μM.

Having now fully described this disclosure, it will be understood by those of ordinary skill in the art that the same can be performed within a wide and equivalent range of conditions, formulations and other parameters without affecting the scope of the disclosure or any embodiment thereof. All patents, patent applications and publications cited herein are fully incorporated by reference herein in their entirety.

What is claimed is:

1. A compound of Formula I: 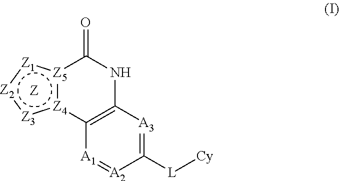
or stereoisomers, tautomers, N-oxides, hydrates, solvates, isotope-substituted derivatives, or pharmaceutically acceptable salts thereof, or prodrugs thereof, or mixtures thereof, wherein:
A₁, A₂ and A₃ are each independently selected from N and CR₁;
the Z ring shown as 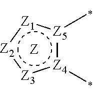
is an optionally substituted 5 membered heteroaryl group, an optionally substituted 5 membered carbocyclic group or an optionally substituted 5 membered heterocyclic group, * indicates the position at which the Z ring is attached to the rest of the compound, the dashed lines indicate optional presence of unsaturated bond(s), wherein, when the Z ring is an optionally substituted 5 membered heteroaryl group, Z₁, Z₂ and Z₃ are each independently CR₂, NR₃, O, N or S, Z₄ and Z₅ are each independently C or N, wherein Z₄ and Z₅ are not N at the same time; when the Z ring is an optionally substituted 5 membered carbocyclic group or an optionally substituted 5 membered heterocyclic group, Z₁, Z₂ and Z₃ are each independently CR₂R₂′, CR₂, NR₃, O or S, Z₄ and Z₅ are each independently C, CH or N; and
when Z₅ is N, at least one of Z₁, Z₂ and Z₃ is N, or when Z₅ is N and all of Z₁, Z₂ and Z₃ are CR₂ or CR₂R₂′, A₁ is CR₁;
L is selected from a bond and an alkylene optionally substituted by R₄ and/or R₅;
Cy is selected from a group consisting of an optionally substituted heterocyclic group, an optionally substituted aryl, and an optionally substituted heteroaryl;
R₁ is selected from a group consisting of hydrogen, halogen, an optionally substituted alkyl, an optionally substituted alkoxy and an optionally substituted carbocyclic group;
R₂ and R₂′ are each independently selected from a group consisting of hydrogen, hydroxyl, halogen, cyano, an optionally substituted alkyl, an optionally substituted alkoxy, an optionally substituted cycloalkyl, an optionally substituted alkenyl, and an optionally substituted alkynyl;
R₃ is selected from a group consisting of hydrogen, an optionally substituted alkyl and an optionally substituted cycloalkyl;
R₄ and R₅ are each independently selected from a group consisting of halogen, cyano, an optionally substituted alkyl, an optionally substituted alkoxy, an optionally substituted cycloalkyl, an optionally substituted alkenyl, and an optionally substituted alkynyl; or R₄ and R₅ together with the attached C form a ring.

2. The compound of claim 1, or stereoisomers, tautomers, N-oxides, hydrates, solvates, isotope-substituted derivatives, or pharmaceutically acceptable salts thereof, or prodrugs thereof, wherein:
R₁ is hydrogen, halogen, an optionally substituted C₁₋₃ alkyl or an optionally substituted C₁₋₃ alkoxy; preferably, R₁ is hydrogen, halogen or C₁₋₃ alkyl; preferably, at least one of A₁ and A₃ is CR₁, and R₁ is halogen, such as fluoro; or at least one of A₂ and A₃ is CR₁, and R₁ is halogen; or both A₂ and A₃ are CR₁, and at least one of the R₁ groups is halogen; or A₃ is CR₁, R₁ is halogen; or all of A₁, A₂ and A₃ are CR₁, wherein each R₁ is independently hydrogen, halogen or C₁₋₃ alkyl; or at least one of A₁ and A₃ is CR₁, wherein R₁ is halogen, preferably at least A₃ is CR₁ and R₁ is a halogen such as fluoro; or A₁ is CR₁, both of A₂ and A₃ are CH; or A₁ is CH, A₂ is CR₁, A₃ is CH; or both of A₁ and A₂ are CH, A₃ is CR₁, wherein R₁ is halogen or C₁₋₃ alkyl; and/or
the Z ring is selected from the following groups: 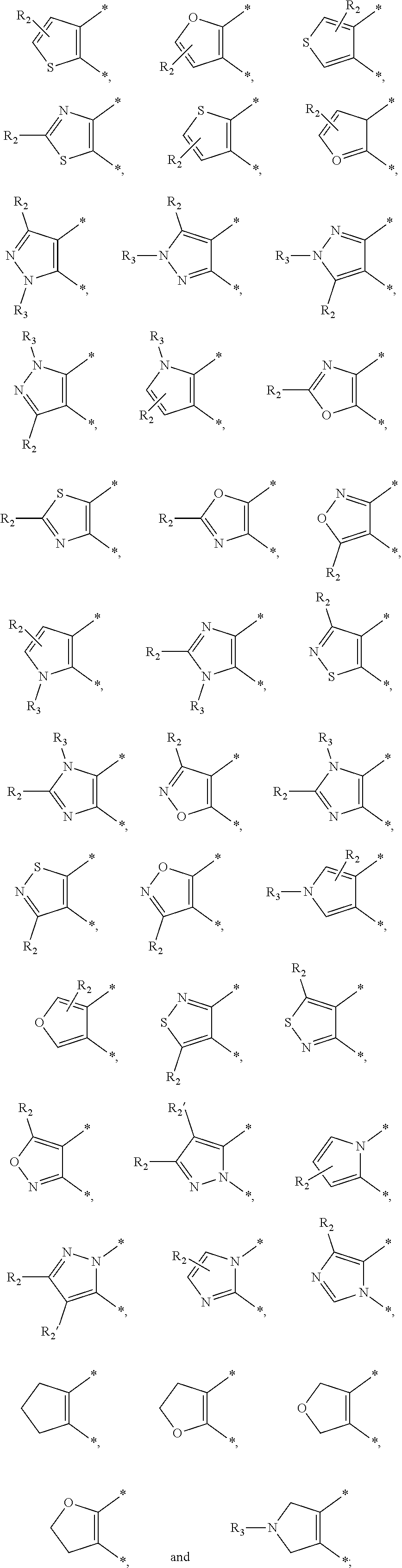
preferably, 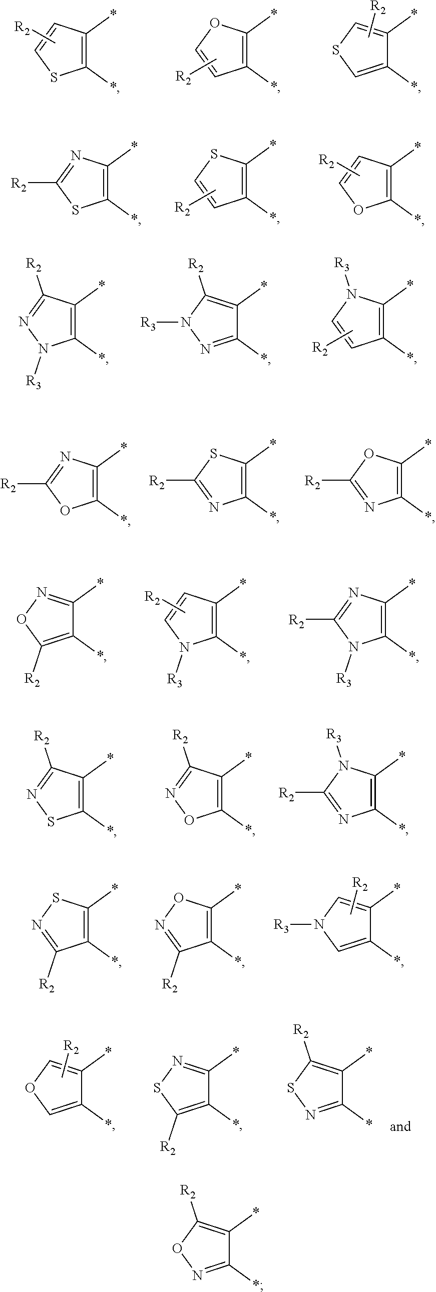
more preferably, 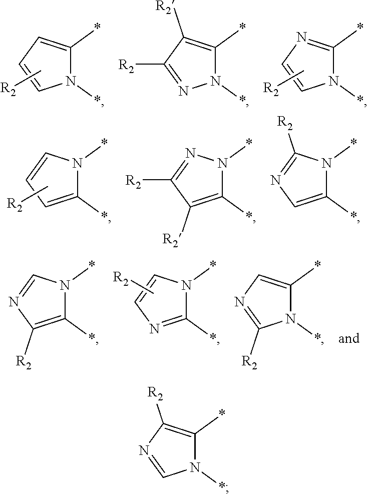
wherein * indicates the position at which the Z ring is attached to the rest of the compound; R₂ is selected from hydrogen, halogen, cyano, an optionally substituted C₁₋₆ alkyl, an optionally substituted C₁₋₃ alkoxy and an optionally substituted C₃₋₆ cycloalkyl, preferably hydrogen, halogen and an optionally substituted C₁₋₃ alkyl; R₂′ is selected from hydrogen, halogen, cyano, an optionally substituted C₁₋₆ alkyl, an optionally substituted C₁₋₃ alkoxy and an optionally substituted C₃₋₆ cycloalkyl, preferably hydrogen, halogen and an optionally substituted C₁₋₃ alkyl; R₃ is selected from a group consisting of hydrogen, an optionally substituted alkyl and an optionally substituted cycloalkyl, preferably hydrogen and an optionally substituted C₁₋₃ alkyl; and/or
L is C₁₋₃ alkylene; and/or
Cy is a 5-7 membered nitrogen-containing heterocyclic group optionally substituted by 1-5 substituents selected from a group consisting of halogen, C₁₋₄ alkyl, C₁₋₄ alkoxy, halogenated C₁₋₄ alkyl, halogenated C₁₋₄ alkoxy, an optionally substituted 6-14 membered aryl, an optionally substituted 5-10 membered heteroaryl, an optionally substituted 4-10 membered heterocyclic group and an optionally substituted C₃₋₈ cycloalkyl, wherein the said 6-14 membered aryl, 5-10 membered heteroaryl, 4-10 membered heterocyclic group and C₃₋₈ cycloalkyl each can be independently substituted by 1-5 substituents selected from a group consisting of halogen, C₁₋₄ alkyl, C₁₋₄ alkoxy, halogenated C₁₋₄ alkyl, halogenated C₁₋₄ alkoxy, —NR′R″, —C(O)—NR′R″ and carboxyl, wherein the said R′ and R″ each are preferably independently H, an optionally substituted C₁₋₁₀ alkyl, an optionally substituted C₃₋₈ cycloalkyl, an optionally substituted 3-6 membered heterocyclic group, an optionally substituted aryl or an optionally substituted heteroaryl, preferably R′ and R″ each are preferably independently H, an optionally substituted C₁₋₄ alkyl, an optionally substituted C₃₋₈ cycloalkyl or an optionally substituted 3-6 membered heterocyclic group; preferably, the substituents on 6-14 membered aryl, 5-10 membered heteroaryl, 4-10 membered heterocyclic group and C₃₋₈ cycloalkyl include at least an —C(O)—NR′R″, and optionally include one or two of substituents selected from a group consisting of halogen, C₁₋₄ alkyl and halogenated C₁₋₄ alkyl;
preferably, Cy is substituted by an optionally substituted 5-10 membered heteroaryl, preferably an optionally substituted 5-10 membered nitrogen-containing heteroaryl, preferably, the 5-10 membered heteroaryl or 5-10 membered nitrogen-containing heteroaryl is at least substituted by an —C(O)—NR′R″, and optionally substituted by one or two substituents selected from a group consisting of halogen, C₁₋₄ alkyl and halogenated C₁₋₄ alkyl, further preferably, the —C(O)—NR′R″ is at the para position;
preferably, Cy is piperazinyl or dihyropyridinyl substituted with an optionally substituted pyridyl, and the said pyridyl is at least substituted with an —C(O)—NR′R″; and Cy is covalently linked to L through its ring nitrogen atom.

3. The compound of any one of claims 1-2, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae Ia, Ib, Ic or Id as shown below: 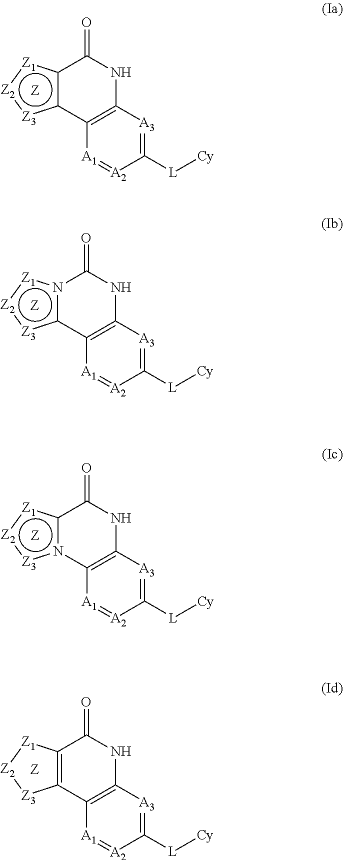
wherein, A₁, A₂, A₃, Z ring, L and Cy are as defined in any one of claims 1-2; or
the said compounds of Formula I are represented by Formulae IIa, IIb, IIc or IId: 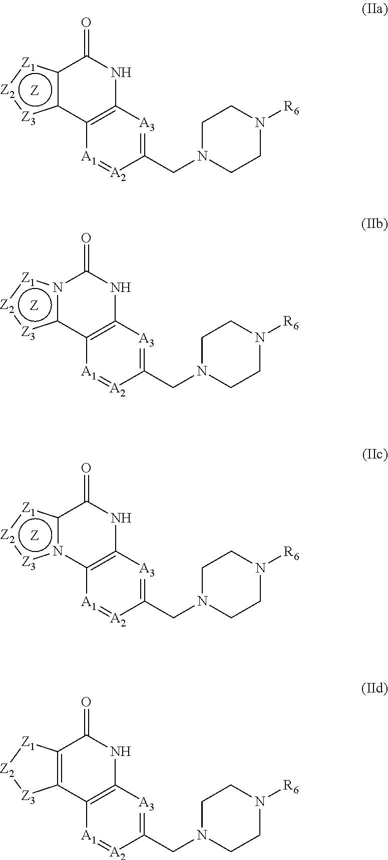
wherein, A₁, A₂, A₃ and Z ring are as defined in any one of claims 1-2; R₆ is selected from an optionally substituted aryl and an optionally substituted heteroaryl.

4. The compound of claim 3, or stereoisomers, tautomers, N-oxides, hydrates, solvates, isotope-substituted derivatives, or pharmaceutically acceptable salts thereof, or prodrugs thereof, wherein:
R₆ is an optionally substituted 6-14 membered aryl or an optionally substituted 5-10 membered heteroaryl, wherein the said optionally substituted 6-14 membered aryl and optionally substituted 5-10 membered heteroaryl each can be independently substituted by 1-5 substituents selected from a group consisting of halogen, C₁₋₄ alkyl, C₁₋₄ alkoxy, halogenated C₁₋₄ alkyl, halogenated C₁₋₄ alkoxy, —NR′R″, —C(O)—NR′R″ and carboxyl; wherein the said R′ and R″ each are preferably independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably R′ and R″ each are preferably independently H, an optionally substituted C₁₋₄ alkyl, an optionally substituted C₃₋₆ cycloalkyl or an optionally substituted 3-6 membered heterocyclic group; preferably, the substituents of R₆ include at least an —C(O)—NR′R″, and optionally include one or two of substituents selected from a group consisting of halogen, C₁₋₄ alkyl and halogenated C₁₋₄ alkyl; preferably, R₆ is an optionally substituted 5-10 membered heteroaryl, preferably an optionally substituted 5-10 membered nitrogen-containing heteroaryl, preferably, the 5-10 membered heteroaryl or 5-10 membered nitrogen-containing heteroaryl is at least substituted by an —C(O)—NR′R″, and optionally substituted by one or two substituents selected from a group consisting of halogen, C₁₋₄ alkyl or halogenated C₁₋₄ alkyl;
preferably, R₆ is preferably the following group: 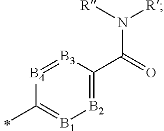
wherein, B₁, B₂, B₃ and B₄ are independently selected from a group consisting of N and CR₇; R₇ is selected from a group consisting of hydrogen, halogen, an optionally substituted alkyl, an optionally substituted alkoxy, an optionally substituted carbocyclic group, an optionally substituted alkenyl and an optionally substituted alkynyl; wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl, preferably, R′ and R″ each are independently hydrogen, an optionally substituted C₁₋₄ alkyl, an optionally substituted C₃₋₆ cycloalkyl or an optionally substituted 3-6 membered heterocyclic group; * indicates the position at which the said group is attached to the rest of the compound; preferably, the group containing B₁, B₂, B₃ and B₄ is phenyl, pyridyl, pyrimidinyl or pyridazinyl; preferably, R₇ is H, halogen, C₁₋₃ alkyl, C₁₋₃ alkoxy or halogenated C₁₋₃ alkyl; preferably, B₃ is N, B₄ is CR₇, both of B₁ and B₂ are CH, wherein R₇ is H, halogen, C₁₋₃ alkyl, C₁₋₃ alkoxy or halogenated C₁₋₃ alkyl; R′ is hydrogen, R″ is hydrogen, C₁₋₃ alkyl, deuterated C₁₋₃ alkyl, C₃₋₆ cycloalkyl or halogenated C₁₋₃ alkyl.

5. The compound of claim 1, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae IIIa, IIIb, IIIc or IIId as shown below: 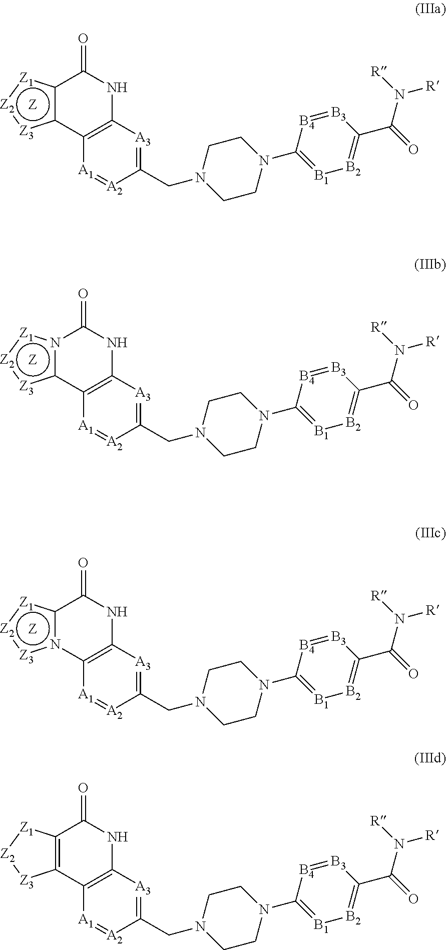
wherein, Z ring, A₁, A₂ and A₃ are as defined in any one of claims 1-2; B₁, B₂, B₃, B₄, R′ and R″ are as defined in claim 4.

6. The compound of claim 1, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae IVa, IVb, IVc or IVd as shown below: 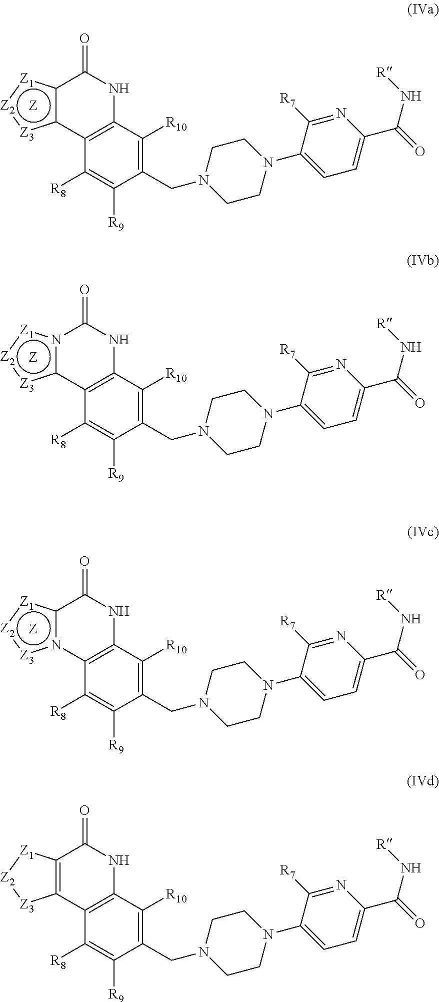
wherein, Z ring is as defined in claim 1 or 2, R″ is as defined in claim 2 or 4; R₇ is as defined in claim 4;
R₈, R₉ and R₁₀ are independently selected from hydrogen, halogen, optionally substituted alkyl, optionally substituted alkoxy and optionally substituted carbocyclic group;
preferably, R₈, R₉ and R₁₀ are each independently hydrogen, halogen or C₁₋₃ alkyl or optionally substituted C₁₋₃ alkoxy; preferably, R₈, R₉ and R₁₀ are each independently hydrogen, halogen or C₁₋₃ alkyl; preferably, R₈ is halogen or C₁₋₃ alkyl, both of R₉ and R₁₀ are CH; or R₈ is H, R₉ is halogen or C₁₋₃ alkyl, R₁₀ is CH; or both of R₈ and R₉ are CH, R₁₀ is halogen or C₁₋₃ alkyl;
preferably, at least one of R₉ and R₁₀ is halogen.

7. The compound of claim 1, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae Va, Vb, Vc or Vd as shown below: 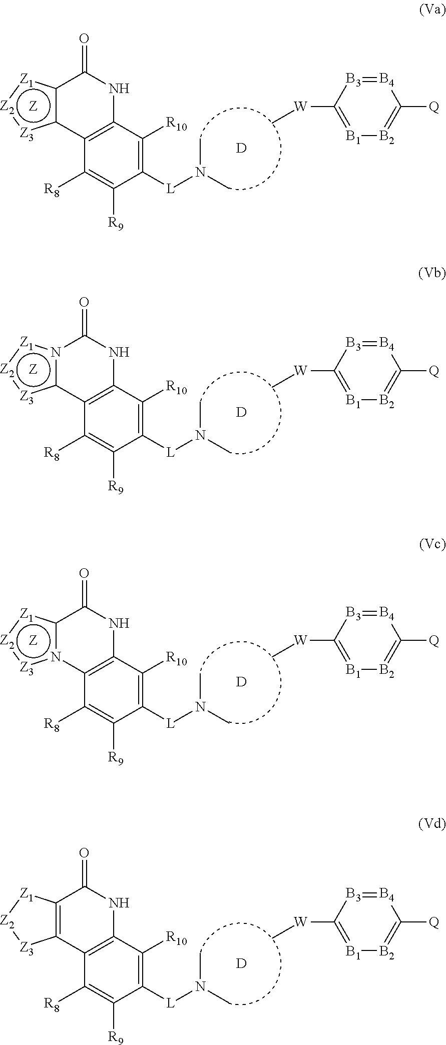
wherein, Z₁, Z₂ and Z₃ are as defined in any one of claims 1-2; R₈, R₉ and R₁₀ are as defined in claim 6; B₁, B₂, B₃ and B₄ are as defined in claim 4;
the D ring is an optionally substituted 4 to 12 membered N-containing heterocyclic group;
the said N-containing heterocyclic group containing at least 1 N atom is selected from 4-12 membered monocyclic group, 5-12 membered spirocyclic group, the said N-containing heterocyclic group can also include 1-4 heteroatoms selected from N, O or S, and can be optionally further substituted by 1 or more R″′;
W is a bond, O or —NR′—;
Q is an optionally substituted aminoacyl (—C(O)—NR′R″) or an optionally substituted heteroaryl;
R′ and R″ are each independently selected from hydrogen, optionally substituted C₁₋₁₀ alkyl, optionally substituted cycloalkyl, optionally substituted heterocyclic group, optionally substituted aryl and optionally substituted heteroaryl;
R″′ is selected from halogen, hydroxyl, cyano or C₁₋₆ alkyl, and the said C₁₋₆ alkyl is optionally further substituted by one or more substituents selected from hydroxyl, halogen or cyano; or any two R″′ can form a 3-8 membered ring.

8. The compound of claim 1, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae VIa, VIb or VIc as shown below: 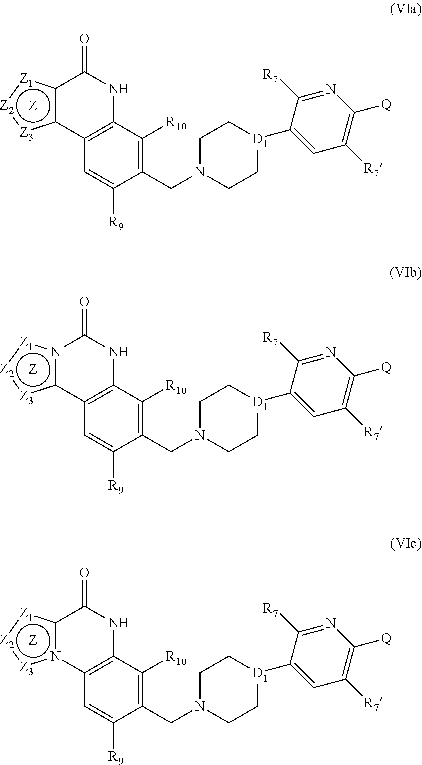
wherein, Z₁, Z₂ and Z₃ are as defined in any one of claims 1-2; R₇ and Q are as defined in claim 7;
R₇′ is hydrogen, halogen, cyano, optionally substituted C₁₋₃ alkyl, optionally substituted C₁₋₃ alkoxy or optionally substituted C₃₋₆ cycloalkyl;
R₉ and R₁₀ are each independently hydrogen, halogen, optionally substituted C₁₋₃ alkyl, or optionally substituted C₁₋₃ alkoxy, and at least one of R₉ and R₁₀ is not hydrogen;
D₁ is N or CR₁₁;
R₁ is H, halogen, cyano, hydroxyl, optionally substituted C₁₋₃ alkyl or optionally substituted C₁₋₃ alkoxy.

9. The compound of any one of claims 6-8, wherein:
Z ring is: 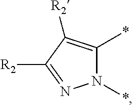
wherein, R₂ is selected from hydrogen, halogen, cyano, optionally substituted alkyl, optionally substituted C₁₋₃ alkoxy and optionally substituted C₃₋₆ cycloalkyl, preferably hydrogen, halogen, cyano, optionally substituted C₁₋₃ alkyl and optionally substituted C₃₋₆ cycloalkyl; R₂′ is selected from hydrogen, halogen, cyano, optionally substituted C₁₋₆ alkyl, optionally substituted C₁₋₃ alkoxy and optionally substituted C₃₋₆ cycloalkyl, preferably hydrogen, halogen, cyano, optionally substituted C₁₋₃ alkyl or C₃₋₆ cycloalkyl; moreover, at least one of R₂ and R₂′ are non-hydrogen substituent, preferably, the non-hydrogen substituent is halogen, cyano, C₁₋₃ alkyl or C₃₋₆ cycloalkyl; and in Formulae IVa-IVd and Va-Vd, one of R₈, R₉ and R₁₀ is halogen or C₁₋₃ alkyl, the other two are hydrogen, and in Formulae VIa-VId, one of R₉ and R₁₀ is halogen or C₁₋₃ alkyl, the other one is hydrogen; or
Z ring is: 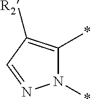
wherein, R₂′ is selected from halogen, cyano, optionally substituted C₁₋₆ alkyl, optionally substituted C₁₋₃ alkoxy and optionally substituted C₃₋₆ cycloalkyl, preferably halogen, cyano and optionally substituted C₁₋₃ alkyl and optionally substituted C₃₋₆ cycloalkyl; and in Formulae IVa-IVd and Va-Vd, R₈ is H, one of R₉ and R₁₀ is halogen or C₁₋₃ alkyl, the other one is hydrogen, and in Formulae VIa-VId, one of R₉ and R₁₀ is halogen or C₁₋₃ alkyl, the other one is hydrogen.

10. The compound of claim 8, wherein:
in Formula VIa, one of Z₁, Z₂ and Z₃ is O, the other two are CR₁, wherein, R₁ is selected from hydrogen, halogen and C₁₋₃ alkyl; preferably, Z₁ is O, both of Z₂ and Z₃ are CH; preferably, R₉ is non-hydrogen substituent, such as halogen, optionally substituted C₁₋₃ alkyl or optionally substituted C₁₋₃ alkoxy, preferably halogen, more preferably F; preferably, R₁₀ is hydrogen, halogen or C₁₋₃ alkyl, preferably hydrogen; D₁ is N or CH; R₇ is hydrogen, C₁₋₃ alkyl, halogenated C₁₋₃ alkyl, halogen, cyano or C₃₋₆ cycloalkyl, preferably hydrogen or halogen; preferably, Q is —C(O)—NR′R″ or pyrrolyl, pyrazolyl, imidazolyl or triazolyl optionally substituted by 1-2 groups selected from halogen and C₁₋₃ alkyl, wherein R′ and R″ are each independently selected from hydrogen, C₁₋₄ alkyl, C₃₋₆ cycloalkyl or 3-6 heterocyclic group; or
in Formula VIa, one of Z₁, Z₂ and Z₃ is O, the other two are CR₁, preferably, Z₁ is O, both of Z₂ and Z₃ are CH; R₁ is selected from hydrogen, halogen and C₁₋₃ alkyl; R₁₀ is non-hydrogen substituent, such as halogen, optionally substituted C₁₋₃ alkyl or optionally substituted C₁₋₃ alkoxy, preferably halogen, more preferably F; R₉ is hydrogen, halogen or C₁₋₃ alkyl, preferably hydrogen; D₁ is N or CH; R₇ is hydrogen, C₁₋₃ alkyl, halogenated C₁₋₃ alkyl, halogen, cyano or C₃₋₆ cycloalkyl, preferably hydrogen, halogen, C₁₋₃ alkyl or halogenated C₁₋₃ alkyl; R₇′ is hydrogen, C₁₋₃ alkyl, halogenated C₁₋₃ alkyl, halogen, cyano or C₃₋₆ cycloalkyl, preferably hydrogen or halogen; Q is —C(O)—NR′R″ or pyrrolyl, pyrazolyl, imidazolyl or triazolyl optionally substituted by 1-2 groups selected from halogen and C₁₋₃ alkyl, wherein, R′ and R″ are each independently selected from hydrogen, C₁₋₄ alkyl, C₃₋₆ cycloalkyl or 3-6 heterocyclic group; preferably, compounds where R₉ is H, R₁₀ is F, R₇ is methyl, D₁ is N, R₇′ is H, Q is —C(O)—NH(CH₃) or —C(O)—NH(CD₃) and compounds where R₉ is H, R₁₀ is F, R₇ is F, D₁ is N, R₇′ is H, Q is —C(O)—NH(CH₃) or —C(O)—NH(CD₃) are not included in formula VIa.

11. The compound of claim 1, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae VII as shown below: 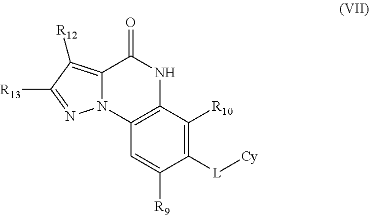
wherein, R₉ and R₁₀ are as defined in claim 8; L and Cy are as defined in any one of claims 1-3;
R₁₂ and R₁₃ are each independently selected from hydrogen, halogen, cyano, an optionally substituted C₁₋₆ alkyl, an optionally substituted C₁₋₃ alkoxy and optionally substituted C₃₋₆ cycloalkyl, and at least one of R₁₂ and R₁₃ is not hydrogen.

12. The compound of claim 1, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae VIII as shown below: 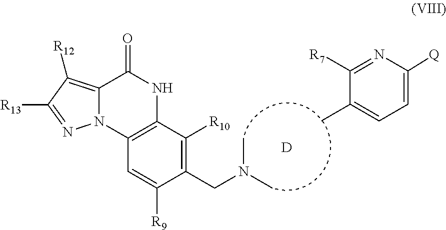
wherein, R₉ and R₁₀ are as defined in claims 8 and 12; R₁₂ and R₁₃ are as defined in claim 12; D ring and Q are as defined in any one of claims 7 and 8; R₇ is as defined in any one of claims 6 and 8.

13. The compound of claim 11, or stereoisomers, tautomers, N-oxides, hydrates, solvates, isotope-substituted derivatives, or pharmaceutically acceptable salts thereof, or prodrugs thereof, wherein:
R₁₂ and R₁₃ are each independently halogen, cyano, an optionally substituted C₁₋₃ alkyl or optionally substituted C₃₋₆ cycloalkyl; preferably, R₁₂ is hydrogen, and R₁₃ is halogen, cyano, an optionally substituted C₁₋₃ alkyl or optionally substituted C₃₋₆ cycloalkyl; more preferably, R₁₂ is halogen, cyano, an optionally substituted C₁₋₃ alkyl or optionally substituted C₃₋₆ cycloalkyl, and R₁₃ is hydrogen; preferably, R₁₂ is halogen, cyano, an optionally substituted C₁₋₃ alkyl or optionally substituted C₃₋₆ cycloalkyl, and R₁₃ is hydrogen; more preferably, R₁₂ is halogen, C₁₋₃ alkyl, halogenated C₁₋₃ alkyl; and R₁₃ is hydrogen;
R₉ is hydrogen, R₁₀ is halogen or C₁₋₃ alkyl; preferably, R₉ is halogen or C₁₋₃ alkyl, R₁₀ is hydrogen; more preferably, R₉ is hydrogen, R₁₀ is halogen; or R₉ is halogen, R₁₀ is hydrogen;
the D ring is an optionally substituted 4-7 membered monocyclic ring containing at least 1 N atom; preferably the D ring is an optionally substituted piperazinyl, an optionally substituted piperidinyl, or an optionally substituted dihyropyridinyl; wherein when the ring D is substituted, the substituents can be 1-3 groups selected from a group consisting of halogen, hydroxyl, cyano, optionally substituted C₁₋₃ alkyl, optionally substituted C₁₋₃ alkoxy and optionally substituted C₃₋₆ cycloalkyl;
R₇ is hydrogen, halogen, cyano, optionally substituted C₁₋₃ alkyl, optionally substituted C₁₋₃ alkoxy or optionally substituted C₃₋₆ cycloalkyl, preferably, R₇ is hydrogen, halogen, cyano, C₁₋₃ alkyl, halogenated C₁₋₃ alkyl or C₃₋₆ cycloalkyl;
Q is —C(O)—NR′R″, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or optionally substituted heteroaryl, preferably hydrogen, an optionally substituted C₁₋₄ alkyl, an optionally substituted C₃₋₆ cycloalkyl or an optionally substituted 3-6 membered heterocyclic group; preferably, Q is a 5 membered heteroaryl optionally substituted by 1-3 substituents selected from halogen, cyano, C₁₋₃ alkyl, halogenated C₁₋₃ alkyl, C₁₋₃ alkoxy and halogenated C₁₋₃ alkoxy, the 5 membered heteroaryl is preferably pyrrolyl, pyrazolyl, imidazolyl or triazolyl.

14. The compound of claim 1, or stereoisomers, tautomers, N-oxides, hydrates, solvates, isotope-substituted derivatives, or pharmaceutically acceptable salts thereof, or prodrugs thereof, or mixtures thereof, wherein the compound of Formula I is represented by Formulae IX as shown below: 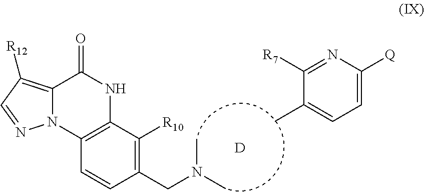
wherein, D ring and Q are as defined in any one of claims 7 and 8; R₇ is as defined in any one of claims 6 and 8;
R₁₀ is halogen, optionally substituted C₁₋₃ alkyl, or optionally substituted C₁₋₃ alkoxy;
R₁₂ is halogen, cyano, an optionally substituted C₁₋₆ alkyl, an optionally substituted C₁₋₃ alkoxy and optionally substituted C₃₋₆ cycloalkyl.

15. The compound of claim 14, or stereoisomers, tautomers, N-oxides, hydrates, solvates, isotope-substituted derivatives, or pharmaceutically acceptable salts thereof, or prodrugs thereof,
R₁₀ is halogen or C₁₋₃ alkyl; preferably, R₁₀ is halogen, preferably F;
R₁₂ is halogen, cyano, an optionally substituted C₁₋₃ alkyl or optionally substituted C₃₋₆ cycloalkyl; preferably, R₁₂ is halogen, C₁₋₃ alkyl, halogenated C₁₋₃ alkyl;
the D ring is an optionally substituted 4-7 membered monocyclic ring containing at least 1 N atom; preferably the D ring is an optionally substituted piperazinyl, an optionally substituted piperidinyl, or an optionally substituted dihyropyridinyl, wherein when the ring D is substituted, the substituents can be 1-3 groups selected from a group consisting of halogen, hydroxyl, cyano, optionally substituted C₁₋₃ alkyl, optionally substituted C₁₋₃ alkoxy and optionally substituted C₃₋₆ cycloalkyl; or
the D ring is selected from the following groups: 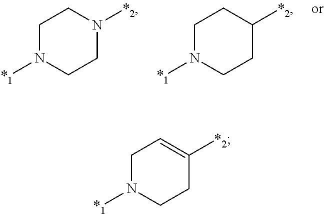
preferably, 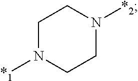
wherein, *1 indicates the position at which the D ring is attached to methylene; *2 indicates the position at which the D ring is attached to the pyridyl;
R₇ is hydrogen, halogen, cyano, optionally substituted C₁₋₃ alkyl, optionally substituted C₁₋₃ alkoxy or optionally substituted C₃₋₆ cycloalkyl, preferably, R₇ is hydrogen, halogen, cyano, C₁₋₃ alkyl, halogenated C₁₋₃ alkyl or C₃₋₆ cycloalkyl;
Q is —C(O)—NR′R″, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or optionally substituted heteroaryl, preferably hydrogen, an optionally substituted C₁₋₄ alkyl, an optionally substituted C₃₋₆ cycloalkyl or an optionally substituted 3-6 membered heterocyclic group.

16. The compound of claim 1, wherein the compound is selected from a group consisting of a group consisting of:
7-((4-(2-methyl-6-(methylcarbamoyl)pyridin-3-yl)piperazin-1-yl)methyl)thieno[3,2-c]quinolin-4(5H)-one;
7-((4-(2-methyl-6-(methylcarbamoyl)pyridin-3-yl)piperazin-1-yl)methyl)furo[2,3-c]quinolin-4(5H)-one;
7-((4-(2-methyl-6-(methylcarbamoyl)pyridin-3-yl)piperazin-1-yl)methyl)thieno[3,4-c]quinolin-4(5H)-one;
7-((4-(2-methyl-6-(methylcarbamoyl)pyridin-3-yl)piperazin-1-yl)methyl)thiazolo[4,5-c]quinolin-4(5H)-one;
7-((4-(2-methyl-6-(methylcarbamoyl)pyridin-3-yl)piperazin-1-yl)methyl)thieno[2,3-c]quinolin-4(5H)-one;
7-((4-(2-methyl-6-(methylcarbamoyl)pyridin-3-yl)piperazin-1-yl)methyl)furo[3,2-c]quinolin-4(5H)-one;
7-((4-(2-methyl-6-(methylcarbamoyl)pyridin-3-yl)piperazin-1-yl)methyl)-1,5-dihydro-4H-pyrazolo[4,3-c]quinolin-4-one;
7-((4-(2-methyl-6-(methylcarbamoyl)pyridin-3-yl)piperazin-1-yl)methyl)-1-methyl-1,5-dihydro-4H-pyrazolo[4,3-c]quinolin-4-one;
7-((4-(2-methyl-6-(methylcarbamoyl)pyridin-3-yl)piperazin-1-yl)methyl)-2-methyl-2,5-dihydro-4H-pyrazolo[4,3-c]quinolin-4-one;
7-((4-(2-methyl-6-(methylcarbamoyl)pyridin-3-yl)piperazin-1-yl)methyl)-3,5-dihydro-4H-pyrrolo[2,3-c]quinolin-4-one;
7-((4-(2-methyl-6-(methylcarbamoyl)pyridin-3-yl)piperazin-1-yl)methyl)-3-methyl-3,5-dihydro-4H-pyrrolo[2,3-c]quinolin-4-one;
7-((4-(2-methyl-6-(methylcarbamoyl)pyridin-3-yl)piperazin-1-yl)methyl)oxazolo[4,5-c]quinolin-4(5H)-one;
7-((4-(2-methyl-6-(methylcarbamoyl)pyridin-3-yl)piperazin-1-yl)methyl)thiazolo[5,4-c]quinolin-4(5H)-one;
7-((4-(2-methyl-6-(methylcarbamoyl)pyridin-3-yl)piperazin-1-yl)methyl)oxazolo[5,4-c]quinolin-4(5H)-one;
7-((4-(2-methyl-6-(methylcarbamoyl)pyridin-3-yl)piperazin-1-yl)methyl)isoxazolo[3,4-c]quinolin-4(5H)-one;
7-((4-(2-fluoro-6-(methylcarbamoyl)pyridin-3-yl)piperazin-1-yl)methyl)thieno[3,2-c]quinolin-4(5H)-one;
7-((4-(2-fluoro-6-(methylcarbamoyl)pyridin-3-yl)piperazin-1-yl)methyl)thieno[3,4-c]quinolin-4(5H)-one;
7-((4-(2-chloro-6-(methylcarbamoyl)pyridin-3-yl)piperazin-1-yl)methyl)thieno[3,2-c]quinolin-4(5H)-one;
7-((4-(2-chloro-6-(methylcarbamoyl)pyridin-3-yl)piperazin-1-yl)methyl)thieno[3,4-c]quinolin-4(5H)-one;
7-((4-(2-methyl-6-(methylcarbamoyl)pyridin-3-yl)piperazin-1-yl)methyl)-9-fluorothieno[3,2-c]quinolin-4(5H)-one;
7-((4-(2-methyl-6-(methylcarbamoyl)pyridin-3-yl)piperazin-1-yl)methyl)-9-fluorothieno[3,4-c]quinolin-4(5H)-one;
7-((4-(2-methyl-6-(methylcarbamoyl)pyridin-3-yl)piperazin-1-yl)methyl)-9-fluorofuro[3,2-c]quinolin-4(5H)-one;
7-((4-(2-methyl-6-(methylcarbamoyl)pyridin-3-yl)piperazin-1-yl)methyl)-1,5-dihydro-4H-pyrrolo[3,2-c]quinolin-4-one;
7-((4-(2-fluoro-6-(methylcarbamoyl)pyridin-3-yl)piperazin-1-yl)methyl)-1-methyl-1,5-dihydro-4H-pyrrolo[3,2-c]quinolin-4-one;
7-((4-(2-methyl-6-(methylcarbamoyl)pyridin-3-yl)piperazin-1-yl)methyl)-1,5-dihydro-4H-imidazo[4,5-c]quinolin-4-one;
7-((4-(2-methyl-6-(methylcarbamoyl)pyridin-3-yl)piperazin-1-yl)methyl)-1-methyl-1,5-dihydro-4H-imidazo[4,5-c]quinolin-4-one;
7-((4-(2-methyl-6-(methylcarbamoyl)pyridin-3-yl)piperazin-1-yl)methyl)isothiazolo[4,5-c]quinolin-4(5H)-one;
7-((4-(2-methyl-6-(methylcarbamoyl)pyridin-3-yl)piperazin-1-yl)methyl)-3-methyl-3,5-dihydro-4H-imidazo[4,5-c]quinolin-4-one;
7-((4-(2-methyl-6-(methylcarbamoyl)pyridin-3-yl)piperazin-1-yl)methyl)isothiazolo[5,4-c]quinolin-4(5H)-one;
7-((4-(2-methyl-6-(methylcarbamoyl)pyridin-3-yl)piperazin-1-yl)methyl)isoxazolo[5,4-c]quinolin-4(5H)-one;
7-((4-(2-methyl-6-(methylcarbamoyl)pyridin-3-yl)piperazin-1-yl)methyl)isothiazolo[3,4-c]quinolin-4(5H)-one;
7-((4-(2-methyl-6-(methylcarbamoyl)pyridin-3-yl)piperazin-1-yl)methyl)isothiazolo[4,3-c]quinolin-4(5H)-one;
7-((4-(2-methyl-6-(methylcarbamoyl)pyridin-3-yl)piperazin-1-yl)methyl)isoxazolo[4,5-c]quinolin-4(5H)-one;
7-((4-(2-methyl-6-(methylcarbamoyl)pyridin-3-yl)piperazin-1-yl)methyl)-2,5-dihydro-4H-pyrrolo[3,4-c]quinolin-4-one;
7-((4-(2-methyl-6-(methylcarbamoyl)pyridin-3-yl)piperazin-1-yl)methyl)-2-methyl-2,5-dihydro-4H-pyrrolo[3,4-c]quinolin-4-one;
7-((4-(2-methyl-6-(methylcarbamoyl)pyridin-3-yl)piperazin-1-yl)methyl)furo[3,4-c]quinolin-4(5H)-one;
7-((4-(2-methyl-6-(methylcarbamoyl)pyridin-3-yl)piperazin-1-yl)methyl)isoxazolo[4,3-c]quinolin-4(5H)-one;
9-fluoro-7-((4-(2-methyl-6-(methylcarbamoyl)pyridin-3-yl)piperazin-1-yl)methyl)thieno[2,3-c]quinolin-4(5H)-one;
9-fluoro-7-((4-(2-methyl-6-(methylcarbamoyl)pyridin-3-yl)piperazin-1-yl)methyl)thieno[3,2-c]quinolin-4(5H)-one;
9-fluoro-7-((4-(2-methyl-6-(methylcarbamoyl)pyridin-3-yl)piperazin-1-yl)methyl)furo[2,3-c]quinolin-4(5H)-one;
9-fluoro-7-((4-(2-methyl-6-(methylcarbamoyl)pyridin-3-yl)piperazin-1-yl)methyl)thiazolo[4,5-c]quinolin-4(5H)-one;
9-fluoro-7-((4-(2-methyl-6-(methylcarbamoyl)pyridin-3-yl)piperazin-1-yl)methyl)-1-methyl-1,5-dihydro-4H-pyrazolo[4,3-c]quinolin-4-one;
7-((4-(2-methyl-6-(methylcarbamoyl)pyridin-3-yl)piperazin-1-yl)methyl)-6-fluoro-furo[3,2-c]quinolin-4(5H)-one;
7-((4-(2-fluoro-6-(methylcarbamoyl)pyridin-3-yl)piperazin-1-yl)methyl)-6-fluoro-furo[3,2-c]quinolin-4(5H)-one;
7-((4-(2-methyl-6-(methylcarbamoyl)pyridin-3-yl)piperazin-1-yl)methyl)-6-fluoro-1,5-dihydro-4H-pyrazolo[4,3-c]quinolin-4-one;
7-((4-(2-fluoro-6-(methylcarbamoyl)pyridin-3-yl)piperazin-1-yl)methyl)-6-fluoro-1,5-dihydro-4H-pyrazolo[4,3-c]quinolin-4-one;
7-((4-(2-methyl-6-(methylcarbamoyl)pyridin-3-yl)piperazin-1-yl)methyl)-3-methylisoxazolo[4,5-c]quinolin-4(5H)-one;
8-((4-(2-methyl-6-(methylcarbamoyl)pyridin-3-yl)piperazin-1-yl)methyl)-7-fluoropyrrolo[1,2-c]quinazolin-5(6H)-one;
7-((4-(2-fluoro-6-(methylcarbamoyl)pyridin-3-yl)piperazin-1-yl)methyl)-3-fluoropyrazolo[1,5-a]quinoxalin-4(5H)-one;
8-((4-(2-methyl-6-(methylcarbamoyl)pyridin-3-yl)piperazin-1-yl)methyl)-7-fluoroimidazo[1,2-c]quinazolin-5(6H)-one;
7-((4-(2-methyl-6-(methylcarbamoyl)pyridin-3-yl)piperazin-1-yl)methyl)-3-fluoro-2-methylpyrazolo[1,5-a]quinoxalin-4(5H)-one;
7-((4-(2-fluoro-6-(methylcarbamoyl)pyridin-3-yl)piperazin-1-yl)methyl)-3-fluoro-2-methylpyrazolo[1,5-a]quinoxalin-4(5H)-one;
7-((4-(2-fluoro-6-(methylcarbamoyl)pyridin-3-yl)piperazin-1-yl)methyl)-9-fluoroimidazo[1,5-a]quinoxalin-4(5H)-one;
7-((4-(2-chloro-6-(methylcarbamoyl)pyridin-3-yl)piperazin-1-yl)methyl)-9-fluoroimidazo[1,5-a]quinoxalin-4(5H)-one;
7-((4-(2-fluoro-6-(methylcarbamoyl)pyridin-3-yl)piperazin-1-yl)methyl)-6-fluoroimidazo[1,5-a]quinoxalin-4(5H)-one;
7-((4-(2-chloro-6-(methylcarbamoyl)pyridin-3-yl)piperazin-1-yl)methyl)-6-fluoroimidazo[1,5-a]quinoxalin-4(5H)-one;
7-((4-(2-methyl-6-(methylcarbamoyl)pyridin-3-yl)piperazin-1-yl)methyl)-6-fluoro-3-methylpyrazolo[1,5-a]quinoxalin-4(5H)-one;
7-((4-(2-fluoro-6-(methylcarbamoyl)pyridin-3-yl)piperazin-1-yl)methyl)-6-fluoro-3-methylpyrazolo[1,5-a]quinoxalin-4(5H)-one;
7-((4-(2-methyl-6-(methylcarbamoyl)pyridin-3-yl)piperazin-1-yl)methyl)-6-fluoro-3-chloro-pyrazolo[1,5-a]quinoxalin-4(5H)-one;
7-((4-(2-fluoro-6-(methylcarbamoyl)pyridin-3-yl)piperazin-1-yl)methyl)-6-fluoro-3-chloro-pyrazolo[1,5-a]quinoxalin-4(5H)-one;
8-((4-(2-fluoro-6-(methylcarbamoyl)pyridin-3-yl)piperazin-1-yl)methyl)-7-fluoroimidazo[1,2-c]quinazolin-5(6H)-one;
8-((4-(2-fluoro-6-(ethylcarbamoyl)pyridin-3-yl)piperazin-1-yl)methyl)-7-fluoroimidazo[1,2-c]quinazolin-5(6H)-one;
8-((4-(2-methyl-6-(ethylcarbamoyl)pyridin-3-yl)piperazin-1-yl)methyl)-7-fluoroimidazo[1,2-c]quinazolin-5(6H)-one;
8-((4-(2-fluoro-6-(cyclopropylcarbamoyl)pyridin-3-yl)piperazin-1-yl)methyl)-7-fluoroimidazo[1,2-c]quinazolin-5(6H)-one;
8-((4-(2-methyl-6-(cyclopropylcarbamoyl)pyridin-3-yl)piperazin-1-yl)methyl)-7-fluoroimidazo[1,2-c]quinazolin-5(6H)-one;
7-((4-(2-fluoro-6-(ethylcarbamoyl)pyridin-3-yl)piperazin-1-yl)methyl)-6-fluorofuro[2,3-c]quinolin-4(5H)-one;
7-((4-(2-fluoro-6-(cyclopropylcarbamoyl)pyridin-3-yl)piperazin-1-yl)methyl)-6-fluorofuro[2,3-c]quinolin-4(5H)-one;
7-((4-(2-chloro-6-(methylcarbamoyl)pyridin-3-yl)piperazin-1-yl)methyl)-6-fluorofuro[2,3-c]quinolin-4(5H)-one;
7-((4-(2-methyl-6-(methylcarbamoyl)pyridin-3-yl)piperazin-1-yl)methyl)-2-methyl-2,5-dihydro-4H-pyrazolo[3,4-c]quinolin-4-one;
7-((4-(2-methyl-6-(methylcarbamoyl)pyridin-3-yl)piperazin-1-yl)methyl)-2,5-dihydro-4H-pyrazolo[3,4-c]quinolin-4-one;
7-((4-(2-methyl-6-(methylcarbamoyl)pyridin-3-yl)piperazin-1-yl)methyl)-3-methyl-3,5-dihydro-4H-pyrazolo[3,4-c]quinolin-4-one;
8-((4-(2-methyl-6-(methylcarbamoyl)pyridin-3-yl)piperazin-1-yl)methyl)-7-fluoropyrazolo[1,5-c]quinazolin-5(6H)-one;
8-((4-(2-fluoro-6-(methylcarbamoyl)pyridin-3-yl)piperazin-1-yl)methyl)-7-fluoropyrazolo[1,5-c]quinazolin-5(6H)-one;
7-((4-(6-methylcarbamoyl-2-methylpyridin-3-yl)piperazin-1-yl)methyl)-1,2,3,5-tetrahydro-4H-pyrrolo[3,4-c]quinolin-4-one;
7-((4-(6-methylcarbamoyl-2-methylpyridin-3-yl)piperazin-1-yl)methyl)-2-methyl-1,2,3,5-tetrahydro-4H-pyrrolo[3,4-c]quinolin-4-one;
7-((4-(6-methylcarbamoyl-2-methylpyridin-3-yl)piperazin-1-yl)methyl)-6-fluoro-1,2,3,5-tetrahydro-4H-pyrrolo[3,4-c]quinolin-4-one;
7-((4-(6-methylcarbamoyl-2-fluoropyridin-3-yl)piperazin-1-yl)methyl)-6-fluoro-1,2,3,5-tetrahydro-4H-pyrrolo[3,4-c]quinolin-4-one;
7-((4-(6-methylcarbamoyl-2-methylpyridin-3-yl)piperazin-1-yl)methyl)-6-fluoro-2-methyl-1,2,3,5-tetrahydro-4H-pyrrolo[3,4-c]quinolin-4-one;
7-((4-(6-methylcarbamoyl-2-fluoropyridin-3-yl)piperazin-1-yl)methyl)-6-fluoro-2-methyl-1,2,3,5-tetrahydro-4H-pyrrolo[3,4-c]quinolin-4-one;
7-((4-(6-methylcarbamoyl-2-methylpyridin-3-yl)piperazin-1-yl)methyl)-6-fluoro-1,2,3,5-tetrahydro-4H-cyclopenta[c]quinolin-4-one;
7-((4-(6-methylcarbamoyl-2-fluoropyridin-3-yl)piperazin-1-yl)methyl)-6-fluoro-1,2,3,5-tetrahydro-4H-cyclopenta[c]quinolin-4-one;
7-((4-(6-methylcarbamoyl-2-methylpyridin-3-yl)piperazin-1-yl)methyl)-6-fluoro-3,5-dihydrofuro[3,2-c]quinolin-4(2H)-one;
7-((4-(6-methylcarbamoyl-2-fluoropyridin-3-yl)piperazin-1-yl)methyl)-6-fluoro-3,5-dihydrofuro[3,2-c]quinolin-4(2H)-one;
7-((4-(6-methylcarbamoyl-2-methylpyridin-3-yl)piperazin-1-yl)methyl)-6-fluoro-3,5-dihydrofuro[3,4-c]quinolin-4(1H)-one;
7-((4-(6-methylcarbamoyl-2-fluoropyridin-3-yl)piperazin-1-yl)methyl)-6-fluoro-3,5-dihydrofuro[3,4-c]quinolin-4(1H)-one;
7-((4-(6-methylcarbamoyl-2-methylpyridin-3-yl)piperazin-1-yl)methyl)-6-fluoro-1,2-dihydrofuro[2,3-c]quinolin-4(5H)-one;
7-((4-(6-methylcarbamoyl-2-fluoropyridin-3-yl)piperazin-1-yl)methyl)-6-fluoro-1,2-dihydrofuro[2,3-c]quinolin-4(5H)-one;
7-((4-(2-methyl-6-(methylcarbamoyl)pyridin-3-yl)piperazin-1-yl)methyl)-6-fluoro-2-methyl-2,5-dihydro-4H-pyrazolo[4,3-c]quinolin-4-one;
7-((4-(2-methyl-6-(methylcarbamoyl)pyridin-3-yl)piperazin-1-yl)methyl)-8-fluoro-2-methyl-2,5-dihydro-4H-pyrazolo[4,3-c]quinolin-4-one;
7-((4-(2-fluoro-6-(methylcarbamoyl)pyridin-3-yl)piperazin-1-yl)methyl)-6-fluoro-2-methyl-2,5-dihydro-4H-pyrazolo[4,3-c]quinolin-4-one;
7-((4-(2-fluoro-6-(methylcarbamoyl)pyridin-3-yl)piperazin-1-yl)methyl)-8-fluoro-2-methyl-2,5-dihydro-4H-pyrazolo[4,3-c]quinolin-4-one;
6-fluoro-7-((4-(2-fluoro-6-(methylcarbamoyl)pyridin-3-yl)piperazin-1-yl)methyl)-1-methyl-1,5-dihydro-4H-pyrazolo[4,3-c]quinolin-4-one;
6-fluoro-7-((4-(2-fluoro-6-(methylcarbamoyl)pyridin-3-yl)piperazin-1-yl)methyl)-2-methyl-2,5-dihydro-4H-pyrazolo[3,4-c]quinolin-4-one;
7-((4-(2-fluoro-6-(methylcarbamoyl)pyridin-3-yl)piperazin-1-yl)methyl)-8-fluorooxazolo[5,4-c]quinolin-4(5H)-one;
7-((4-(2-fluoro-6-(methylcarbamoyl)pyridin-3-yl)piperazin-1-yl)methyl)-6-fluorooxazolo[5,4-c]quinolin-4(5H)-one;
7-((4-(2-fluoro-6-(methylcarbamoyl)pyridin-3-yl)piperazin-1-yl)methyl)-3-methyl-6-fluoroisoxazolo[4,5-c]quinolin-4(5H)-one;
7-((4-(2-fluoro-6-(methylcarbamoyl)pyridin-3-yl)piperazin-1-yl)methyl)-3-methyl-8-fluoroisoxazolo[4,5-c]quinolin-4(5H)-one;
7-((4-(2-fluoro-6-(methylcarbamoyl)pyridin-3-yl)piperazin-1-yl)methyl)isoxazolo[4,5-c]quinolin-4(5H)-one;
7-((4-(2-fluoro-6-(methylcarbamoyl)pyridin-3-yl)piperazin-1-yl)methyl)-3,5-dihydro-4H-pyrrolo[2,3-c]quinolin-4-one;
7-((4-(2-fluoro-6-(methylcarbamoyl)pyridin-3-yl)piperazin-1-yl)methyl)-6-fluoro-1-methyl-1,5-dihydro-4H-imidazo[4,5-c]quinolin-4-one;
7-((4-(2-(difluoromethyl)-6-(methylcarbamoyl)pyridin-3-yl)piperazin-1-yl)methyl)-6-fluorofuro[2,3-c]quinolin-4(5H)-one;
7-((4-(2-fluoro-6-(methylcarbamoyl)pyridin-3-yl)piperazin-1-yl)methyl)-8-fluorofuro[2,3-c]quinolin-4(5H)-one;
7-((4-(2-fluoro-6-(methylcarbamoyl)pyridin-3-yl)piperazin-1-yl)methyl)-6-methylfuro[2,3-c]quinolin-4(5H)-one;
7-((4-(2-fluoro-6-(methylcarbamoyl)pyridin-3-yl)piperazin-1-yl)methyl)-8-methylfuro[2,3-c]quinolin-4(5H)-one;
7-((4-(2-fluoro-6-(methylcarbamoyl)pyridin-3-yl)piperazin-1-yl)methyl)-6-chlorofuro[2,3-c]quinolin-4(5H)-one;
7-((4-(2-fluoro-6-carbamoylpyridin-3-yl)piperazin-1-yl)methyl)-6-fluorofuro[2,3-c]quinolin-4(5H)-one;
6-fluoro-7-((4-(5-fluoro-6-(methylcarbamoyl)pyridin-3-yl)piperazin-1-yl)methyl)furo[2,3-c]quinolin-4(5H)-one;
6-fluoro-7-((4-(6-(methylcarbamoyl)-2-(trifluoromethyl)pyridin-3-yl)piperazin-1-yl)methyl)furo[2,3-c]quinolin-4(5H)-one;
7-((4-(2-fluoro-6-(methylcarbamoyl)pyridin-3-yl)piperidin-1-yl)methyl)-6-fluorofuro[2,3-c]quinolin-4(5H)-one;
7-((4-(2-fluoro-6-(4H-1,2,4-triazol-3-yl)pyridin-3-yl)piperazin-1-yl)methyl)-6-fluorofuro[2,3-c]quinolin-4(5H)-one;
7-((4-(2-fluoro-6-(5-methyl-1H-imidazol-2-yl)pyridin-3-yl)piperazin-1-yl)methyl)-6-fluorofuro[2,3-c]quinolin-4(5H)-one;
7-((4-(2-fluoro-6-(methylcarbamoyl)pyridin-3-yl)piperazin-1-yl)methyl)furo[2,3-c]quinolin-4(5H)-one;
7-((4-(6-(methylcarbamoyl)pyridin-3-yl)piperazin-1-yl)methyl)furo[2,3-c]quinolin-4(5H)-one;
7-((4-(2-fluoro-6-(methylcarbamoyl)pyridin-3-yl)piperazin-1-yl)methyl)furo[3,2-c]quinolin-4(5H)-one;
7-((4-(2-fluoro-6-(methylcarbamoyl)pyridin-3-yl)piperazin-1-yl)methyl)-6-fluorofuro[3,4-c]quinolin-4(5H)-one;
7-((4-(2-fluoro-6-(methylcarbamoyl)pyridin-3-yl)piperazin-1-yl)methyl)-6-fluoro-2-methylfuro[2,3-c]quinolin-4(5H)-one;
7-((4-(2-fluoro-6-(methylcarbamoyl)pyridin-3-yl)piperazin-1-yl)methyl)-2,6-difluorofuro[2,3-c]quinolin-4(5H)-one;
7-((4-(6-(methylcarbamoyl)pyridin-3-yl)piperazin-1-yl)methyl)furo[3,2-c]quinolin-4(5H)-one;
8-((4-(2-methyl-6-(methylcarbamoyl)pyridin-3-yl)piperazin-1-yl)methyl)-7-fluoropyrazolo[1,5-c]quinazolin-5(6H)-one;
8-((4-(2-fluoro-6-(methylcarbamoyl)pyridin-3-yl)piperazin-1-yl)methyl)-9-fluoropyrazolo[1,5-c]quinazolin-5(6H)-one;
7-((4-(2-methyl-6-(methylcarbamoyl)pyridin-3-yl)piperazin-1-yl)methyl)-3,6-difluoropyrazolo[1,5-a]quinoxalin-4(5H)-one;
7-((4-(2-fluoro-6-(methylcarbamoyl)pyridin-3-yl)piperazin-1-yl)methyl)-3,6-difluoropyrazolo[1,5-a]quinoxalin-4(5H)-one;
7-((4-(2-fluoro-6-(methylcarbamoyl)pyridin-3-yl)piperazin-1-yl)methyl)-2,6-difluoropyrazolo[1,5-a]quinoxalin-4(5H)-one;
6-fluoro-7-((4-(2-fluoro-6-(methylcarbamoyl)pyridin-3-yl)piperidin-1-yl)methyl)-3-methylpyrazolo[1,5-a]quinoxalin-4(5H)-one;
7-((4-(2-fluoro-6-(ethylcarbamoyl)pyridin-3-yl)piperazin-1-yl)methyl)-6-fluoro-3-methylpyrazolo[1,5-a]quinoxalin-4(5H)-one;
7-((4-(2-fluoro-6-(cyclopropylcarbamoyl)pyridin-3-yl)piperazin-1-yl)methyl)-6-fluoro-3-methylpyrazolo[1,5-a]quinoxalin-4(5H)-one;
7-((4-(5-fluoro-6-(methylcarbamoyl)pyridin-3-yl)piperazin-1-yl)methyl)-6-fluoro-3-methylpyrazolo[1,5-a]quinoxalin-4(5H)-one;
7-((4-(2-cyano-6-(methylcarbamoyl)pyridin-3-yl)piperazin-1-yl)methyl)-6-fluoro-3-methylpyrazolo[1,5-a]quinoxalin-4(5H)-one;
7-((4-(2-fluoro-6-(2,2-difluoroethylcarbamoyl)pyridin-3-yl)piperazin-1-yl)methyl)-6-fluoro-3-methylpyrazolo[1,5-a]quinoxalin-4(5H)-one;
7-((4-(2-fluoro-6-(1H-imidazol-5-yl)pyridin-3-yl)piperazin-1-yl)methyl)-6-fluoro-3-methylpyrazolo[1,5-a]quinoxalin-4(5H)-one;
7-((4-(2-chloro-6-(methylcarbamoyl)pyridin-3-yl)piperazin-1-yl)methyl)-6-fluoro-3-methylpyrazolo[1,5-a]quinoxalin-4(5H)-one;
7-((4-(2-difluoromethyl-6-(methylcarbamoyl)pyridin-3-yl)piperazin-1-yl)methyl)-6-fluoro-3-methylpyrazolo[1,5-a]quinoxalin-4(5H)-one;
7-((4-(2-trifluoromethyl-6-(methylcarbamoyl)pyridin-3-yl)piperazin-1-yl)methyl)-6-fluoro-3-methylpyrazolo[1,5-a]quinoxalin-4(5H)-one;
(R)-6-fluoro-3-methyl-7-((3-((6-(methylcarbamoyl)pyridin-3-yl)amino)pyrrolidin-1-yl)methyl)pyrazolo[1,5-a]quinoxalin-4(5H)-one;
(R)-6-fluoro-7-((3-((2-fluoro-6-(methylcarbamoyl)pyridin-3-yl)amino)pyrrolidin-1-yl)methyl)-3-methylpyrazolo[1,5-a]quinoxalin-4(5H)-one;
8-((4-(2-methyl-6-(methylcarbamoyl)pyridin-3-yl)piperazin-1-yl)methyl)-2-methyl-10-fluoroimidazo[1,2-c]quinazolin-5(6H)-one;
8-((4-(2-fluoro-6-(methylcarbamoyl)pyridin-3-yl)piperazin-1-yl)methyl)-2-methyl-10-fluoroimidazo[1,2-c]quinazolin-5(6H)-one;
8-((4-(2-fluoro-6-(methylcarbamoyl)pyridin-3-yl)piperazin-1-yl)methyl)-3,7-difluoroimidazo[1,2-c]quinazolin-5(6H)-one;
8-((4-(2-fluoro-6-(methylcarbamoyl)pyridin-3-yl)piperazin-1-yl)methyl)-2,7-difluoroimidazo[1,2-c]quinazolin-5(6H)-one;
8-((4-(2-fluoro-6-(methylcarbamoyl)pyridin-3-yl)piperazin-1-yl)methyl)-2-methyl-7-fluoroimidazo[1,2-c]quinazolin-5(6H)-one;
8-((4-(2-fluoro-6-(methylcarbamoyl)pyridin-3-yl)piperazin-1-yl)methyl)-3-methyl-7-fluoroimidazo[1,2-c]quinazolin-5(6H)-one;
7-((4-(2-fluoro-6-(methylcarbamoyl)pyridin-3-yl)piperazin-1-yl)methyl)-6-fluorooxazolo[4,5-c]quinolin-4(5H)-one;
7-((4-(2-fluoro-6-(methylcarbamoyl)pyridin-3-yl)piperazin-1-yl)methyl)furo[3,4-c]quinolin-4(5H)-one;
7-((4-(2-fluoro-6-(methylcarbamoyl)pyridin-3-yl)piperazin-1-yl)methyl)isoxazolo[3,4-c]quinolin-4(5H)-one;
7-((4-(6-(methylcarbamoyl)pyridin-3-yl)piperazin-1-yl)methyl)-6-fluoroimidazo[1,5-a]quinoxalin-4(5H)-one;
(R)-7-((3-((6-(methylcarbamoyl)pyridin-3-yl)amino)pyrrolidin-1-yl)methyl)-6-fluorofuro[2,3-c]quinolin-4(5H)-one;
6-fluoro-7-((4-(2-fluoro-6-(2,2-difluoroethylcarbamoyl)pyridin-3-yl)piperazin-1-yl)methyl)furo[2,3-c]quinolin-4(5H)-one;
6-fluoro-7-((4-(6-(methylcarbamoyl)pyridin-3-yl)piperazin-1-yl)methyl)furo[2,3-c]quinolin-4(5H)-one;
6-fluoro-7-((4-(6-(methylcarbamoyl)pyridin-3-yl)piperazin-1-yl)methyl)furo[3,4-c]quinolin-4(5H)-one;
6-fluoro-7-((4-(6-(methylcarbamoyl)pyridin-3-yl)piperazin-1-yl)methyl)furo[3,2-c]quinolin-4(5H)-one;
7-((4-(2-chloro-6-(methylcarbamoyl)pyridin-3-yl)piperazin-1-yl)methyl)-6-fluoro-2-methylpyrazolo[1,5-a]quinoxalin-4(5H)-one;
6-fluoro-3-methyl-7-((6-methylcarbamoyl-3′,6′-dihydro-[3,4′-bipyridin]-1′(2′H)-yl)methyl)pyrazolo[1,5-a]quinoxalin-4(5H)-one;
6-fluoro-3-methyl-7-((4-(6-(methylcarbamoyl)pyridin-3-yl)piperazin-1-yl)methyl)pyrazolo[1,5-a]quinoxalin-4(5H)-one;
6-fluoro-7-((4-(2-fluoro-4-(methylcarbamoyl)phenyl)piperazin-1-yl)methyl)-3-methylpyrazolo[1,5-a]quinoxalin-4(5H)-one;
6-fluoro-7-((4-(2-chloro-4-(methylcarbamoyl)phenyl)piperazin-1-yl)methyl)-3-methylpyrazolo[1,5-a]quinoxalin-4(5H)-one;
(R)-6-fluoro-7-((4-(2-fluoro-6-((tetrahydrofuran-3-yl)carbamoyl)pyridin-3-yl)piperazin-1-yl)methyl)-3-methylpyrazolo[1,5-a]quinoxalin-4(5H)-one;
6-fluoro-3-methyl-7-((4-(8-(methylamino)-1,7-naphthyridin-3-yl)piperazin-1-yl)methyl)pyrazolo[1,5-a]quinoxalin-4(5H)-one;
7-((4-(6-(1H-imidazol-2-yl)pyridin-3-yl)piperazin-1-yl)methyl)-6-fluoro-3-methylpyrazolo[1,5-a]quinoxalin-4(5H)-one;
6-fluoro-7-((4-(2-fluoro-6-((methyl-d3)carbamoyl)pyridin-3-yl)piperazin-1-yl)methyl)-3-methylpyrazolo[1,5-a]quinoxalin-4(5H)-one;
7-((4-(2-cyclopropyl-6-(methylcarbamoyl)pyridin-3-yl)piperazin-1-yl)methyl)-6-fluoro-3-methylpyrazolo[1,5-a]quinoxalin-4(5H)-one;
6-fluoro-7-((4-(3-fluoro-4-(methylcarbamoyl)phenyl)piperazin-1-yl)methyl)-3-methylpyrazolo[1,5-a]quinoxalin-4(5H)-one;
6-fluoro-7-((4-(2-methyl-4-(methylcarbamoyl)phenyl)piperazin-1-yl)methyl)-3-methylpyrazolo[1,5-a]quinoxalin-4(5H)-one;
7-((4-(2-fluoro-6-(methylcarbamoyl)pyridin-3-yl)piperazin-1-yl)methyl)-3-ethyl-6-fluoropyrazolo[1,5-a]quinoxalin-4(5H)-one;
6-fluoro-7-((4-(2-fluoro-6-cyanopyridin-3-yl)piperazin-1-yl)methyl)-3-methylpyrazolo[1,5-a]quinoxalin-4(5H)-one;
6-fluoro-7-((2-fluoro-6-methylcarbamoyl-3′,6′-dihydro-[3,4′-bipyridin]-1′(2′H)-yl)methyl)-3-methylpyrazolo[1,5-a]quinoxalin-4(5H)-one;
(R)-6-fluoro-7-((4-(2-fluoro-6-(methylcarbamoyl)pyridin-3-yl)-3-methylpiperazin-1-yl)methyl)-3-methylpyrazolo[1,5-a]quinoxalin-4(5H)-one;
(R)-6-fluoro-3-methyl-7-((8-methylcarbamoyl-1,2,4a,5-tetrahydropyrazino[1,2-d]pyrido[2,3-b][1,4]oxazin-3(4H)-yl)methyl)pyrazolo[1,5-a]quinoxalin-4(5H)-one;
8-fluoro-7-((4-(2-fluoro-6-(methylcarbamoyl)pyridin-3-yl)piperazin-1-yl)methyl)-3-methylpyrazolo[1,5-a]quinoxalin-4(5H)-one;
8-fluoro-7-((4-(2-methyl-6-(methylcarbamoyl)pyridin-3-yl)piperazin-1-yl)methyl)-3-methylpyrazolo[1,5-a]quinoxalin-4(5H)-one;
8-fluoro-7-((4-(2-chloro-6-(methylcarbamoyl)pyridin-3-yl)piperazin-1-yl)methyl)-3-methylpyrazolo[1,5-a]quinoxalin-4(5H)-one;
7-((4-(2-fluoro-6-(methylcarbamoyl)pyridin-3-yl)piperazin-1-yl)methyl)-3,6-dimethylpyrazolo[1,5-a]quinoxalin-4(5H)-one;
7-((4-(2-methyl-6-(methylcarbamoyl)pyridin-3-yl)piperazin-1-yl)methyl)-3,6-dimethylpyrazolo[1,5-a]quinoxalin-4(5H)-one;
7-((4-(2-chloro-6-(methylcarbamoyl)pyridin-3-yl)piperazin-1-yl)methyl)-3,6-dimethylpyrazolo[1,5-a]quinoxalin-4(5H)-one;
6-chloro-7-((4-(2-fluoro-6-(methylcarbamoyl)pyridin-3-yl)piperazin-1-yl)methyl)-3-methylpyrazolo[1,5-a]quinoxalin-4(5H)-one;
6-chloro-7-((4-(2-methyl-6-(methylcarbamoyl)pyridin-3-yl)piperazin-1-yl)methyl)-3-methylpyrazolo[1,5-a]quinoxalin-4(5H)-one;
6-chloro-7-((4-(2-chloro-6-(methylcarbamoyl)pyridin-3-yl)piperazin-1-yl)methyl)-3-methylpyrazolo[1,5-a]quinoxalin-4(5H)-one;
3,9-difluoro-7-((4-(2-fluoro-6-(methylcarbamoyl)pyridin-3-yl)piperazin-1-yl)methyl)pyrazolo[1,5-a]quinoxalin-4(5H)-one;
7-((4-(2-chloro-6-(methylcarbamoyl)pyridin-3-yl)piperazin-1-yl)methyl)-3,6-difluoropyrazolo[1,5-a]quinoxalin-4(5H)-one;
7-((4-(2-fluoro-6-(methylcarbamoyl)pyridin-3-yl)piperazin-1-yl)methyl)-3-trifluoromethyl-6-fluoropyrazolo[1,5-a]quinoxalin-4(5H)-one;
7-((4-(2-fluoro-6-(ethylcarbamoyl)pyridin-3-yl)piperazin-1-yl)methyl)-3,6-difluoropyrazolo[1,5-a]quinoxalin-4(5H)-one;
7-((4-(2-fluoro-6-(cyclopropylcarbamoyl)pyridin-3-yl)piperazin-1-yl)methyl)-3,6-difluoropyrazolo[1,5-a]quinoxalin-4(5H)-one;
7-((4-(2-fluoro-6-(methylcarbamoyl)pyridin-3-yl)piperazin-1-yl)methyl)-3,8-difluoropyrazolo[1,5-a]quinoxalin-4(5H)-one;
7-((4-(2-methyl-6-(methylcarbamoyl)pyridin-3-yl)piperazin-1-yl)methyl)-3,8-difluoropyrazolo[1,5-a]quinoxalin-4(5H)-one;
7-((4-(2-chloro-6-(methylcarbamoyl)pyridin-3-yl)piperazin-1-yl)methyl)-3,8-difluoropyrazolo[1,5-a]quinoxalin-4(5H)-one;
7-((4-(2-fluoro-6-((methyl-d3)carbamoyl)pyridin-3-yl)piperazin-1-yl)methyl)-3,6-difluoropyrazolo[1,5-a]quinoxalin-4(5H)-one;
7-((4-(2-fluoro-6-(2,2-difluoroethylcarbamoyl)pyridin-3-yl)piperazin-1-yl)methyl)-3,6-difluoropyrazolo[1,5-a]quinoxalin-4(5H)-one;
7-((4-(2-cyano-6-(methylcarbamoyl)pyridin-3-yl)piperazin-1-yl)methyl)-3,6-difluoropyrazolo[1,5-a]quinoxalin-4(5H)-one;
7-((4-(2-fluoro-6-(methylcarbamoyl)pyridin-3-yl)piperazin-1-yl)methyl)-3,6-difluoropyrazolo[1,5-a]quinoxalin-4(5H)-one;
3-fluoro-7-((4-(2-fluoro-6-(methylcarbamoyl)pyridin-3-yl)piperazin-1-yl)methyl)-6-methylpyrazolo[1,5-a]quinoxalin-4(5H)-one;
3-fluoro-7-((4-(2-methyl-6-(methylcarbamoyl)pyridin-3-yl)piperazin-1-yl)methyl)-6-methylpyrazolo[1,5-a]quinoxalin-4(5H)-one;
3-fluoro-7-((4-(2-chloro-6-(methylcarbamoyl)pyridin-3-yl)piperazin-1-yl)methyl)-6-methylpyrazolo[1,5-a]quinoxalin-4(5H)-one;
3-fluoro-7-((4-(2-fluoro-6-(methylcarbamoyl)pyridin-3-yl)piperazin-1-yl)methyl)-6-chloropyrazolo[1,5-a]quinoxalin-4(5H)-one;
3-fluoro-7-((4-(2-methyl-6-(methylcarbamoyl)pyridin-3-yl)piperazin-1-yl)methyl)-6-chloropyrazolo[1,5-a]quinoxalin-4(5H)-one;
3-fluoro-7-((4-(2-chloro-6-(methylcarbamoyl)pyridin-3-yl)piperazin-1-yl)methyl)-6-chloropyrazolo[1,5-a]quinoxalin-4(5H)-one;
7-((4-(2-fluoro-6-cyanopyridin-3-yl)piperazin-1-yl)methyl)-3,6-difluoropyrazolo[1,5-a]quinoxalin-4(5H)-one;
(R)-7-((4-(2-fluoro-6-(methylcarbamoyl)pyridin-3-yl)-3-methylpiperazin-1-yl)methyl)-3-6-difluoropyrazolo[1,5-a]quinoxalin-4(5H)-one;
7-((2-fluoro-6-methylcarbamoyl-3′,6′-dihydro-[3,4′-bipyridin]-1′(2′H)-yl)methyl)-3,6-difluoropyrazolo[1,5-a]quinoxalin-4(5H)-one;
(R)-7-((8-methylcarbamoyl-1,2,4a,5-tetrahydropyrazino[1,2-d]pyrido[2,3-b][1,4]oxazin-3(4H)-yl)methyl)-3,6-difluoropyrazolo[1,5-a]quinoxalin-4(5H)-one;
6-fluoro-7-((4-(2-fluoro-6-(methylcarbamoyl)pyridin-3-yl)piperazin-1-yl)methyl)-2-methyl-3-chloropyrazolo[1,5-a]quinoxalin-4(5H)-one;
6-fluoro-7-((4-(2-fluoro-6-(methylcarbamoyl)pyridin-3-yl)piperazin-1-yl)methyl)-2,3-dimethylpyrazolo[1,5-a]quinoxalin-4(5H)-one;
7-((4-(2-cyano-6-(methylcarbamoyl)pyridin-3-yl)piperazin-1-yl)methyl)-6-fluoro-3-(trifluoromethyl)pyrazolo[1,5-a]quinoxalin-4(5H)-one;
7-((4-(2-fluoro-6-(methylcarbamoyl)pyridin-3-yl)piperazin-1-yl)methyl)-3-cyano-6-fluoropyrazolo[1,5-a]quinoxalin-4(5H)-one;
7-((4-(2-fluoro-6-(methylcarbamoyl)pyridin-3-yl)piperazin-1-yl)methyl)-3-isopropyl-6-fluoropyrazolo[1,5-a]quinoxalin-4(5H)-one;
6-fluoro-7-(1-(4-(2-fluoro-6-(methylcarbamoyl)pyridin-3-yl)piperazin-1-yl)ethyl)-3-methylpyrazolo[1,5-a]quinoxalin-4(5H)-one;
7-fluoro-8-((4-(2-fluoro-6-(methylcarbamoyl)pyridin-3-yl)piperazin-1-yl)methyl)imidazo[1,5-c]quinazolin-5(6H)-one;
or stereoisomers, tautomers, N-oxides, hydrates, isotope-substituted derivatives, solvates or pharmaceutically acceptable salts thereof, or mixtures thereof, or prodrugs thereof.

17. Use of the compound of any one of claims 1-16, or stereoisomers, tautomers, N-oxides, hydrates, isotope-substituted derivatives, solvates or pharmaceutically acceptable salts thereof, or prodrugs thereof, or mixtures thereof in the manufacture of a medicament for treatment or prevention of a disease or condition responsive to the inhibition of PARP activity;
preferably, the disease or condition is cancer; preferably, the cancer is selected from liver cancer, melanoma, Hodgkin's disease, non-Hodgkin's lymphomas, acute lymphocytic leukemia, chronic lymphocytic leukemia, multiple myeloma, neuroblastoma, breast cancer, ovarian cancer, lung cancer (such as small cell lung cancer), Wilms' tumor, cervical cancer, testicular cancer, soft-tissue sarcoma, primary macroglobulinemia, bladder cancer, chronic myeloid leukemia, primary brain cancer, malignant melanoma, gastric cancer, colon cancer, malignant pancreatic islet tumor, malignant carcinoid cancer, choriocarcinoma, mycosis fungoide, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, or prostatic cancer;
preferably, the medicament further comprises at least one known anticancer drug or a pharmaceutically acceptable salt thereof; preferably, the anticancer drug is selected from a group consisting of a group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin, imatinib, gefitinib, erlotinib, osimertinib, afatinib, ceritinib, alectinib, crizotinib, erlotinib, lafenib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, idelalisib, pazopanib, Torisel, everolimus, tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine);
preferably, the medicament is used in combination with radiotherapy.

18. A pharmaceutical composition comprising the compound of any one of claims 1-16, or stereoisomers, tautomers, N-oxides, hydrates, isotope-substituted derivatives, solvates or pharmaceutically acceptable salts thereof, or prodrugs thereof or mixtures thereof and a pharmaceutically acceptable carrier.

19. The pharmaceutical composition of claim 18, wherein the composition further includes at least one known anticancer drug or pharmaceutically acceptable salts thereof; preferably, the at least one known anticancer drug is selected from a group consisting of the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, imatinib, gefitinib, erlotinib, ostinib, afatinib, ceritinib, alectinib, crizotinib, erlotinib, lapatinib, solutinib lafenib, regorafenib, vemurafenib, dabrafenib, aflibercept, sunitinib, nilotinib, dasatinib, bosutinib, pratinib, brutinib, cabozantinib, lenvatinib, vandetanib, trametinib, cabitinib, axitinib, temsirolimus, idelalisib, pazopanib, everolimus, tamoxifen, letrozole, fulvestrant, mitoguanhydrazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine).
